SEQUENCE LISTING (177 sequences)

SEQ ID NO: 1 - protein, 588 aa, Ajuga reptans
MSLSFTIKVTPFSGQRVHSSTESFPIQQFPTITTKSAMAVKCSSLSTATVSFQDFVGKIRDTINGKVDNSPAATTIHPADIPSNLCVVDTLQRLGVDRYFQSEIDSVLNDTYRFWQQKGEDIFTDVACRAMAFRLLRVKGYEVSSDELASYAEQEHVNLQPSDITTVIELYRASQTRLYEDEGNLEKLHTWTSNFLKQQLQSETISDEKLHKQVEYYLKNYHGILDRAGVRQSLDLYDINQYQNLKSTDRFPTLSNEDLLEFAKQDFNFCQAQHQKELQQLQRWYADCKLDTLTYGRDVVRVASFLTAAIFGEPEFSDARLAFAKHIILVTRIDDFFDHGGSIEESYKILDLVKEWEDKPAEEYPSKEVEILFTAVYNTVNDLAEMAYIEQGRSIKPLLIKLWVEILTSFKKELDSWTEDTELTLEEYLASSWVSIGCRICSLNSLQFLGITLSEEMLSSEECMELCRHVSSVDRLLNDVQTFEKERLENTINSVSLQLAEAQREGRTITEEEAMSKIKDLADYHRRQLMQMVYKDGTIFPRQCKDVFLRVCRIGYYLYASGDEFTTPQQMMGDMKSLVYEPLNTSSS

SEQ ID NO: 2 - DNA, 1767 bp, Ajuga reptans
  source 1..1767: mol_type = other DNA
atgtcactctcgttcaccatcaaagtcacccccttttcgggccagagagttcacagcagcacagaaagctttccaatccaacaatttccaacgatcaccaccaaatccgccatggctgtcaaatgcagcagcctcagtaccgcaacagtaagcttccaggatttcgtcggaaaaatcagagatacgatcaacgggaaagttgacaattctccagcagcgaccactattcatcctgcagatataccctccaatctctgcgtggtggataccctccaaagattgggagttgaccgttacttccaatctgaaatcgacagcgttcttaacgacacatacaggttctggcagcagaaaggagaagatatcttcactgatgttgcttgtcgtgcaatggcatttcgacttttgcgagttaaaggatatgaagtttcatcagatgaactggcttcgtatgctgaacaagagcatgttaacctgcaaccaagtgacataactacggttatcgagctttacagagcatcacagacaagattatatgaagacgagggcaatcttgagaagttacatacttggactagcaattttctgaagcaacaattgcagagtgaaactatttctgacgagaaattgcacaaacaggtggagtattacttgaagaactaccacggcatactagaccgtgctggagttagacaaagtctcgatttatatgacataaaccaataccagaatctaaaatctacagatagattccctactttaagtaacgaagatttacttgaattcgcgaagcaagattttaacttttgccaagctcaacaccagaaagagcttcagcaactgcaaaggtggtatgcggattgtaaattggatacattgacttacggaagagatgtggtacgtgttgcaagtttcctgacagctgcaatttttggtgagcctgaattctctgatgctcgtctagccttcgccaaacacatcatcctcgtgacacgtattgatgatttcttcgatcatggtgggtctatagaagagtcatacaagatcctggatttagtaaaagaatgggaagataagccagctgaggaatatccttccaaggaagttgaaatcctctttacagcagtatataatacagtaaatgacttggcagaaatggcttatattgagcaaggccgttccattaaacctcttctaattaaactgtgggttgaaatactgacaagtttcaagaaagaactggattcatggacagaagacacagaactaaccttggaggagtacttggcttcctcctgggtgtcgatcggttgcagaatctgcagtctcaattcgctgcagttccttggtataacattatccgaagaaatgctttcaagcgaagagtgcatggagttgtgtaggcatgtttcttcagtcgacaggctactcaatgacgtgcaaactttcgagaaggaacgcctagaaaatacgataaacagtgtgagcctacagctagcagaagctcagagagaaggaagaaccattacagaagaggaggctatgtcaaagattaaagacctggctgattatcacaggagacaactgatgcagatggtttataaggatgggaccatatttccgagacaatgcaaagatgtctttttgagggtatgcaggattggctactacttatacgcgagcggcgatgaattcactactccacaacaaatgatgggggatatgaaatcattggtttatgaacccctaaacacttcatcctcttga

SEQ ID NO: 3 - protein, 567 aa, Leonotis leonurus
MSVAFNLIVVRFPGHGIQSSRETFPAKIITRTKSSMRFQSSLNTSTDFVGKIREMIRGKTDNSINPLDIPSTLCVIDTLHSFGIDRYFQSEINSVLHHTYRLWNDRNNIIFKDVICCAIAFRLLRVKGYQVSSDELAPFAQQQVTGLQTSDIATILELYRASQERLHEDDDTLDKLHDWSSNLLKLHLLNENIPDHKLHKRVGYFLKNYHGMLDRVAVRRNIDLHNINHYQIPEVADRFPTEAFLEFSRQDFNICQAQHQKELQQLHRWYADCRLDTLNHGTDVVHFANFLTSAIFGEPEFSEARLAFAKQVILITRMDDFFDHDGSREESHKILHLVQQWKEKPAEEYGSKEVEILFTAVYTTVNSLAEKACMEQGRSVKQLLIKLWVELLTSFKKELDSWTEKMALTLDEYLSFSWVSIGCRLCILNSLQFLGIKLSEEMLWSQECLDLCRHVSSVVRLLNDLQTFKKERIENTINGVDVQLAARKGERAITEEEAMSKIKEMADHHRRKLMQIVYKEGTIFPRECKDVFLRVCRIGYYLYSGDELTSPQQMKEDMKALVHESSS

SEQ ID NO: 4 - DNA, 1704 bp, Leonotis leonurus
  source 1..1704: mol_type = other DNA
atgtcggtggcgttcaacctcatagtcgtccgttttccgggccatggaattcagagcagtagagaaacttttccagccaaaattattaccagaactaaatcaagcatgagattccaaagcagcctcaacacttcaacagatttcgtgggaaaaataagagagatgatcagagggaaaactgataattctattaatcccctggatattccctccactctatgcgtaatcgacaccctacacagcttcggaattgatcgctactttcaatccgaaatcaactctgttcttcaccacacatacagattatggaacgacagaaataatatcatcttcaaagatgtcatttgctgcgcaattgcctttagacttttgcgagtgaaaggatatcaagtctcatcagatgaactggcgccatttgcccaacaacaggtgactggactacaaacaagcgacattgccacgattctagagctctacagagcatcacaggagagattacacgaagacgacgacactcttgacaaactacatgattggagcagcaaccttctgaagctgcatctgctgaatgagaacattcctgatcataaactgcacaaacgggtggggtatttcttgaagaactaccatggcatgctagatcgcgttgcggttagacgaaacatcgaccttcacaacataaaccattaccaaatcccagaagttgcagataggttccctactgaagcttttcttgaattttcaaggcaagattttaatatttgccaagctcaacaccagaaagaacttcagcaactgcataggtggtatgcagattgtagattggacacactgaatcacggaacagacgtagtacattttgctaattttctaacttcagcaattttcggagagcctgaattctccgaggctcgtctagcctttgctaaacaggttatcctaataacacgtatggatgatttcttcgatcacgatgggtctagagaagaatcacacaagatcctccatctagttcaacaatggaaagagaagcccgccgaagaatatggttcaaaggaagttgagatcctctttacagcagtgtacactacagtaaatagcttggcagaaaaggcttgtatggagcaaggccgtagtgtcaaacaacttctaattaagctgtgggtcgagctgctaacaagtttcaagaaagaattggattcatggacggagaagatggcgctaaccttggatgagtacttgtctttctcctgggtgtcaattggctgcagactctgcattctcaattccctgcaatttcttgggataaaattatctgaagaaatgctgtggagtcaagagtgtctggatttatgccggcatgtttcatcagtggttcgcctgctcaacgatttacaaactttcaagaaggagcgcatagaaaatacgataaacggtgtggacgttcagctagctgctcgtaaaggcgaaagagccattacagaagaggaggccatgtccaagattaaggaaatggctgaccatcacaggagaaaactgatgcaaattgtgtataaagaaggaaccatttttccaagagaatgcaaagatgtgtttttgagagtgtgcaggattggctactatctctactcgggcgatgagttaacttctccacaacaaatgaaggaggatatgaaagcgttggtacatgaatcatcctcttga

SEQ ID NO: 5 - protein, 751 aa, Mentha spicata
  VARIANT 1..751: note = Xaa = Any Amino Acid
MSSIRNLSLHIDLPKAEKKLVEKIRERIRNGRVEMSPSAYDTAWVAMVPSRGYSGRPGFPECVDWIIENQNPDGSWGLDSDQPLLVKDSLSSTLACLLALRKWKTHNQLVQRGMEFIDSRGWAATDDDNQISPIGFNIAFPAMINYAKELNLTLPLHPPSIHSLLHIRDSEIRKRNWEYVAEGVVDDTSNWKQIIGTHQRNNGSLFNSPATTAAAVIHSHDDKCFRYLISTLENSNGGWVPTIYPYDIYAPLCMIDTLERLGIHTYFEVELSGIFDDIYRNWQEREEEIFCNVMCRALAFRLLRMRGYHVSSDELAEFVDKEEFFNSVSMQESGEGTVLELYRASLTKINEEERILDKIHAWTKPFLKHQLLNRSIRDKRLEKQVEYDLKNFYGALVRFQNRRTIDSYDAKSIQISKTAYRCSTVYNEDFIHLSVEDFKISRAQYLKELEEMNKWYSDCRLDLLTKGRNACRESYILTAAIIVDPHESMARISYAQSILLITVFDDFFDHYGSKEEALNIIDLVKEWKPAGSYCSKEVEILFTALHDTINEIAAKADAEQGFSSKQQLINMWVELLESAVREKDSLSXNKVSTLEEYLSFAPITIGCKLCVLTSVHFLGIKLSEEIWTSEELSSLCRHGNVVCRLLNDLKTYEREREENTLNSVSVQTVGGGVSEEEAVTKVEEVLEFHRRKVMQLACRRGGSSVPRECKELVWKTCTIGYCLYGHDGGDELSSPKDILKDINAMMFEPLK

SEQ ID NO: 6 - DNA, 2256 bp, Mentha spicata
  variation 1..2256: note = X - A, T, G or C
  source 1..2256: mol_type = other DNA
atgagttccattcgaaatttaagtttgcatattgatctgccaaaggccgagaagaagttggttgagaaaatcagagagaggataagaaatgggagggtggagatgtcgccgtcggcttacgacaccgcgtgggtggccatggtgccgtctcgaggatattccggcaggccgggtttcccggagtgcgtggattggataatcgagaaccagaatccggacgggtcgtggggtttggattcggatcaaccacttctggtcaaagactccctctcgtccaccttggcatgcctacttgccctgcgtaaatggaaaacacacaaccaactagtgcaaaggggcatggagttcatcgactcccgtggttgggctgcaactgatgatgacaatcagatttctcctattggattcaatattgcctttcctgcaatgattaattacgccaaagagcttaatttaactctgcctctacatccaccttcgattcattcattgttacacattagagattcagaaataagaaagcgaaactgggaatacgtagctgaaggagtagtcgacgatacaagcaattggaagcaaataatcggcacgcatcaaagaaataatggatccttgttcaactcacctgctaccactgcagctgctgttattcactctcacgacgataaatgtttccgatatttgatctccactcttgagaattctaacggtggatgggtaccaactatctatccatacgatatatacgctcctctctgcatgatcgatacgctagaaagattaggaatacacacatattttgaagttgaactcagcggcatttttgatgacatatacaggaattggcaagagagagaagaagagatcttttgtaatgttatgtgtcgagctctggcatttcggcttctacgaatgaggggatatcatgtttcatctgatgaactagcagaatttgtggacaaggaggagttttttaatagcgtgagcatgcaagagagcggcgaaggcacagtgcttgagctttacagagcttcactcacaaaaatcaacgaagaagaaaggattctcgacaaaattcatgcatggaccaaaccatttctcaagcaccagcttctcaaccgcagcattcgcgacaaacgattagagaagcaggtggaatacgacttgaagaacttctacggcgcactagtccgattccagaacagaagaaccatcgactcatacgatgctaaatcaatccaaatttcgaaaacagcatataggtgctctacagtttacaatgaagacttcatccatttatccgttgaggacttcaaaatctcccgagcacaatacctaaaagaacttgaagaaatgaacaagtggtactctgattgtaggttggacctcttaactaaaggaagaaatgcatgtcgagaatcttacattttaacagctgcaatcattgtcgatcctcacgaatccatggctcgaatctcttacgctcaatctattcttcttataactgttttcgacgactttttcgatcattatgggtctaaagaagaggctctcaatattattgatctagtcaaggaatggaagccagctggcagttactgctccaaagaagtggagattttgtttactgcattacacgacacgataaatgagattgcagccaaggctgatgcagagcaaggcttttcttccaaacaacagcttatcaacatgtgggtggagctacttgagagcgccgtgagagaaaaggactcgctgagtggnaacaaagtgtcgactctagaagagtacttatctttcgcaccaatcaccatcggctgcaaactttgcgtcctgacgtctgtccatttcctcggaatcaaactgtccgaggaaatctggacttccgaggagttgagcagtctgtgcaggcacggcaatgttgtctgcagactgctcaacgacctcaagacttacgagagagagcgcgaagagaacacgctcaacagcgtgagcgtgcagacagtgggaggaggcgtttcggaggaagaggcggtgacgaaggtggaggaggtgttggaatttcatagaagaaaagtgatgcagctcgcgtgtcgaagaggaggaagcagtgttccgagagaatgtaaggagctggtgtggaagacgtgcacgataggttactgcttgtacggtcacgacggaggcgatgagttatcgtctccgaaggatattctaaaggacattaatgcaatgatgtttgagcctctcaagtga

SEQ ID NO: 7 - protein, 800 aa, Nepeta mussinii
MSLPLSSCVLFPPNDSRFPVSRFSRASASLEVGLQGATSAKVSSQSSCFEETKRRITKLFHKDELSVSTYDTAWVAMVPSPTSSEEPCFPGCLTWLLENQCRDGSWARPHHHSLLKKDVLSSTLACILALKKWGVGEEQINKGLHFIELNCASATEKCQITPVGFDIIFPAMLDYARDFSLNLRLEPTTFNDLMDKRDLELKRCYQNYTPEREAYLAYIVEGMGRLQDWELVMKYQRKNGSLFNCPSTTAAAFIALRDSACLNYLNLSLKKFGNAVPAVYPLDIYSQLCTVDNLERLGINQYFIAEIQSVLDETYRCWIQGNEDIFLDTSTCALAFRILRMNGYDVTSDSLTKILEECFSSSFRGNMTDINTTLDLYRASELMLYPDEKDLEKHNLRLKLLLKQKLSTVLIQSFQLGRNINEEVKQTLEHPFYASLDRIAKRKNIEHYNFDNTRILKTSYCSPNFGNKDFFFLSIEDFNWCQVIHRQELAELERWLIENRLDELKFARSKSAYCYFSAAATFFAPELSDARMSWAKSGVLTTVVDDFFDVGGSMEELKNLIQLVELWDVDASTKCSSHNVHIIFSALRRTIYEIGNKGFKLQGRNITNHIIDIWLDLLNSMMKETEWARDNFVPTIDEYMSNAYTSFALGPIVLPTLYLVGPKLSEEMINHSEYHNLFKLMSTCGRLLNDIRGYERELKDGKLNALSLYIINNGGKVSKEAGISEMKSWIEAQRRELLRLVLESNKSVLPKSCKELFWHMCSVVHLFYCKDDGFTSQDLIQVVNAVIHEPIALKDFKVHE

SEQ ID NO: 8 - DNA, 2403 bp, Nepeta mussinii
  source 1..2403: mol_type = other DNA
atgtctcttccgctctcctcttgtgtcttatttccccccaatgactcacgttttccggtctcccgcttttctcgcgcttcagcttctttggaagtcgggcttcaaggagctacttcagcaaaagtctcctcacaatcatcgtgttttgaggagacaaagagaaggataacaaagttgtttcataaggacgaactttcggtttcgacatatgacacagcatgggttgctatggtcccttctccaacttcttcagaggaaccttgcttcccaggttgtttgacttggttgcttgaaaaccagtgtcgagatggttcatgggctcgtccccaccatcactctttgttaaaaaaagatgtcctttcttctaccttggcatgcattctcgcacttaaaaaatggggggttggtgaagaacaaatcaacaagggtttgcattttatagagctaaattgtgcttcagctaccgagaagtgtcaaattactcccgtggggtttgacattatatttcctgccatgcttgattatgcaagagacttctctttgaacttgcgtttagagccaactacgtttaatgatttgatggataaaagggatttagagctcaaaaggtgttaccaaaattacacaccggagagggaagcatacttggcatatatagttgaaggaatgggaagattgcaagattgggaattggtgatgaaatatcaaagaaagaatggatctcttttcaattgtccatctacaactgcagcagcttttattgcccttcgggattctgcgtgcctcaactatctgaatttgtctttgaaaaagttcgggaatgcagttcctgcagtttatcctctagatatatattctcaactttgcacggttgataatcttgaaaggctggggatcaaccaatattttatagcagaaattcagagtgtgttggatgaaacgtacagatgttggatacagggaaacgaagacatatttttggacacctcaacttgtgctttagcattccgaatattgagaatgaatggctatgatgtgacttcagattcacttacaaaaatcctagaagagtgcttttcaagttcctttcgtggaaatatgacagacattaacacaactcttgacttatatagggcatcagaacttatgttatatccagatgaaaaggatctggagaaacataatttaaggcttaaactcttacttaagcaaaaactatccactgttttaatccaatcatttcaacttggaagaaatatcaatgaagaggtgaaacagactctcgagcatcccttttatgcaagtttggataggattgcaaagcggaaaaatatagagcattacaactttgataacacaagaattcttaaaacttcatattgttcgccaaattttggcaacaaggatttcttttttctttccatagaagacttcaattggtgtcaagtcatacatcgacaagaactcgcagaacttgaaagatggttaattgaaaatagattggatgagctgaagtttgcaaggagtaagtctgcatactgttatttttctgcggcagcaactttttttgctccagaattgtcggatgcccgcatgtcatgggctaaaagtggtgttctaaccacagtggtagatgacttttttgatgttggaggttctatggaggaattgaagaacttaattcaattggttgaactatgggatgtggatgctagcacaaaatgctcttctcataatgtccatataatattttcagcacttaggcgcaccatctatgagatagggaacaaaggatttaagctacaaggacgtaacattaccaatcatataattgacatttggctagatttactaaactctatgatgaaagaaaccgaatgggccagagacaactttgtcccaacaattgatgaatacatgagcaatgcatatacatcgtttgctctggggccaattgtccttccaactctctatcttgtcgggcccaagctctcagaagagatgattaaccactccgaataccataacctattcaaattgatgagtacgtgcggacgtcttctaaatgacatccgtggttatgagagagaactgaaagatggtaaattgaacgcgttatcattgtacataattaataatggtggtaaagtaagtaaagaagctggcatctcggagatgaaaagttggatcgaggcacaacgaagagagttactgagattagttttggagagcaacaaaagcgtccttccgaagtcgtgcaaggaattgttttggcatatgtgctcagtggtgcatctattctactgcaaagatgatggattcacctcgcaggatttgattcaagttgtaaatgcagttattcatgaacctattgctctcaaggattttaaggtgcatgaataa

SEQ ID NO: 9 - protein, 579 aa, Origanum majorana
MASLAFTPGAATFSGNVVRRRKDNFPVHGFPTTIRSSVSVTVKCYVSTTNLMVKIKEKFKGKNVNSLTVEAADDDMPSNLCIIDTLQRLGIDRYFQPQVDSVLDHAYKLWQGKEKDTVYSDISIHAMAFRLLRVKGYQVSSEELDPYIDVERMKKLKTVDVPTVIELYRAAQERMYEEEGSLERLHVWSTNFLMHQLQANSIPDEKLHKLVEYYLKNYHGILDRVGVRRNLDLFDISHYPTLRARVPNLCTEDFLSFAKEDFNTCQAQHQKEHEQLQRWFEDCRFDTLKFGRETAVGAAHFLSSAILGESELCNVRLALAKHMVLVVFIDDFFDHYGSREDSFKILHLLKEWKEKPAGEYGSEEVEILFTAVYNTVNELAEMAHVEQGRNIKGFLIELWVEIVSIFKIELDTWSNDTTLTLDEYLSSSWVSVGCRICILVSMQLLGVQLTDEMLLSDECINLCKHVSMVDRLLNDVGTFEKERKENTGNSVSLLLAAAVKEGRPITEEEAIIKIKKMAENERRKLMQIVYKRESVFPRKCKDMFLKVCRIGCYLYASGDEFTSPQKMKEDVKSLIYESL

SEQ ID NO: 10 - DNA, 1740 bp, Origanum majorana
  source 1..1740: mol_type = other DNA
atggcgtcgctcgcgttcacacccggagccgccactttctccggcaacgtagttcggaggaggaaagataactttccggtccacggatttccgacgacgatcaggtcatcggtctccgtcaccgtcaaatgctacgtcagtacaacgaatttgatggtgaaaatcaaagagaagttcaagggtaaaaacgtcaattcgctgacagttgaagctgctgatgacgatatgccctctaatctgtgcataattgacaccctccaacgattgggaatcgaccgttacttccaaccccaagtcgactctgttctcgaccacgcctacaaactatggcaagggaaagagaaagatacggtgtattcggacattagtattcatgcgatggcatttagacttttacgagtcaaaggctatcaagtctcttcggaggaactggatccatacatcgatgtggagcgaatgaagaaactgaaaacagttgatgttccgacggttatcgaactgtacagagcggcacaggagagaatgtatgaagaagaaggtagccttgagagactccatgtttggagcaccaacttcctcatgcaccagctgcaggctaactcaattcctgatgaaaagctacacaaactggtggaatactacttgaagaactaccatggcatactggatagagttggagttcgacgaaacctcgacctattcgacataagccattatccaacactcagagctagggttccgaacctatgtaccgaagattttctatcgttcgcgaaggaagatttcaatacttgccaagcccaacaccagaaagaacatgagcaactacaaaggtggttcgaagattgtaggttcgatacgttgaagttcggaagggagacagccgtaggcgctgctcattttctatcttcagcaatacttggtgaatctgaactatgtaatgttcgtcttgcccttgctaagcatatggtgcttgtggtattcatcgatgacttcttcgaccattatggctctagagaagactccttcaagatcctccacctcttaaaagaatggaaagagaagccggccggagaatacggttccgaggaagtcgaaatcctcttcacagccgtatacaatacagtaaacgagttggcggagatggctcatgtcgaacaaggacgtaatatcaaaggatttctaattgaattgtgggttgaaatagtgtcaattttcaagatagaactggatacatggagcaatgacacaacactaaccttggatgagtacttgtcctcctcatgggtgtcggtcggttgcagaatctgcatcctcgtctcaatgcagctcctcggtgtacaactaaccgacgaaatgcttctgagcgacgagtgcataaacctgtgtaagcatgtctcgatggtcgatcgcctcctcaacgacgtcggaacattcgagaaggaacggaaggagaatacaggaaacagtgtgagccttctgctagcagcagctgtgaaagaaggaaggcctattaccgaagaggaagctattattaaaattaaaaaaatggcggaaaacgagaggaggaaactaatgcagattgtgtataaaagagagagtgttttccccagaaaatgcaaggatatgttcttgaaggtgtgtagaattgggtgctatctatacgcgagcggcgacgaatttacgtctcctcagaaaatgaaggaagatgtgaaatccttaatttatgaatccttgtag

SEQ ID NO: 11 - protein, 563 aa, Origanum majorana
MSLAFSHVSTFFSGQRVVGSRREIIPVNGVPTTANKPSFAVKCNLTTKDLMVKMKEKLKGQDGNLTVGVADMPSSLCVIDTLERLGVDRYFRSEIHVILHDTYRLWQQKDKDICSNVTTHAMAFRLLRVNGYEVSSEELAPYANLEHFSQQKVDTAMAIELYRAAQERIHEDESGLDKILAWTTTFLEQQLLTNSILDNKLHKLVEYYLNNYHGQTNRVGARRHLDLYEMSHYQNLKPSHSLCNEDLLAFAKQGFRDFQIQQQKEFEQLQRWYEDCRLDKLSYGRDVVKISSFMASILMDDPELADVRLSIAKQMVLVTRIDDFFDHGGSREDSYKIIELVKEWKEKAEYDSEEVKILFTAVYTTVNELAEACVQQGRNSTTVKEFLVQLWIEILSAFKVELDTWSDGTEVSLDEYLSWSWISNGCRVSIVTTMHLLPTKLCSDEMLRSEECKDLCRHVSMVGRLLNDIHSFEKEHEENTGNSVSILVAGEDTEEEAIGKIKEIVEYERRKLMQIVYKRGTILPRECKDIFLKACRATFYVYSSTDEFTSPRQVMEDMKTLSS

SEQ ID NO: 12 - DNA, 1692 bp, Origanum majorana
  source 1..1692: mol_type = other DNA
atgtcactcgccttcagccatgttagtacctttttctccggccaaagagtcgtcggaagcaggagagagattattccagttaacggagttccgacgacggccaataagccgtcgttcgccgttaagtgcaaccttactacaaaggatttgatggtgaaaatgaaggagaagttgaaggggcaagacggtaatttgactgtcggagtagccgatatgccctctagcctgtgcgtgatcgacactcttgaaaggttgggagttgaccgatacttccgatctgaaatccacgttattctacacgacacttaccggttatggcaacaaaaggacaaagatatatgttccaacgttactactcatgcaatggcgtttagacttctgagagtgaatggatacgaggtttcatcagaggaactggctccatatgctaacctagagcactttagccagcaaaaagttgatactgcaatggctatagagctctacagagcagcacaggagagaatacacgaagacgagagcggtctcgacaaaatacttgcttggaccaccacttttctcgagcaacagctgctcactaactccattcttgacaataaattgcataaactggtggagtactacttgaacaactaccacggccaaacgaatagggtcggagctagacgacacctcgacctatatgagatgagccattaccaaaatctaaaaccttcacatagtctatgcaatgaagaccttctagcatttgcaaagcaaggttttcgagattttcaaatccagcagcagaaagaattcgagcaactgcaaaggtggtatgaagattgcaggttggacaagttgagttatgggagagatgtagtaaaaatttctagtttcatggcttcaatattgatggatgatccagaattagccgatgttcgtctctccatcgccaaacagatggtgctcgtgacacgtatcgatgatttcttcgaccacggtggctctagagaagactcctacaagatcattgaactagtaaaagaatggaaggagaaggcagaatacgattccgaggaagtaaaaatcctttttacagcagtatacaccacagtaaatgagctagcagaggcttgtgttcaacaaggaaggaatagtactactgtcaaagaattcctagttcagttgtggattgaaatactatcagctttcaaggtcgagctagatacgtggagcgatggcacggaagtaagcctggacgagtacttgtcgtggtcgtggatttcgaatggctgcagagtgtctatagtaacgacgatgcatttgctccctacgaaattatgcagtgatgaaatgcttaggagtgaagagtgcaaggatttgtgtaggcatgtttctatggttggccgcttgctcaacgacatccactcttttgagaaggagcatgaggagaatacgggaaacagtgtgagcattctagtagcaggtgaggataccgaagaggaagctattggaaagatcaaagagatagttgagtatgagaggagaaaattgatgcaaattgtgtacaagagaggaaccattctcccaagagaatgcaaagacatattcttgaaggcgtgtagggctacattttacgtgtactcgagcacggatgagtttacgtctcctcgacaagtgatggaagatatgaaaaccctaagctcctag

SEQ ID NO: 13 - protein, 604 aa, Origanum majorana
MVSACLKLKNNPFLDHRFRKSSNGFSVNFPATMLTTVKCSRDNSEDLIAKIKERMNEKFVTVPAREYSVIEHRNPKPAWCGGLQSKTVIEEEVCSRLFLVEHLQDLGVDRFFQSEIQHILHHTFRLWQQKDEQVFKDVTCRAMAFRLLRLEGYHVSSGELGEYVDEEKFFRTVRLEWRSTDTILELYKASQVRLPEDDNDNSNILKNLHEWTFIFLKEQLRRKTILDKGLERKVEFYLKNYHGILDAVKHRRSLDHTRFWKTTAYNPAVYDEDLFRLSAQDFMARQAQSQKELEMLLKWYDECRLDKMEYGRNVIHVSHFLNANNFPDPRLSETRLSFAKTMTLVTRLDDFFDHHGSREDSVLIIELIRQWNEPSTITTIFPSEEVEILYSALHSTVTDIAEKAYPIQGRCIKSLIIHLWVEILSSFMSEMDSCTAETQPDFHEYLGFAWISIGCRICILIAIHFLGEKVSQQMVMGAECTELCRHVSTIARLLNDLQTFKKEREERKVNSVIIQLKGDKISEEVAVSNIERMVEYHRKELLKMVVRREGSLVPKRCKDVFWKSCNIAYYLYAFTDEFTSPQQMKEDMKLLFRDPINCVPSIPS

SEQ ID NO: 14 - DNA, 1815 bp, Origanum majorana
  source 1..1815: mol_type = other DNA
atggtatctgcatgtctaaaactcaaaaataatcctttcttggaccatcgattcaggaaaagcagcaatggattttcagttaattttccggcgaccatgctcaccactgtcaagtgcagccgcgataattcagaagacttgatagcaaagataaaagaaaggatgaatgaaaaatttgttacggtgccggcgagggaatattccgtcattgagcatcggaatccgaagccggcgtggtgcggtggtttgcaatccaaaacagtaatagaagaagaagtgtgcagccgtctgtttctggtcgaacaccttcaagatttaggagtagaccgcttctttcaatcagaaatccaacatattctacatcacacattcagattatggcagcaaaaagatgaacaagtttttaaagacgtgacatgtcgcgccatggcattcagactcctgcgtctcgaaggttatcatgtctcgtcaggagaattgggggagtatgttgatgaggaaaaattctttagaacggtaaggttagaatggagaagtacggatacaattcttgagctgtacaaagcatcacaggtaagactacctgaagacgacaacgacaattccaatatcctcaaaaacttgcacgaatggaccttcatatttttgaaggagcagttgcggcgtaaaactattcttgataaaggtttagagagaaaggtagaattttacttgaagaattaccacggcatattagacgcggttaagcatagacgaagcctcgatcacacacgattctggaaaactactgcgtataaccctgcagtgtatgatgaggatcttttccgattgtcggcccaagatttcatggctcgccaagctcagagccagaaggaacttgagatgttgctcaagtggtacgatgaatgtagactggacaagatggagtatgggcgaaacgtgatacacgtttcccatttcttaaacgcaaacaacttccccgatcctcgcctgtccgaaactcgtctatcctttgcgaaaaccatgactctcgtcacgcgtttggatgatttcttcgatcaccatggctctagagaagattcggtcctcatcatcgaattaataaggcagtggaatgagccttcaactattacaacaatattcccctccgaagaagtggagattctctactctgcactccactccaccgtaacagatatagcagagaaggcttatcccatccagggtcgctgcatcaaatcgctcataattcatctgtgggtcgagatactgtcgagcttcatgagcgaaatggactcgtgcaccgcggaaactcagccggactttcacgagtacttagggtttgcatggatctcgatcggctgcagaatctgcattctcatagctatacatttcttgggggagaaggtatctcaacaaatggttatgggtgctgagtgcaccgagttatgtaggcacgtttctacgatcgcacgccttctcaacgatctccaaacctttaagaaggagagagaagagaggaaggtaaacagcgtgataatccagctcaaaggggataagatatcggaggaggtggccgtgtcgaatatagagagaatggttgaatatcacaggaaagagctgctgaagatggtggttcggagagaaggaagcttggttcctaagaggtgtaaggacgtgttctggaaatcctgcaacattgcttactatctgtacgcttttacagatgaattcacttcgcctcaacaaatgaaggaagatatgaaactactctttcgtgatccaatcaactgcgttccttcaattccttcatga

SEQ ID NO: 15 - protein, 584 aa, Perovskia atriplicifolia
MLLAFNISDVPLSQHRVILSRREHFPRHAFQEFPMIAATKSSVNAICSLATPTDLMGKIKEKFKAKDGDPLAAAAIQLAADIPSSLCIIDTLQRLGVDRYFQSEIDSILEETHKLWKVKDRDIYSEVTTHAMAFRLLRVKGYEVSSEELAPYAEQERFDLQTIDLATVIELYRAAQERTCEENDNSLEKLLAWTTTFLKHQLLTNSIPDTKLHKQVEYYLKNYHGILDRMGVRRSLDLYDISHYRPLRARFPNLCNEDFLSFARQDFSMCQAQHQKELEQLQRWYSDCRLDALLKFGRNVVRVSSFLTSAIIGEPELSEVRLVFAKHIILVTLIDDLFDHGGTREESYKILELVTEWKEKTAAEYGSEEVEILFTAVYNTVNELVERAHVEQGRSVKEFLIKLWVQILSIFKIELDTWSDETALTLDEYLSSSWVSIGCRICILMSMQFIGIKLTDEMLLSEECTDLCRHVSMVDRLLNDVQTFEKERKENTGNSVSLLLAANKDVTEEEAIRRAKEMAECNRRQLMQIVYKTGTIFPRKCKDMFLKVCRIGCYLYASGDEFTSPQQMMEDMKSLVYEPLYLPN

SEQ ID NO: 16 - DNA, 1755 bp, Perovskia atriplicifolia
  source 1..1755: mol_type = other DNA
atgttacttgcgttcaacataagcgatgtccctctctcgcagcatagagtaattctgagcaggagggaacattttccacgtcatgcattccaggaatttccgatgatcgccgctactaagtcatctgttaatgccatttgcagcctcgctactccaactgatttgatgggaaaaataaaagagaagttcaaggccaaggacggcgatcctcttgccgccgcggctattcaactcgcggcggatataccctcgagtctgtgtataatcgacaccctccagaggttgggagtcgaccgatacttccaatccgaaatcgactctattctagaggaaacacacaagttatggaaagtgaaagatagagatatatactctgaggttactactcatgcaatggcgtttagacttctgcgagtgaagggatatgaagtttcatcagaggaactagctccgtatgctgagcaagagcgctttgacctgcaaacgattgatctggcgacggttatcgagctttacagagcagcacaggagagaacatgcgaagaaaacgacaacagtcttgagaaactacttgcttggaccaccacctttctcaagcaccaattgctcaccaactccatacctgacaccaaattgcacaaacaggtggaatactacttgaagaactaccacgggatattagatagaatgggagttagacgaagcctcgacctatacgacataagccattatcgacctctgagagcaagattccctaatctgtgtaatgaagatttcctatcatttgcgaggcaagatttcagtatgtgccaagcccaacaccagaaggaacttgagcaactgcaaaggtggtattctgattgtaggttggacgcgttgttgaagtttggaagaaatgtagtgcgcgtttctagctttctgacttcagcaattattggtgaacccgaattgtctgaagttcgactagtctttgccaaacatattattctcgttacacttattgatgatttattcgatcatggtggaactagagaagagtcatacaagatccttgaattagtaacagaatggaaagagaagaccgcagcagaatatggttccgaggaagttgaaatcctttttacagcggtctacaacacagtaaatgagttggtagagagggctcatgtcgaacaagggcgcagtgtcaaagaatttcttattaaactgtgggttcaaatactatcaattttcaagatagaattagatacatggagcgatgagactgcgctaaccttggatgaatacttgtcttcgtcgtgggtgtcaattggttgcagaatctgcattctcatgtcgatgcaattcatcggtataaaattaactgatgaaatgcttctgagtgaagagtgcactgatttgtgtaggcatgtttcgatggttgaccggctgctcaacgatgtgcaaaccttcgagaaggaacgcaaagaaaatacaggaaacagtgtaagccttctgctagcagctaacaaagatgttactgaagaggaagcaattagaagagcaaaagaaatggcggaatgcaacaggagacaactgatgcagattgtgtataaaacaggaaccattttcccaagaaaatgcaaagatatgtttctcaaggtatgcaggattggctgttatttgtatgcaagcggcgacgaattcacatctccacaacaaatgatggaagatatgaaatccttggtttatgaacccctctacctacctaattaa

SEQ ID NO: 17 - protein, 593 aa, Perovskia atriplicifolia
MSLTFNAGVVRFSSHRVRSTKDCFTVYGFPMIANKAAFAVKCSLTPTDLMGRVEEKFKGKNGNSLAASTTVESADIPSNLCIIDTLQRLGVDRYFQTEINAILEDTYRLWERKDKDIYSDATTHAMAFRLLRVKGYEVSSEELAPYADQECVNVQTADVATVIELYRAAQVRISEEESSLKKLHAWTTTFLKYQLQSNSIPEKKLHKLVEYYLKNYHGILDRMGVRMDLDLFDISHYRTLQASDRFSSLRNEDFLEFARQDFNICQAKHQKELQQLQRWYADCRLDTLKFGRDVVRVANFLTSAIFGEPELSDARLIFAKHIVLVTCIDEFFDHGGSKEESYKILELVEEWKEKPTGEYGCEEVEILFTAVYSTVNELAEMAHVEQGRSVKEFLVKLWVQILSIFKIELDTWSDDTELTLDSYLNNSWVSIGCRICILMSMQFAGVKLSDEMLLSEECVDLCRHVSMVDRLLNDVQTFEKERKENTGNSVSLLQAAAEREGRAITEEEAITQIKELAEYHRRKLMQIVYKTDTIFPRKCKDMFLKVCRIGCYLYASGDEFTTPQQMMEDMKSLVYQPLTVDDMSAKELTSVRN

SEQ ID NO: 18 - DNA, 1782 bp, Perovskia atriplicifolia
  source 1..1782: mol_type = other DNA
atgtcactcactttcaacgctggagtcgtccgtttctccagccaccgcgttcggagcacgaaagattgctttacagtttacggatttccgatgattgcaaataaggcagctttcgcagttaaatgcagccttactccaaccgatttgatggggagagtagaggagaagttcaagggcaaaaatggtaattcactagcagcctcgacgacggttgaatccgcggatataccctcgaacctgtgtataatcgacaccctccaaagattgggagtcgaccgatactttcaaactgaaatcaatgccattctagaggacacttacagattatgggaacgaaaagacaaagacatatattccgatgccacaactcacgcgatggcgtttaggttactacgagtgaaaggatacgaagtttcatcagaggaactggctccttacgctgatcaagagtgcgtgaacgtgcaaacggctgatgtggcaacagttatcgagctttacagagcagcgcaggtgagaataagcgaagaagagagcagtcttaagaagcttcatgcttggaccaccacctttctcaaatatcagttgcagagtaactccatacctgaaaagaaactgcacaaactggtggaatattacttgaagaactaccatggcatattggatagaatgggagttcgaatggacctcgacttattcgacatcagccattatcgaactctacaagcttccgataggttctctagtctgcgtaacgaagattttctagagtttgcaaggcaagatttcaatatctgccaagccaagcaccagaaagaactccaacaactgcaaaggtggtatgcagattgcaggctcgacaccttgaagttcgggagagacgtcgtacgcgttgctaattttctgacttcagcaatctttggcgaacccgagctatccgatgctcgtctgatctttgccaagcatatcgtgctcgtaacatgtatcgatgaattcttcgatcatggtgggtctaaagaagagtcctacaagatccttgaattagtagaagaatggaaagagaagccaactggagaatatgggtgtgaggaggttgagatccttttcacagcagtgtacagtacagtgaatgagttggcagagatggctcatgtcgaacaaggacgtagtgtgaaagagtttctagttaaactgtgggtgcagatactgtcgattttcaagatagaactggatacatggagtgatgacacggaactgacgttggacagctacttgaacaactcgtgggtgtcgatcggatgcagaatctgcattctcatgtcgatgcagttcgccggtgtaaaactgtccgacgaaatgcttctgagtgaagagtgtgttgacttgtgcaggcacgtctccatggtcgatcgcctcctgaacgatgtgcaaactttcgagaaggaacgcaaggaaaatacaggaaacagtgtgagccttctgcaagcagcagctgagagagaaggaagagccattacagaagaggaagctattacacagatcaaagaattggctgaataccacaggagaaaactgatgcagattgtgtacaaaacagacaccattttcccaagaaaatgcaaagatatgttcttgaaggtgtgcaggattgggtgctatctgtacgcaagtggagacgaattcacaactccacaacaaatgatggaagacatgaaatcattggtttatcaacccctaacagttgatgacatgagtgccaaagaattgacttctgtgagaaactag

SEQ ID NO: 19 - protein, 590 aa, Salvia officinalis
MSLAFNAAVATFSGHRIRSRREILPGQGFPMITNKSSFAVKCNLTTTDLMGKITEKFKGRDSNFSAATAVQPAADIPSNLCIIDTLQRLGVDRYFQSEIDTILEDTYRLWQRKEREIFSDITIHAMAFRLLRVKGYVVSSEELAPYADQERINLQRIDVATVIELYRAAQERISEDESSLEKLHAWTATYLKQQLLTNSIPDKKLNKLVECYLKNYHGILDRMGVRQNLDLYDISHYQTLKAADRFSNLRNEDFLAFARQDFNICQEQHQKELQQLQRWYADCRLDTLKYGRDVVRVANFLTSAIIGDPELSEVRLVFAKHIVLVTRIDDFFDHGGSREESYKILELLKEWKEKPAAEYGSKEVEILFTAVYNTVNELAEMAHIEQGRSVKEFLIKLWVQIISIFKIELDTWSDETALTLDEYLSSSWVSIGCRICILMSMQFIGIKLSDEMLLSEECIDLCRHVSMVDRLLNDVQTFEKERKENTGNSVSLLLAANKDDSAFTEEEAITKAKEMAECNRRQLMKIVYKTGTIFPRKCKDMFLKVCRIGCYLYASGDEFTSPQQMMEDMKSLVYEPLTVDPLEAKNVSGK

SEQ ID NO: 20 - DNA, 1773 bp, Salvia officinalis
  source 1..1773: mol_type = other DNA
atgtccctcgccttcaacgcagcagttgccactttctccggccacagaattcggagcaggagagaaattcttccggggcaaggatttccgatgatcaccaacaagtcgtctttcgccgtgaaatgtaaccttactacaacagatttgatgggcaagataacagagaaattcaagggaagagacagtaatttttcagcagcaacggctgttcaacctgcggcggatataccctctaacctgtgcataatcgacaccctccaaaggttgggagtcgaccgatacttccaatctgaaatcgacactattctagaggacacatacaggttatggcaaaggaaagagagagagatattttcggatataactattcatgcaatggcatttagacttttgcgagttaaaggatatgtagtttcatcagaggaactggctccgtatgctgaccaagagcgcattaacctgcaaaggattgatgtagcgacagttatcgagctttacagagcagcacaggagagaataagtgaagacgagagcagtcttgagaaactacatgcttggaccgccacctatctcaagcagcagctgctcactaactccattcctgacaagaaattgaacaaactggtggaatgctacttgaagaactatcacgggatattagatagaatgggagttagacaaaacctcgacctctacgacataagccactatcaaactctaaaagctgcagataggttctctaatctacgtaatgaagattttctagcatttgcgaggcaagattttaatatttgccaagaacaacaccaaaaagaacttcagcaactgcaaaggtggtatgcagattgtaggttggacacattgaagtatggaagagatgtcgtgcgggttgctaattttctaacatcagcaattattggtgatcctgaattgtctgaagtccgtctagtcttcgccaaacatattgtgcttgtaacacgtattgatgattttttcgatcatggtggatctagagaagagtcctacaagatccttgaattactaaaagaatggaaagagaagccagctgcagaatatggttccaaagaagttgaaattcttttcacagcagtatacaatacagtaaacgagttggcagagatggctcacatcgaacaaggacgtagtgttaaagaatttctaataaagctgtgggttcaaatcatatcgattttcaagatagaattagatacatggagcgatgagacagcgctgaccttggatgagtacttgtcttcgtcgtgggtgtcaattgggtgcagaatctgcattctcatgtcgatgcaattcattggtataaaattatctgatgaaatgcttctgagtgaagagtgtattgatttgtgtcggcatgtctccatggttgaccggctgctcaacgacgtgcagactttcgagaaggaacgcaaggaaaatacaggaaatagcgtgagccttctgctagcagctaacaaagacgacagcgcctttactgaagaggaagctattacaaaagcaaaagaaatggcggaatgtaacaggagacaactgatgaagattgtgtataaaacaggaaccattttcccaagaaaatgcaaagatatgtttctgaaggtatgcaggattggctgttacttgtatgcaagcggcgatgaattcacatctccacaacaaatgatggaagatatgaaatccttggtctatgaacccctaacagttgatcctctcgaggccaaaaatgtgagtggcaaatga

SEQ ID NO: 21 - protein, 798 aa, Ajuga reptans
MASLSTFHLYSSSLLHRKTLQSSPKLNLSSECFSTRTWMNSSKNLSLNYQVNQKIGKLTGTRVATVDAPQQLEHDDSTAKGHDIVDIETQDPIEYIRMLLNTTGDGRISVSPYDTAWIALIKDVEGRDFPQFPSSLEWIANHQLADGSWGDEGFFCVYDRLVNTIACVVALRSWNVHHDKSQRGIQYIKENVHQLKDGNAEHMMCGFEVVFPALLQKAKNMGIDDLPYEAPVIQDIYHTREQKLKRIPLEMMHKVPTSLLFSLEGLENLDWDKLLKLQSADGSFLTSPSSTAFAFMQTKDEKCFQFIKNTVETFNGGAPHTYPVDVFGRLWAVDRLQRLGISRFFEAEIADCLSHIHRYWNDKGLFSGRESDFVDIDDTSMGFRLLRMQGYDVSPNVLRNFKNGDKFSCYGGQTIESSTPIYNLYRASQFRFPGEEILEEADKFAHEFLSEQLGNNQLLDKWVISDRLQEEISIGLGMPFYATLPRVEASYYIQHYAGADDVWIGKTLYRMPEISNDTYLELARNDFKRCQAQHQFEWIYMQEWYESCNIEEFGISRKELLRVYFLACSSIFEVERTKERMAWAKSQIISRMITSFFNKQTTSSEEKETLLTEFRNINGLHKSNNTRDGDMNIVLATLHQFFAGFDRYTSHQLKNAWGVWLSKLQRGAVDGGADAELITTTINVCAGHIALKEDILSHDEYKTLTDLTSKICQQLSHIQNEKVVEIDGGITAKSRLKNEELQRDMQSLVKLVLEKSVGLNRNIKQTFLTVAKTYYYRAYNAEETMDAHIFKVLFEPVA

SEQ ID NO: 22 - DNA, 2397 bp, Ajuga reptans
  source 1..2397: mol_type = other DNA
atggcctctttgtccactttccacctctactcttcctcactccttcaccgcaaaacactgcaatcttcaccaaagcttaacctgtcttcagaatgcttctccaccagaacttggatgaacagcagcaaaaacttgtcgttaaattaccaagttaatcagaaaataggaaagctgacagggactcgagttgccactgtggatgcgccacaacaacttgaacacgatgattcaactgctaaaggccatgatatagtcgatattgaaactcaggatccaattgaatatattagaatgctgttgaacacaacaggcgatggcagaatcagcgtttcgccttacgacacagcatggattgctcttattaaggacgtggaaggacgtgattttcctcaatttccatccagccttgagtggatcgcgaaccatcaactcgctgatggttcatggggagacgaaggatttttctgtgtgtatgatcggctcgtaaatactatagcatgtgtcgtagcattgagatcatggaatgtccatcacgacaagagccaaagaggaatacaatatatcaaggaaaatgtgcatcaacttaaggatggaaatgctgagcacatgatgtgtggtttcgaagtagtgtttcctgcacttcttcaaaaagccaaaaatatgggcattgatgatcttccatatgaggctcctgtcatccaggatatttaccatacaagggagcagaaattgaaaaggataccattggagatgatgcacaaagtgcctacttctctgctgtttagtttggaaggactggagaatttagattgggataaactccttaagttgcagtcagctgatggctctttcctcacttctccctcctctactgctttcgcattcatgcaaacaaaagacgaaaaatgcttccagttcatcaagaacactgttgaaacctttaatggaggagcaccacatacttatccggtcgatgtttttggaagactttgggcggttgataggctgcagcgcctcggaatttctcgattctttgaggctgagattgctgattgcttaagtcacattcatagatattggaatgataaggggcttttcagtggacgtgaatcggactttgtcgatattgacgacacatccatgggtttcagacttctaagaatgcaaggctatgatgttagtccaaatgtactgaggaatttcaagaatggtgacaagttttcatgttacggaggtcaaacgatcgagtcatcaactccaatatacaatctgtacagagcttctcaattccggtttccaggagaagaaattcttgaagaagccgacaagttcgcccatgagttcttgtccgaacagcttggcaacaaccaattgcttgataaatgggttatatccgaccgcttgcaggaagagataagtattggattggggatgccattttatgccacccttcccagagttgaagcaagctactatatacaacattacgctggtgccgacgacgtgtggatcggcaagacactctacaggatgccggaaataagtaatgatacatacctggagctagcaagaaatgatttcaagagatgccaagcacaacatcagttcgagtggatctacatgcaagaatggtatgagagttgcaacattgaagaattcgggataagccgaaaggagctccttcgcgtttactttttggcttgctctagcatctttgaggtcgagaggactaaagagagaatggcatgggcaaaatctcaaattatttctagaatgatcacttctttctttaataaacaaactacttcatctgaggaaaaagaaacacttttaaccgaattcagaaacatcaacggtctgcacaaatcaaacaatacaagagatggagatatgaacattgtgcttgcaaccctccatcaattcttcgctggatttgacagatatactagccatcaactgaaaaatgcttggggagtatggttgagcaagctgcaacgaggagcagtagacggtggagcagacgcagagctgataacaaccaccataaacgtatgcgccggtcatatagctcttaaggaagacatattgtcccacgatgagtacaagactctcaccgacctcaccagcaagatttgtcagcagctttctcatattcaaaacgaaaaggttgtggaaattgacggtgggattacagcaaaatctaggttgaagaatgaggaactgcaacgtgacatgcaatcattggtgaaattagtacttgagaaatcagttgggctcaaccggaatataaagcaaacatttctaacggttgcaaaaacatactactacagagcctacaatgctgaggaaactatggatgcccatatattcaaagttcttttcgaaccagttgcgtga

SEQ ID NO: 23 - protein, 798 aa, Ajuga reptans
MSFASQATSLLSSPNRLGHVPTPSSPARFAAGGAPFWKILFTARSNGQYKAISRARNQGNVEYIDEIQKGPQVVLEAENSLEDDTQKDTDQIRELVENVRVKLQNIGGGGISISAYDTAWVALVEDINGSGQPQFPTSLDWISNHQFPDGSWGSSKFLYYDRILCTLACIVALKTWNVHPDKYHKGLDFIRENIHKLADEEEVHMPIGFEVAFPSIIETAKKVGIEIPEDFPGKKEIYAKRDLKLKKIPMDILHKMPTPLLFSIEGMEGLDWQKLFKFRDDGSFLTSPSSTAYALQQTKDELCLKYLTDLVKKDNGGVPNAFPVDLFDRNYTVDRLRRLGISRYFQPEIEECMKYVYRFWDKRGISWARNTNVQDLDDTAQGFRNLRMHGYEVTLDVFKQFEKCGEFFSFHGQSSDAVLGMFNLYRASQVLFPGEHMLADARKYAANYLHKRRLNNRVVDKWIINKDLEGEVAYGLDVPFYASLPRLEARFYIEQYGGSDDVWIGKALYRMVNVSCDTYLELAKLDYNKCQSVHQNEWKSFQKWYKSCSLGEFGFSEGSLLQAYYIAASTIFEPEKSGERLAWAKTAALMETIQQLSSQQKREFVDEFKHKNILKNENGERYRSSTSLVETLISTVNQLSSDILLEQGRDVHQELCHVWLKWLSTWEERGNLVEAEAELLLRTLHLNSGLDESSFSHPKYQQLLEVSTKVCHLLRLFQKRKVYDPEGCTTDIATGTTFQIEACMQELVKLVFSRSSEDLDSLTKLRFLDVARSFYYTAHCDPQVVESHIDKVLFEKVV

SEQ ID NO: 24 - DNA, 2397 bp, Ajuga reptans
  source 1..2397: mol_type = other DNA
atgtcatttgcttcccaagccacctccctcctatcatcccccaaccgtctcggccatgttccgacgccaagctcgccggctcgtttcgctgccggtggtgccccattttggaagatattatttacagctaggtctaatgggcagtataaagctatttcaagagctcgtaaccaaggaaatgtagagtacattgatgagattcagaaaggcccgcaagtcgtattggaggcagaaaacagcttggaagatgacacacaaaaagatactgatcagataagggaactagtggaaaatgtccgagtaaagctgcagaatatcggtggtggagggataagcatatcggcgtacgacaccgcatgggtggcgctggtggaggacatcaacggcagtggccagccacagtttccgacgagcctcgattggatatcgaaccatcagttccctgatgggtcatggggcagcagcaagtttttgtattatgatcggattctatgcacattagcatgtatagttgcattgaaaacctggaatgtgcatcctgataagtaccacaaagggttggatttcatcagagagaacattcacaagcttgcggacgaagaagaagtgcacatgccaattgggttcgaagtggcattcccatcaattattgaaacagctaaaaaagtaggaatcgaaatccctgaggattttcctggcaagaaagaaatttatgcaaaaagagatttaaagctaaaaaaaataccaatggatatactgcataaaatgcccacaccattgctcttcagcatagaaggaatggaaggccttgactggcaaaagctattcaaattccgcgatgatggctcgtttcttacgtctccgtcctcaacagcctatgcactccagcaaacaaaggatgagctatgcctcaagtatctaacagatcttgtcaagaaagacaacggaggagttccgaatgcatttccagtagacctgtttgatcgtaactatacagtagaccgcttgcgaaggctaggaatttcacggtactttcaacctgaaattgaagaatgcatgaaatatgtttacagattttgggataaaagaggaattagctgggcaagaaataccaatgttcaggaccttgatgacactgcacagggattcaggaatttaaggatgcatggttatgaagtcactctagatgttttcaaacaatttgagaaatgtggagagtttttcagttttcatgggcaatccagcgatgctgttttaggaatgttcaacttgtaccgggcttctcaggttttatttccgggagaacacatgcttgcagatgcgaggaagtatgcagccaactatttgcataaacgaagacttaataatagggtggtcgacaaatggattatcaacaaagaccttgaaggcgaggtggcatatgggctagatgttccgttctacgccagcctacctcgactcgaagcaaggttctacatagaacaatatgggggtagtgatgatgtgtggattggaaaagctttatacagaatggtaaatgtaagctgcgacacttaccttgagctagcaaaattagactacaacaaatgccaatccgtgcatcagaatgagtggaaaagctttcaaaaatggtacaaaagttgcagtcttggggagtttgggttcagtgaaggaagcctactccaagcttactacatagcagcctcaactatattcgagccagagaaatcaggagaacgcctagcttgggctaaaacagcagctctaatggagacaattcaacaactttccagccagcaaaaacgtgaatttgttgatgaattcaaacataaaaacatactgaagaatgaaaatggagaaaggtatagatcaagtaccagtttggtagagactctgataagcactgtaaatcagctctcatcagacatactattggagcaaggcagagacgttcatcaagaattatgtcacgtgtggctaaaatggctgagtacatgggaggaaagaggaaacctggtggaagcggaagccgagcttcttctgcgaaccttacatctcaacagcggattggatgaatcatcattttcccaccctaaatatcaacagctcttggaggtgtctaccaaagtttgccacctccttcgcctatttcagaaacgaaaggtgtatgatcccgaagggtgtacaaccgacatagcaacaggaacaacgttccagatagaagcatgcatgcaagaactagtgaaattagtgttcagcagatcctcagaagatttagattctcttactaagttgagatttttggatgttgctagaagtttctattacactgcccattgtgatccacaggtggtcgagtcccacatcgataaagtattgtttgagaaggtagtctag

SEQ ID NO: 25 - protein, 800 aa, Plectranthus barbatus
  VARIANT 1..800: note = Xaa = Any Amino Acid
MQASMSSLNLNNAPAVCSSRSQLSAKLHPPEYSTVGAWLNRGNKNQRLGYRIRPKQLSKLTECRVASADVSQEIGKVGQSVRTPEEVNKKIEESIKYVKELLMTSGDGRISVAPYDTAIVALIKDLEGRDAPEFPSCLEWIANNQKDDGSWGDDFFCIYDRIVNTIASVVALKSWNVHPDKIERGVSYIKENAHKLKGGNLEHMTSGFEFVVPGCFDRAKALGIEGLPYDDPIIKEIYATKERRLSKVPKDMIYKVPTTLLFSLEGLGMEDLDWQKILKLQSGDGSFLTSPSSTAYAFMQTGDEKCYKFLQNAVRNCNGGAPHTYPVDVFARLWAVDRLQRLGISRFFQPEIKFCLDHIKNVWTKNGVFSGRDSEFVDIDDTSMGIRLLKMHGYDVDPNALKHFKQEDGRFSCYGGQMIESASPIYNLYRAAQLRFPGEEILEEATKFAYNFLQQKLANNQIQEKWVISEHLIDEIKMGLKMPWYATLPRVEASYYLQYYAASGDVWIGKTFYRMPEISNDTYKELALLDFNRCQAQHQFEWIYMQEWYQSNNIKEFGISKKELLLAYFLAAATIFEPERSQERIVWAKTQVVSKMITSFLSQENALSSXQKTALFIDFGHSINGLNQITSVEKENGLAQTVLATFGQLLEEFDRYTRHQLKNAWSQWFMKLQQGDDNGGADAELLANTLNICAGHIAFNEDILSHNEYTSLSSLTNKICQRLSQIRDNKILEIEDGSIKDKELEQEMQALVKLVLEETGGIDRNIKQTFLSVFKMFYYRAYHDAEAIDXHIFKVMFEPV

SEQ ID NO: 26 - DNA, 2406 bp, Plectranthus barbatus
  variation 1..2406: note = X =A, T, G, or C
  source 1..2406: mol_type = other DNA
atgcaggcttctatgtcatctctgaacttgaacaatgcaccggccgtctgcagcagcaggtcacagctatccgctaaacttcacccgccggaatattccaccgtgggtgcatggctgaatcgtggcaacaaaaaccagcggttgggctaccggattcgtccaaagcaactatcaaaactaactgagtgtcgagtagcaagtgcagatgtgtcacaagagattggaaaagtcggccaatctgttcggactcctgaagaggtaaataaaaagatagaggaatccatcaagtacgtgaaggagctgctgatgacgtcgggcgacgggcgaatcagtgtggcgccctacgacacggccatagttgcccttatcaaggacttggaagggcgcgatgccccggagtttccatcttgcttggagtggattgcaaacaatcaaaaagacgatggttcttggggggatgacttcttctgcatctatgatcggatcgttaataccatagcatccgtcgtcgccttaaaatcatggaatgtgcacccagacaagattgagagaggagtatcctacatcaaggaaaacgcgcataaactaaaaggtgggaatctcgaacacatgacatcagggttcgagttcgtggttcccggctgttttgacagagccaaagccttggggatcgaaggccttccctatgatgatcccatcatcaaggagatttatgctacaaaagaaaggagattgagcaaggtaccgaaggacatgatctacaaagttccgacaactctattgtttagtttagagggactgggcatggaggatttggactggcaaaagatactgaaactgcagtcgggcgacggctcattcctcacctctccgtcgtccaccgcctacgcattcatgcagaccggagacgaaaaatgctacaaattcctccagaacgccgtcagaaattgcaacggcggagcgccgcacacttatccagtcgacgtctttgcacggctctgggcggtcgaccgacttcagcgactcggaatttctcgcttctttcagcccgagatcaagttttgcctagaccacatcaaaaatgtgtggactaagaacggagttttcagtggacgggattcagagtttgtggatatcgacgacacatccatgggcatcaggcttctgaaaatgcacggatacgatgtcgacccaaatgcactgaaacatttcaagcaggaggatgggaggttttcatgctacggtggtcaaatgatcgagtctgcatctccgatttacaatctctacagggctgctcagcttcgttttccaggagaagaaattcttgaagaagccactaaatttgcctacaacttcctgcaacagaagctggccaacaatcaaattcaagaaaagtgggtcatatccgagcacctaattgatgagataaaaatgggattgaagatgccatggtacgccaccctacctagagttgaggcttcatactatctccaatattatgcagcttctggcgacgtatggattggcaagactttttacaggatgccagaaataagtaatgacacgtacaaagagcttgcactattggatttcaaccgatgccaagcacaacatcagttcgaatggatttacatgcaagagtggtatcaaagcaacaacattaaagaatttgggataagcaagaaagagcttcttcttgcttacttcttggctgctgcaaccatttttgaacccgaacgatcgcaagagcggatcgtgtgggctaaaacccaagttgtttctaagatgatcacatcgtttctgtctcaagaaaacgctttgtcatcggancaaaagactgcacttttcatcgattttgggcatagtatcaatggcctcaatcaaataactagtgttgagaaagagaatgggcttgctcagactgtgctggcaaccttcggacaactactcgaggaattcgacagatacacaaggcatcaactgaaaaatgcttggagccaatggttcatgaaactgcagcaaggagatgacaatggcggggcagacgcagagctcctagcaaacacattgaacatctgcgctggtcatattgcttttaacgaagacatattatctcacaacgaatacacctctctctcctccctcacaaacaaaatctgtcagcggctaagtcaaattcgagataataagatactggaaattgaggatgggagcataaaagataaggaactagaacaggaaatgcaggcgctggtgaagttagtcctggaagaaaccggtggcatcgacaggaacatcaagcaaacatttttgtcagttttcaaaatgttttactacagagcctaccacgatgctgaggctatcgatgnccatattttcaaagtaatgtttgaaccagtcgtatga

SEQ ID NO: 27 - protein, 776 aa, Hyptis suaveolens
MAYMISISNLNCSSLLNTNLSAKIQLHQGLKGTWLKTSKRMCMDQQVHGKQIAKVIESRVTDKDVSTAQDFEVLKVNRVEDLISSIKSSLKTMEDGRISVSPYSTSWIALIPSIDGRQTPQFPSSLEWIVKHQLSDGSWGDALFFCVYDRLVNTIACIIALHTWKVHADKVKKGVSFVKENIWKLEDANEVHMTSGFEVIFPILLRRARDMGIDGLPSDDTPVVRMISAARDHKLKKIPREVMHQVTTTLLYSLEGLEDLDWSRLFKLQSADGSFLTSPSSTAFAFMQTNNHNCLRFITSVVQTFNGGAPDNYPIDIFARLWAVDRLQRLGISRFFEQEINDCLSYVYRFWNANGVFSAGATNFCDLDDTSMAFRLLRLHGYDVDPNVLRKFKEGDRFCCHSGEVAMSTSPTYALYRASQIQFPGEEILDEAFSFTRDYLQDWLARDQVLDKWIVSKDLPDEIKVGLEVPWYASLPRVEAAYYMQRHYGGSTDAWVAKTCYRMPDVSNDDYLELARLDFKRCQAQHQSELSYMQRWYDSCNVEEFGISRKELLVAYFVAAATIFEPERATERIVWAKTEIVSKMIKAFFGEDSLDQKTMLLKEFRNSINNGSHRFMKSEHRIVNILLQALQELLHGSDDCRIGQLKNAWYEWLMKFEGGDEASLWGEGELLVTTLNICTAHFLQHHDLLLNHDYITLSELTNKICLKLSQIQVGEMNEMREDMQALTKLVIGESCIVNKNIKQTFLAVAKTFYYRAYFDADTVDLHIFKVLFEPIV

SEQ ID NO: 28 - DNA, 2331 bp, Hyptis suaveolens
  source 1..2331: mol_type = other DNA
atggcgtatatgatatctatttcaaatctcaactgttcctcgctactaaacaccaatctttcagcaaagattcagctgcaccaaggtctcaaaggaacatggctaaaaaccagcaaacgcatgtgcatggatcaacaggttcatggcaagcagatagcaaaagtgatcgagagccgagttactgataaggatgtttccactgctcaggactttgaagtgttaaaggtcaatagagtggaggatctgatatcaagcattaagagttcattgaagacaatggaagatggaagaataagcgtgtcgccctacagcacatcatggatcgcactcattccaagtattgatgggcgccagacgccccagtttccatcttcactggagtggatcgtgaagcatcagctatcagatggttcatggggtgatgccctttttttctgcgtttatgatcgtctcgtaaatacgattgcatgcatcattgccctgcacacctggaaggttcatgcagacaaggttaaaaaaggagtaagttttgtgaaggaaaatatatggaaacttgaagacgccaacgaggtccacatgactagtggtttcgaagttatatttcccatccttcttcgaagagcacgagacatgggaattgatggtcttccttctgatgatactccagttgttaggatgatttctgctgctagggatcacaaattgaaaaagattccgagggaggtgatgcaccaagtgacaacaactctattatatagtttggaagggttggaagatttagactggtcaaggcttttcaaacttcagtcagctgatggttcattcttaacttctccatcttcaactgccttcgcattcatgcaaactaataaccacaattgcttgagattcatcactagcgttgtccaaacattcaatggaggagctccagataactatccaatcgacatctttgcgagactgtgggcagttgacaggttacagcggttagggatttctcgtttcttcgagcaggagataaatgattgcctaagctatgtatatagattttggaatgcaaatggagttttcagtgcaggagccactaatttttgtgatcttgacgacacatccatggctttccggctactacgtttgcatggatatgatgtcgacccaaatgttctgaggaaattcaaagagggagacagattctgttgccacagtggtgaagtggcgatgtcgacatcgccaacgtacgctctctacagagcttcccaaattcagtttccaggagaagaaattctggatgaagccttcagcttcactcgcgactatctacaggactggttagcaagagatcaagttcttgataagtggattgtatccaaggaccttccagatgagattaaggtaggactagaggtgccatggtatgccagcctgccacgggtagaggctgcttattacatgcaacgacattacggcgggtctactgatgcgtgggtggccaagacttgttacaggatgcctgatgtgagcaacgatgattacctggagcttgcaagattggatttcaagagatgtcaagcccaacatcagagtgaattgagttacatgcaacgatggtatgacagttgcaatgtcgaagaattcggaataagcagaaaagagttgcttgtagcttattttgtggctgctgcaactatttttgaacctgagagagcaactgagagaattgtgtgggcaaaaactgaaatagtttctaagatgatcaaagcattttttggtgaagactcattagaccaaaaaactatgttgttaaaagaattcagaaacagcatcaataatggctcccacagattcatgaagagtgagcatagaatcgtcaacattctactacaagccttgcaggagctattacatggatctgatgattgtcgtattggtcaactcaaaaatgcttggtatgagtggctgatgaaattcgagggaggagatgaagcaagtttgtggggagaaggagagcttcttgtcaccaccttaaacatttgcacagctcatttccttcaacaccatgatttactgttgaatcatgactacataactctttctgagctcacaaacaagatctgcctcaagctttctcagattcaggtaggagaaatgaatgaaatgagagaagatatgcaggcgttgacgaaattagtgattggggaatcatgcatcgtcaacaaaaacattaagcaaacatttcttgcagttgcaaagactttctattacagagcctacttcgatgccgacaccgttgatctccatatatttaaagttctatttgagcccattgtctga

SEQ ID NO: 29 - protein, 787 aa, Leonotis leonurus
MASTASTLNLTINSTPFVSTKTQAKVSLPACLWMQDRSSSRHVSLKHKFCRNQQLKCRASLDVQQVRDEVFSTAQSPESVDKKIEERKKWVKNLLSTMDDGRINWSAYDTAWISLIKEFEGRDAPQFPSTLMRIAENQLADGSWGDPDYDCSYDRIINTLACVVALTTWNAHPEHNKKGIKYIKENMYKLEETPVVLMTSAFEVVFPALLNRAKNLGIQDLPYDMPIVKEICKIGDEKLARIPKKMMEKEPTSLMYAAEGVENLDWEKLLKQRTPENGSFLSSPAATAVAFMHTKDENCLRYIMYLLDKFNGGAPNVYPIDLWSRLWATDRIQRLGISRFFKEEIKEILSYVYSYWTDIGVYCTRDSKYADIDDTSMGFRLLRMHGFKMDPNVFKYFQKDDRFVCLGGQMNDSPTATYNLYRAAQYQFPGEKILEDARKFSQEFLQHCIDTNNLLDKWVISPRFPEELKFGMEMTWYSCLPRIEARYYVQHYGATEDVWLGKTFFRMEEISNENYKELAKLDFSKCQAQHQTEWIHMQEWYESSNAKEFGISRKDLLFAYFLAAASIFETERAKERILWAKSQIICKMVKSYLENQTASLEHKIAFLTGFGDNNNGLHTINKGSGPVNNVMRTLQQLLGEFDGYISSQLENAWAAWLTKLEQGEANDGELLATTLNICSGRIVYNEDTLSNKEYKAFADLTNKICQNLAQIQNKKGDEIKDPNEGEKDKEVEQGMQALAKLVFEESGLERSIKETFLAVVRTYHYGAYVADEKIDVHMFKVLFEPVE

SEQ ID NO: 30 - DNA, 2364 bp, Leonotis leonurus
  source 1..2364: mol_type = other DNA
atggcctccactgcatccactctaaatttgaccatcaatagtacaccatttgtaagcaccaaaacgcaagcaaaggtttccttgcccgcatgtttatggatgcaggatagaagcagcagtagacacgtgtcgttaaaacacaaattctgtcgaaatcaacaacttaagtgtcgagcaagtctggatgttcagcaagtacgtgatgaagttttttccactgctcaatcccctgaatcggtggataaaaaaatagaggaacgtaaaaaatgggtgaagaatttgttgagtacaatggacgatggacgaataaattggtcagcctatgacacggcatggatttcacttattaaagaatttgaaggacgagatgctccccagtttccgtcgactctcatgcgcatcgcggagaaccaattggccgacgggtcatggggcgatccagattacgactgctcctatgatcggataataaacacactagcgtgtgttgtagccttgacaacatggaatgctcatcctgaacacaataaaaaaggaataaaatacatcaaggaaaatatgtataaactagaagagacgcctgttgtactcatgactagtgcatttgaagttgtgtttccggcgcttcttaacagagctaaaaacttgggcattcaagatcttccctatgatatgcccatcgtgaaggagatttgtaaaataggggatgagaagttggcaaggataccaaagaaaatgatggagaaagagccaacatcgctgatgtatgccgcggaaggagtcgaaaacttggactgggaaaagcttctgaaacagcggacacccgagaatggctcgttcctctcttccccggccgcaactgccgttgcatttatgcacacaaaagatgaaaattgcttaagatacatcatgtaccttttggacaaatttaatggaggagcaccaaatgtttatccgatcgacctctggtcaagactttgggcaacggacaggatacaacgtctgggaatttcccgcttctttaaggaagagattaaggaaatcttaagttatgtctatagctattggacagacattggagtctattgtacacgagattccaaatatgctgacattgacgacacatccatgggattcaggcttctgaggatgcacggatttaaaatggacccaaatgtatttaaatacttccagaaagacgacagatttgtttgtctaggtggtcaaatgaatgattctccaactgcaacatacaatctttacagggctgctcaataccaatttccaggtgaaaaaattctagaagatgctagaaagttctctcaagagtttctacaacattgtatagacaccaataaccttctagataaatgggtgatatccccgcgctttccggaagagttgaaatttggaatggagatgacatggtattcctgcctaccacgaattgaggctagatactacgtacaacattatggtgctacagaggacgtctggcttggaaagacttttttcaggatggaagaaatcagtaatgagaactataaggagcttgcaaaacttgatttcagtaaatgccaagcacaacatcagacagagtggattcatatgcaagagtggtatgaaagtagcaatgctaaggaatttgggataagcagaaaagacctactttttgcttactttttggctgcagcttccatatttgaaaccgaaagggcaaaagagagaattctgtgggcaaaatctcaaattatttgcaagatggttaagtcatatctggaaaaccaaacggcgtcgttggagcacaaaatcgcctttttaactggattcggagataacaacaatggcctgcacacaattaataaggggtctggacctgttaacaatgtcatgagaaccctccaacagctccttggagaattcgacggatatattagtagtcaattggaaaatgcttgggcagcatggttgacgaaactcgagcaaggcgaggccaacgatggcgagctcctcgcaaccacactaaacatttgttctgggcgtattgtgtataacgaggatacattatcgaacaaggagtacaaggctttcgcagacctcacaaataaaatttgtcaaaatcttgctcaaatccaaaataaaaagggtgacgaaattaaggatccgaatgaaggcgaaaaggacaaggaagtcgagcaaggcatgcaggcattggctaagttagtttttgaggaatctgggcttgagaggagtatcaaagaaacattcttagcagtggtgagaacttatcactatggggcctatgttgctgatgagaagattgatgtccacatgttcaaggttttgttcgaaccagttgaatga

SEQ ID NO: 31 - protein, 799 aa, Nepeta mussinii
MTSISSLNLSNAAAARRRLQLPANVHLPEFHSVCAWLNSSSKHDPFSCRIHRKQKSKVTECRVASVDASPVSDHKMSSPVQTQEEANKNMEESIEYIKNLLMTSGDGRISVSAYDTSIVALIKDIEGRDAPQFPSCLEWIGQNQKADGSWGDDFFCIYDRFVNTLACIVALKSWNLHPHKIQKGVTYIKKNVHKLKDGRPELMTSGFEICVPAILQRAKDLGIQDLPYDDPMIKQITDTKERRLKKIPKDFIYQLPTTLLFSLEGQENLDWEKILKLQSADGSFLTSPSSTAAVFMHTKDEKCLKFIENAVKNCDGGVPHTYPVDVFARLWAVDRLQRLGISRFFQPEIKYFLDHIQSVWTENGVFSGRDSQFCDIDDTSMGIRLLKMHGYKIDPNALEHFKQEDGKFSCYGGQMIESASPIYNLYRAAQLRFPGEEILEEAIKFSYNFLQEKLAKDEIQEKWVISEHLIDEIKIGLKMPWYATLPRVEAAYYLDYYAGSGDVWIGKTFYRMPEISNDTYKEMAILDFNRCQAQHQFEWIYMQEWYESSNVKEFGISKKELLVAYFLAASTIFEPERAQERIMWAKTKIVSKMIASSLNKQTTLSLDQKTALFTQLEHSLNGLDSDEKDNGVAETKNLVATFQQLLDGFDKYTRHQLKNAWSQWLKQVQQGEATGGADAELEANTLNICAGHIAFNEQVLSHNEYTTLSTLTNKICHRLTQIQDKKTLEIIDGGIRYKELEQEMQALVKLVVEENDGGGIDRNIKQTFLSVFKNYYYSAYHDAHTTDVHIFKVLFGPVV

SEQ ID NO: 32 - DNA, 2400 bp, Nepeta mussinii
  source 1..2400: mol_type = other DNA
atgacttcaatatcctctctaaatttgagcaatgcagcagctgctcgccgcaggttacaactaccagcaaacgttcacctgccggaatttcactccgtctgtgcatggctgaatagcagcagcaaacacgatccctttagttgccgaattcatcgaaagcaaaaatcgaaagtaaccgagtgtcgagtagcaagcgtggatgcatcaccagtgagtgatcataaaatgagttctcctgttcaaactcaagaagaggcaaataaaaatatggaggagtcaatcgagtacataaagaatttgttgatgacatctggagacgggcgaataagcgtgtcggcatacgacacgtcaatagtcgccctaattaaggacatagaaggacgggacgccccgcaatttccatcatgcctggagtggatcgggcaaaaccaaaaggccgatggctcgtggggggacgacttcttctgtatttacgaccgcttcgtaaatacactagcatgtatcgtggccttgaaatcatggaaccttcaccctcacaagattcaaaaaggagtgacatacatcaagaaaaacgtgcataagcttaaagatgggaggcctgagctgatgacgtcagggttcgaaatttgtgttcccgccattcttcaaagagccaaagacttgggcatccaagatcttccctatgatgatcccatgattaaacagatcactgatacgaaagagcgacgactcaaaaagataccgaaggattttatataccaattgccgacgactttactcttcagtttggaagggcaggagaatttggactgggaaaagatactcaaactgcagtcagctgacggctccttccttacttcgccgtcctccaccgccgccgtcttcatgcataccaaagatgaaaaatgcttgaagttcatagagaacgccgtcaaaaattgcgacggcggagtgccccatacctacccagtagacgtgtttgcaagactttgggcagttgacagactacaacgcctagggatttctcgcttttttcagcctgagattaaatatttcttagatcacatacaaagcgtttggactgagaacggagttttcagtggacgagattcacaattttgcgacattgatgatacgtccatggggataaggcttctgaaaatgcatggatacaaaatcgacccaaatgcacttgagcatttcaagcaggaggatggtaaattttcgtgctacggtggtcaaatgatcgagtctgcatcaccgatatacaatctgtaccgagctgctcaactccgatttccaggagaagaaattcttgaagaggccattaaattttcctataactttttgcaagaaaagctagccaaggatgaaattcaagaaaaatgggtcatatcggagcacttaattgatgagattaagatcgggctaaagatgccatggtacgccactctaccccgagttgaagctgcatattacctggactattatgcaggatccggcgatgtgtggattggcaagactttctacaggatgccagaaatcagtaatgatacatacaaagaaatggccattttggatttcaaccgatgccaagcacaacatcagtttgaatggatttacatgcaagagtggtatgaaagtagcaacgtaaaggaatttgggataagcaaaaaagagctacttgttgcttatttcttggctgcatcaaccatatttgaaccggaaagagcacaagagaggattatgtgggcaaaaacaaaaattgtttccaaaatgatcgcatcatctcttaacaaacaaaccactctatcgttagaccaaaagactgcactttttacccaactcgaacatagtctcaatggcctcgacagtgatgagaaagataatggagtagctgagacgaaaaatctagtggcaaccttccagcagctgctagatggattcgacaaatacactcgccatcaattgaaaaatgcttggagccagtggttgaagcaagtgcagcaaggagaggcgaccgggggcgcagacgcggagctggaagcaaacacgttgaacatctgtgccggtcatatcgcattcaacgaacaagtattatcgcacaacgaatacacaactctctccacactcacaaacaagatctgccaccggcttacccaaattcaagacaaaaagacgcttgagataatcgacggcggcataagatataaggagctggagcaggagatgcaggcgttggtgaaattagttgttgaagaaaacgacggcggcggcatagacaggaatattaaacaaacatttttatcagttttcaagaattattactacagtgcctaccacgatgctcacacaaccgatgttcatattttcaaagtattatttggaccggtcgtctga

SEQ ID NO: 33 - protein, 796 aa, Origanum majorana
MTDVSSLRLSNAPAAGGRLPLPGKVHLPEFRTVCAWLNNGCKYEPLTCRISRRKISECRVASLNSSQLIEKVGSPAQSLEEANKKIEDSIEYIKNLLMTSGDGRISVSAYDTSLVALIKDVKGRDAPQFPSCLEWIAQNQMADGSWGDEFFCIYDRIVNTLACLVALKSWNLHPDKIEKGVTYINENVHKLKDGSTEHMTSGFEIVVPATLERAKVLGIQGLPYDHPFIKEIINTKERRLSKIPKDLIYKLPTTLLFSLEGQGELDWEKILKLQSSDGSFLTSPSSTASVFMRTKDEKCLKFIENAVKNCGGGAPHTYPVDVFARLWAVDRLQRLGISRFFQHEIKYFLDHINSVWTENGVFSGRDSQFCDIDDTSMGVRLLKMHGYNVDPNALKHFKQEDGKFSCYPGQMIESASPIYNLYRAAQLRFPGEEILEEASRFAFNFLQEKIANHEIQEKWVISEHLIDEIKLGLKMPWYATLPRVEAAYYLEYYAGSGDVWIGKTFYRMPEISNDTYKEVAILDFNTCQAQHQFEWIYMQEWYESSKVKDFGISKKDLLVAYFLAASTIFEPERTQERIIWAKTLILSRMITSFLNKQATLSSQQKNAILTQLGESVDGLDKIYSGEKDSGLAETLLATFQQLLDGFDRYTRHQLRNAWGQWLMKVQQGEANGGADAELIANTLNICAGLIAFNEDVLLHSEYTTLSSLTNKICQRLSQIEDEKTLEVIEGGIKDKELEEDIQALVKLALEENGGCGVDRRIKQSFLSVFKTFYYRAYHDAETTDLHIFKVLFGPVM

SEQ ID NO: 34 - DNA, 2391 bp, Origanum majorana
  source 1..2391: mol_type = other DNA
atgaccgatgtatcctctcttcgtttgagcaatgcaccagctgccggcggcaggttgccgctgccgggaaaggttcacctgcctgaatttcgcaccgtttgtgcatggttgaacaatggctgcaaatacgagcccttgacttgtcgaattagtcgacggaagatatctgaatgtcgagtagcaagtctgaattcgtcgcaactaattgaaaaggtcggttctcctgctcaatctctagaagaggcaaacaaaaagatcgaggactccatcgagtacattaagaatctattgatgacatctggcgacgggcggataagtgtgtcggcttacgacacgtcgctagtcgccctaataaaggacgtgaaaggacgagatgcccctcagttcccgtcgtgcctggagtggatagcgcaaaaccaaatggccgacgggtcgtggggggatgagttcttctgtatttacgaccggatcgtgaatacattagcatgcctcgttgccttgaaatcatggaaccttcaccccgacaagatcgaaaaaggagtgacgtacatcaacgaaaatgtgcacaaactgaaagacgggagcaccgagcacatgacgtcagggttcgaaatcgtggtccccgccactctagaaagagccaaagtcttgggcatccaaggcctcccttatgatcatcccttcattaaggagattattaatactaaggagcgaagattaagcaaaatacccaaggatttgatatacaaactgccaacgacgctgctgttcagtttagaagggcagggagaattagattgggaaaagatactgaaactgcagtcaagcgatggctccttccttacttcgccctcgtcgaccgcctccgtcttcatgcggacgaaagacgagaaatgcctcaagttcattgagaacgccgttaagaattgcggcgggggagcgccgcatacttacccagtggatgtgtttgcaagactttgggcagttgacagactacagcgattagggatttctcgattcttccaacacgagattaaatacttcttagatcacattaacagtgtatggaccgagaatggagttttcagtggacgagattcacaattttgtgatatcgacgacacttctatgggagttaggcttctaaaaatgcatggatacaatgttgatccaaatgcgctcaagcatttcaagcaggaggatggcaaattctcttgctaccctggccaaatgatcgagtctgcatctccgatatacaatctctaccgagccgctcaactccggttccccggagaagaaattctcgaagaagcaagtcgattcgccttcaactttctgcaggaaaagatagccaaccatgaaattcaagaaaaatgggtcatatctgagcacttaattgatgagataaagttgggactgaagatgccatggtacgcgactctgccccgagttgaggccgcttattatctagagtattatgctggctcaggcgacgtatggattggaaagactttctaccggatgccggaaatcagtaacgatacgtataaagaggtggccattttggatttcaacacatgccaagctcaacaccagtttgaatggatttacatgcaagagtggtacgaaagtagcaaggttaaagatttcgggataagcaaaaaggacctacttgttgcttactttctggcggcatcgactatatttgaacccgaaagaacacaagagaggattatttgggcaaaaaccctaattctttctaggatgatcacatcatttctcaacaaacaagctacactttcatcccaacaaaagaatgccatcttaacacaacttggagagagtgtcgatggcctcgataaaatatatagtggtgagaaagattctgggctggctgagactctgctggctaccttccagcaactgctcgacggattcgatagatacactcgccatcaactgagaaatgcttgggggcaatggttgatgaaagtgcagcaaggagaggccaacggtggcgccgacgctgagctcatagcaaacacactcaatatctgcgccggccttatcgccttcaacgaagacgtattgttgcacagcgaatacacgactctctcctccctcaccaacaaaatatgccagcgccttagccagattgaagatgaaaagacgcttgaagtgattgaagggggcataaaagataaggaactggaggaggatattcaggcgttggtgaagctagccctcgaagaaaacggcggctgcggcgtcgacagaagaatcaagcagtcattcttatcagtattcaagactttttactacagagcctaccatgatgctgagaccaccgatcttcatattttcaaagtactgtttgggccggttatgtga

SEQ ID NO: 35 - protein, 798 aa, Perovskia atriplicifolia
MTSMSSLNLSRAPATTHRLQLQAKVHVPEFYAVCAWLNSSSKQAPLSCQIRCKQLSRVTECRVASLDASQVSEKDTSHVQTPDEVNKKIEDYIEYVKNLLMTSGDGRISVSPYDTSIVALIKDSKGRNIPQFPSCLEWIAQHQMADGSWGDQFFCIYDRILNTLACVVALKSWNVHGDMIEKGVTYVKENVHKLKDGNIEHMTSGFEIVVPALVQRAKDLGIQGLPYDDPLIKEIADTKERRLKKIPKDMIYQTPTTLLFSLEGQGDLEWEKILKLQSGDGSFLTSPSSTAHVFVQTKDEKCLKFIENAVKNCSGGAPHTYPVDVFARLWAIDRLQRLGISRFFQPEIKYFIDHINSVWTENGVFSGRDSEFCDIDDTSMGIRLLKMHGYKVDPNALNHFKQQDGKFSCYGGQMIESASPIYNLYRAAQLRFPGEEILEEASKFAFNFLQEKIANDQFQEKWVISDHLIDEVKLGLKMPWYATLPRVEAAYYLQYYAGSGDVWIGKVFYRMPEISNDTYKELAILDFNRCQAQHQFEWIYMQEWYHRSSVSEFGISKKELLRTYFLAAATIFEPERTQERLVWAKTQIVSRMITSFVNNGTTLSLDQMTALATQIGHNFDGLDQIISAMKDHGLAGTLLTTFQQLLDGFDRYTRHQLKNAWSQWFMKLQQGEANGGEDAELLANTLNICAGFIAFNEDVLSHDEYTTLSTLTNKICKRLSQIQDKKALEVVDGSIKDKELEQDMQALVKLVLEENGGGVDRNIKQTFLSVFKTFYYTAYHDDETTDVHIFKVLFGPVV

SEQ ID NO: 36 - DNA, 2397 bp, Perovskia atriplicifolia
  source 1..2397: mol_type = other DNA
atgacctctatgtcctctctaaatttgagcagagcaccagctaccacccaccggttacagctacaggcaaaggttcacgtgccggaattttatgccgtgtgtgcatggctgaatagcagcagcaaacaggcacccttgagttgccaaattcgctgcaagcaactatcaagagtaactgaatgtcgggtagcaagtctggatgcgtcgcaagtgagtgaaaaagacacttctcatgtccaaactcccgatgaggtgaacaaaaagatcgaggactatatcgagtacgtcaagaatctgttgatgacgtcgggcgacgggcgaataagcgtgtcgccctacgacacgtcaatagtcgcccttattaaggactcgaaagggcgcaacatcccgcagtttccgtcgtgcctcgagtggatagcgcagcaccaaatggcggatggctcatggggggatcaattcttctgcatttacgaccggattctaaatacattagcatgtgtcgtagctttgaaatcctggaacgttcacggtgacatgatcgaaaaaggagtgacgtacgtcaaggaaaatgtgcataagcttaaagatgggaatattgagcacatgacgtcggggttcgaaattgtggttcccgcccttgttcaaagagccaaagacttgggcatccaaggcctgccctatgatgatcccctcatcaaggagattgctgatacaaaagaaagaagattgaaaaagatacccaaggatatgatttaccaaacgccaacgacattactattcagtttagaagggcagggagatttggagtgggaaaagatactgaaactgcagtcaggcgatggctccttcctcacttcgccgtcatccaccgcccacgtgttcgtgcagaccaaagatgaaaaatgcttgaaattcatcgagaacgccgtcaagaattgcagtggaggagcgccgcatacttatccagtcgatgtcttcgcaagactttgggcaattgacagactacaacgcctaggaatttctcgtttcttccagccggaaattaagtatttcatagaccacatcaacagcgtttggacagagaacggagttttcagtgggcgagattcggaattttgcgatattgatgacacgtccatgggcatcaggcttctcaaaatgcacggatacaaagtcgacccaaatgcactcaatcatttcaagcagcaagatggtaaattttcttgctacggtggtcaaatgatcgagtctgcatctccaatatacaatctctacagggctgctcagctacgatttccaggagaagaaattcttgaagaagccagtaaatttgcctttaactttttgcaagaaaaaatagccaacgatcaatttcaagaaaaatgggtgatatccgaccacttaatcgatgaggtgaagctcgggctgaagatgccatggtacgccactctaccccgggttgaggctgcatattatctacaatactatgctggttctggcgacgtatggattggcaaggttttctacaggatgccggaaatcagcaatgatacatacaaagagctggccatattggatttcaacagatgccaagcacagcatcagttcgaatggatttatatgcaagagtggtatcacagaagcagcgttagtgaattcgggataagcaaaaaagagctgcttcgtacttactttctggctgcagcaaccatattcgaacccgagagaacacaagagaggcttgtgtgggcaaaaacccaaattgtctctaggatgatcacatcatttgttaacaatggaactacactatctttggaccaaatgactgcacttgcaacacaaatcggccataatttcgatggcctcgatcaaataattagtgctatgaaagatcatggactggctgggactctgctgacaaccttccagcaacttctagatggattcgacagatacactcgccatcaactcaaaaatgcttggagccaatggttcatgaaactccagcaaggggaggcgaacggcggggaagacgcggagctcctagcaaacacgctcaacatctgcgcgggtttcattgctttcaacgaagacgtattgtcgcacgatgaatacacgactctctccacccttacaaacaaaatctgcaagcgccttagccaaattcaagataaaaaggcgctggaagttgtcgacgggagcataaaggataaggagctcgaacaggatatgcaggcgttggtgaagttggtccttgaagaaaatggcggcggcgtcgacaggaacatcaaacagacatttttgtccgttttcaagactttttactacaccgcctaccacgatgatgagaccactgatgttcatattttcaaagtactgtttggaccggtcgtatga

SEQ ID NO: 37 - protein, 783 aa, Pogostemon cablin
MSFASQSHVAFVLRRPSAVAPPPPTRIPTTAALSPLKPGDFSHGRSSFMPTSIKCNAISTSRVEEYKYTDDHNQSGLLEHDGLISDKINELVTKIQLMLQNMDDGEISISPYDTAWVSLVEDVGGNDRPQFPTSLEWISNNQLPDGSWGDPNAFLVHDRILNTLACVVALKSWKMHPHKCNRGVSFVRENIYRMDDEKEEHMPNGFEVVFPALLQKAKTLNIDIPYEFPGIQKFYAKRDLKFARIPMDILHSVPTTLLFSLEGVRCGLDLDWGKLLELQAADGSFLYSPSSTAFALEQTKDQNCLKYLSKLVRKFDGGVPNVYPVDLFEHNWAVDRLQRLGISRYFTPEINQCLDYSYRYWSNSKGMYSASNSQIQDVDDTAMGFRLLRLNGYDVSTQGFRQFEAGGDFFCFAGQSSQAVTGMYNLYRASQVMFPGEKLLEDAKKFSTNFLQQKRANNQLTDKWVIAKDVPAEVGYALDIPWYASLPRLEARFFIQQYGGDDDVWIGKTLYRMGYVNNNTYLELAKLDYNTCQRLHQHEWITIQRWYEINLKITSVGLSKRGVLLSYYLAAANLFEPQNSTHRIAWAKTSILVSAIQLSPLQKRDFINQFHRSTANNGYETSNVLVKSVIKGVHELSMDAMLTHNKDIHRQLFNAWRKWMSVWEEGGDGEAELLLSTLNTCDGVDESTFSDPKYEHLLEITVRVTHQLHLIQNAETKRVGDREEIDLSMQQLVKLVFTKSSSDLDSCIKQRFFAIARSFYYVAHCDPEMVDSHIAKVLFERVM

SEQ ID NO: 38 - DNA, 2352 bp, Pogostemon cablin
  source 1..2352: mol_type = other DNA
atgtcatttgcttctcaatcacatgtcgcctttgtactccgacggccatctgccgttgctccgccaccaccgactagaattccgacaacagccgctctttctcctctcaaaccaggtgatttttcccatggcagatcatcatttatgcccacttccattaaatgtaatgcaatttccacatctcgcgtcgaagaatacaagtacacggatgatcataatcagagtggtttattggagcatgatggtttgatatcagacaagataaatgaattggtgaccaagatacaattgatgctacaaaacatggatgacggagagataagcatctccccatatgacaccgcatgggtgtcgttggtggaggatgtgggcggcaacgaccgcccacagtttcctacgagcctggagtggatatcgaataaccagctccccgacggctcgtggggcgacccgaatgcctttttggtgcacgaccgtatcctcaacacattggcatgcgtcgttgcactcaaatcctggaaaatgcacccccacaaatgcaatagaggagttagtttcgtgagagaaaatatatacagaatggatgatgaaaaagaggaacacatgccaaatggattcgaagtggtatttccagcactccttcaaaaagcgaaaaccctaaacattgatatcccgtacgagtttccaggaatacaaaaattttatgccaaaagagatttaaaattcgccaggattccaatggatatattgcatagcgttccgacaacattactgttcagcttagaaggtgtaagatgtggtcttgatctggattgggggaagcttctagaattgcaagctgctgatggctcatttctctactctccatcctctactgcctttgcactagaacaaaccaaggatcaaaactgcctcaaatatctatctaaacttgttcgaaaattcgatggcggagtacccaacgtgtacccggtggacttgttcgaacataattgggcagttgatcgtctccaaaggctcggaatttctcgttattttacgcctgaaatcaaccaatgtcttgattattcttacagatattggtcaaatagtaaagggatgtactcggcaagcaattcccagattcaggacgttgatgacaccgccatgggattcaggcttttgagactcaacggctacgatgtctctacacaagggtttaggcaattcgaggcagggggggacttcttctgcttcgcggggcagtcgagccaagctgtaaccggaatgtacaacctctacagagcttcccaagtgatgttccctggagagaagctactggaagatgccaagaaattctccaccaacttcttgcaacaaaaacgagccaataaccagctcactgacaagtgggttattgccaaagatgttccagctgaggtgggatatgccttggatattccctggtatgccagtctgccccgactggaagcaagatttttcatacaacaatacggtggagacgacgacgtttggatcggcaaaaccttgtatagaatgggatatgtgaacaacaacacttatctggaactcgcaaagctagactacaacacctgccaaaggttgcatcagcatgagtggataaccattcaacgatggtacgaaattaatttaaaaattactagtgttgggttgagcaaaagaggggtcctgttgagttattacttagccgcagccaatctgtttgagcctcaaaactcaacacaccgcatcgcttgggccaaaacttcgattttagtaagcgctattcaactttctcccctccaaaagcgcgactttattaaccaattccaccgctccaccgcaaataatgggtatgaaacaagtaatgtgttggtgaagagtgtaatcaagggtgtgcatgagctctccatggacgctatgttgacgcacaataaagacatacatcgccaactttttaatgcttggcgaaagtggatgtcagtgtgggaagagggaggtgatggagaagcggagctgttattgtcgacgcttaacacgtgcgacggagtagatgaatccacattcagcgatcccaaatacgagcacctcttagagatcaccgtcagagtcacccaccagcttcatctcattcagaatgcagagacgaagcgtgtgggtgaccgtgaggaaatagatttgagcatgcaacaacttgttaagttggtgttcactaaatcatcatcggatctggattcttgtatcaagcaaagattttttgcgattgccagaagtttctattacgtggctcattgtgatccggagatggtggactcccacatagccaaagtattgtttgagagggtgatgtag

SEQ ID NO: 39 - protein, 599 aa, Prunella vulgaris
MSSLSIPFSSAICTSSIPKISTGHHRRTARMPAHDTSRLVFRPSAVMVEGSPMTTSSNGKEVQRLITTFKPSMWKDIFSTFSFDNQVQEKYLKEIEELKKEVRSTLMSATHRKLFDLIDNLERMGIAYHFETEIEDKLKQAHASLEEEDDYDLFTTALRFRLLRQHRYHVSCDPFAKFVDQDNKLKESLSSDVEGLLSLFEASHLRIHNEDVLDEAIVFTTHHLNRMMPQLESPLKEEVKHALRYPLHKCLGILSLRFHIDRYENDKSRDEVVLRLGQVNFNYMQNIYMNELYEITTWWNKLQMTSKVPYFRDRLVECYMWGLAYHFEPEYAPVRVLITKYYMTATTVDDTYDNYATLEEIELFTQAIDRWSEDEIDQLPDEYLKIVYKGLMNFTEEFRRDAEERGKGYVIPYFIEETKRATQGYANEQRWIMKREMPSFEEYMVNSRVTSLMYVTYVAVVAVIESATKETVDWALSDSDIFVYTNDIGRLIDDLATHRRERKDGTMLTSMDYYMKEYGGTMEEGEAAFRKLMEEKWKLLNAAWVDTINGKESKEIVVQVLDLARICGTLYGDEEDGFTYPEKNFAPLVAALLMNPIHI

SEQ ID NO: 40 - DNA, 1800 bp, Prunella vulgaris
  source 1..1800: mol_type = other DNA
atgagctctctctcaattcccttttcttccgccatttgcacttcatcaatcccaaagatcagtactgggcatcatcgccgcaccgcgaggatgcccgcgcacgacacatcgcgtctcgtctttcgcccttcagctgtgatggtggaaggaagtccgatgactacttcaagcaacgggaaggaagtccaacgacttataaccactttcaagcctagcatgtggaaagatattttttctaccttctctttcgataatcaggtgcaagaaaagtatttgaaagaaattgaggaattgaagaaagaagtaagaagcacactaatgagtgctacgcataggaaattgtttgacttgatcgacaatctcgagcgtatgggaatcgcctatcatttcgagacagaaatcgaagacaagctcaaacaagctcatgcttctctagaggaggaagatgactacgacttgttcactactgcacttcgctttcgtctgctcagacaacatcgctatcatgtttcttgcgatccctttgcgaaatttgttgaccaagacaacaaattgaaagagagtcttagtagcgacgtcgaggggctattaagcttgttcgaggcatcccatcttcggatccacaacgaggatgttctagatgaagctatagtgttcacaacccatcacttgaatcgaatgatgccacaattggaatcgccccttaaagaagaagtgaagcatgctcttcgatacccccttcacaagtgtcttggaatccttagccttcgttttcatatcgacagatatgagaatgataagtcgagggatgaagttgttctcagactaggccaagttaatttcaattacatgcagaacatttacatgaacgagctctatgaaatcaccacgtggtggaacaagttgcagatgacttcaaaagtaccttactttagagatagattggtagagtgctatatgtggggtttggcatatcatttcgaaccagaatacgctcccgttcgagtcctcattaccaagtactatatgaccgccacaactgtcgacgatacctatgataattatgctacactcgaagaaatcgaactcttcactcaggccattgacaggtggagcgaggatgagattgatcagctacctgatgaatacctaaaaatagtgtacaaaggtctaatgaacttcactgaagagtttagacgtgacgcagaagagcgagggaaaggctatgtgattccttactttattgaagaaacgaagagagcaacacagggttatgcaaacgagcagaggtggataatgaagagagaaatgccgagttttgaagagtatatggtgaactcaagggtaacatcacttatgtatgtgacctacgttgctgttgtggcagtcatagaatcagctaccaaagaaaccgtagattgggcgctaagtgactccgatatctttgtctacactaacgatatcggccgacttatcgacgaccttgccactcatcgacgcgagaggaaagacgggacaatgcttacatcgatggattattacatgaaggaatatggcggtacgatggaagagggggaagctgcatttaggaaattgatggaggagaaatggaaacttttgaatgcagcatgggtagatactattaatggaaaagagtcgaaggaaatagttgtgcaagttctcgacctcgccaggatatgcggaacgctctatggggacgaagaagatggcttcacctacccagagaagaattttgcaccactcgttgctgctctattgatgaatcctatacatatttga

SEQ ID NO: 41 - protein, 821 aa, Chiococca alba
MSSSTSAAATLLGLSPASRRFVSFPPANGPIETITGIWSPGKALHHFNFRLRCSTVSSPRTQELGQVSQNGMSGIKWHDIVEEGVTEKGTLEANTSSWIKESIEAIRWMLRTMDDGDISISAYDTAWVALVEDINGSGGPQFPSSLEWIANNQLPDGSWGDSDIFSAHDRILNTLGCVVALKSWNMHPEKSEKGLLYLRDNIHKLEDENVEHMPIGFEVAFPSLIEIAKKLSIDIPDDSAILQEIYARRNLKLTRIPKDIMHTVPTTLLHSLEGMPELDWKRLISLKCEDGSFLFSPSSTAFALTQTKDADCLRYLTKTVQKFNGGVPNVYPVDLFEHIWAVDRLQRLGISRYFQSEIRECIDYVHRYWTDKGICWARNTHVYDIDDTAMGFRLLRLHGYDVSADVFRYYEKDGEFVCFAGQSNQAVTGMYNLYRASQVMFPGENILSDARKFSSEFLHDKRANNELLDKWIITKDLPGEVAYALDVPWYASLPRLETRLYLEQYGGEDDVWIGKTLYRMQKVNNNIYLELGKLDYNNCQALHQLEWRSIQKWYNECGLGEYGLSERSLLLSYYLAAASIFEPERSKERLAWAKTTMLIRTIESYLSSEQMVEDHNGAFVSEFQYYCSNLDYVNGGRHKPTQRLVRTLLGTLNQISLDAVLVHGRDIHQYLRQAWEKWLIALQEGDDSDMGQEEAELLVRTLNLCAGRYASEELLLSHPKYQQLLHITTRVCNQIRHFQHKKVQDGENGRANMGDGITSISSIESDMQELTKLVVGNTQNDLDADTKQTFLTVAKSFYYTAHCNPGTINCHIAKVLFERVL

SEQ ID NO: 42 - DNA, 2466 bp, Chiococca alba
  source 1..2466: mol_type = other DNA
atgtcttcttctacctcagcagcagcaacccttctcggattatcgccggcaagccgccggtttgtatcatttcctccggcaaatggacctatagaaactattaccggtatttggtcgcccggcaaagctcttcatcactttaatttccgtctgcgttgtagcacggtgtccagtcctcgcacccaagaattgggccaggtgtcacaaaatggcatgtctggtataaagtggcatgacatagtggaagaaggagtcacagaaaaaggaactcttgaggcgaacacatcaagctggataaaagaaagcatagaagccattcgttggatgctgcgtaccatggatgacggggatatcagcatatctgcttatgatactgcatgggttgcccttgtggaagatatcaacggaagtggcggtcctcaatttccttcaagcctcgagtggattgccaacaatcagcttcctgatggttcatggggcgacagcgacatcttttcagctcacgatcggattctcaacactttgggatgcgttgttgcattaaaatcttggaacatgcaccctgaaaagagtgaaaaaggattattatatttaagggataacattcacaagcttgaggatgaaaatgtcgagcacatgcctatcggttttgaagtggcatttccttcactaattgagatagccaaaaagttgagcattgatattccggatgattctgcaatcttgcaggagatatatgccagaagaaatctaaagctaacaaggataccgaaggacattatgcacacagtgcccacaacattgctccacagcttggaaggcatgccagaactagactggaaaaggctaatatctctaaagtgtgaggatggttcctttctgttttctccatcctccactgcttttgccctcacgcaaactaaagatgctgattgcctcagatatttaactaaaaccgtacaaaaattcaatggaggagttcccaatgtttaccccgtggacttattcgaacacatctgggctgttgatcgacttcaaagactaggaatttctcgatacttccagtcagaaatccgcgagtgcatcgattatgttcaccgatattggacggataaaggtatctgttgggctagaaatacccacgtttatgacattgatgatacagctatgggttttagacttctaaggttgcatggctacgatgtttctgcagatgttttcagatactatgagaaggatggcgaattcgtttgctttgccggacagtcaaaccaggcggtgaccggaatgtataacctgtatagagcttctcaagtgatgtttccaggggagaatatactttcggatgctaggaaattctcgtccgaattcttgcatgataagcgagccaacaatgagctcctagataaatggatcataaccaaagatttgcctggggaggtagcatatgctttagatgttccatggtatgccagtttacctcgtttagaaaccagattgtatttggaacaatatggcggcgaagatgatgtctggattggcaagacattgtacaggatgcaaaaagttaacaacaacatctatcttgaacttggcaaattagattacaacaactgtcaggcattgcatcagcttgagtggagaagcatccaaaaatggtacaatgaatgcggtcttggagagtacggattaagcgagagaagcctccttctttcgtattatttggccgcagccagtatatttgaaccggagaggtcaaaggaacggcttgcctgggccaaaactactatgctaatccgcacaattgaatcttatttgagtagtgaacaaatggttgaggatcacaatggagcctttgttagcgagttccaatactattgcagtaaccttgactacgtaaatggtggaaggcataagccaacacaaaggctagtgaggactctactcggaactttaaatcagatttctttggacgcagtgttagtccacggcagagatatccatcaatatttgcgtcaagcctgggaaaagtggttgatagctttgcaagagggagatgatagtgacatgggtcaagaggaagcagaacttttagtgcgcacactaaacctatgcgccggtcgctacgcatcggaggagctattgttgtcccatcccaagtatcaacaacttttgcacatcactactagagtctgtaaccaaattcgtcatttccaacacaaaaaggtgcaagatggggaaaatggaagagcaaacatgggtgatggcatcacaagcatcagctcaatagagtcggacatgcaagaactaacgaaattagttgtcggcaatacccaaaacgatctagatgctgatacgaagcaaacatttctcacggtggcaaaaagcttctactacaccgcccactgcaatcccggaacaatcaattgccatattgctaaagtattatttgagagagtactttga

SEQ ID NO: 43 - protein, 747 aa, Chiococca alba
MPVIKSHEFIEEVGPEKGTLKLSRSSRINELVESIQTMLQSMDDGEISMSAYDTAWVALVEDINGSSYPQFPMSLEWIANNQLPDGSWGDGSIFSVHDRIISTLGCVLALKSWNMHPDKSEKGLLFIRDNIHKVGDESAEHMPIGFEVVFPSLIERAKNLDIDIPDISAILQEIYARRNLKLARIPKDILYTVPTTLLHSLEGMPELDWQKLLPLKCEDGSFLFSPSCTAFALMQTKDGDCLRYLTNTIEKFNGGVPGVYPVDLFEHIWAVDRLQRLGISRYFQTEIEECMSYVYRYWTDKGICWARNSKVEDIDDTAMGFRLLRLHGYMVSADVFAQFEKGGEFVCFAGQSNQALTGMFNLYRASQVMFPGEKILADAKKFSSNFLHEKRANNELLDKWIITKDLPGEVTYALDVPWYASLPRVETRLYLEQYGGEDDVWIAKTLYRMRKVNNKIYLELGILDYNNCQALHQLEWRSIQKWYKDSGLEEYGLSERNLLLAYYLATACIFEPERLVERLSWAKTTALIYTTKSYFRTECNSGEQRKAFLHEFQQYCNDLDYVSGARHKPTIRLIEALLGTLEQVSLDAILDHGRYIHQDLRNAWEKWLIALQEGVDMDQEEAELTVLTLHLCAGSYTSEELLLSHPKYQQLLNITSRVCHQIRQFQREKAQDTDNGRENLVAITSIKAIESDMQELAKLVLTKSTGDLAAKIKQTFLIVAKSFYYTAHCLPGIISTHIAKVLFEKVF

SEQ ID NO: 44 - DNA, 2244 bp, Chiococca alba
  source 1..2244: mol_type = other DNA
atgccagtaataaagtcgcatgagtttattgaagaggtcggcccggaaaaaggaactctgaagctgagcagatcaagtaggataaacgaacttgtagaatcaattcaaacgatgcttcaatcgatggatgatggggaaataagcatgtctgcttatgacaccgcgtgggttgcccttgtggaagatattaatggaagcagctaccctcaattccctatgagcctcgagtggattgccaacaatcagcttcctgatggttcatggggtgacggcagtatcttttcggttcatgatcggataatcagcacattaggatgtgttcttgcattaaaatcatggaacatgcacccggacaaaagcgaaaaaggactgttatttataagggacaatattcacaaggttggagatgagagcgctgagcacatgcctattggttttgaggtggtatttccttcgcttattgagagagccaaaaacttggacattgatattccagatatttctgctatcttgcaagagatttatgcacgaagaaatctaaagctcgcaaggattccaaaggatatactgtataccgtgcccacgacattacttcatagcttagaaggaatgccagaactggactggcaaaagctactgccattaaaatgtgaggatggttcatttctattttctccatcgtgcactgcttttgccctcatgcagactaaggatggtgattgcctcagatatctaactaataccatagaaaaattcaatgggggagttcccggtgtataccctgtggacttgttcgaacacatttgggctgttgatcgcttgcaaagactaggaatttcccggtattttcagacagaaattgaagaatgtatgagttatgtttaccgatattggacggataaaggtatctgttgggctagaaactccaaagttgaagacatcgatgacacagccatgggttttagacttctaaggttgcatggttacatggtttctgcagatgtgtttgcacagtttgagaaagggggtgaattcgtttgctttgctggacagtcgaaccaggcgctgactggaatgtttaacctgtatagagcttctcaagtaatgtttccaggggagaagatacttgctgatgccaagaaattctcatcgaacttcttacatgaaaagcgtgcaaacaacgagcttctagataaatggatcataactaaagatttgcctggagaggtgacgtatgcgctagatgttccatggtacgccagtttacctcgtgtagaaacgagattatatctggaacaatatggaggagaggatgatgtctggattgccaagacattgtacaggatgagaaaagttaacaacaaaatttaccttgaacttggcatattagattacaataactgtcaagcattgcatcagctggagtggagaagcatccaaaaatggtataaggattctggccttgaagagtacgggttgagcgagaggaaccttctcctggcatattatctggccacagcttgtatatttgaacccgaaaggttggtggagcgcctttcctgggcgaaaacaaccgccttaatctacacaacaaaatcttatttcagaactgaatgcaactctggggaacagagaaaagcttttcttcatgagttccaacagtactgcaatgacctggactacgttagtggcgcaaggcacaagccaacaataagattgatcgaagctctacttggaaccctagagcaggtctctttggatgcaatattagatcatggccgatatatccatcaagatttgcgtaatgcttgggagaaatggttgatagctttgcaagagggagttgacatggaccaagaagaagcagaacttacagtgctcacactacacctgtgtgccggcagctacacatcggaggagttactgttatctcatcccaagtatcaacaacttttaaatatcactagtagagtctgccaccaaattcgtcaattccagcgcgaaaaggcacaggatacggataatggaagagaaaacttggttgccatcacaagcatcaaggcgatagaatcagacatgcaagaacttgcgaaattagttctgaccaaatccactggcgatttagctgctaaaatcaagcaaacatttcttatagtggcaaagagcttctactacaccgcacattgccttcctggaattatcagtacccacattgccaaagtactatttgagaaagttttctga

SEQ ID NO: 45 - protein, 822 aa, Chiococca alba
MMMMMVVMNTAPAHSYHPFPFAGPKSSATLFSNYYCSSRKKSSPPRISASVSLLTGVESTTAINSSDPEIKERIRKLFHDVDISLSSYDTAWVAMVPAPHSSQSPLFPQCINWLLDNQLPDGSWSLPPPHHHPLLLKDALSSTLACVLALRRWGIGQEQVDKGIRFVELNFASASDQNQHLPVGFDIIFPGMLEYARDLNLNLQLESATVNALLLKRDQELTRFFKSYSDESKAYLAYVSEGIVKLQNWDTVMKFQRKNGSLFNSPSATAAAVMHVHNPGCLDYLHSVLEKHGNAVPTVYPLDIYPRLCLVDNLERLGICGHFRKEILSVLDDTYRCWMQGDEEIFAEKSTCAIAFTLLRKHGYNISADPLTPFLKEECFSNSLGGCLKDTSAVLELYRALEMIISQNESALVKKSLWSRSFLKEHISGGCDLKGFSNQISILVDDILNFPSHATLQRVANRRSIEQYNLDSTKILKTSYCSSNFSNKDLLILAVKDFNHCQLIHREELKELERWVTDNRLDKLKFARQKSAYCYFSAAATIFSPELSDARMSWAKNGVLATLVDDFFDVGGSLEELKKLIELVEKWDINVSDGCCSEPVQILFSALHSTIQEIGDKAFKWQARSVTNHIFKIWLDLLNSMLREAEWARNATVPTVEEYMTNGYVSFALGPIILPALYLVGPKLSEEVVKDSEFHSLFKLVSTCGRLLNDVHSFERESKSGQLNALSLRLIHGGVGITEAAAVAEMKSSIENLRRELLRLVLRKEGSVVPRACKDLFWNMSKVLHQFYNKDDGFTSEEMIQLVKSIIYEPIAVNEFLNSCHT

SEQ ID NO: 46 - DNA, 2469 bp, Chiococca alba
  source 1..2469: mol_type = other DNA
atgatgatgatgatggtggtgatgaacacagctcccgcccactcttaccatcctttcccctttgccggcccaaaatcctcagccacacttttttccaattattattgttccagtaggaagaaatcatcgccacctcgcatctctgcctcagtttctttgctaactggagttgaaagcacaactgcaattaattcttcagacccggagatcaaagaaagaataaggaaactatttcatgatgttgatatctcgctttcttcatatgacactgcatgggtggcaatggtccctgctccacattcttcccagtctcccctttttccccagtgcattaattggttattggacaatcagcttcctgatggctcatggagtcttcctcctcctcatcatcatcctctattacttaaagatgcattatcctctacccttgcatgtgttcttgcgctcaggagatggggaattggtcaagaacaagttgacaagggtattcgttttgttgagttaaattttgcttcagcatctgaccagaaccagcatttgccagttggatttgacattatattccctggcatgctcgaatatgctagagatttaaatttaaatcttcaactagaatctgcaacagtaaatgccttacttcttaaaagagatcaggagcttacaagattctttaaaagctactcagacgagagtaaagcataccttgcatatgtatcagaaggtatagtaaagttacagaactgggatacagttatgaagttccaaagaaagaacgggtcactattcaattcaccttcagctacagcagctgctgttatgcatgtccacaatcctggttgcctcgattaccttcactcagtgttggagaagcatggaaatgctgttccaacagtttaccctttggatatatatccacgcctctgcttggttgacaaccttgagagactgggtatttgtggtcattttaggaaggaaattctgagtgtattggatgatacatacagatgctggatgcagggggatgaagagatatttgcagaaaaatcaacttgtgccatagcatttacattattgcgaaagcatgggtacaacatctctgcagatccattgaccccattcttaaaggaagagtgtttttccaattctttgggtggatgtttgaaagatactagtgctgtacttgaattataccgggcattagagatgattattagccagaatgaatcagctctggtgaaaaaaagcttgtggtccagaagcttcctgaaagagcatatttctggtggttgtgatttaaagggattcagcaatcaaatttccatactggtggatgatatcctcaactttccatcgcatgctactttgcaacgggttgctaacaggagaagcatagagcaatacaacttagacagtacaaaaattttaaaaacttcatattgctcgtcgaattttagcaacaaagatttattgatcctggcagtcaaagattttaatcattgccaactcatacaccgtgaagaactgaaagaactagaaaggtgggtcacagacaatagattggacaagttaaagtttgctaggcagaagtctgcatactgttacttttctgctgcagcaaccatattctcacctgaactttctgatgcccgcatgtcatgggccaagaatggtgtacttgctactttggttgatgacttctttgacgtgggaggttctctagaggaattaaagaaactgattgagttggttgaaaagtgggatataaatgtcagtgatggttgttgctctgaaccagtgcaaatcctcttctcagcactacatagtacaatccaggagattggagataaagcattcaaatggcaagcacgcagtgtaacaaaccacatatttaagatatggttagatttgcttaattctatgttgagggaagctgagtgggctagaaatgcaacagtgcctacagttgaagaatatatgacaaatggttatgtatcatttgctttggggccaattatcctccctgctctttatcttgttggacctaagctgtcagaggaagtagttaaggattctgaattccactccctttttaagctagtgagtacctgtgggcggcttctgaatgatgtccacagcttcgagagggaatcaaagtccggccaactaaatgctctgtctctgcgcctgattcatggtggtgttggcattactgaagcagctgctgttgcagagatgaagagttcaattgagaatctaaggagagaactgctgagactagtcttgcgcaaagagggtagtgtagttccaagagcttgcaaggatttgttttggaatatgagtaaagtgctacatcaattttacaacaaagatgatggatttacttcagaggagatgattcagcttgtgaagtcgatcatttatgagccaattgcggtcaatgaatttttgaatagttgccatacatga

SEQ ID NO: 47 - protein, 817 aa, Chiococca alba
MMIMVMNTAPVHAYHALPIPTQKSSTTLFPNYNCSSRKKSSPPRISAASVSLQTGVERTTAIHSSDLEIKERIRKLFHDVDISLSSYDTAWVAMVPAPHSSQSPLFPQCINWLLDNQLPDGSWSLPPHHHHHHPLLLKDALSSTLACVLALRRWGIGQEQVDKGIRFVELNFASASDQNQHLPVGFDIIFPGMLEYARDLNLNLQLESATVDALLLKRDQELIRFFKSYSDESKAYLAYVSEGIIKLQNWDTVMKFQRKNGSLFNSPSATAAAVMHVHNPGCLDYLHSVLEKHGNAVPTVYPLDIYPRLCLVDNLERLGICGHFRKEILSVLDDTYRCWMQGDEEIFAEKSTCAIAFTLLRKHGYNISADPLTPFLKEECFSNSLGGCLKDTSAVLELYRALEMIISQNESALVKKSLWSRSFLKEHISGGCDLKGFSNQISKQVDDILNFPSHATLQRVANRRSIEQYNLDSTKILKTSYCSSNFSNKDLLILAVKDFNHCQLIHREELKELERWVADNRLDKLKFARQKSAYCYFSAAATIFSPELSDARISWAKNGVLTTLVDDFFDVGGSLEELKKLIELVEKWDINVSDGCCSEPVQILFSALHSTIQEIGDKAFKWQARSVTNHIIKIWLDLLNSMLREAEWARNATVPTVEEYMTNGYVSFALGPIILPALYLVGPKLSEELVKDSEFHSLFKLVSTCGRLLNDVHSFERESKAGQLNALSLRLIHGGVGITEAAAVAEMKSSIEKQRRELLRLVLRKEGSVVPRACKDLFWNMSRVLHQFYVKDDGFTSEEMIELVKSIIYEPIAVNEF

SEQ ID NO: 48 - DNA, 2454 bp, Chiococca alba
  source 1..2454: mol_type = other DNA
atgatgataatggtgatgaacacagctcccgtccacgcttaccacgctttacccattcccacccaaaaatcctcaaccacactttttcccaattataactgttccagtaggaagaaatcatcgccacctcgcatctctgccgcctcagtttctttgcaaactggagttgaaagaacgacggcaattcattcttcagacctagagatcaaagaaagaataaggaaactatttcatgatgttgatatctcgctttcttcatatgacactgcatgggtggcaatggtccctgctccacattcttcccagtctcccctttttccccagtgcattaattggttattggacaatcagcttcctgatggctcatggagtcttcctcctcatcatcatcatcatcatcccctattacttaaagatgcattatcctctacgcttgcatgtgttcttgcgctcaggagatggggaattggtcaagaacaagttgacaagggtattcgttttgttgagttaaattttgcttctgcatctgaccagaaccagcatttgccagttggatttgacattatattccctggcatgctcgaatatgctagagatttaaatttaaatcttcaactagaatccgcaactgtagatgccttacttctcaaaagagatcaggagcttataagattctttaaaagctactcagacgagagtaaagcataccttgcatatgtatcagaaggtatcataaagttacagaactgggatacagttatgaagttccaaagaaagaacgggtcactgttcaattcaccttcagctacagcagctgctgttatgcatgtccacaatcctggctgcctcgattaccttcactcagtgttggagaagcatggcaatgctgttccaacagtttaccctttggatatatatccacgcctctgcttggttgacaaccttgagagactgggtatttgtggtcattttaggaaggaaattctgagtgtattggatgatacatacagatgctggatgcagggggatgaagagatatttgcagaaaaatcaacttgtgccatagcatttacattattgcgaaagcatgggtacaacatctctgcagatccattgaccccattcttaaaggaagagtgtttttccaattctttgggtggatgtttgaaagatactagtgctgtacttgaattataccgggcattagagatgattattagccagaatgaatcagctctggtgaaaaaaagcttgtggtccagaagcttcctgaaagagcatatttctggtggttgtgatttaaagggattcagcaatcaaatttccaaacaggtggatgatatcctcaactttccatcgcatgctactttgcaacgggttgctaacaggagaagcatagagcaatacaacttagacagtacaaaaattttaaaaacttcatattgctcgtcgaattttagtaacaaagatttattgatcctggcagtcaaagattttaatcattgccaactcatacaccgtgaagaactgaaagaactagaaaggtgggtcgcagacaatagattggacaagttaaagtttgctaggcagaagtctgcatactgttacttttctgctgcagcaaccatattctcacctgaactttctgatgcccgcatctcatgggccaaaaatggtgtacttactactttggttgatgacttctttgacgtgggaggttctctagaggaattaaagaaactgattgagttggttgaaaagtgggatataaatgtcagtgatggttgttgctctgaaccagtgcaaatcctcttctcagcactacatagtacaatccaggagattggagataaagcattcaaatggcaagcacgcagtgtaacaaaccacataattaagatatggttagatttgcttaattctatgttgagggaagctgagtgggctagaaatgcaacagtgcctacagttgaagaatatatgacaaatggttatgtatcatttgccttggggccaattatcctccctgctctttatcttgttggacctaagctgtcagaggaattagttaaggattctgaattccactccctttttaagctagtgagtacctgtgggcggcttctgaatgatgtccacagcttcgagagggaatcaaaggccggccaactaaatgctctttctctgcgcctgattcatggtggagttggcattactgaagcagctgctgttgcagagatgaagagttcaattgagaagcaaaggagagaactgctgagactagtcttgcgcaaagagggtagtgtagttccaagagcttgcaaggatttgttttggaatatgagtagggtgctacatcaattttacgtcaaagatgatggatttacttcagaggagatgattgagcttgtgaagtcgatcatttatgagccaattgcggtcaatgaattttga

SEQ ID NO: 49 - protein, 796 aa, Chiococca alba
MIHTLPHGGQAHFISHKTQPYYSSRPRFSSAASLDTRVRRTSPSNSSVLDFNETKERITKLFHNVDYSISSYDTAWVAMVPDPHSSQAPLFPECINWLLDNQFHDGSWSLPHHNSLLLKDVLSSTLACVLALKRWGIGGRQIDKGVRFIEMNFGSASDNCQHTPIGFDIIFPGMLENARDLDLNLRLEPRIVTDMQRKRDMQLTRLHESDLKGDQAYLAYVSEGMQKLQNWDLAMKFQRKNGSLFNSPSATAAAVMHVQNPASLNYLHSVVDKFGHAVPAVYPLDLYARLCLVDNLERLGICRHFTNEIEIVMEDTYRCWLQDDEDIFAEISTCALAFRLLRKHGYVVSPDPLTKIIEEEDVSNSSGNGYWNDIHAVMEVHRASEVVIHENESDLKNQNTISKHLLRHHLFNGSDVKPFPNPIYKQVDYALKFPTPLILQRVENKTLIQNYDVDSTRLLKTSYRSSNFCNEDLLRLAVKDFNDCQLLHRKELKELERWSADNRLHELKFARQKAIYCSFSAAATIFIPEWYEARMSLAKNSVLATVVDDFFDVGGSMEELKKLIEFVEKWDIDITKESCSEPLKIIFSALHSTISEIGEQAVKWQGRNVTSHIIEIWLDLLNSMLRESEWTTDVHMPTLDEYMEAAYVSFAMGPIIIPALYFVGPKLSDEIVRDPEIRSLHKLVSICGRLLNDMQGFEREKKAGKPNAVSIRISQNGDGITESAAFEEVKMELEDARRELLRLVVQKDGSVVPRACKDAFWSVSRMLHHFYFNNDGYTSEVEMVELVNSIIHEPLK

SEQ ID NO: 50 - DNA, 2391 bp, Chiococca alba
  source 1..2391: mol_type = other DNA
atgattcatactctccctcatggcggccaggctcacttcatttcccacaaaacacagccttattattccagtagacctcgcttttcttcagcagcttctttggacacacgagtccggagaacatcgccctctaattcctctgtcctagacttcaacgagaccaaagaaagaatcacaaaattatttcataatgttgattattcaatttcttcatatgatacagcatgggttgctatggtcccggacccacattcttctcaggctccccttttcccagagtgcataaattggttgctagataatcaatttcatgatggctcctggagtcttcctcatcacaattctctattgcttaaggatgttttatcctctacgcttgcgtgtgttcttgctcttaagagatggggaataggaggaaggcagattgacaaaggtgttcgctttattgagatgaattttggctcagcatctgacaattgccagcatactccaataggatttgacataatatttccaggaatgcttgaaaatgccagagatttggatctaaatcttagactagaacccagaattgtaactgacatgcaacgtaaaagagacatgcagcttacaagactccatgaaagcgatctaaagggggaccaagcatacttggcatatgtatccgaagggatgcaaaagttacagaattgggatttggcgatgaagtttcaaaggaagaatggatcgctcttcaactcaccatcagctacagcagccgctgttatgcatgtccaaaatcctgcttccctcaattatcttcattcagtcgtcgacaaattcggccatgcagttccggctgtttaccctttggatctctatgcgcgcctttgcttggttgacaatcttgagaggctgggtatctgtcgacattttactaatgaaattgaaattgtaatggaggacacgtacaggtgctggctgcaggatgatgaagatatatttgccgaaatatcaacttgtgccttagcttttcggttattgagaaaacatggctatgttgtctccccagatccactgacaaaaatcatagaagaagaagatgtttccaattcttctggtaatggatattggaatgatatacatgctgtaatggaagtgcatcgggcatcagaggtggttatacatgaaaatgaatcagatttaaagaatcaaaataccatatcaaaacaccttctcagacaccatcttttcaatggttctgatgtgaagccctttcctaatccaatatacaagcaggtggactatgctctcaagtttccaacccccttaattctacaacgtgttgaaaacaagaccctcatacagaactacgacgtagacagtacaagacttcttaaaacttcatatcgatcatcaaatttctgcaatgaagatttactgaggttagcagtgaaagattttaatgactgtcaactcctgcaccggaaagaactaaaagaactagaaagatggtccgcagataacagactgcacgaactaaaatttgctcggcagaaagctatatactgctccttttctgctgcagcaacgattttcatacctgaatggtacgaagcccgcatgtcattggccaaaaatagtgtacttgctactgtggttgatgacttctttgatgtgggtggttcgatggaggaattaaagaagctaattgaatttgttgaaaagtgggatattgacatcaccaaggaatcctgctctgagccactcaaaatcatattttcagcactgcacagtacaatctctgagattggagagcaagcagttaaatggcaaggacgcaatgtaacaagccacataattgagatctggttggatttgctcaattcgatgttgagggagtctgaatggactacagatgtgcacatgccaacattggatgaatatatggaagctgcttatgtatcattcgccatggggccaattatcatccctgctctgtattttgttgggcctaagctatctgatgaaattgttcgggatcctgaaatacgatccctccataagcttgtgagcatttgtgggcggcttctaaatgatatgcaagggttcgagagggaaaagaaggctggtaaaccaaatgccgtgtctatacgcattagtcaaaatggtgatggcattaccgaatcagcagctttcgaagaagtgaagatggaattagaggatgcaaggagagaattgctaagattagttgtgcaaaaagatggtagtgtagttccaagagcttgcaaggatgcgttttggagcgtaagcagaatgttgcatcatttctacttcaataatgatggatacacgtcagaggtggagatggttgagctcgtgaattcaattattcatgaaccactaaaataa

SEQ ID NO: 51 - protein, 792 aa, Salvia hispanica
MSIQANMSFATSLHRSTTPGVGLPLKPCISPSPSLSFSPNFGTFNNTSLRLKPEAGSKSYEGIRRSHQLAASTILEGQTPITPEVESEKTRLIERIRSMLQDMDNDGQISVSPYDTAWVALVEDIGGSGGPQFPTSLEWISNHQYDDGSWGDRKFVLYDRILNTLACVVALTNWKMHPNKCEKGLRFIHENIKKLADEDEELMPVGFEIALPSVIDLAKRLGIEIPENSASIKRIYELRDSKLKKIPMDLVHKRPTSLLFSLEGMEGLNWDKLMNFLAEGSFLSSPSSTAYALQHTKNELCLEYLLKAVKRFNGGVPNAYPVDMFEHLWSVDRLQRLGISRYFQAEIEENMAYAYRYWTNKGITWARNMVVQDSDDSAQGFRLLRLYGYDIPIDVFKHFEQGGQFCSIPGQMTHAITGMYNLYRASELLFPGEHILSDARKYTGNFLHQRRITNTVVDKWIITKDLHGEVAYALDVPFYASLPRLEARFFIEQYGGDEDVWIGKTLYRMFKVNSDTYLEMAKLDYKQCQSVHQLEWNSMQRLYRDCNLGEFGLSERSLLLAYYIAASTTFEPEKSSERLAWAITTILVEIIASQKLSDEQKREFVDEFVKGSIVNNQNGGRHKPGNRLVEVLINNITLMAEGRGTYQQLSNAWKKWLKTWEEGGDLGEAEARLLLHTIHLSSGLDDSSFSHPKYQQLLEATSKVCHQLRVFQSVKVYDDQESTSQLVTRTTFQIEAGMQELVKLVFTKTLEDLPSTTKQSFFSVARSFYYTACIHADTIDSHINKVLFEKIV

SEQ ID NO: 52 - DNA, 2379 bp, Salvia hispanica
  source 1..2379: mol_type = other DNA
atgagtattcaagcaaacatgtcatttgccacctccctccaccgatcaaccacccccggagttggccttccgctaaaaccatgtatctctccctctccctctctttccttttccccaaactttggcacttttaacaacacaagtttgagactcaaaccagaggctgggagcaaaagttatgaggggattcgaagaagtcatcaattagcagcatcaacaattttggagggtcaaactccgattactccggaggttgaatcggagaaaacacgcctgattgaaaggattcgttcgatgttacaagacatggacaacgatggccagataagtgtgtcaccatacgacacagcatgggtggcgctcgtggaagatattggtggcagcggagggccacagtttccaacgagcctagagtggatttctaaccaccagtacgacgatggatcgtggggggatcgcaaatttgttctctatgaccggatactcaatacattagcatgtgttgtcgcactcacgaattggaaaatgcatcctaacaaatgcgaaaaagggttgaggtttattcatgagaatattaagaaactcgcggatgaagatgaagagctcatgcccgtaggattcgaaatcgcactgccatcagtcattgatttagctaaaagactgggtatagaaatcccagaaaattctgcaagcataaaaagaatttatgaattgagagattcaaaacttaaaaaaataccaatggatttagtgcacaaaaggcccacatcactactcttcagcttggaaggcatggaaggccttaactgggacaaactaatgaattttctagccgagggttcgtttctttcatcgccatcgtccactgcctacgctctccaacacaccaagaatgagttatgcctagagtatttactcaaggcagtcaagagattcaatggtggagttccaaatgcataccctgtcgacatgtttgagcatctgtggtccgtggatcgcttacagagattaggaatttctcggtattttcaagctgaaattgaagaaaacatggcctatgcttacagatactggacaaataaaggaatcacctgggcaagaaatatggttgtccaagacagtgacgacagcgcacagggattcaggctcttaaggttgtacggatacgatattcctatagatgttttcaaacatttcgagcaaggtggacaattctgcagcataccaggacagatgacacacgctattacaggaatgtacaacttgtatagagcttctgaacttctgttccctggagaacacatactttctgatgctagaaaatacacaggtaacttcttgcatcaaagaagaattactaacacggtagtagacaagtggatcattaccaaagaccttcacggcgaggtggcttatgcattggatgtgccattctacgccagtctgccacgactggaagcacgattcttcatagaacaatatgggggtgatgaagatgtttggattgggaaaacattgtacaggatgtttaaagtaaactccgacacataccttgagatggcaaaattagattacaaacaatgccagtctgtgcatcagttagagtggaatagcatgcaaagattgtatagagattgcaatctaggagagtttgggttgagcgaaagaagccttctcctagcttactacatagcagcctcaactacatttgagccggaaaaatcaagtgaaagactggcttgggctataacaacaattttagtcgaaataatcgcatcccaaaaactctctgatgagcaaaagagagagtttgttgatgaatttgtaaaaggaagcatcgtcaataaccaaaatggaggaagacataaaccgggaaacagattggttgaagttttgatcaacaatataacactgatggcagaaggcagaggcacatatcagcagttgtctaatgcgtggaaaaaatggctaaagacatgggaagagggaggtgacctgggggaagcagaagcacggcttctcctgcacacgatacatttgagctccggattggatgattcatcattttcccatccaaaatatcagcagctcttggaggcaaccagcaaagtctgccaccaacttcgcgtattccagagtgtaaaggtgtatgatgaccaagagtctacaagccaactggtaactaggacaactttccaaatagaagcaggcatgcaagaactagtgaaattagttttcacaaaaaccttggaagatttgccttctactaccaagcaaagcttttttagtgttgctagaagtttctattacactgcctgtattcatgcagacactatagactcccacataaacaaagtattgtttgaaaaaattgtctag

SEQ ID NO: 53 - protein, 790 aa, Teucrium canadense
MSFASQATSLLLSSHNATALPPLSAARLPPLTAGAAPFGRISFTTTSLRQYKLVSRAQSQEVDEIEKVTQVVLEAEKDIDQEAKVRELVENVRVKLQNIGEGGISISPYDTAWVALVEDVGGSGRPQFPESLDWISNHQFPDGSWGSHKFLYYDRVLCTLACIVALKTWNLHPHKFDKGLKFVRENIGKLADEEDVHMPIGFEVAFPSLIETAKRKGIDIPEDFPGKKEIYAKRDLKLKKIPMDILHKIPTPLLFSIEGIEGLDWQKLFKFRDHGSFLTSPSSTAHALQQTKDELCLKYLTNLVKKNNGGVPNAFPVDLFDRNYTVDRLRRLGILRYFQPEIEECMKYVYRFWDKRGISWARNTHVQDLDDTVQGFRNLRMHGYDVTLDVFKQFERCGEFFSFHGQSSDAVLGMFNLYRASQVLFPGEDMLADARKYAANYLHKRRVSNRVVDKWIINKDLPGEVAYGLDVPFYASLPRLEARFYVEQYGGNDDVWIGKALYRMLNVSCDTYLELAKLDYNICQAVHQKEWKSFQKWHRDGEFGLDEKSLLLAYYIAASTVFEPEKSLERLAWAKTAVLMEAILSQQLPSTKKHELVDEFKHASILNNQNGGSYKTRTPLVETLVNAISELSTTILLEQDRDIHLQLSNAWLKWLSRWEARGNLVEAEAELLLQTLHLSNGLEESSFSHPKYQQLLQVTSKVCHLLRLFQKRKVHDPEGCTTDIATGTTFQIEACMQQVVKLVFTKSSHDLDSVVKQRFLDVARSFYYTAHCDPQVIQSHINKVLFEKVV

SEQ ID NO: 54 - DNA, 2373 bp, Teucrium canadense
  source 1..2373: mol_type = other DNA
atgtcatttgcttcccaagccacctccctcctcctttcttcccacaacgccaccgctcttccgcctctctctgccgcccgccttccgcctctcactgccggtgctgctccattcggaagaatatcatttactactacctctcttcggcagtataaactggtgtcaagagctcaaagccaagaggtggatgagattgaaaaagtgacacaagtggtattggaggcagaaaaagacatcgatcaagaggcgaaggtaagggagctggtggaaaatgtccgagtgaagctgcaaaatatcggggaaggagggataagcatatcgccgtacgacaccgcatgggtggcgctggtggaggatgtcggcggcagcggcagaccgcagttcccggagagcctggattggatatcaaaccaccagttcccggacgggtcgtggggcagccacaaattcttgtactatgaccgggttttgtgcacgttagcatgtatagttgcattgaaaacttggaatctgcatcctcacaaattcgacaaagggttgaaattcgtcagagagaacattggaaagctcgcggatgaagaagacgtgcacatgccgattgggttcgaagtggcattcccatcacttatagagactgcaaagagaaaaggaattgacatcccggaagatttccctggcaagaaagaaatctatgcaaaaagagacctaaagctgaaaaagatacctatggatatactgcacaaaatccccacaccattactgttcagcatagaagggatagaaggccttgattggcagaagctattcaaattccgcgatcacggctccttcctcacgtccccgtcctcaacggcccacgctctccagcaaacaaaggacgagttatgcctcaaatatctgaccaatcttgtcaaaaagaacaatgggggagttccaaatgcatttccggtggacctatttgatcgtaactatacagtagatcgcctgaggaggctgggaattttgcgctattttcaacctgaaatcgaggaatgcatgaaatatgtatacagattctgggataaaagaggaatcagctgggcaagaaatacccatgttcaggaccttgatgataccgtacagggattcaggaacttaaggatgcatggttatgatgtcaccttagatgttttcaaacagttcgagagatgtggagaattctttagcttccacgggcaatcaagtgatgctgtcttaggaatgttcaacttgtaccgagcttctcaggttctgtttccaggagaagacatgcttgcagatgcaaggaagtacgcggccaactatttgcataaaagaagagttagtaatagggtcgtggacaaatggattattaacaaagatcttccaggcgaggtggcgtatgggctagatgttccgttctacgccagtctacctcgactggaagcaagattctacgtcgaacaatatgggggtaacgatgatgtctggattggaaaagctttatatagaatgttgaatgtgagctgtgatacttaccttgagctagcaaaattagactacaatatttgccaggctgtgcatcagaaagagtggaaaagctttcaaaaatggcacagggatggggagtttggattggatgaaaaaagcttacttttagcttactacatagcagcctcgactgttttcgagcctgaaaaatctctagagcgactggcttgggctaaaaccgcagttctaatggaggcaattttgtcccaacaacttcctagcacaaaaaaacatgagcttgttgacgaatttaaacatgcaagcatcctcaacaaccaaaatggaggaagctataaaacaagaactcctttggtagagactctagtaaacgccataagtgagctctcaactaccatactattggagcaagacagagacattcatctgcaattatctaatgcgtggctgaagtggctaagtagatgggaggcaagaggcaacctagtggaagcagaagcagagcttcttctgcaaaccttacatctgagcaatggattagaagaatcatcattttctcatccaaaatatcaacaactcttacaggttaccagcaaagtctgtcacctacttcggctattccagaaacgaaaggtgcatgatccggaagggtgtacaacagacattgcaacagggacaactttccaaatagaagcatgcatgcaacaagtagtgaaattagtgttcaccaaatcctcacatgatttagattctgttgttaagcagagatttttggatgttgccagaagtttctattacacagcccactgtgatccacaagtgatccagtcccacattaataaagtgttgtttgaaaaagtagtctag

SEQ ID NO: 55 - protein, 785 aa, Salvia officinalis
MSFASTTSLLRPSVTGFGVSPRVTSTSILSRSYGQILKGKTKYITDNRRNRQLAVKFEGQIALDLEDGVAKQTNQEAESEKIRQLKGKIRWILQNMEDGEMSVSPYDTAWVALVEDISGGGGPQFPTSLEWISKNQLADGSWGDPNYFLLYDRILNTLACVVALTTWNMHPHKCDQGLRFIRDNIEKLEDEDEELILVGFEIALPSLIDYAQNLGIQIQYDSPFIKKICAKRDLKLRKIPMDLMHRKPTSLLYSLEGMEGLEWEKLMNLRSEGSFLSSPSSTAYALQHTKDELCLDYLVKAVNKFNGGVPNVYPVDMYEHLWCVDRLQRLGISRYFQLEIQQCLDYVYRYWTNEGISWARYTNIRDSDDTAMGFRLLRLYGYDVSIDAFKPFEESGEFYSMAGQMNHAVTGMYNLYRASQLMFPQEHILSDARNFSAKFLHQKRRTNALVDKWIITKDLPGEVGYALDVPFYASLPRLEARFFLEQYGGDDDVWIGKTLYRMPYVNSNTYLELAKVDYKNCQSVHQLEWKSMQKWYRECNIGEFGLSERSLLLAYYIAASTTFEPEKSGERLAWATTAILIETIASQQLSDEQKREFVDEFENSIIIKNQNGGRYKARNRLVKVLINTVTLVAEGRGINQQLFNAWQKWLKTWEEGGDMGEAEAQLLLRTLHLSSGFDQSSFSHPKYEQLLEATSKVCHQLRLFQNRKVDDGQGCISRLVIGTTSQIEAGMQEVVKLVFTKTSQDLTSATKQSFFNIARSFYYTAYFHADTIDSHIYKVLFQTIV

SEQ ID NO: 56 - DNA, 2358 bp, Salvia officinalis
  source 1..2358: mol_type = other DNA
atgtcatttgcttccaccacctccctcctccgaccaagcgtcactgggttcggtgtttctccaagggttacttccacctccattcttagccgaagttatggtcaaatattaaaaggaaaaacaaaatacataactgataaccgtagaaatcgacaattggcggtaaaatttgagggccaaattgctttggatttggaggatggcgtagcaaagcagacgaatcaagaggcggaatctgagaagataaggcaactgaagggaaagatccgatggattctgcaaaacatggaggacggcgagatgagcgtgtcgccgtacgacaccgcatgggtggcgctggtggaagatatcagcggcggcggcgggccgcagttcccgacgagcctcgagtggatttccaagaatcagttggcggatgggtcatggggggatcctaattatttccttctctacgacagaatactcaatactttagcatgtgtagtcgcactcacgacttggaatatgcatcctcacaaatgcgatcaagggttgaggtttataagagacaacattgagaaacttgaggatgaagatgaggagctaattctcgtaggattcgagatcgcactgccttcactcattgattatgctcaaaaccttgggatacaaatccaatatgattctccattcattaaaaaaatttgtgcaaagagagatctaaaactcagaaaaataccaatggatttaatgcacagaaagccaacatcattgctctacagcttggaaggcatggaaggccttgagtgggaaaagctaatgaatttgcgatcggagggttcgtttctgtcatcgccgtcgtccacggcctacgctctccaacacaccaaggatgagttatgccttgactatctggtcaaggcggtcaacaaattcaatggtggagttcccaacgtgtaccctgtcgacatgtatgagcatctatggtgcgtagaccgcttgcagaggttgggaatttctcgctattttcaacttgaaattcaacaatgcctcgactatgtttacagatactggacaaatgaaggaatttcgtgggcaagatatactaatatccgggatagtgacgacaccgcaatgggattcaggcttctaaggttgtacggctatgatgtctctatagatgcttttaaaccattcgaggaaagcggagaattctatagcatggcagggcagatgaaccacgctgttacaggaatgtacaacttgtacagagcttctcaacttatgttccctcaagaacacatactttccgatgccagaaacttctctgccaaattcttgcatcaaaagaggcgtactaatgcactagtagacaagtggatcattaccaaagaccttcccggcgaggttggatatgcattggatgtgccgttctacgccagtctgcctcgactggaagcacgattcttcttagaacaatatgggggtgatgatgatgtttggattggaaaaactttgtacaggatgccatatgtgaactccaacacataccttgagcttgcaaaagtagactacaaaaactgccagtccgtgcatcagttggagtggaagagcatgcaaaaatggtacagagaatgcaatataggtgagtttgggttgagcgaaagaagccttctcctagcttactacatagcagcctcaactacattcgagccagaaaaatcaggtgagcggctcgcttgggctacaacagcaattttaatcgagacaatcgcgtcccaacaactctccgatgaacaaaagagagagttcgttgatgaatttgaaaacagcatcattatcaagaatcaaaatggagggagatataaagcaagaaacagattggtcaaggttttgatcaacactgtaacactggtagcagaaggcagaggcataaatcagcagttgtttaatgcgtggcaaaaatggctaaagacatgggaagaaggaggtgacatgggggaagcagaagcccagcttcttctgcgcacgctacatttgagctccggattcgatcaatcatcattttcccatccaaaatatgagcagctcttggaggcgaccagcaaagtttgccaccaacttcgcctattccagaatcgaaaggtggatgatggccaagggtgtataagtcgattggtaattgggacaacttcccaaatagaagcaggcatgcaagaagtagtgaaattagttttcaccaaaacctcacaagacttgacttctgctaccaagcaaagctttttcaatattgctagaagtttctattatactgcctactttcatgcagacactatagactcccacatatacaaagtattgtttcaaacaatagtatag

SEQ ID NO: 57 - protein, 808 aa, Scutellaria baicalensis
MPFLLPSSATSSPAFYTPAAPLAGHHVFPSFKPLIISRSSLQCNAISRPRTQEYIDVIQNGLPVIKWHEAVEEDETDKDSLNKEATSDKIRELVNLIRSMLQSMGDGEISSSPYDAAWVALVPDVGGSGGPQFPSSLEWISKNQLPDGSWGDTCTFSIYDRIINTLACVVALKSWNIHPHKTYQGISFIKANMDKLEDENEEHMPIGFEVALPSLIEIAKRLDIDISSDSRGLQEIYTRREVKLKRIPKEIMHQVPTTLLHSLEGMAELTWHKLLKLQCQDGSFLFSPSSTAFALHQTKDHNCLHYLTKYVHKFHGGVPNVYPVDLFEHLWAVDRIQRLGISRHFKPQVDECIAYVYRYWTDKGICWARNSVVQDLDDTAMGFRLLRLHGYDVSADVFKHFENGGEFFCFKGQSTQAVTGMYNLYRASQLMFPGESILEDAKTFSSKFLQRKRANNELLDKWIITKDLPGEVGYALDVPWYASLPRVETRFYLEQYGGEDDVWIGKTLYRMPYVNNNKYLELAKLDYSNCQSLHQQEWKNIQKWYESCNLGEFGLSERRVLLAYYVAAACIYEPEKSNQRLAWAKTVILMETITSYFEHQQLSAEQRRAFVNEFEHGSILKYANGGRYKRRSVLGTLLKTLNQLSLDILLTHGRNVHQPFKNAWHKWLKTWEEGGDIEEGEAEVLVRTLNLSGEGRHDSYVLEQSLLSQPIYEQLLKATMSVCKKLRLFQHRKDENGCMTKMRGITTLEIESEMQELVKLVFTKSSDDLDCEIKQNFFTIARSFYYVAYCNQGTINFHIAKVLFERVL

SEQ ID NO: 58 - DNA, 2427 bp, Scutellaria baicalensis
  source 1..2427: mol_type = other DNA
atgcctttcctcctcccttcctccgccaccagctcccccgcgttctatactccggccgcgcctctcgccggtcatcatgtttttccatctttcaagccactcattatttcccgttcttcactccaatgcaatgcaatctctcgacctcgtacccaagaatacatagatgtgattcagaatggattgccagtaataaagtggcacgaagctgtggaagaagatgagacagataaagattctcttaataaggaggccacgtcagacaagataagagagttggtaaatctgatccgttcgatgctccaatcaatgggcgacggagagataagctcgtcgccgtacgacgccgcatgggtggcgctggtgccggacgtcggcggctccggcgggccccagttcccctccagcctcgaatggatatccaaaaaccaactccccgacggctcctggggcgacacgtgtaccttttccatttatgatcgaatcatcaacacactggcttgcgttgttgctttgaaatcttggaacatacatccccacaaaacttatcaagggatttcattcataaaggcaaatatggacaaacttgaagacgagaacgaggagcacatgccgatcggatttgaagtggcactcccgtcgctaatcgagatagcgaaaaggctcgatatcgatatttccagcgattcgagagggctgcaagagatatacacgaggagggaggtaaagctgaaaaggataccgaaagagataatgcaccaagtgcccacaacactgcttcatagcttggagggtatggccgagctgacgtggcacaagcttttgaaattacagtgccaagatggctcctttcttttctctccatcttcaactgcctttgctcttcaccaaactaaggaccataattgtctccattatttgaccaaatatgttcacaaatttcatggtggagtgccaaatgtgtatccggtggacttgttcgagcatctatgggcagttgatcggatccaacggctggggatttcccggcatttcaagccccaagttgatgaatgtattgcctatgtttatagatattggacagataaaggaatatgctgggcaagaaattcagtagttcaagatcttgatgacacagccatgggattcaggcttcttaggttgcatggctacgatgtttcagcagatgttttcaaacattttgaaaatggtggagagttcttctgcttcaaagggcaaagcacgcaggcagtgactggaatgtacaatctgtacagagcttctcagttgatgtttcctggagaaagcatactggaagatgctaagaccttctcatctaagtttttgcaacgaaaacgagccaataacgagttgttagataagtggattattaccaaggatcttcctggagaggtgggatatgctctagatgtaccatggtatgctagcttacctagagttgaaactagattctacttggaacaatatggtggtgaagatgatgtttggattggcaaaactttatacaggatgccatatgttaacaataataaatatctagaactggcaaaattagactatagtaactgccagtcattacatcaacaagagtggaaaaacattcaaaaatggtatgagagttgcaatctgggagaatttggtttgagtgaaagaagggttctactagcctactacgtagctgctgcgtgtatatatgagcccgaaaagtcaaaccagcgcttggcttgggccaaaaccgtaattttaatggagactattacttcctattttgagcaccaacaactctccgcagaacagagacgcgcctttgttaatgaatttgaacatgggagtatcctcaaatatgcaaatggaggaagatacaaaaggaggagtgttttggggactttgctcaaaacactaaatcagctttcattggatatattattgacacacggtcgaaacgtccatcagcctttcaaaaatgcgtggcacaagtggctaaaaacgtgggaagaaggaggtgacattgaagaaggcgaagcagaggtattggtccgaaccctaaacctaagcggcgaagggaggcacgactcctatgtattggagcaatcattattgtcacaacctatatatgaacaacttttgaaagccaccatgagtgtttgcaagaagcttcgattgttccaacatcgaaaggatgagaatggatgtatgacgaagatgagaggcattacaacgttagagatagaatcggagatgcaagaattagtgaaattagtatttactaaatcctcagatgatttagattgtgaaattaaacaaaacttttttacaattgctaggagtttctattatgtggcttattgtaaccaaggaactatcaactttcacattgctaaggtgctctttgaaagagttctttag

SEQ ID NO: 59 - protein, 802 aa, Scutellaria baicalensis
MASLSTLSLNFSPAIHRKIQQSSAKLQFQGHCFTISSCMNNSKRLSLNHQSNHKRTSNVSELQVATLDAPQIREKEDYSTAQGYEKVDEVEDPIEYIRMLLNTTGDGRISVSPYDTAWIALIKDVEGRDAPQFPSSLEWIANNQLSDGSWGDEKFFCVYDRLVNTLACVVALRSWNIDAEKSEKGIRYIKENVDKLKDGNPEHMTCGFEVVFPSLLQRAQSMGIHDLPYDAPVIQDIYNTRESKLKRIPMEVMHKVPTSLLFSLEGLENLEWDKLLKLQSSDGSFLTSPSSTAYAFMHTKDPKCFEFIKNTVETFNGGAPHTYPVDVFGRLWAIDRLQRLGISRFFESEIADCLDHIYKYWTDKGVFSGRESDFVDVDDTSMGVRLLRMHGYQVDPNVLRNFKQGDKFSCYGGQMIESSSPIYNLYRASQLRFPGEDILEDANKFAYEFLQEQLSNNQLLDKWVISKHLPDEIKLGLQMPWYATLPRVEAKYYLQYYAGADDVWIGKTLYRMPEISNDTYLELARMDFKRCQAQHQFEWISMQEWYESCNIEEFGISRKELLQAYFLACSSVFELERTTERIGWAKSQIISRMIASFFNNETTTADEKDALLTRFRNINGPNKTKSGQRESEAVNMLVATLQQYLAGFDRYTRHQLKDAWSVWFRKVQEEEAIYGAEAELLTTTLNICAGHIAFDENIMANKDYTTLSSLTSKICQKLSEIRNEKVEEMESGIKAKSSIKDKEVEHDMQSLVKLVLERCEGINNRKLKQTFLSVAKTYYYRAYNADETMDIHMFKVLFEPVM

SEQ ID NO: 60 - DNA, 2409 bp, Scutellaria baicalensis
  source 1..2409: mol_type = other DNA
atggcctctctatcaactctgagcctcaacttttccccagcaattcaccgcaaaatacagcaatcatctgcaaaacttcagttccagggacattgtttcaccataagttcatgcatgaacaacagtaaaagactgtctttgaaccaccaatctaatcacaaaagaacgtcaaacgtatctgagctgcaagttgccactttggatgcgccccaaatacgtgaaaaagaagactactccactgctcaaggctatgagaaggtggatgaagtagaggatcctatcgaatatattagaatgctgttgaacacaacaggtgatgggcgaataagtgtgtcgccatacgacacagcctggatcgctcttattaaagacgtggaaggacgtgatgctccccagttcccatctagtctcgaatggattgccaataatcaactgagtgatgggtcgtggggcgatgagaagtttttctgtgtgtatgatcgccttgttaatacacttgcatgtgtcgtggcattgagatcatggaatattgatgctgaaaagagcgagaaaggaataagatacataaaagaaaacgtggataaactgaaagatgggaatccagagcacatgacctgtggttttgaggtggtgtttccttcccttcttcagagagcccaaagtatgggaattcatgatcttccctatgatgctcctgtcatccaagacatttacaataccagggagagtaaattgaaaaggattccaatggaggttatgcacaaggtgccaacatctctattgttcagcttggaaggattggagaatttggagtgggataagctcctcaaacttcagtcttctgatggttcattcctcacttctccatcctcaactgcctatgctttcatgcacactaaggaccctaaatgcttcgaattcatcaaaaacaccgtcgaaacatttaatggaggagcacctcatacttatccggtggatgtttttggaagactgtgggccattgacaggctgcagcgcctcggaatctctcgcttctttgagtccgagattgctgattgcttagatcacatctataaatattggacagacaaaggagtgttcagtggaagagaatcagattttgtggatgtggatgacacatccatgggtgttaggcttctaaggatgcacggatatcaagttgatccaaatgtattgaggaacttcaagcagggtgacaaattttcatgctatggtggtcaaatgatagagtcatcatctccgatatacaatctctatagggcttctcaactccgatttccaggagaagacattcttgaagatgccaacaaattcgcatacgagttcttgcaagaacagctatccaacaatcaacttttggacaaatgggttatatccaagcacttgcctgatgagataaagcttggattgcagatgccatggtatgccaccctaccccgagtggaggctaaatactacctacagtattatgctggtgctgatgatgtctggatcggcaagactctctacagaatgccagaaatcagtaatgatacatatctggagttagcaagaatggatttcaagagatgccaagcacagcatcaatttgagtggatttccatgcaagaatggtatgaaagttgcaacattgaagaatttgggataagcagaaaagagcttcttcaggcttactttttggcctgctcaagtgtatttgaactcgagaggacaacagagagaataggatgggccaaatcccaaattatttcaaggatgatagcttctttcttcaacaatgaaactacaacagccgatgaaaaagatgcacttttaaccagattcagaaacatcaatggcccaaacaaaacaaaaagtggtcagagagagagtgaagctgtgaacatgttggtagcaacgctccaacaatacctggcaggatttgatagatataccagacatcaattgaaagatgcttggagtgtgtggttcagaaaagtgcaagaagaagaggccatctacggggcagaagcggagcttctaacaaccaccttaaacatctgtgctggtcatattgctttcgacgaaaacataatggccaacaaagattacaccactctttccagccttacaagcaaaatttgccagaagctttctgaaattcgaaatgaaaaggttgaggaaatggagagtggaattaaagcaaaatcaagcatcaaagacaaggaagtggaacatgatatgcagtcactggtgaaattagtcctggagagatgtgaaggcataaacaacagaaaactgaagcaaacatttctatcggttgcaaaaacatattactacagagcctataatgctgatgaaaccatggacatccatatgttcaaagtacttttcgaaccagtcatgtga

SEQ ID NO: 61 - DNA, 24 bp, synthetic construct
  source 1..24: mol_type = other DNA
atggttctttcatcgtcttgcaca

SEQ ID NO: 62 - DNA, 25 bp, synthetic construct
  misc_feature 1..25: note = A synthetic oligonucleotide
  source 1..25: mol_type = other DNA
ttattttgcggcggaaacaggttca

SEQ ID NO: 63 - DNA, 30 bp, synthetic construct
  misc_feature 1..30: note = A synthetic oligonucleotide
  source 1..30: mol_type = other DNA
agattgaggattccattgagtacgtgaagg

SEQ ID NO: 64 - DNA, 30 bp, synthetic construct
  misc_feature 1..30: note = A synthetic oligonucleotide
  source 1..30: mol_type = other DNA
gaagtttaatatccttcattctttattaca

SEQ ID NO: 65 - DNA, 27 bp, synthetic construct
  misc_feature 1..27: note = A synthetic oligonucleotide
  source 1..27: mol_type = other DNA
agctccattcaactagagtcatgtcgt

SEQ ID NO: 66 - DNA, 28 bp, synthetic construct
  misc_feature 1..28: note = A synthetic oligonucleotide
  source 1..28: mol_type = other DNA
ttcatctggcttaactagttgctgacac

SEQ ID NO: 67 - DNA, 29 bp, synthetic construct
  misc_feature 1..29: note = A synthetic oligonucleotide
  source 1..29: mol_type = other DNA
ttaaagtactctctcaaagagtactttgg

SEQ ID NO: 68 - DNA, 21 bp, synthetic construct
  misc_feature 1..21: note = A synthetic oligonucleotide
  source 1..21: mol_type = other DNA
gcgaccaaccatcatacgact

SEQ ID NO: 69 - DNA, 25 bp, synthetic construct
  misc_feature 1..25: note = A synthetic oligonucleotide
  source 1..25: mol_type = other DNA
aatggcctccactgcatccactcta

SEQ ID NO: 70 - DNA, 26 bp, synthetic construct
  misc_feature 1..26: note = A synthetic oligonucleotide
  source 1..26: mol_type = other DNA
ccatactcattcaactggttcgaaca

SEQ ID NO: 71 - DNA, 21 bp, synthetic construct
  misc_feature 1..21: note = A synthetic oligonucleotide
  source 1..21: mol_type = other DNA
agcctgtgtactcgaaatgtc

SEQ ID NO: 72 - DNA, 24 bp, synthetic construct
  misc_feature 1..24: note = A synthetic oligonucleotide
  source 1..24: mol_type = other DNA
caagaggatgattcatgtaccaac

SEQ ID NO: 73 - DNA, 24 bp, synthetic construct
  misc_feature 1..24: note = A synthetic oligonucleotide
  source 1..24: mol_type = other DNA
tctctttcaagaatatcccctctc

SEQ ID NO: 74 - DNA, 22 bp, synthetic construct
  misc_feature 1..22: note = A synthetic oligonucleotide
  source 1..22: mol_type = other DNA
ggcattcaatgattttgagtcg

SEQ ID NO: 75 - DNA, 23 bp, synthetic construct
  misc_feature 1..23: note = A synthetic oligonucleotide
  source 1..23: mol_type = other DNA
aaatggcctctttgtccactctc

SEQ ID NO: 76 - DNA, 23 bp, synthetic construct
  misc_feature 1..23: note = A synthetic oligonucleotide
  source 1..23: mol_type = other DNA
ttacgcaactggttcgaaaagca

SEQ ID NO: 77 - DNA, 24 bp, synthetic construct
  misc_feature 1..24: note = A synthetic oligonucleotide
  source 1..24: mol_type = other DNA
taatgtcatttgcttcccaagcca

SEQ ID NO: 78 - DNA, 24 bp, synthetic construct
  misc_feature 1..24: note = A synthetic oligonucleotide
  source 1..24: mol_type = other DNA
ggcctagactaccttctcaaacaa

SEQ ID NO: 79 - DNA, 24 bp, synthetic construct
  misc_feature 1..24: note = A synthetic oligonucleotide
  source 1..24: mol_type = other DNA
aatgtcactctcgttcaccatcaa

SEQ ID NO: 80 - DNA, 25 bp, synthetic construct
  misc_feature 1..25: note = A synthetic oligonucleotide
  source 1..25: mol_type = other DNA
acttcaagaggatgaagtgtttagg

SEQ ID NO: 81 - DNA, 23 bp, synthetic construct
  misc_feature 1..23: note = A synthetic oligonucleotide
  source 1..23: mol_type = other DNA
ctccaaaactcgggccggtaaat

SEQ ID NO: 82 - DNA, 24 bp, synthetic construct
  misc_feature 1..24: note = A synthetic oligonucleotide
  source 1..24: mol_type = other DNA
tacgtatttcctcacaatcgagca

SEQ ID NO: 83 - DNA, 24 bp, synthetic construct
  misc_feature 1..24: note = A synthetic oligonucleotide
  source 1..24: mol_type = other DNA
ctagaaatgttacttgcgttcaac

SEQ ID NO: 84 - DNA, 25 bp, synthetic construct
  misc_feature 1..25: note = A synthetic oligonucleotide
  source 1..25: mol_type = other DNA
gggtaagagttgaatttagatgtct

SEQ ID NO: 85 - DNA, 30 bp, synthetic construct
  misc_feature 1..30: note = A synthetic oligonucleotide
  source 1..30: mol_type = other DNA
atgacttcaatatcctctctaaatttgagc

SEQ ID NO: 86 - DNA, 27 bp, synthetic construct
  misc_feature 1..27: note = A synthetic oligonucleotide
  source 1..27: mol_type = other DNA
gaatatagtaatcagacgaccggtcca

SEQ ID NO: 87 - DNA, 24 bp, synthetic construct
  misc_feature 1..24: note = A synthetic oligonucleotide
  source 1..24: mol_type = other DNA
gccatatcatgtctcttccgctct

SEQ ID NO: 88 - DNA, 29 bp, synthetic construct
  misc_feature 1..29: note = A synthetic oligonucleotide
  source 1..29: mol_type = other DNA
ttattcatgcaccttaaaatccttgagag

SEQ ID NO: 89 - DNA, 24 bp, synthetic construct
  misc_feature 1..24: note = A synthetic oligonucleotide
  source 1..24: mol_type = other DNA
atgaccgatgtatcctctcttcgt

SEQ ID NO: 90 - DNA, 23 bp, synthetic construct
  misc_feature 1..23: note = A synthetic oligonucleotide
  source 1..23: mol_type = other DNA
aaacactcacataaccggcccaa

SEQ ID NO: 91 - DNA, 20 bp, synthetic construct
  misc_feature 1..20: note = A synthetic oligonucleotide
  source 1..20: mol_type = other DNA
gtccttgctttcggaatact

SEQ ID NO: 92 - DNA, 25 bp, synthetic construct
  misc_feature 1..25: note = A synthetic oligonucleotide
  source 1..25: mol_type = other DNA
gaagtgatctacaaggattcataaa

SEQ ID NO: 93 - DNA, 23 bp, synthetic construct
  misc_feature 1..23: note = A synthetic oligonucleotide
  source 1..23: mol_type = other DNA
tcattgatttgccctgcatccac

SEQ ID NO: 94 - DNA, 23 bp, synthetic construct
  misc_feature 1..23: note = A synthetic oligonucleotide
  source 1..23: mol_type = other DNA
caaagctagtgctgcttctgatt

SEQ ID NO: 95 - DNA, 26 bp, synthetic construct
  misc_feature 1..26: note = A synthetic oligonucleotide
  source 1..26: mol_type = other DNA
atggtatctgcatgtctaaaactcaa

SEQ ID NO: 96 - DNA, 25 bp, synthetic construct
  misc_feature 1..25: note = A synthetic oligonucleotide
  source 1..25: mol_type = other DNA
ctttctctctcttgtgcatcttagt

SEQ ID NO: 97 - DNA, 23 bp, synthetic construct
  misc_feature 1..23: note = A synthetic oligonucleotide
  source 1..23: mol_type = other DNA
acgttcatcttcaatgagttcca

SEQ ID NO: 98 - DNA, 25 bp, synthetic construct
  misc_feature 1..25: note = A synthetic oligonucleotide
  source 1..25: mol_type = other DNA
tacgtgtatgtcgatctgttccaat

SEQ ID NO: 99 - DNA, 23 bp, synthetic construct
  misc_feature 1..23: note = A synthetic oligonucleotide
  source 1..23: mol_type = other DNA
catgtcatttgcttctcaatcac

SEQ ID NO: 100 - DNA, 26 bp, synthetic construct
  misc_feature 1..26: note = A synthetic oligonucleotide
  source 1..26: mol_type = other DNA
cccattatctaaaagtctacatcacc

SEQ ID NO: 101 - DNA, 23 bp, synthetic construct
  misc_feature 1..23: note = A synthetic oligonucleotide
  source 1..23: mol_type = other DNA
tcctcataaagcaatggcgtata

SEQ ID NO: 102 - DNA, 23 bp, synthetic construct
  misc_feature 1..23: note = A synthetic oligonucleotide
  source 1..23: mol_type = other DNA
ctaagattcagacaatgggctca

SEQ ID NO: 103 - DNA, 22 bp, synthetic construct
  misc_feature 1..22: note = A synthetic oligonucleotide
  source 1..22: mol_type = other DNA
gcagacgccaatctttcttggt

SEQ ID NO: 104 - DNA, 30 bp, synthetic construct
  misc_feature 1..30: note = A synthetic oligonucleotide
  source 1..30: mol_type = other DNA
ttatgaagttaaaaggagtggttcgttgac

SEQ ID NO: 105 - DNA, 21 bp, synthetic construct
  misc_feature 1..21: note = A synthetic oligonucleotide
  source 1..21: mol_type = other DNA
ggaacgagaaatgtcactcac

SEQ ID NO: 106 - DNA, 23 bp, synthetic construct
  misc_feature 1..23: note = A synthetic oligonucleotide
  source 1..23: mol_type = other DNA
ttctagtttctcacagaagtcaa

SEQ ID NO: 107 - DNA, 87 bp, synthetic construct
  misc_feature 1..87: note = A synthetic oligonucleotide
  source 1..87: mol_type = other DNA
tcaaatgcagcagacgaagttgctactcaacttttgaattttgacttgctgaagttggctggtgatgttgagtcaaaccctggacct

SEQ ID NO: 108 - DNA, 37 bp, synthetic construct
  misc_feature 1..37: note = A synthetic oligonucleotide
  source 1..37: mol_type = other DNA
ttctgcccaaattcgatggggtctctatccactatga

SEQ ID NO: 109 - DNA, 38 bp, synthetic construct
  misc_feature 1..38: note = A synthetic oligonucleotide
  source 1..38: mol_type = other DNA
agttaaaggcctcgatcaggcgactggttcgaaaagta

SEQ ID NO: 110 - DNA, 33 bp, synthetic construct
  misc_feature 1..33: note = A synthetic oligonucleotide
  source 1..33: mol_type = other DNA
ttctgcccaaattcgatgtcgctcgccttcaac

SEQ ID NO: 111 - DNA, 37 bp, synthetic construct
  misc_feature 1..37: note = A synthetic oligonucleotide
  source 1..37: mol_type = other DNA
agttaaaggcctcgatcaaaagacaaaggatttcata

SEQ ID NO: 112 - DNA, 38 bp, synthetic construct
  misc_feature 1..38: note = A synthetic oligonucleotide
  source 1..38: mol_type = other DNA
ttctgcccaaattcgatggttctttcatcgtcttgcac

SEQ ID NO: 113 - DNA, 37 bp, synthetic construct
  misc_feature 1..37: note = A synthetic oligonucleotide
  source 1..37: mol_type = other DNA
agttaaaggcctcgattattttgcggcggaaacaggt

SEQ ID NO: 114 - DNA, 39 bp, synthetic construct
  misc_feature 1..39: note = A synthetic oligonucleotide
  source 1..39: mol_type = other DNA
ttctgcccaaattcgatgaaaatgttgatgatcaaaagt

SEQ ID NO: 115 - DNA, 38 bp, synthetic construct
  misc_feature 1..38: note = A synthetic oligonucleotide
  source 1..38: mol_type = other DNA
agttaaaggcctcgatcagaccactggttcaaatagta

SEQ ID NO: 116 - DNA, 38 bp, synthetic construct
  misc_feature 1..38: note = A synthetic oligonucleotide
  source 1..38: mol_type = other DNA
ttctgcccaaattcgatgtcgtccctcgccggcaacct

SEQ ID NO: 117 - DNA, 37 bp, synthetic construct
  misc_feature 1..37: note = A synthetic oligonucleotide
  source 1..37: mol_type = other DNA
agttaaaggcctcgactagttgctgacacaactcatt

SEQ ID NO: 118 - DNA, 36 bp, synthetic construct
  misc_feature 1..36: note = A synthetic oligonucleotide
  source 1..36: mol_type = other DNA
ttctgcccaaattcgatgcaggcttctatgtcatct

SEQ ID NO: 119 - DNA, 37 bp, synthetic construct
  misc_feature 1..37: note = A synthetic oligonucleotide
  source 1..37: mol_type = other DNA
agttaaaggcctcgatcatacgactggttcaaacatt

SEQ ID NO: 120 - DNA, 33 bp, synthetic construct
  misc_feature 1..33: note = A synthetic oligonucleotide
  source 1..33: mol_type = other DNA
ttctgcccaaattcgatggcctccactgcatcc

SEQ ID NO: 121 - DNA, 36 bp, synthetic construct
  misc_feature 1..36: note = A synthetic oligonucleotide
  source 1..36: mol_type = other DNA
agttaaaggcctcgatcattcaactggttcgaacaa

SEQ ID NO: 122 - DNA, 34 bp, synthetic construct
  misc_feature 1..34: note = A synthetic oligonucleotide
  source 1..34: mol_type = other DNA
ttctgcccaaattcgatgattcctaatcccgaaa

SEQ ID NO: 123 - DNA, 36 bp, synthetic construct
  misc_feature 1..36: note = A synthetic oligonucleotide
  source 1..36: mol_type = other DNA
agttaaaggcctcgattacattggcaatccgatgaa

SEQ ID NO: 124 - DNA, 35 bp, synthetic construct
  misc_feature 1..35: note = A synthetic oligonucleotide
  source 1..35: mol_type = other DNA
ttctgcccaaattcgatgtcggtggcgttcaacct

SEQ ID NO: 125 - DNA, 37 bp, synthetic construct
  misc_feature 1..37: note = A synthetic oligonucleotide
  source 1..37: mol_type = other DNA
agttaaaggcctcgatcaagaggatgattcatgtacc

SEQ ID NO: 126 - DNA, 34 bp, synthetic construct
  misc_feature 1..34: note = A synthetic oligonucleotide
  source 1..34: mol_type = other DNA
ttctgcccaaattcgatgtccctcgccttcaacg

SEQ ID NO: 127 - DNA, 34 bp, synthetic construct
  misc_feature 1..34: note = A synthetic oligonucleotide
  source 1..34: mol_type = other DNA
agttaaaggcctcgatcatttgccactcacattt

SEQ ID NO: 128 - DNA, 37 bp, synthetic construct
  misc_feature 1..37: note = A synthetic oligonucleotide
  source 1..37: mol_type = other DNA
ttctgcccaaattcgatggcctctttgtccactttcc

SEQ ID NO: 129 - DNA, 37 bp, synthetic construct
  misc_feature 1..37: note = A synthetic oligonucleotide
  source 1..37: mol_type = other DNA
agttaaaggcctcgatcacgcaactggttcgaaaaga

SEQ ID NO: 130 - DNA, 38 bp, synthetic construct
  misc_feature 1..38: note = A synthetic oligonucleotide
  source 1..38: mol_type = other DNA
ttctgcccaaattcgatgtcatttgcttcccaagccac

SEQ ID NO: 131 - DNA, 39 bp, synthetic construct
  misc_feature 1..39: note = A synthetic oligonucleotide
  source 1..39: mol_type = other DNA
agttaaaggcctcgactagactaccttctcaaacaatac

SEQ ID NO: 132 - DNA, 37 bp, synthetic construct
  misc_feature 1..37: note = A synthetic oligonucleotide
  source 1..37: mol_type = other DNA
ttctgcccaaattcgatgtcactctcgttcaccatca

SEQ ID NO: 133 - DNA, 36 bp, synthetic construct
  misc_feature 1..36: note = A synthetic oligonucleotide
  source 1..36: mol_type = other DNA
agttaaaggcctcgatcaagaggatgaagtgtttag

SEQ ID NO: 134 - DNA, 37 bp, synthetic construct
  misc_feature 1..37: note = A synthetic oligonucleotide
  source 1..37: mol_type = other DNA
ttctgcccaaattcgatgacctctatgtcctctctaa

SEQ ID NO: 135 - DNA, 37 bp, synthetic construct
  misc_feature 1..37: note = A synthetic oligonucleotide
  source 1..37: mol_type = other DNA
agttaaaggcctcgatcatacgaccggtccaaacagt

SEQ ID NO: 136 - DNA, 39 bp, synthetic construct
  misc_feature 1..39: note = A synthetic oligonucleotide
  source 1..39: mol_type = other DNA
ttctgcccaaattcgatgttacttgcgttcaacataagc

SEQ ID NO: 137 - DNA, 38 bp, synthetic construct
  misc_feature 1..38: note = A synthetic oligonucleotide
  source 1..38: mol_type = other DNA
agttaaaggcctcgattaattaggtaggtagaggggtt

SEQ ID NO: 138 - DNA, 52 bp, synthetic construct
  misc_feature 1..52: note = A synthetic oligonucleotide
  source 1..52: mol_type = other DNA
atattctgcccaaattcgatgacttcaatatcctctctaaatttgagcaatg

SEQ ID NO: 139 - DNA, 35 bp, synthetic construct
  misc_feature 1..35: note = A synthetic oligonucleotide
  source 1..35: mol_type = other DNA
cagagttaaaggcctcgatcagacgaccggtccaa

SEQ ID NO: 140 - DNA, 36 bp, synthetic construct
  misc_feature 1..36: note = A synthetic oligonucleotide
  source 1..36: mol_type = other DNA
ttctgcccaaattcgatgtctcttccgctctcctct

SEQ ID NO: 141 - DNA, 49 bp, synthetic construct
  misc_feature 1..49: note = A synthetic oligonucleotide
  source 1..49: mol_type = other DNA
gataagttaaaggcctcgattattcatgcaccttaaaatccttgagagc

SEQ ID NO: 142 - DNA, 37 bp, synthetic construct
  misc_feature 1..37: note = A synthetic oligonucleotide
  source 1..37: mol_type = other DNA
ttctgcccaaattcgatgaccgatgtatcctctcttc

SEQ ID NO: 143 - DNA, 35 bp, synthetic construct
  misc_feature 1..35: note = A synthetic oligonucleotide
  source 1..35: mol_type = other DNA
agttaaaggcctcgatcacataaccggcccaaaca

SEQ ID NO: 144 - DNA, 35 bp, synthetic construct
  misc_feature 1..35: note = A synthetic oligonucleotide
  source 1..35: mol_type = other DNA
ttctgcccaaattcgatggcgtcgctcgcgttcac

SEQ ID NO: 145 - DNA, 39 bp, synthetic construct
  misc_feature 1..39: note = A synthetic oligonucleotide
  source 1..39: mol_type = other DNA
agttaaaggcctcgactacaaggattcataaattaagga

SEQ ID NO: 146 - DNA, 36 bp, synthetic construct
  misc_feature 1..36: note = A synthetic oligonucleotide
  source 1..36: mol_type = other DNA
ttctgcccaaattcgcgaatgtcactcgccttcagc

SEQ ID NO: 147 - DNA, 37 bp, synthetic construct
  misc_feature 1..37: note = A synthetic oligonucleotide
  source 1..37: mol_type = other DNA
agttaaaggcctcgagctaggagcttagggttttcat

SEQ ID NO: 148 - DNA, 35 bp, synthetic construct
  misc_feature 1..35: note = A synthetic oligonucleotide
  source 1..35: mol_type = other DNA
ttctgcccaaattcgatggtatctgcatgtctaaa

SEQ ID NO: 149 - DNA, 35 bp, synthetic construct
  misc_feature 1..35: note = A synthetic oligonucleotide
  source 1..35: mol_type = other DNA
agttaaaggcctcgatcatgaaggaattgaaggaa

SEQ ID NO: 150 - DNA, 39 bp, synthetic construct
  misc_feature 1..39: note = A synthetic oligonucleotide
  source 1..39: mol_type = other DNA
ttctgcccaaattcgatgagttccattcgaaatttaagt

SEQ ID NO: 151 - DNA, 39 bp, synthetic construct
  misc_feature 1..39: note = A synthetic oligonucleotide
  source 1..39: mol_type = other DNA
agttaaaggcctcgatcacttgagaggctcaaacatcat

SEQ ID NO: 152 - DNA, 37 bp, synthetic construct
  misc_feature 1..37: note = A synthetic oligonucleotide
  source 1..37: mol_type = other DNA
ttctgcccaaattcgatgtcatttgcttctcaatcac

SEQ ID NO: 153 - DNA, 39 bp, synthetic construct
  misc_feature 1..39: note = A synthetic oligonucleotide
  source 1..39: mol_type = other DNA
agttaaaggcctcgactacatcaccctctcaaacaatac

SEQ ID NO: 154 - DNA, 45 bp, synthetic construct
  misc_feature 1..45: note = A synthetic oligonucleotide
  source 1..45: mol_type = other DNA
ttctgcccaaattcgatggcgtatatgatatctatttcaaatctc

SEQ ID NO: 155 - DNA, 39 bp, synthetic construct
  misc_feature 1..39: note = A synthetic oligonucleotide
  source 1..39: mol_type = other DNA
agttaaaggcctcgatcagacaatgggctcaaatagaac

SEQ ID NO: 156 - DNA, 37 bp, synthetic construct
  misc_feature 1..37: note = A synthetic oligonucleotide
  source 1..37: mol_type = other DNA
ttctgcccaaattcgatgcaagtctctctctccctca

SEQ ID NO: 157 - DNA, 38 bp, synthetic construct
  misc_feature 1..38: note = A synthetic oligonucleotide
  source 1..38: mol_type = other DNA
agttaaaggcctcgattatgaagttaaaaggagtggtt

SEQ ID NO: 158 - DNA, 37 bp, synthetic construct
  misc_feature 1..37: note = A synthetic oligonucleotide
  source 1..37: mol_type = other DNA
ttctgcccaaattcgcgaatgtcactcactttcaacg

SEQ ID NO: 159 - DNA, 37 bp, synthetic construct
  misc_feature 1..37: note = A synthetic oligonucleotide
  source 1..37: mol_type = other DNA
agttaaaggcctcgagctagtttctcacagaagtcaa

SEQ ID NO: 160 - DNA, 35 bp, synthetic construct
  misc_feature 1..35: note = A synthetic oligonucleotide
  source 1..35: mol_type = other DNA
aggagatataccatggccgagattcgagttgccac

SEQ ID NO: 161 - DNA, 37 bp, synthetic construct
  misc_feature 1..37: note = A synthetic oligonucleotide
  source 1..37: mol_type = other DNA
ggtggtggtgctcgaaggcgactggttcgaaaagtac

SEQ ID NO: 162 - DNA, 40 bp, synthetic construct
  misc_feature 1..40: note = A synthetic oligonucleotide
  source 1..40: mol_type = other DNA
aggagatataccatggatttcatggcgaaaatgaaagaga

SEQ ID NO: 163 - DNA, 36 bp, synthetic construct
  misc_feature 1..36: note = A synthetic oligonucleotide
  source 1..36: mol_type = other DNA
ggtggtggtgctcgaaaaagacaaaggatttcatat

SEQ ID NO: 164 - DNA, 40 bp, synthetic construct
  misc_feature 1..40: note = A synthetic oligonucleotide
  source 1..40: mol_type = other DNA
aggagatataccatgcaaattcgtggaaagcaaagatcac

SEQ ID NO: 165 - DNA, 38 bp, synthetic construct
  misc_feature 1..38: note = A synthetic oligonucleotide
  source 1..38: mol_type = other DNA
ggtggtggtgctcgaagaccactggttcaaatagaact

SEQ ID NO: 166 - DNA, 35 bp, synthetic construct
  misc_feature 1..35: note = A synthetic oligonucleotide
  source 1..35: mol_type = other DNA
aggagatataccatgtctaaatcatctgcagctgt

SEQ ID NO: 167 - DNA, 35 bp, synthetic construct
  misc_feature 1..35: note = A synthetic oligonucleotide
  source 1..35: mol_type = other DNA
ggtggtggtgctcgaagttgctgacacaactcatt

SEQ ID NO: 168 - DNA, 30 bp, synthetic construct
  misc_feature 1..30: note = A synthetic oligonucleotide
  source 1..30: mol_type = other DNA
aggagatataccatgaccgtcaaatgctac

SEQ ID NO: 169 - DNA, 35 bp, synthetic construct
  misc_feature 1..35: note = A synthetic oligonucleotide
  source 1..35: mol_type = other DNA
ggtggtggtgctcgaacaaggattcataaattaag

SEQ ID NO: 170 - DNA, 30 bp, synthetic construct
  misc_feature 1..30: note = A synthetic oligonucleotide
  source 1..30: mol_type = other DNA
aggagatataccatgactgtcaagtgcagc

SEQ ID NO: 171 - DNA, 30 bp, synthetic construct
  misc_feature 1..30: note = A synthetic oligonucleotide
  source 1..30: mol_type = other DNA
ggtggtggtgctcgaatgaaggaattgaag

SEQ ID NO: 172 - DNA, 40 bp, synthetic construct
  misc_feature 1..40: note = A synthetic oligonucleotide
  source 1..40: mol_type = other DNA
aggagatataccatgtttatgcccacttccattaaatgta

SEQ ID NO: 173 - DNA, 42 bp, synthetic construct
  misc_feature 1..42: note = A synthetic oligonucleotide
  source 1..42: mol_type = other DNA
ggtggtggtgctcgaacatcaccctctcaaacaatactttgg

SEQ ID NO: 174 - DNA, 43 bp, synthetic construct
  misc_feature 1..43: note = A synthetic oligonucleotide
  source 1..43: mol_type = other DNA
aggagatataccatggtagcaaaagtgatcgagagccgagtta

SEQ ID NO: 175 - DNA, 44 bp, synthetic construct
  misc_feature 1..44: note = A synthetic oligonucleotide
  source 1..44: mol_type = other DNA
ggtggtggtgctcgaagacaatgggctcaaatagaactttaaat

SEQ ID NO: 176 - protein, 575 aa, Salvia sclarea
MSLAFNVGVTPFSGQRVGSRKEKFPVQGFPVTTPNRSRLIVNCSLTTIDFMAKMKENFKREDDKFPTTTTLRSEDIPSNLCIIDTLQRLGVDQFFQYEINTILDNTFRLWQEKHKVIYGNVTTHAMAFRLLRVKGYEVSSEELAPYGNQEAVSQQTNDLPMIIELYRAANERIYEEERSLEKILAWTTIFLNKQVQDNSIPDKKLHKLVEFYLRNYKGITIRLGARRNLELYDMTYYQALKSTNRFSNLCNEDFLVFAKQDFDIHEAQNQKGLQQLQRWYADCRLDTLNFGRDVVIIANYLASLIIGDHAFDYVRLAFAKTSVLVTIMDDFFDCHGSSQECDKIIELVKEWKENPDAEYGSEELEILFMALYNTVNELAERARVEQGRSVKEFLVKLWVEILSAFKIELDTWSNGTQQSFDEYISSSWLSNGSRLTGLLTMQFVGVKLSDEMLMSEECTDLARHVCMVGRLLNDVCSSEREREENIAGKSYSILLATEKDGRKVSEDEAIAEINEMVEYHWRKVLQIVYKKESILPRRCKDVFLEMAKGTFYAYGINDELTSPQQSKEDMKSFVF

SEQ ID NO: 177 - DNA, 1728 bp, Salvia sclarea
  source 1..1728: mol_type = other DNA
atgtcgctcgccttcaacgtcggagttacgcctttctccggccaaagagttgggagcaggaaagaaaaatttccagtccaaggatttcctgtgaccacccccaataggtcacgtctcatcgttaactgcagccttactacaatagatttcatggcgaaaatgaaagagaatttcaagagggaagacgataaatttccaacgacaacgactcttcgatccgaagatataccctctaatttgtgtataatcgacacccttcaaaggttgggggtcgatcaattcttccaatatgaaatcaacactattctagataacacattcaggttgtggcaagaaaaacacaaagttatatatggcaatgttactactcatgcaatggcatttaggcttttgcgagtgaaaggatacgaagtttcatcagaggagttggctccatatggtaaccaagaggctgttagccagcaaacaaatgacctgccgatgattattgagctttatagagcagcaaatgagagaatatatgaagaagagaggagtcttgaaaaaattcttgcttggactaccatctttctcaataagcaagtgcaagataactcaattcccgacaaaaaactgcacaaactggtggaattctacttgaggaattacaaaggcataaccataagattgggagctagacgaaacctcgagctatatgacatgacctactatcaagctctgaaatctacaaacaggttctctaatttatgcaacgaagattttctagttttcgcaaagcaagatttcgatatacatgaagcccagaaccagaaaggacttcaacaactgcaaaggtggtatgcagattgtaggttggacaccttaaactttggaagagatgtagttattattgctaattatttggcttcattaattattggtgatcatgcgtttgactatgttcgtctcgcatttgccaaaacatctgtgcttgtaacaattatggatgattttttcgactgtcatggctctagtcaagagtgtgacaagatcattgaattagtaaaagaatggaaggagaatccggatgcagagtacggatctgaggagcttgagatcctttttatggcgttgtacaatacagtaaatgagttggcggagagggctcgtgttgaacaggggcgtagtgtcaaagagtttctagtcaaactgtgggttgaaatactctcagctttcaagatagaattagatacatggagcaatggcacgcagcaaagcttcgatgaatacatttcttcgtcgtggttgtcgaacggttcccggctgacaggtctcctgacgatgcaattcgtcggagtaaaattgtccgatgaaatgcttatgagtgaagagtgcactgatttggctaggcatgtctgtatggtcggccggctgctcaacgacgtgtgcagttctgagagggagcgcgaggaaaatattgcaggaaaaagttatagcattctactagcaactgagaaagatggaagaaaagttagtgaagatgaagccattgcagagatcaatgaaatggttgaatatcactggagaaaagtgttgcagattgtgtataaaaaagaaagcattttgccaagaagatgcaaagatgtatttttggagatggctaagggtacgttttatgcttatgggatcaacgatgaattgacttctcctcagcaatccaaggaagatatgaaatcctttgtcttttga

(19) United States
(12) Patent Application Publication
(10) Pub. No.: US 2024/0124899 A1
(43) Pub. Date: Apr. 18, 2024

(54) METHODS FOR PRODUCTION OF NOVEL DITERPENE SCAFFOLDS

(71) Applicant: Board of Trustees of Michigan State University, East Lansing, MI (US)

(72) Inventors: Björn Hamberger, Okemos, MI (US); Sean Johnson, Bedford, MA (US); Wajid Waheed Bhat, Haslett, MI (US)

(21) Appl. No.: 18/458,762

(22) Filed: Aug. 30, 2023

Related U.S. Application Data
(62) Division of application No. 17/265,482, filed on Feb. 2, 2021, now Pat. No. 11,827,915.
(60) Provisional application No. 62/714,216, filed on Aug. 3, 2018.

(51) Int. Cl. C12P 5/00; C12N 9/10; C12N 15/82; C12P 17/02; C12P 17/06

(52) U.S. Cl. CPC C12P 5/002; C12N 9/1022; C12N 9/1085; C12N 15/8243; C12P 17/02; C12P 17/06; C12Y 202/01007; C12Y 205/01029

(57) ABSTRACT

Enzymes and methods are described herein for manufacturing terpenes, including terpenes.

This application is a divisional of U.S. application Ser. No. 17/265,482, filed Feb. 2, 2021, which is a U.S. national stage filing under 35 U.S.C. 371 from International Application No. PCT/US2019/044887, filed on 2 Aug. 2019, and published as WO 2020/028795 A1 on 6 Feb. 2020, which claims the benefit of U.S. Provisional Application Ser. No. 62/714,216, filed Aug. 3, 2018, which applications are incorporated by reference herein their entirety.

### GOVERNMENT FUNDING

This invention was made with government support under 1737898 awarded by the National Science Foundation, and under DE-FC02-07ER64494 and DE-SC0018409 awarded by the U.S. Department of Energy. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Dec. 4, 2023, is named “2390069.xml” and is 293,571 bytes in size.

### BACKGROUND

Plant-derived terpenoids have a wide range of commercial and industrial uses. Examples of uses for terpenoids include specialty fuels, agrochemicals, fragrances, nutraceuticals and pharmaceuticals. However, currently available methods for petrochemical synthesis, extraction, and purification of terpenoids from the native plant sources have limited economic sustainability.

### SUMMARY

Described herein are enzymes useful for production of a variety of terpenes, diterpenes and terpenoids. In some cases, the enzymes synthesize diterpenes. The enzymes were isolated from the mint family (Lamiaceae). Members of the mint family accumulate a wide variety of industrially and medicinally relevant diterpenes. While there are more than 7000 plant species in Lamiaceae, diterpene synthase (diTPS) genes have been characterized from just eleven. The Mint Evolutionary Genomics Consortium (see website at mints.plantbiology.msu.edu) has now sequenced leaf transcriptomes from at least 48 phylogenetically diverse Lamiaceae species, more than doubling the number of mint species for which transcriptomes are available. The available chemotaxonomic and enzyme activity data are described herein for diterpene synthases (diTPSs) in Lamiaceae. The diTPS sequences and terpenes produced are also described herein. One of the new enzymes produces neo-cleroda-4(18),13E-dienyl diphosphate, a molecule with promising applications in agricultural biotechnology as a precursor to potent insect anti-feedants.

Described herein are expression systems that include at least one expression cassette having at least one heterologous promoter operably linked to at least one nucleic acid segment encoding an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176. In some cases, the expression systems can have more than one expression cassettes or expression vectors, each expression cassette or expression vector can have at least one nucleic acid segment encoding an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176. Host cells that include such expression systems are also described herein.

Methods are also described herein that include incubating a host cell comprising a heterologous expression system that includes at least one expression cassette having a heterologous promoter operably linked to a nucleic acid segment encoding an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176. The expression system within host cell can include more than one expression cassettes or expression vectors.

In addition, methods are described herein for synthesizing a diterpene comprising incubating a terpene precursor with at least one enzyme having at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176. Such methods can include incubating more than one terpene precursor and/or incubating more than one enzyme in a mixture to produce one or more terpenes or terpenoid compounds.

A variety of diterpenes are also described herein.

### DESCRIPTION OF THE FIGURES

FIG. 1A-1D illustrate the distribution of diterpenes in Lamiaceae. Note that Table 4 provides a comparison of different sources for data about Lamiaceae diterpene chemotaxonomy. FIG. 1A illustrates diterpene skeletons per genus according to both the Dictionary of Natural Products (DNP) and SISTEMAT. FIG. 1B illustrates the distribution of skeletons among Lamiaceae clades and genera, based on the DNP. Structures are shown for selected skeletons, where black structures are those where a biosynthetic route is known from Lamiaceae, and gray structures are those for which the pathway remains unknown. FIG. 1C illustrates the distribution of compounds among skeletons, based on the DNP. FIG. 1D illustrates diterpene structures per genus according to both the DNP and the NAPRALERT database. Darker spots indicate overlapping data points, some labels omitted due to space constraints.

FIG. 2A-2B illustrate maximum likelihood trees of diterpene synthase (diTPS) enzymes. FIG. 2A shows a maximum likelihood tree of newly characterized (blue) class II diTPS enzymes. FIG. 2B shows a maximum likelihood tree of newly characterized (blue) class I diTPS enzymes. The maximum likelihood tree of newly characterized (blue) class 11 and class I diTPS enzymes are shown in the context of previously reported (black) diTPSs from Lamiaceae. The bifunctional ent-kaurene synthase from Physcomitrella patens was used as an outgroup. After each enzyme type are listed the experimentally verified substrates (green) and their products, where the numbers correspond to compound numbers in FIG. 3. Units for scale bars are substitutions per site. Abbreviations for species are listed in Table 5 and those not listed in Table 5 are as follows: Ie, Isodon eriocalyx, Ir, Isodon rubescens; Mv, Marrubium vulgare, Sd, Salvia divinorum; Sm, Salvia miltiorriza, Sp, Salvia pomifera, Ss, Salvia sclarea, Vac, Vitex agnus-castus.

FIG. 3A-3B show structures of products of diterpene synthases from Lamiaceae and a phylogenetic tree was generated from the peptide sequences. FIG. 3A shows products of diterpene synthases from Lamiaceae. Blue numbers indicate compounds experimentally verified to be products of new enzymes identified using the methods described herein. At the center is geranylgeranyl diphosphate (GGPP), a precursor to all of these diterpenes. The inner ring are class II products, the product show in the outer ring are class I products derived from the compound in the connected segment of the inner ring. FIG. 3B(A) to 3B(H) show overlapping portions of a phylogenetic tree generated from the peptide sequences from the reference set, alongside those from the new transcriptome data, including established substrates and products for each enzyme.

FIG. 4A-4C illustrate results of activity assays for several enzymes. FIG. 4A shows products detected by gas chromatography from activity assays of Ajuga reptans cleroda-4(18),13E-dienyl diphosphate synthase (ArTPS2) and Salvia sclarea sclareol synthase (SsSS) in-vitro with purified protein contacted with GGPP, and in-vivo of N. benthamiana cells that transiently expressed the gene combinations. FIG. 4B shows products detected by gas chromatography from activity assays of PcTPS1+SsSS, In-vitro with purified protein contacted with GGPP, and in-vivo of N. benthamiana cells that transiently expressed the gene combinations. FIG. 4C shows mass spectra for the products of ArTPS2 and PcTPS1, and their combinations with SsSS.

FIG. 5A-5B illustrates the structures that can be produced by the activities of new class I diTPSs. FIG. 5A shows structures that can be generated by the activities of new class I diTPSs. Filled in blue boxes indicate which enzymes are capable of each conversion. FIG. 5B illustrates structures that can be produced by the newly characterized enzyme activities including some of the new class II enzymes. Blue genes are newly characterized. Blue square: TPS-e from that position on the key catalyzes the shown transformation. White square: corresponding TPS-e does not catalyze the shown activity. Grey square: corresponding TPS-e was not tested on the substrate.

FIG. 6A-6C illustrate analysis of compounds from O. majorana. FIG. 6A shows GC total ion chromatograms of extracts from N. benthamiana expressing OmTPS1 and OmTPS5, compared to extracts of various tissues of O. majorana. FIG. 6B shows a mass spectrum of peak B, from O. majorana leaf (where peak B is shown in FIG. 6A). FIG. 6C shows a mass spectrum of peak C from a O. majorana leaf compared to reference spectrum for palustrinol from the NIST17 library (where peak C is shown in FIG. 6A).

FIG. 7A-7C illustrate the activities of novel Chiococca alba terpene synthases CaTPS1-5. FIG. 7A shows GC-MS-total ion and extracted ion chromatograms illustrating production of ent-kaurene (identified from peak 1) from in vivo assays in N. benthamiana transiently expressing the gene combinations shown. The mass spectrum of peak 1 is shown below the chromatograms, demonstrating that peak 1 is ent-kaurene as identified through direct comparison with biosynthesized authentic standards with reference enzymes. FIG. 7B shows GC-MS-total ion and extracted ion chromatograms illustrating production of ent-dolabradiene (identified from peak 2) from in vivo assays in N. benthamiana transiently expressing the gene combinations shown. The mass spectrum of peak 2 is shown below the chromatograms, demonstrating that peak 2 is ent-dolabradiene as identified through direct comparison with biosynthesized authentic standards with reference enzymes. FIG. 7C shows GC-MS-total ion and extracted ion chromatograms illustrating production of (13R)-ent-manoyl oxide (identified from peak 3) from in vivo assays in N. benthamiana transiently expressing the gene combinations shown. The mass spectrum of peak 3 is shown below the chromatograms, demonstrating that peak 3 is (13R)-ent-manoyl oxide as identified through direct comparison with biosynthesized authentic standards with reference enzymes.

### DETAILED DESCRIPTION

Described herein are new enzymes and compounds, as well as methods that are useful for manufacturing such compounds. The compounds that can be made by the enzymes and methods are new compounds and compounds that were previously difficult to make.

The enzymes described herein are from a variety of mint plant species and can synthesize a variety of terpene skeletons and terpenes.

### Terpenes

The enzymes described herein can facilitate synthesis of a variety of terpenes, diterpenes, and terpenoids. For example, the enzymes described herein can facilitate synthesis of terpenes, diterpenes, and terpenoids can generally have the structure of Formula I:

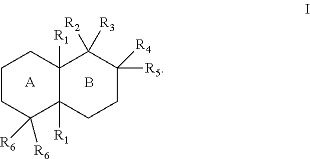

In some cases, the terpenes, diterpenes, and terpenoids can generally have the structure of Formula II:

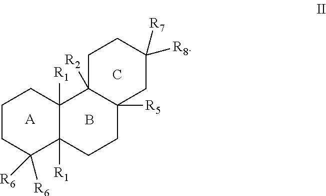

In some cases, the terpenes, diterpenes, and terpenoids can generally have the structure of Formula III:

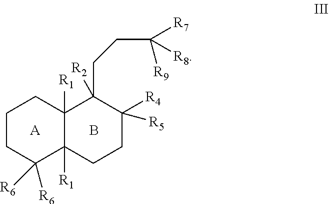

The substituents of Formulae I, II, and III can be as follows:
each R₁ can separately be hydrogen or lower alkyl;
R₂ can be hydrogen, lower alkyl, hydroxy, a bond to an adjacent ring carbon, or form a C₄-C₆ cycloheteroalkyl with R₃;
R₃ can be a branched C₅-C₆ alkyl with 0-2 double bonds, can form a C₄-C₆ cycloheteroalkyl with R₂; can form a cycloalkyl with R₄, or can form a cycloheteroalkyl ring with R₄, wherein the C₅-C₆ alkyl can optionally have one hydroxy, phosphate or diphosphate substituent, and wherein each cycloalkyl or cycloheteroalkyl ring can have 0-2 double bonds, and each cycloalkyl or cycloheteroalkyl ring can have 0-2 alkyl or 0-2 alkene substituents;
R₄ can be hydrogen, lower alkyl, lower alkene, hydroxy, a carbon bonded to R₉, an oxygen bonded to R₉, form a cycloalkyl ring with R₃, or form a cycloheteroalkyl ring with R₃, wherein each cycloalkyl ring or cycloheteroalkyl ring can have 0-2 double bonds, and each cycloalkyl ring or cycloheteroalkyl ring can have 0-2 alkyl or 0-2 alkene substituents;
R₅ can be hydrogen, hydroxy, lower alkyl, a lower alkene, a bond with an adjacent carbon, form a cycloalkyl ring with a ring atom of a ring formed by R₃ and R₄, wherein the cycloalkyl ring can have 0-2 double bonds, and the cycloalkyl ring can have 0-2 alkyl or 0-2 alkene substituents;
each R₆ can separately be hydrogen, lower alkyl, lower alkene, or form a bond with an adjacent carbon;
R₇ can be lower alkyl, lower alkene, or form a cycloalkyl ring with a R₅,
R₈ can be lower alkyl, hydroxy, phosphate, diphosphate, or form a bond with an adjacent carbon; or
R₉ can be hydrogen, lower alkyl, lower alkene, ═CH₂, hydroxy, phosphate, diphosphate, form a bond with an adjacent carbon, form a cycloalkyl ring with R₄, or form a cycloheteroalkyl ring with R₄, wherein each cycloalkyl ring or cycloheteroalkyl ring can have 0-2 double bonds, and each cycloalkyl ring or cycloheteroalkyl ring can have 0-2 alkyl or 0-2 alkene substituents.

The alkyl group(s) can have one to ten carbon atoms. In some cases, the alkyl groups can be lower alkyl group(s) (e.g., C₁-C₆ alkyl groups). In some cases, where substituents such as R₁, R₂, R₅, and R₆ are lower alkyl groups, they can be a C1-C3 lower alkyl. In some cases, where substituents such as R₁, R₂, R₅, and R_{b} are lower alkyl groups, they are an ethyl or methyl group.

Cycloalkyl groups are cyclic alkyl groups such as, but not limited to, cyclopropyl, cyclobutyl, cyclopentyl, cyclohexyl, cycloheptyl, and cyclooctyl groups. In some cases, the cycloalkyl group can have 3 to about 8-12 ring members, whereas in other cases the number of ring carbon atoms range from 4, 5, 6, or 7. Cycloalkyl groups can include cycloalkyl rings having at least one double bond between 2 carbons (i.e., cycloalkenyl rings). Thus, for example, the A, B and/or C rings can also be a cycloalkenyl group such as a cyclohexenyl, cyclopentenyl, or cyclohexadienyl group. Cycloalkenyl groups can have from 4 to about 8-12 ring members.

Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined above. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups. The term “cycloalkenyl” alone or in combination denotes a cyclic alkenyl group.

Heterocycloalkyl groups include ring groups containing 3 or more ring members, of which, one or more is a heteroatom such as, but not limited to, N, O, and S. The compounds described herein that have heteroatoms typically have an oxygen heteroatom. In some embodiments, heterocyclyl groups include 3 to about 15 ring members, whereas other such groups have 3 to about 10 ring members. A heterocyclyl group designated as a C₂-heterocyclyl can be a 5-ring with two carbon atoms and three heteroatoms, 6-ring with two carbon atoms and four heteroatoms and so forth. A C₃-heterocyclyl can be a 5-ring with three carbons and two heteroatoms, a 6-ring with three carbons and three heteroatoms, and so forth. A C₄-heterocyclyl can be a 5-ring four carbons and one heteroatom, a 6-ring with four carbons and two heteroatoms, and so forth. The number of carbon atoms plus the number of heteroatoms sums up to equal the total number of ring atoms. A heterocyclyl ring can also include one or more double bonds. The phrase “heterocyclyl group” includes fused ring species including those comprising fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or they can be substituted. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups

In some cases, only one of the R₁ groups is a lower alkyl, while the other is hydrogen.

In some cases, R₂ is hydrogen when R₃ forms a ring with R₄. Although in many diterpenes, each R₆ is a lower alkyl, in some cases one R₆ is a lower alkene white the other is bond that contributes to lower alkene. For example, in some cases the two R₆ groups form a lower alkene together, for example, a ═CH₂ group.

The compounds produced by the enzymes described herein are typically terpenes or diterpenes. Diterpenes are a class of chemical compounds composed of two terpene units, often with the molecular formula C₂₀H₃₂, though some can include 1-2 heteroatoms or other substituents. Diterpenes generally consist of four isoprene subunits. The positions of various atoms in a diterpene can, for example, be numbered as shown below.

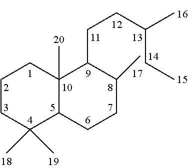 [SMILES: CCC(C)CCC1C(C)CCC2C(C)(C)CCCC12C]

The enzymes described herein can produce compounds with the following skeletons (Sk1-Sk14), where 1-2 of the ring atoms can in some cases be heteroatoms (e.g., oxygen or nitrogen). If a heteroatom is present in it is usually an oxygen atom.

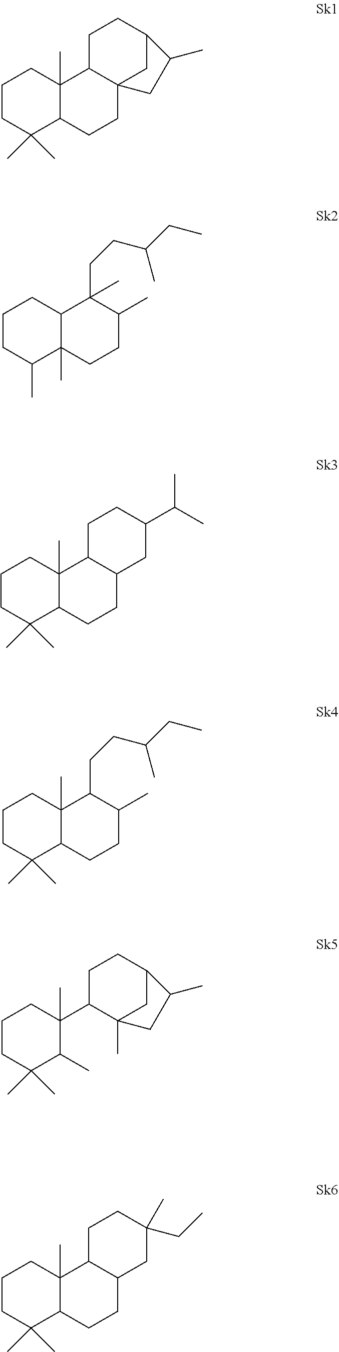

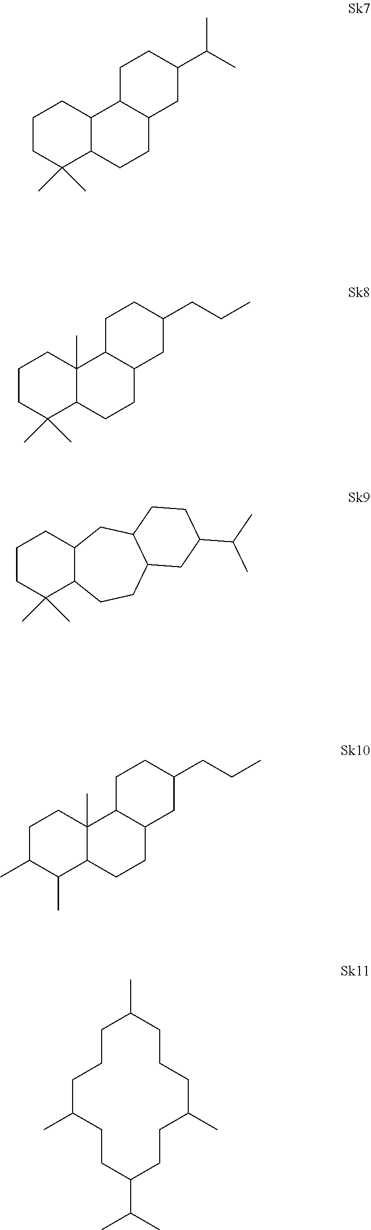

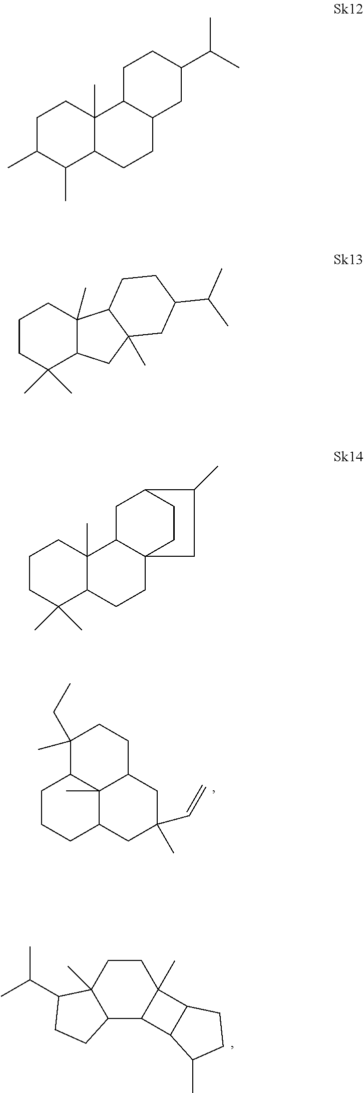

or a combination thereof.

### Enzymes

The enzymes described herein are from a variety of mint plant species and can synthesize a variety of terpenes, diterpene skeletons, and terpenoid compounds.

For example, an Ajuga reptans miltiradiene synthase (ArTPS3), a Leonotis leonurus sandaracopimaradiene synthase (LITPS4), a Mentha spicata class I diterpene synthase (MsTPS1), an Origanum majorana trans-abienol synthase (OmTPS3), an Origanum majorana manool synthase (OmTPS4), an Origanum majorana palustradiene synthase (OmTPS5), Perovskia atriplicifolia miltiradiene synthase (PaTPS3), Prunella vulgaris miltiradiene synthase (PvTPS1), Salvia officinalis miltiradiene synthase (SoTPS1) were identified and isolated as described herein.

Eight of these enzymes, ArTPS3, LITPS4, MsTPS1, OmTPS4, OmTPS5, PaTPS3, PvTPS1, and SoTPS1 can convert a labda-13-en-8-ol diphosphate ((+)-8-LPP) [compound 10]) to 13R-(+)-manoyl oxide [8].

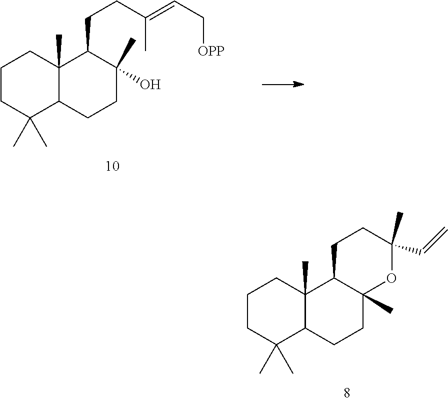

The ArTPS3, LITPS4, OmTPS4, OmTPS5, PaTPS3, PvTPS1, and SoYPS1 enzymes can also convert peregrinol diphosphate (PgPP) [5] to a combination of compounds 1, 2, and 3, as illustrated below.

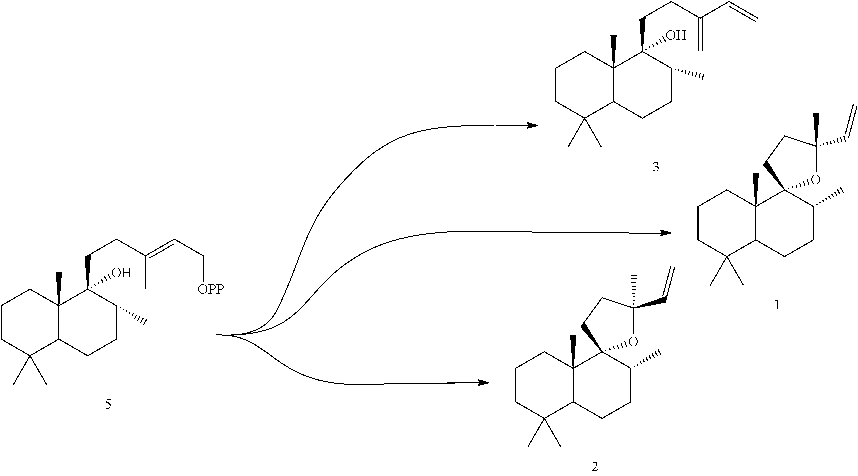

However, MsTPS1 produced only compound 3 from compound 5, while the OmTPS3 enzyme produced only 1, and 2. The OmTPS4 enzyme produced compound 4 (shown below) in addition to compounds 1, 2, and 3.

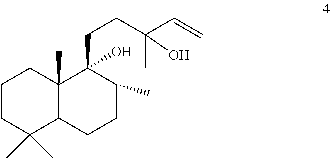 [SMILES: C=CC(C)(O)CC[C@@]1(O)[C@H](C)CCC2C(C)(C)CCC[C@@]21C]

The ArTPS3, PaTPS3, PvTPS1, and SoTPS1 enzymes can also convert (+)-copalyl diphosphate ((+)-CPP) [31]) to miltiradiene [32].

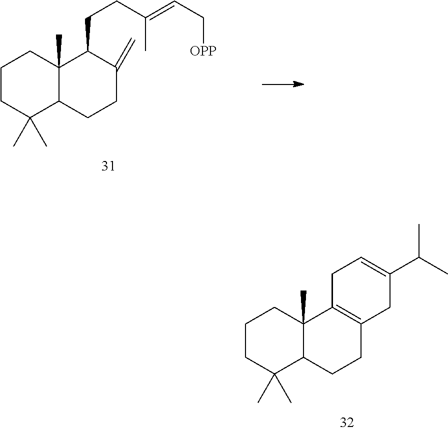

However, LITPS4 and MsTPS1 converted (+)-copalyl diphosphate ((+)-CPP) [31]) to sadaracopimaradiene [27], while OmTPS3 converted (+)-copalyl di ho hate ((+)-CPP) [31]) to trans-biformene [34].

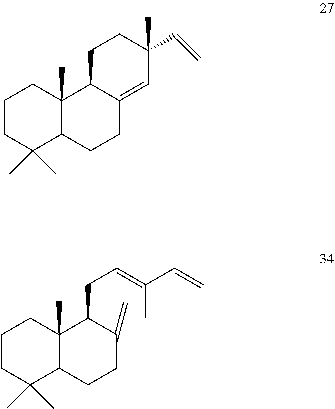

The Ajuga reptans miltiradiene synthase (ArTPS3) has the amino acid sequence shown below (SEQ ID NO:1).

| | |
|---|---|
| 1 | MSLSFTIKVT PFSGQRVHSS TESFPIQQFP TITTKSAMAV |
| 41 | KCSSLSTATV SFQDFVGKIR DTINGKVDNS PAATTIHPAD |
| 81 | IPSNLCVVDT LQRLGVDRYF QSEIDSVLND TYRFWQQKGE |
| 121 | DIFTDVACRA MAFRLLRVKG YEVSSDELAS YAEQEHVNLQ |
| 161 | PSDITTVIEL YRASQTRLYE DEGNLEKLHT WTSNFLKQQL |
| 201 | QSETISDEKL HKQVEYYLKN YHGILDRAGV RQSLDLYDIN |
| 241 | QYQNLKSTDR FPTLSNEDLL EFAKQDFNFC QAQHQKELQQ |
| 281 | LQRWYADCKL DTLTYGRDVV RVASFLTAAI FGEPEFSDAR |
| 321 | LAFAKHIILV TRIDDFFDHG GSIEESYKIL DLVKEWEDKP |
| 361 | AEEYPSKEVE ILFTAVYNTV NDLAEMAYIE QGRSIKPLLI |
| 401 | KLWVEILTSF KKELDSWTED TELTLEEYLA SSWVSIGCRI |
| 441 | CSLNSLQFLG ITLSEEMLSS EECMELCRHV SSVDRLLNDV |
| 481 | QTFEKERLEN TINSVSLQLA EAQREGRTIT EEEAMSKIKD |
| 521 | LADYHRRQLM QMVYKDGTIF PRQCKDVFLR VCRIGYYLYA |
| 561 | SGDEFTTPQQ MMGDMKSLVY EPLNTSSS |

A nucleic acid encoding the Ajuga reptans miltiradiene synthase (ArTPS3) with SEQ ID NO:1 is shown below as SEQ ID NO:2.

| | |
|---|---|
| 1 | ATGTCACTCT CGTTCACCAT CAAAGTCACC CCCTTTTCGG |
| 41 | GCCAGAGAGT TCACAGCAGC ACAGAAAGCT TTCCAATCCA |
| 81 | ACAATTTCCA ACGATCACCA CCAAATCCGC CATGGCTGTC |
| 121 | AAATGCAGCA GCCTCAGTAC CGCAACAGTA AGCTTCCAGG |
| 161 | ATTTCGTCGG AAAAATCAGA GATACGATCA ACGGGAAAGT |
| 201 | TGACAATTCT CCAGCAGCGA CCACTATTCA TCCTGCAGAT |
| 241 | ATACCCTCCA ATCTCTGCGT GGTGGATACC CTCCAAAGAT |
| 281 | TGGGAGTTGA CCGTTACTTC CAATCTGAAA TCGACAGCGT |
| 321 | TCTTAACGAC ACATACAGGT TCTGGCAGCA GAAAGGAGAA |
| 361 | GATATCTTCA CTGATGTTGC TTGTCGTGCA ATGGCATTTC |
| 401 | GACTTTTGCG AGTTAAAGGA TATGAAGTTT CATCAGATGA |
| 521 | ACTCGCTTCG TATGCTGAAC AAGAGCATGT TAACCTGCAA |
| 561 | CCAAGTGACA TAACTACGGT TATCGAGCTT TACAGAGCAT |
| 601 | CACAGACAAG ATTATATGAA GACGAGGGCA ATCTTGAGAA |
| 641 | GTTACATACT TGGACTAGCA ATTTTCTGAA GCAACAATTG |
| 681 | CAGAGTGAAA CTATTTCTGA CGAGAAATTG CACAAACAGG |
| 721 | TGGAGTATTA CTTGAAGAAC TACCACGGCA TACTAGACCG |
| 761 | TGCTGGAGTT AGACAAAGTC TCGATTTATA TGACATAAAC |
| 801 | CAATACCAGA ATCTAAAATC TACAGATAGA TTCCCTACTT |
| 841 | TAAGTAACGA AGATTTACTT GAATTCGCGA AGCAAGATTT |
| 881 | TAACTTTTGC CAAGCTCAAC ACCAGAAAGA GCTTCAGCAA |
| 921 | CTGCAAAGGT GGTATGCGGA TTGTAAATTG GATACATTGA |
| 961 | CTTACGGAAG AGATGTGGTA CGTGTTGCAA GTTTCCTGAC |
| 1001 | AGCTGCAATT TTTGGTGAGC CTGAATTCTC TGATGCTCGT |
| 1041 | CTAGCCTTCG CCAAACACAT CATCCTCGTG ACACGTATTG |
| 1081 | ATGATTTCTT CGATCATGGT GGGTCTATAG AAGAGTCATA |
| 1121 | CAAGATCCTG GATTTAGTAA AAGAATGGGA AGATAAGCCA |
| 1161 | GCTGAGGAAT ATCCTTCCAA GGAAGTTGAA ATCCTCTTTA |
| 1201 | CAGCAGTATA TAATACAGTA AATGACTTGG CAGAAATGGC |
| 1241 | TTATATTGAG CAAGGCCGTT CCATTAAACC TCTTCTAATT |
| 1281 | AAACTGTGGG TTGAAATACT GACAAGTTTC AAGAAAGAAC |
| 1321 | TGGATTCATG GACAGAAGAC ACAGAACTAA CCTTGGAGGA |
| 1361 | GTACTTGGCT TCCTCCTGGG TGTCGATCGG TTGCAGAATC |
| 1401 | TGCAGTCTCA ATTCGCTGCA GTTCCTTGGT ATAACATTAT |
| 1441 | CCGAAGAAAT GCTTTCAAGC GAAGAGTGCA TGGAGTTGTG |
| 1481 | TAGGCATGTT TCTTCAGTCG ACAGGCTACT CAATGACGTG |
| 1521 | CAAACTTTCG AGAAGGAACG CCTAGAAAAT ACGATAAACA |
| 1561 | GTGTGAGCCT ACAGCTAGCA GAAGCTCAGA GAGAAGGAAG |
| 1601 | AACCATTACA GAAGAGGAGG CTATGTCAAA GATTAAAGAC |
| 1641 | CTGGCTGATT ATCACAGGAG ACAACTGATG CAGATGGTTT |
| 1681 | ATAAGGATGG GACCATATTT CCGAGACAAT GCAAAGATGT |
| 1721 | CTTTTTGAGG GTATGCAGGA TTGGCTACTA CTTATACGCG |
| 1761 | AGCGGCGATG AATTCACTAC TCCACAACAA ATGATGGGGG |
| 1801 | ATATGAAATC ATTGGTTTAT GAACCCCTAA ACACTTCATC |
| 1841 | CTCTTGA |

The Leonotis leonarus sandaracopimaradiene synthase (LITPS4) has the amino acid sequence shown below (SEQ ID NO:3).

| | |
|---|---|
| 1 | MSVAFNLIVV RFPGHGIQSS RETFPAKIIT RTKSSMRFQS |
| 41 | SLNTSTDFVG KIREMIRGKT DNSINPLDIP STLCVIDTLH |
| 81 | SFGIDRYFQS EINSVLHHTY RLWNDRNNII FKDVICCAIA |
| 121 | FRLLRVKGYQ VSSDELAPFA QQQVTGLQTS DIATILELYR |
| 161 | ASQERLHEDD DTLDKLHDWS SNLLKLHLLN ENIPDHKLHK |
| 201 | RVGYFLKNYH GMLDRVAVRR NIDLHNINHY QIPEVADRFP |
| 241 | TEAFLEFSRQ DFNICQAQHQ KELQQLHRWY ADCRLDTLNH |
| 281 | GTDVVHFANF LTSAIFGEPE FSEARLAFAK QVILITRMDD |
| 321 | FFDHDGSREE SHKILHLVQQ WKEKPAEEYG SKEVEILFTA |
| 361 | VYTTVNSLAE KACMEQGRSV KQLLIKLWVE LLTSFKKELD |
| 401 | SWTEKMALTL DEYLSFSWVS IGCRLCILNS LQFLGIKLSE |
| 441 | EMLWSQECLD LCRHVSSVVR LLNDLQTFKK ERIENTINGV |
| 481 | DVQLAARKGE RAITEEEAMS KIKEMADHHR RKLMQIVYKE |
| 521 | GTIFPRECKD VFLRVCRIGY YLYSGDELTS PQQMKEDMKA |
| 561 | LVHESSS |

A nucleic acid encoding the Leonotis leonurus sandaracopimaradiene synthase (LITPS4) with SEQ ID NO:3 is shown below as SEQ ID NO:4.

| | |
|---|---|
| 1 | ATGTCGGTGG CGTTCAACCT CATAGTCGTC CGTTTTCCGG |
| 41 | GCCATGGAAT TCAGAGCAGT AGAGAAACTT TTCCAGCCAA |
| 81 | AATTATTACC AGAACTAAAT CAAGCATGAG ATTCCAAAGC |
| 121 | AGCCTCAACA CTTCAACAGA TTTCGTGGGA AAAATAAGAG |
| 161 | AGATGATCAG AGGGAAAACT GATAATTCTA TTAATCCCCT |
| 201 | GGATATTCCC TCCACTCTAT GCGTAATCGA CACCCTACAC |
| 241 | AGCTTCGGAA TTGATCGCTA CTTTCAATCC GAAATCAACT |
| 281 | CTGTTCTTCA CCACACATAC AGATTATGGA ACGACAGAAA |
| 321 | TAATATCATC TTCAAAGATG TCATTTGCTG CGCAATTGCC |
| 361 | TTTAGACTTT TGCGAGTGAA AGGATATCAA GTCTCATCAG |
| 401 | ATGAACTGGC GCCATTTGCC CAACAACAGG TGACTGGACT |
| 441 | ACAAACAAGC GACATTGCCA CGATTCTAGA GCTCTACAGA |
| 481 | GCATCACAGG AGAGATTACA CGAAGACGAC GACACTCTTG |
| 521 | ACAAACTACA TGATTGGAGC AGCAACCTTC TGAAGCTGCA |
| 561 | TCTGCTGAAT GAGAACATTC CTGATCATAA ACTGCACAAA |
| 601 | CGGGTGGGGT ATTTCTTGAA GAACTACCAT GGCATGCTAG |
| 641 | ATCGCGTTGC GGTTAGACGA AACATCGACC TTCACAACAT |
| 681 | AAACCATTAC CAAATCCCAG AAGTTGCAGA TAGGTTCCCT |
| 721 | ACTGAAGCTT TTCTTGAATT TTCAAGGCAA GATTTTAATA |
| 761 | TTTGCCAAGC TCAACACCAG AAAGAACTTC AGCAACTGCA |
| 801 | TAGGTGGTAT GCAGATTGTA GATTGGACAC ACTGAATCAC |
| 841 | GGAACAGACG TAGTACATTT TGCTAATTTT CTAACTTCAG |
| 881 | CAATTTTCGG AGAGCCTGAA TTCTCCGAGG CTCGTCTAGC |
| 921 | CTTTGCTAAA CAGGTTATCC TAATAACACG TATGGATGAT |
| 961 | TTCTTCGATC ACGATGGGTC TAGAGAAGAA TCACACAAGA |
| 1001 | TCCTCCATCT AGTTCAACAA TGGAAAGAGA AGCCCGCCGA |
| 1041 | AGAATATGGT TCAAAGGAAG TTGAGATCCT CTTTACAGCA |
| 1081 | GTGTACACTA CAGTAAATAG CTTGGCAGAA AAGGCTTGTA |
| 1121 | TGGAGCAAGG CCGTAGTGTC AAACAACTTC TAATTAAGCT |
| 1161 | GTGGGTCGAG CTGCTAACAA GTTTCAAGAA AGAATTGGAT |
| 1201 | TCATGGACGG AGAAGATGGC GCTAACCTTG GATGAGTACT |
| 1241 | TGTCTTTCTC CTGGGTGTCA ATTGGCTGCA GACTCTGCAT |
| 1281 | TCTCAATTCC CTGCAATTTC TTGGGATAAA ATTATCTGAA |
| 1321 | GAAATGCTGT GGAGTCAAGA GTGTCTGGAT TTATGCCGGC |
| 1361 | ATGTTTCATC AGTGGTTCGC CTGCTCAACG ATTTACAAAC |
| 1401 | TTTCAAGAAG GAGCGCATAG AAAATACGAT AAACGGTGTG |
| 1441 | GACGTTCAGC TAGCTGCTCG TAAAGGCGAA AGAGCCATTA |
| 1481 | CAGAAGAGGA GGCCATGTCC AAGATTAAGG AAATGGCTGA |
| 1521 | CCATCACAGG AGAAAACTGA TGCAAATTGT GTATAAAGAA |
| 1561 | GGAACCATTT TTCCAAGAGA ATGCAAAGAT GTGTTTTTGA |
| 1601 | GAGTGTGCAG GATTGGCTAC TATCTCTACT CGGGCGATGA |
| 1641 | GTTAACTTCT CCACAACAAA TGAAGGAGGA TATGAAAGCG |
| 1681 | TTGGTACATG AATCATCCTC TTGA |

The Mentha spicata class I diterpene synthase (MsTPS1) has the amino acid sequence shown below (SEQ ID NO:5).

| | |
|---|---|
| 1 | MSSIRNLSLH IDLPKAEKKL VEKIRERIRN GRVEMSPSAY |
| 41 | DTAWVAMVPS RGYSGRPGFP ECVDWIIENQ NPDGSWGLDS |
| 81 | DQPLLVKDSL SSTLACLLAL RKWKTHNQLV QRGMEFIDSR |
| 121 | GWAATDDDNQ ISPIGFNIAF PAMINYAKEL NLTLPLHPPS |
| 161 | IHSLLHIRDS EIRKRNWEYV AEGVVDDTSN WKQIIGTHQR |
| 201 | NNGSLFNSPA TTAAAVIHSH DDKCFRYLIS TLENSNGGWV |
| 241 | PTIYPYDIYA PLCMIDTLER LGIHTYFEVE LSGIFDDIYR |
| 281 | NWQEREEEIF CNVMCRALAF RLLRMRGYHV SSDELAEFVD |
| 321 | KEEFFNSVSM QESGEGTVLE LYRASLTKIN EEERILDKIH |
| 361 | AWTKPFLKHQ LLNRSIRDKR LEKQVEYDLK NFYGALVRFQ |
| 401 | NRRTIDSYDA KSIQTSKTAY RCSTVYNEDF IHLSVEDFKI |
| 441 | SRAQYLKELE EMNKWYSDCR LDLLTKGRNA CRESYILTAA |
| 481 | IIVDPHESMA RISYAQSILL ITVFDDFFDH YGSKEEALNI |
| 521 | IDLVKEWKPA GSYCSKEVEI LFTALHDTIN EIAAKADAEQ |
| 561 | GFSSKQQLIN MWVELLESAV REKDSLSXNK VSTLEEYLSF |
| 601 | APITIGCKLC VLTSVHFLGI KLSEEIWTSE ELSSLCRHGN |
| 641 | VVCRLLNDLK TYEREREENT LNSVSVQTVG GGVSEEEAVT |
| 681 | KVEEVLEFHR RKVMQLACRR GGSSVPRECK ELVWKTCTIG |
| 721 | YCLYGHDGGD ELSSPKDILK DINAMMFEPL K |

A nucleic acid encoding the Mentha spicata class I diterpene synthase (MsTPS1) with SEQ ID NO:5 is shown below as SEQ ID NO:6

| | |
|---|---|
| 1 | ATGAGTTCCA TTCGAAATTT AAGTTTGCAT ATTGATCTGC |
| 41 | CAAAGGCCGA GAAGAAGTTG GTTGAGAAAA TCAGAGAGAG |
| 81 | GATAAGAAAT GGGAGGGTGG AGATGTCGCC GTCGGCTTAC |
| 121 | GACACCGCGT GGGTGGCCAT GGTGCCGTCT CGAGGATATT |
| 161 | CCGGCAGGCC GGGTTTCCCG GAGTGCGTGG ATTGGATAAT |
| 201 | CGAGAACCAG AATCCCGACG CGTCGTGGGG TTTGGATTCG |
| 241 | GATCAACCAC TTCTGGTCAA AGACTCCCTC TCGTCCACCT |
| 281 | TGGCATGCCT ACTTGCCCTG CGTAAATGGA AAACACACAA |
| 321 | CCAACTAGTG CAAAGGGGCA TGGAGTTCAT CGACTCCCGT |
| 361 | GGTTGGGCTG CAACTGATGA TGACAATCAG ATTTCTCCTA |
| 401 | TTGGATTCAA TATTGCCTTT CCTGCAATGA TTAATTACGC |
| 441 | CAAAGAGCTT AATTTAACTC TGCCTCTACA TCCACCTTCG |
| 481 | ATTCATTCAT TGTTACACAT TAGAGATTCA GAAATAAGAA |
| 521 | AGCGAAACTG GGAATACGTA GCTGAAGGAG TAGTCGACGA |
| 561 | TACAAGCAAT TGGAAGCAAA TAATCGGCAC GCATCAAAGA |
| 601 | AATAATGGAT CCTTGTTCAA CTCACCTGCT ACCACTGCAG |
| 641 | CTGCTGTTAT TCACTCTCAC GACGATAAAT GTTTCCGATA |
| 681 | TTTGATCTCC ACTCTTGAGA ATTCTAACGG TGGATGGGTA |
| 721 | CCAACTATCT ATCCATACGA TATATACGCT CCTCTCTGCA |
| 761 | TGATCGATAC GCTAGAAAGA TTAGGAATAC ACACATATTT |
| 801 | TGAAGTTGAA CTCACCGGCA TTTTTGATGA CATATACAGG |
| 841 | AATTGGCAAG AGAGAGAAGA AGAGATCTTT TGTAATGTTA |
| 881 | TGTGTCGACC TCTGGCATTT CGGCTTCTAC GAATGAGGGG |
| 921 | ATATCATGTT TCATCTGATG AACTAGCAGA ATTTGTGGAC |
| 961 | AAGGAGGAGT TTTTTAATAG CGTGAGCATG CAAGAGAGCG |
| 1001 | GCGAAGGCAC AGTGCTTGAG CTTTACAGAG CTTCACTCAC |
| 1041 | AAAAATCAAC GAAGAAGAAA GGATTCTCGA CAAAATTCAT |
| 1081 | GCATGGACCA AACCATTTCT CAAGCACCAG CTTCTCAACC |
| 1121 | GCAGCATTCG CGACAAACGA TTAGAGAAGC AGGTGGAATA |
| 1161 | CGACTTGAAG AACTTCTACG GCGCACTAGT CCGATTCCAG |
| 1201 | AACAGAAGAA CCATCGACTC ATACGATGCT AAATCAATCC |
| 1241 | AAATTTCGAA AACAGCATAT AGGTGCTCTA CAGTTTACAA |
| 1281 | TGAAGACTTC ATCCATTTAT CCGTTGAGGA CTTCAAAATC |
| 1321 | TCCCGAGCAC AATACCTAAA AGAACTTGAA GAAATGAACA |
| 1361 | AGTGGTACTC TGATTGTAGG TTGGACCTCT TAACTAAAGG |
| 1401 | AAGAAATGCA TGTCGAGAAT CTTACATTTT AACAGCTGCA |
| 1441 | ATCATTGTCG ATCCTCACGA ATCCATGGCT CGAATCTCTT |
| 1481 | ACGCTCAATC TATTCTTCTT ATAACTGTTT TCGACGACTT |
| 1521 | TTTCGATCAT TATGGGTCTA AAGAAGAGGC TCTCAATATT |
| 1561 | ATTGATCTAG TCAAGGAATG GAAGCCAGCT GGCAGTTACT |
| 1601 | GCTCCAAAGA AGTGGAGATT TTGTTTACTG CATTACACGA |
| 1641 | CACGATAAAT GAGATTGCAG CCAAGGCTGA TGCAGAGCAA |
| 1681 | GGCTTTTCTT CCAAACAACA GCTTATCAAC ATGTGGGTGG |
| 1721 | AGCTACTTGA GAGCGCCGTG AGAGAAAAGG ACTCGCTGAG |
| 1761 | TGGNAACAAA GTGTCGACTC TAGAAGAGTA CTTATCTTTC |
| 1801 | GCACCAATCA CCATCGGCTG CAAACTTTGC GTCCTGACGT |
| 1841 | CTGTCCATTT CCTCGGAATC AAACTGTCCG AGGAAATCTG |
| 1881 | GACTTCCGAG GAGTTGAGCA GTCTGTGCAG GCACGGCAAT |
| 1921 | GTTGTCTGCA GACTGCTCAA CGACCTCAAG ACTTACGAGA |
| 1961 | GAGAGCGCGA AGAGAACACG CTCAACAGCG TGAGCGTGCA |
| 2001 | GACAGTGGGA GGAGGCGTTT CGGAGGAAGA GGCGGTGACG |
| 2041 | AAGGTGGAGG AGGTGTTGGA ATTTCATAGA AGAAAAGTGA |
| 2081 | TGCAGCTCGC GTGTCGAAGA GGAGGAAGCA GTGTTCCGAG |
| 2121 | AGAATGTAAG GAGCTGGTGT GGAAGACGTG CACGATAGGT |
| 2161 | TACTGCTTGT ACGGTCACGA CGGAGGCGAT GAGTTATCGT |
| 2201 | CTCCGAAGGA TATTCTAAAG GACATTAATG CAATGATGTT |
| 2241 | TGAGCCTCTC AAGTGA |

A Nepeta mussinii ent-kaurene synthase (NmTPS2) was identified and isolated as described herein. This NmTPS2 enzyme was identified as an ent-kaurene synthase, which converts ent-CPP [16] into ent-kaurene [19].

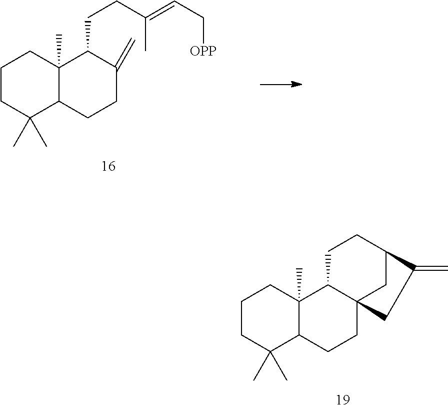

The Nepeta mussinii ent-kaurene synthase (NmTPS2) has the amino acid sequence shown below (SEQ ID NO:7).

| | |
|---|---|
| 1 | MSLPLSSCVL FPPNDSRFPV SRFSRASASL EVGLQGATSA |
| 41 | KVSSQSSCFE ETKRRITKLF HKDELSVSTY DTAWVAMVPS |
| 81 | PTSSEEPCFP GCLTWLLENQ CRDGSWARPH HHSLLKKDVL |
| 121 | SSTLACILAL KKWGVCEEQI NKGLHFIELN CASATEKCQI |
| 161 | TPVGFDIIFP AMLDYARDFS LNLRLEPTTF NDLMDKRDLE |
| 201 | LKRCYQNYTP EREAYLAYIV EGMGRLQDWE LVMKYQRKNG |
| 241 | SLFNCPSTTA AAFIALRDSA CLNYLNLSLK KFGNAVPAVY |
| 281 | PLDIYSQLCT VDNLERLGIN QYFIAEIQSV LDETYRCWIQ |
| 321 | GNEDIFLDTS TCALAFRILR MNGYDVTSDS TTKILEECFS |
| 361 | SSFRGNMTDI NTTLDLYRAS ELMLYPDEKD LEKHNLRLKL |
| 401 | LLKQKLSTVL IQSFQLGRNI NEEVKQTLEH PFYASLDRIA |
| 441 | KRKNIEHYNF DNTRILKTSY CSPNFGNKDF FFLSIEDFNW |
| 481 | CQVIHRQELA ELERWLIENR LDELKFARSK SAYCYFSAAA |
| 521 | TFFAPELSDA RMSWAKSGVL TTVVDDFFDV GGSMEELKNL |
| 561 | IQLVELWDVD ASTKCSSHNV HIIFSALRRT IYEIGNKGFK |
| 601 | LQGRNITNHI IDIWLDLLNS MMKETEWARD NFVPTIDEYM |
| 641 | SNAYTSFALG PIVLPTLYLV GPKLSEEMIN HSEYHNLFKL |
| 681 | MSTCGRLLND IRGYERELKD GKLNALSLYI INNGGKVSKE |
| 721 | AGISEMKSWI EAQRRELLRL VLESNKSVLP KSCKELFWHM |
| 761 | CSVVHLFYCK DDGFTSQDLI QVVNAVIHEP IALKDFKVHE |

A nucleic acid encoding the Nepeta mussinii ent-kaurene synthase (NmTPS2) with SEQ ID NO:7 is shown below as SEQ ID NO:8.

| | |
|---|---|
| 1 | ATGTCTCTTC CGCTCTCCTC TTGTGTCTTA TTTCCCCCCA |
| 41 | ATGACTCACG TTTTCCGCTC TCCCGCTTTT CTCGCGCTTC |
| 81 | AGCTTCTTTG GAAGTCGGGC TTCAAGGAGC TACTTCAGCA |
| 121 | AAAGTCTCCT CACAATCATC GTGTTTTGAG GAGACAAAGA |
| 161 | GAAGGATAAC AAAGTTGTTT CATAAGGACG AACTTTCGGT |
| 201 | TTCGACATAT GACACAGCAT GGGTTGCTAT GGTCCCTTCT |
| 241 | CCAACTTCTT CAGAGGAACC TTGCTTCCCA GGTTGTTTGA |
| 281 | CTTGGTTGCT TGAAAACCAG TGTCGAGATG GTTCATGGGC |
| 321 | TCGTCCCCAC CATCACTCTT TGTTAAAAAA AGATGTCCTT |
| 361 | TCTTCTACCT TGGCATGCAT TCTCGCACTT AAAAAATGGG |
| 401 | GGGTTGGTGA AGAACAAATC AACAAGGGTT TGCATTTTAT |
| 441 | AGAGCTAAAT TGTGCTTCAG CTACCGAGAA GTGTCAAATT |
| 481 | ACTCCCGTGG GGTTTGACAT TATATTTCCT GCCATGCTTG |
| 521 | ATTATGCAAG AGACTTCTCT TTGAACTTGC GTTTAGAGCC |
| 561 | AACTACGTTT AATGATTTGA TGGATAAAAG GGATTTAGAG |
| 601 | CTCAAAAGGT GTTACCAAAA TTACACACCG GAGAGGGAAG |
| 641 | CATACTTGGC ATATATAGTT GAAGGAATGG GAAGATTGCA |
| 681 | AGATTGGGAA TTGGTGATGA AATATCAAAG AAAGAATGGA |
| 721 | TCTCTTTTCA ATTGTCCATC TACAACTGCA GCAGCTTTTA |
| 761 | TTGCCCTTCG GGATTCTGCG TGCCTCAACT ATCTGAATTT |
| 801 | GTCTTTGAAA AAGTTCGGGA ATGCAGTTCC TGCAGTTTAT |
| 841 | CCTCTAGATA TATATTCTCA ACTTTGCACG GTTGATAATC |
| 881 | TTGAAAGGCT GGGGATCAAC CAATATTTTA TAGCAGAAAT |
| 921 | TCAGAGTGTG TTGGATGAAA CGTACAGATG TTGGATACAG |
| 961 | CGAAACGAAG ACATATTTTT GGACACCTCA ACTTGTCCTT |
| 1001 | TAGCATTCCG AATATTGAGA ATGAATGGCT ATGATGTGAC |
| 1041 | TTCAGATTCA CTTACAAAAA TCCTAGAAGA GTGCTTTTCA |
| 1081 | AGTTCCTTTC GTGGAAATAT GACAGACATT AACACAACTC |
| 1121 | TTGACTTATA TAGGGCATCA GAACTTATGT TATATCCAGA |
| 1161 | TGAAAAGGAT CTGGAGAAAC ATAATTTAAG GCTTAAACTC |
| 1201 | TTACTTAAGC AAAAACTATC CACTGTTTTA ATCCAATCAT |
| 1241 | TTCAACTTGG AAGAAATATC AATGAAGAGG TGAAACAGAC |
| 1281 | TCTCGAGCAT CCCTTTTATG CAAGTTTGGA TAGGATTGCA |
| 1321 | AAGCGGAAAA ATATAGAGCA TTACAACTTT GATAACACAA |
| 1361 | GAATTCTTAA AACTTCATAT TGTTCGCCAA ATTTTGGCAA |
| 1401 | CAAGGATTTC TTTTTTCTTT CCATAGAAGA CTTCAATTGG |
| 1441 | TGTCAAGTCA TACATCGACA AGAACTCGGA GAACTTGAAA |
| 1481 | GATGGTTAAT TGAAAATAGA TTGGATGAGC TGAAGTTTGC |
| 1521 | AAGGAGTAAG TCTGCATACT GTTATTTTTC TGCGGCAGCA |
| 1561 | ACTTTTTTTG CTCCAGAATT GTCGGATGCC CGCATGTCAT |
| 1601 | GGGCTAAAAG TGGTGTTCTA ACCACAGTGG TAGATGACTT |
| 1641 | TTTTGATGTT GGAGGTTCTA TGGAGGAATT GAAGAACTTA |
| 1681 | ATTCAATTGG TTGAACTATG GGATGTGGAT GCTAGCACAA |
| 1721 | AATGCTCTTC TCATAATGTC CATATAATAT TTTCAGCACT |
| 1761 | TAGGCGCACC ATCTATGAGA TAGGGAACAA AGGATTTAAG |
| 1801 | CTACAAGGAC GTAACATTAC CAATCATATA ATTGACATTT |
| 1841 | GGCTAGATTT ACTAAACTCT ATGATGAAAG AAACCGAATG |
| 1881 | GGCCAGAGAC AACTTTGTCC CAACAATTGA TGAATACATG |
| 1921 | AGCAATGCAT ATACATCGTT TGCTCTGGGG CCAATTGTCC |
| 1961 | TTCCAACTCT CTATCTTGTC GGGCCCAAGC TCTCAGAAGA |
| 2001 | GATGATTAAC CACTCCGAAT ACCATAACCT ATTCAAATTG |
| 2041 | ATGAGTACGT GCGGACGTCT TCTAAATGAC ATCCGTGGTT |
| 2081 | ATGAGAGAGA ACTGAAAGAT GGTAAATTGA ACGCGTTATC |
| 2121 | ATTGTACATA ATTAATAATG GTGGTAAAGT AAGTAAAGAA |
| 2161 | GCTGGCATCT CGGAGATGAA AAGTTGGATC GAGGCACAAC |
| 2201 | GAAGAGAGTT ACTGAGATTA GTTTTGGAGA GCAACAAAAG |
| 2241 | CGTCCTTCCG AAGTCGTGCA AGGAATTGTT TTGGCATATG |
| 2281 | TGCTCAGTGG TGCATCTATT CTACTGCAAA GATGATGGAT |
| 2321 | TCACCTCGCA GGATTTGATT CAAGTTGTAA ATGCAGTTAT |
| 2361 | TCATGAACCT ATTGCTCTCA AGGATTTTAA GGTGCATGAA |
| 2401 | TAA |

An Origanum majorana trans-abienol synthase (OmTPS3) was identified and isolated. When this OmTPS3 enzyme was expressed in N. benthamiana with Hyptis suaveolens labda-7,13E-dienyl diphosphate synthase (HsTPS1) a new compound, labda-7,12E,14-triene [24], was produced. The HsTPS1 enzyme produced labda-7,13(16),14-triene [22] when HsTPS1 was expressed in N. benthamiana.

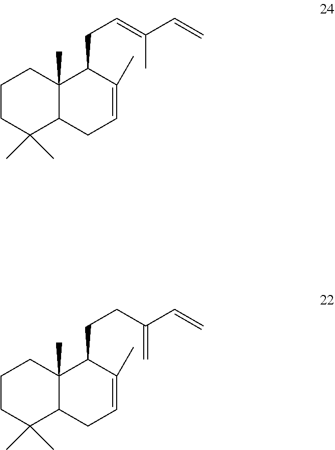

OmTPS3 also produced trans-abienol [11] from labda-13-en-8-ol diphosphate ((+)-8-LPP) [10]).

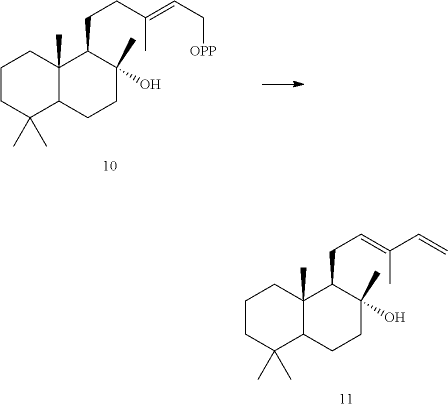

The Origanum majorana trans-abienol synthase (OmTPS3) has the amino acid sequence shown below (SEQ ID NO:9.

MASLAFTPGA ATFSGNVVRR RKDNFPVHGF PTTIRSSVSV
TVKCYVSTTN LMVKIKEKFK GKNVNSLTVE AADDDMPSNL
CIIDTLQRLG IDRYFQPQVD SVLDHAYKLW QGKEKDTVYS
DISIHAMAFR LLRVKGYQVS SEELDPYIDV ERMKKLKTVD
VPTVIELYRA AQERMYEEEG SLERLHVWST NFLMHQLQAN
SIPDEKLHKL VEYYLKNYHG ILDRVGVRRN LDLFDISHYP
TLRARVPNLC TEDFLSFAKE DFNTCQAQHQ KEHEQLQRWF
EDCRFDTLKF GRETAVGAAH FLSSAILGES ELCNVRLALA
KHMVLVVFID DFFDHYGSRE DSFKILHLLK EWKEKPAGEY
GSEEVEILFT AVYNTVNELA EMAHVEQGRN IKGFLIELWV
EIVSIFKIEL DTWSNDTTLT LDEYLSSSWV SVGCRICILV
SMQLLGVQLT DEMLLSDECI NLCKHVSMVD RLLNDVGTFE
KERKENTGNS VSLLLAAAVK EGRPITEEEA IIKIKKMAEN
ERRKLMQIVY KRESVFPRKC KDMFLKVCRI GCYLYASGDR
FTSPQKMKED VKSLIYESL

A nucleic acid encoding the Origanum majorana trans-abienol synthase (OmTPS3) with SEQ ID NO:9 is shown below as SEQ ID NO:10.

ATGGCGTCGC TCGCGTTCAC ACCCGGAGCC GCCACTTTCT
CCCCCAACCT AGTTCGGAGG AGGAAAGATA ACTTTCCGGT
CCACGGATTT CCGACGACGA TCAGGTCATC GGTCTCCGTC
ACCGTCAAAT GCTACGTCAG TACAACGAAT TTGATGGTGA
AAATCAAAGA GAAGTTCAAG GGTAAAAACG TCAATTCGCT
GACAGTTGAA GCTGCTGATG ACGATATGCC CTCTAATCTG
TGCATAATTG ACACCCTCCA ACGATTGGGA ATCGACCGTT
ACTTCCAACC CCAACTCGAC TCTGTTCTCG ACCACGCCTA
CAAACTATGG CAAGGGAAAG AGAAAGATAC GGTGTATTCG
GACATTAGTA TTCATGCGAT GGCATTTAGA CTTTTACGAG
TCAAAGGCTA TCAAGTCTCT TCGGAGGAAC TGGATCCATA
CATCGATGTG GAGCGAATGA AGAAACTGAA AACAGTTGAT
GTTCCGACGG TTATCGAACT GTACAGAGCG GCACAGGAGA
GAATGTATGA AGAAGAAGGT AGCCTTGAGA GACTCCATGT
TTGGAGCACC AACTTCCTCA TGCACCAGCT GCAGGCTAAC
TCAATTCCTG ATGAAAAGCT ACACAAACTG GTGGAATACT
ACTTGAAGAA CTACCATGGC ATACTGGATA GAGTTGGAGT
TCGACGAAAC CTCGACCTAT TCGACATAAG CCATTATCCA
ACACTCAGAG CTAGGGTTCC GAACCTATGT ACCGAAGATT
TTCTATCGTT CGCGAAGGAA GATTTCAATA CTTGCCAAGC
CCAACACCAG AAAGAACATG AGCAACTACA AAGGTGGTTC
GAAGATTGTA GGTTCGATAC GTTGAAGTTC GCAAGGGAGA
CAGCCGTAGG CGCTGCTCAT TTTCTATCTT CAGCAATACT
TGGTGAATCT GAACTATGTA ATGTTCGTCT TGCCCTTGCT
AAGCATATGG TGCTTGTGGT ATTCATCGAT GACTTCTTCG
ACCATTATGG CTCTAGAGAA GACTCCTTCA AGATCCTCCA
CCTCTTAAAA GAATGGAAAG AGAAGCCGGC CGGAGAATAC
GGTTCCGAGG AAGTCGAAAT CCTCTTCACA GCCGTATACA
ATACAGTAAA CGAGTTGGCG GAGATGGCTC ATGTCGAACA
AGGACGTAAT ATCAAAGGAT TTCTAATTGA ATTGTGGGTT
GAAATAGTGT CAATTTTCAA GATAGAACTG GATACATGGA
GCAATGACAC AACACTAACC TTGGATGAGT ACTTGTCCTC
CTCATGGGTG TCGGTCGGTT GCAGAATCTG CATCCTCGTC
TCAATGCACC TCCTCGGTGT ACAACTAACC GACGAAATGC
TTCTGAGCGA CGAGTGCATA AACCTGTGTA AGCATGTCTC
GATGGTCGAT CGCCTCCTCA ACGACGTCGG AACATTCGAG
AAGGAACGGA AGGAGAATAC AGGAAACAGT GTGAGCCTTC
TGCTAGCAGC AGCTGTGAAA GAAGGAAGGC CTATTACCGA
AGAGGAAGCT ATTATTAAAA TTAAAAAAAT GGCGGAAAAC
GAGAGGAGGA AACTAATGCA GATTGTGTAT AAAAGAGAGA
GTGTTTTCCC CAGAAAATGC AAGGATATGT TCTTGAAGGT
GTGTAGAATT GGGTGCTATC TATACGCGAG CGGCGACGAA
TTTACGTCTC CTCAGAAAAT GAAGGAAGAT GTGAAATCCT
TAATTTATGA ATCCTTGTAG

The Origanum majorana manool synthase (OmTPS4) can also convert ent-copalyl diphosphate (ent-CPP) [16] to ent-manool [20].

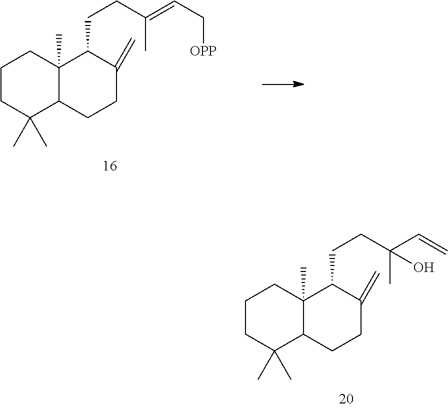

In addition, Origanum majorana manool synthase (OmTPS4) can also convert (+)-copalyl diphosphate ((+)-CPP) [31]) to manool [33].

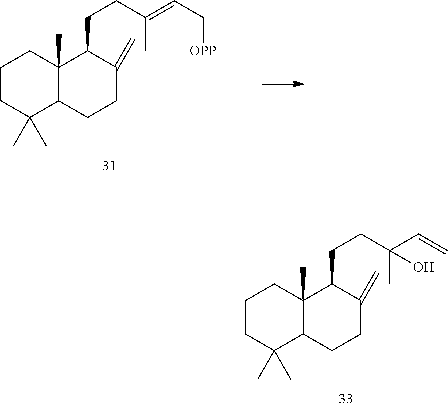

The Origanum majorana manool synthase (OmTPS4) can have the amino acid sequence shown below (SEQ ID NO:11).

MSLAFSHVST FFSGQRVVGS RREIIPVNGV PTTANKPSFA
VKCNLTTKDL MVKMKEKLKG QDGNLTVGVA DMPSSLCVID
TLERLGVDRY FRSEIHVILH DTYRLWQQKD KDICSNVTTH
AMAFRLLRVN GYEVSSEELA PYANLEHFSQ QKVDTAMAIE
LYRAAQERIH EDESGLDKIL AWTTTFLEQQ LLTNSILDNK
LHKLVEYYLN NYHGQTNRVG ARRHLDLYEM SHYQNLKPSH
SLCNEDLLAF AKQGFRDFQI QQQKEFEQLQ RWYEDCRLDK
LSYGRDVVKI SSFMASILMD DPELADVRLS IAKQMVLVTR
IDDFFDHGGS REDSYKIIEL VKEWKEKAEY DSEEVKILFT
AVYTTVNELA EACVQQGRNS TTVKEFLVQL WIEILSAFKV
ELDTWSDGTE VSLDEYLSWS WISNGCRVSI VTTMHLLPTK
LCSDEMLRSE ECKDLCRHVS MVCRLLNDIH SFEKEHEENT
GNSVSILVAG EDTEEEAIGK IKEIVEYERR KLMQIVYKRG
TILPRECKDI FLKACRATFY VYSSTDEFTS PRQVMEDMKT
LSS

A nucleic acid encoding Origanum majorana manool synthase (OmTPS4) with SEQ ID NO:11 is shown below as SEQ ID NO:12.

ATGTCACTCG CCTTCAGCCA TGTTAGTACC TTTTTCTCCC
GCCAAAGAGT CGTCGGAAGC AGGAGAGAGA TTATTCCAGT
TAACCGAGTT CCGACGACGG CCAATAAGCC GTCGTTCGCC
GTTAAGTGCA ACCTTACTAC AAAGGATTTG ATGGTGAAAA
TGAAGGAGAA GTTGAAGGGG CAAGACCGTA ATTTGACTGT
CGGAGTAGCC GATATGCCCT CTAGCCTGTG CGTGATCGAC
ACTCTTGAAA GGTTGGGAGT TGACCGATAC TTCCGATCTG
AAATCCACGT TATTCTACAC GACACTTACC GGTTATGGCA
ACAAAAGGAC AAAGATATAT GTTCCAACGT TACTACTCAT
GCAATGGCGT TTAGACTTCT GAGAGTGAAT GGATACGAGG
TTTCATCAGA GGAACTGGCT CCATATGCTA ACCTAGAGCA
CTTTAGCCAG CAAAAAGTTG ATACTGCAAT GGCTATAGAG
CTCTACAGAG CAGCACAGGA GAGAATACAC GAAGACGAGA
GCGGTCTCGA CAAAATACTT GCTTGGACCA CCACTTTTCT
CGAGCAACAG CTGCTCACTA ACTCCATTCT TGACAATAAA
TTGCATAAAC TGGTGGAGTA CTACTTGAAC AACTACCACG
GCCAAACGAA TAGGGTCGGA GCTAGACGAC ACCTCGACCT
ATATGAGATG AGCCATTACC AAAATCTAAA ACCTTCACAT
AGTCTATGCA ATGAAGACCT TCTAGCATTT GCAAAGCAAG
GTTTTCGAGA TTTTCAAATC CAGCAGCAGA AAGAATTCGA
GCAACTGCAA AGGTGGTATG AAGATTGCAG GTTGGACAAG
TTGAGTTATG GGAGAGATGT AGTAAAAATT TCTAGTTTCA
TGGCTTCAAT ATTGATGGAT GATCCAGAAT TAGCCGATGT
TCGTCTCTCC ATCGCCAAAC AGATGGTGCT CGTGACACGT
ATCGATGATT tCTTCGACCA CGGTGGCTCT ACAgAaGACT
CCTACAAGAT CATTGAACTA GTAAAAGAAT GGAAGGAGAA
GGCaGAATAC GATTCCGAGG AAGTAAAAAT CCTTTTTACA
GCAGTATACA CCACAGTAAA TGAGCTAGCA GAGGCTTGTG
TTCAACAAGG AAGGAATAGT ACTACTGTCA AAGAATTCCT
AGTTCAGTTG TGGATTGAAA TACTATCAGC TTTCAAGGTC
GAGCTAGATA CGTGGAGCGA TGGCACGGAA GTAAGCCTGG
ACGAGTACTT GTCGTGGTCG TGGATTTCGA ATGGCTGCAG
AGTGTCTATA GTAACGACGA TGCATTTGCT CCCTACGAAA
TTATGCAGTG ATGAAATGCT TAGGAGTGAA GAGTGCAAGG
ATTTGTGTAG GCATGTTTCT ATGGTTGGCC GCTTGCTCAA
CGACATCCAC TCTTTTGAGA AGGAGCATGA GGAGAATACG
GGAAACAGTG TGAGCATTCT AGTAGCAGGT GAGGATACCG
AAGAGGAAGC TATTGGAAAG ATCAAAGAGA TAGTTGAGTA
TGAGAGGAGA AAATTGATGC AAATTGTGTA CAAGAGAGGA
ACCATTCTCC CAAGAGAATG CAAAGACATA TTCTTGAAGG
CGTGTAGGGC TACATTTTAC GTGTACTCGA GCACGGATGA
GTTTACGTCT CCTCGACAAG TGATGGAAGA TATGAAAACC
CTAAGCTCCT AG

Origanum majorana palustradiene synthase (OmTPS5) can also convert (+)-copalyl diphosphate ((+)-CPP) [31]) to palustradiene [29].

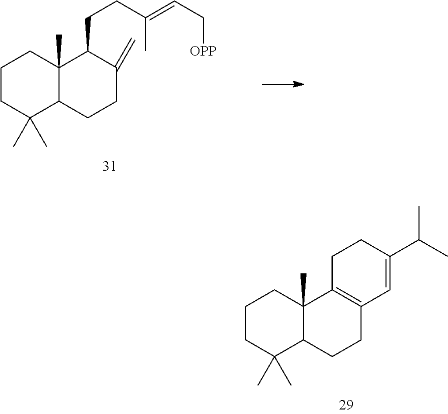

The Origanum majorana palustradiene synthase (OmTPS5) can have the amino acid sequence shown below (SEQ ID NO:13).

MVSACLKLKN NPFLDHRFRK SSNGFSVNFP ATMLTTVKCS
RDNSEDLIAK IKERMNEKFV TVPAREYSVI EHRNPKPAWC
GGLQSKTVIE EEVCSRLFLV EHLQDLGVDR FFQSEIQHIL
HHTFRLWQQK DEQVFKDVTC RAMAFRLLRL EGYHVSSGEL
GEYVDEEKFF RTVRLEWRST DTILELYKAS QVRLPEDDND
NSNILKNLHE WTFIFLKEQL RRKTILDKGL ERKVEFYLKN
YHGILDAVKH RRSLDHTRFW KTTAYNPAVY DEDLERLSAQ
DFMARQAQSQ KELEMLLKWY DECRLDKMEY GRNVIHVSHF
LNANNFPDPR LSETRLSFAK TMTLVTRLDD FFDHHGSRED
SVLIIELIRQ WNEPSTITTI FPSEEVEILY SALHSTVTDI
AEKAYPIQGR CIKSLIIHLW VEILSSFMSE MDSCTAETQP
DFHEYLGFAW ISIGCRICIL IAIHFLGEKV SQQMVMGAEC
TELCRHVSTI ARLLNDLQTF KKEREERKVN SVIIQLKGDK
ISEEVAVSNI ERMVEYHRKE LLKMVVRREG SLVPKRCKDV
FWKSCNIAYY LYAFTDEFTS PQQMKEDMKL LFRDPINCVP
SIPS

A nucleic acid encoding the Origanum majorana palustradiene synthase (OmTPS5) with SEQ ID NO:13 is shown below as SEQ ID NO:14.

ATGGTATCTG CATGTCTAAA ACTCAAAAAT AATCCTTTCT
TGGACCATCG ATTCAGGAAA AGCAGCAATG GATTTTCAGT
TAATTTTCCG GCGACCATGC TCACCACTGT CAAGTGCAGC
CGCGATAATT CAGAAGACTT GATAGCAAAG ATAAAAGAAA
GGATGAATGA AAAATTTGTT ACGGTGCCGG CGAGGGAATA
TTCCGTCATT GAGCATCGGA ATCCGAAGCC GGCGTGGTGC
GGTCGTTTGC AATCCAAAAC AGTAATAGAA GAAGAAGTGT
GCAGCCGTCT GTTTCTGGTC GAACACCTTC AAGATTTAGG
AGTAGACCGC TTCTTTCAAT CAGAAATCCA ACATATTCTA
CATCACACAT TCAGATTATG GCAGCAAAAA GATGAACAAG
TTTTTAAAGA CGTGACATGT CGCGCCATGG CATTCAGACT
CCTGCGTCTC GAAGGTTATC ATGTCTCGTC AGGAGAATTG
GGGGAGTATG TTGATGAGGA AAAATTCTTT AGAACGGTAA
GGTTAGAATG GAGAAGTACG GATACAATTC TTGAGCTGTA
CAAAGCATCA CAGGTAAGAC TACCTGAAGA CGACAACGAC
AATTCCAATA TCCTCAAAAA CTTGCACGAA TGGACCTTCA
TATTTTTGAA GGAGCAGTTG CGGCGTAAAA CTATTCTTGA
TAAAGGTTTA GAGAGAAAGG TAGAATTTTA CTTGAAGAAT
TACCACGGCA TATTAGACGC GGTTAAGCAT AGACGAAGCC
TCGATCACAC ACGATTCTGG AAAACTACTG CGTATAACCC
TGCAGTGTAT GATGAGGATC TTTTCCGATT GTCGGCCCAA
GATTTCATGG CTCGCCAAGC TCAGAGCCAG AAGGAACTTG
AGATGTTGCT CAAGTGGTAC GATGAATGTA GACTGGACAA
GATGGAGTAT GGGCGAAACG TGATACACGT TTCCCATTTC
TTAAACGCAA ACAACTTCCC CGATCCTCGC CTGTCCGAAA
CTCGTCTATC CTTTGCGAAA ACCATGACTC TCGTCACGCG
TTTGGATGAT TTCTTCGATC ACCATGGCTC TAGAGAAGAT
TCGGTCCTCA TCATCGAATT AATAAGGCAG TGGAATGAGC
CTTCAACTAT TACAACAATA TTCCCCTCCG AAGAAGTGGA
GATTCTCTAC TCTGCACTCC ACTCCACCGT AACAGATATA
GCAGAGAAGG CTTATCCCAT CCAGGGTCGC TGCATCAAAT
CGCTCATAAT TCATCTGTGG GTCGAGATAC TGTCGAGCTT
CATGAGCGAA ATGGACTCGT GCACCGCGGA AACTCAGCCG
GACTTTCACG AGTACTTAGG GTTTGCATGG ATCTCGATCG
GCTGCAGAAT CTGCATTCTC ATAGCTATAC ATTTCTTGGG
GGAGAAGGTA TCTCAACAAA TGGTTATGGG TGCTGAGTGC
ACCGAGTTAT GTAGGCACGT TTCTACGATC GCACGCCTTC
TCAACGATCT CCAAACCTTT AAGAAGGAGA GAGAAGAGAG
GAAGGTAAAC AGCGTGATAA TCCAGCTCAA AGGGGATAAG
ATATCGGAGG AGGTGGCCGT GTCGAATATA GAGAGAATGG
TTGAATATCA CAGGAAAGAG CTGCTGAAGA TGGTGGTTCG
GAGAGAAGGA AGCTTGGTTC CTAAGAGGTG TAAGGACGTG
TTCTGGAAAT CCTGCAACAT TGCTTACTAT CTGTACGCTT
TTACAGATGA ATTCACTTCG CCTCAACAAA TGAAGGAAGA
TATGAAACTA CTCTTTCGTC ATCCAATCAA CTGCGTTCCT
TCAATTCCTT CATGA

The Perovskia atriplicifolia miltiradiene synthase (PaTPS3) can have the amino acid sequence shown below (SEQ ID NO:15).

MLLAFNISDV PLSQHRVILS RREHFPRHAF QEFPMIAATK
SSVNAICSLA TPTDLMGKIK EKFKAKDGDP LAAAAIQLAA
DIPSSLCIID TLQRLGVDRY FQSEIDSILE ETHKLWKVKD
RDIYSEVTTH AMAFRLLRVK GYEVSSEELA PYAEQERFDL
QTIDLATVIE LYRAAQERTC EENDNSLEKL LAWTTTFLKH
QLLTNSIPDT KLHKQVEYYL KNYHGILDRM GVRRSLDLYD
ISHYRPLRAR FPNLCNEDFL SFARQDFSMC QAQHQKELEQ
LQRWYSDCRL DALLKFGRNV VRVSSFLTSA IIGEPELSEV
RLVFAKHIIL VTLIDDLFDH GGTREESYKI LELVTEWKEK
TAAEYGSEEV EILETAVYNT VNELVERAHV EQGRSVKEFL
IKLWVQILSI FKIELDTWSD ETALTLDEYL SSSWVSIGCR
ICILMSMQFI GIKLTDEMLL SEECTDLCRH VSMVDRLLND
VQTFEKERKE NTGNSVSLLL AANKDVTEEE AIRRAKEMAE
CNRRQLMQIV YKTGTIFPRK CKDMFLKVCR IGCYLYASGD
EFTSPQQMME DMKSLVYEPL YLPN

A nucleic acid encoding the Perovskia atriplicifolia miltiradiene synthase (PaTPS3) with SEQ ID NO:13 is shown below as SEQ ID NO:16.

ATGTTACTTG CGTTCAACAT AAGCGATGTC CCTCTCTCGC
AGGATAGAGT AATTCTGAGC AGGAGGGAAC ATTTTCCACC
TCATGCATTC CAGGAATTTC CGATGATCGC CGCTACTAAG
TCATCTGTTA ATGCCATTTG CAGCCTCGCT ACTCCAACTG
ATTTGATGGG AAAAATAAAA GAGAAGTTCA AGGCCAAGGA
CGGCGATCCT CTTGCCGCCG CGGCTATTCA ACTCGCGGCG
GATATACCCT CGAGTCTGTG TATAATCGAC ACCCTCCAGA
GGTTGGGAGT CGACCGATAC TTCCAATCCG AAATCGACTC
TATTCTAGAG GAAACACACA AGTTATGGAA AGTGAAAGAT
AGAGATATAT ACTCTGAGGT TACTACTCAT GCAATGGCGT
TTAGACTTCT GCGAGTGAAG GGATATGAAG TTTCATCAGA
GGAACTAGCT CCGTATGCTC AGCAAGAGCG CTTTGACCTG
CAAACGATTG ATCTGGCGAC GGTTATCGAG CTTTACAGAG
CAGCACAGGA GAGAACATGC GAAGAAAACG ACAACAGTCT
TGAGAAACTA CTTGCTTGGA CCACCACCTT TCTCAAGCAC
CAATTGCTCA CCAACTCCAT ACCTGACACC AAATTGCACA
AACAGGTGGA ATACTACTTG AAGAACTACC ACGGGATATT
AGATAGAATG GGAGTTAGAC GAAGCCTCGA CCTATACGAC
ATAAGCCATT ATCGACCTCT GAGAGCAAGA TTCCCTAATC
TGTGTAATGA AGATTTCCTA TCATTTGCGA GGCAAGATTT
CAGTATGTGC CAACCCCAAC ACCAGAAGGA ACTTGAGCAA
CTGCAAAGGT GGTATTCTGA TTGTAGGTTG GACGCGTTGT
TGAAGTTTGG AAGAAATGTA GTGCGCGTTT CTAGCTTTCT
GACTTCAGCA ATTATTGGTG AACCCGAATT GTCTGAAGTT
CGACTAGTCT TTGCCAAACA TATTATTCTC GTTACACTTA
TTCATGATTT ATTCGATCAT GGTGGAACTA GAGAAGAGTC
ATACAAGATC CTTGAATTAG TAACAGAATG GAAAGAGAAG
ACCGCAGCAG AATATGGTTC CGAGGAAGTT GAAATCCTTT
TTACAGCGGT CTACAACACA GTAAATGAGT TGGTAGAGAG
GGCTCATGTC GAACAAGGGC GCAGTGTCAA AGAATTTCTT
ATTAAACTGT GGGTTCAAAT ACTATCAATT TTCAAGATAG
AATTAGATAC ATGGAGCGAT GAGACTGCGC TAACCTTGGA
TGAATACTTG TCTTCGTCGT GGGTGTCAAT TGGTTGCAGA
ATCTGCATTC TCATGTCGAT GCAATTCATC GGTATAAAAT
TAACTGATGA AATGCTTCTG AGTGAAGAGT GCACTGATTT
GTGTAGGCAT GTTTCGATGG TTGACCGGCT GCTCAACGAT
GTGCAAACCT TCGAGAAGGA ACGCAAAGAA AATACAGGAA
ACAGTGTAAG CCTTCTGCTA GCAGCTAACA AAGATGTTAC
TGAAGAGGAA GCAATTAGAA GAGCAAAAGA AATGGCGGAA
TGCAACAGGA GACAACTGAT GCAGATTGTG TATAAAACAG
GAACCATTTT CCCAAGAAAA TGCAAAGATA TGTTTCTCAA
GGTATGCAGG ATTGGCTGTT ATTTGTATGC AAGCGGCGAC
GAATTCACAT CTCCACAACA AATGATGGAA GATATGAAAT
CCTTCGTTTA TGAACCCCTC TACCTACCTA ATTAA

A Perovskia atriplicifolia miltiradiene synthase (PaTPS1) can have the amino acid sequence shown below (SEQ ID NO:17).

MSLTFNAGVV RFSSHRVRST KDCFTVYGFP MIANKAAFAV
KCSLTPTDLM GRVEEKFKGK NGNSLAASTT VESADIPSNL
CIIDTLQRLG VDRYFQTEIN AILEDTYRLW ERKDKDIYSD
ATTHAMAFRL LRVKGYEVSS EELAPYADQE CVNVQTADVA
TVIELYRAAQ VRISEEESSL KKLHAWTTTF LKYQLQSNSI
PEKKLHKLVE YYLKNYHGIL DRMGVRMDLD LFDISHYRTL
QASDRFSSLR NEDFLEFARQ DFNICQAKHQ KELQQLQRWY
ADCRLDTLKF GRDVVRVANF LTSAIFGEPE LSDARLIFAK
HIVLVTCIDE FFDHGGSKEE SYKILELVEE WKEKPTGEYG
CEEVEILFTA VYSTVNELAE MAHVEQGRSV KEFLVKLWVQ
ILSIFKIELD TWSDDTELTL DSYLNNSWVS IGCRICILMS
MQFAGVKLSD EMLLSEECVD LCRHVSMVDR LLNDVQTFEK
ERKENTGNSV SLLQAAAERE GRAITEEEAI TQIKELAEYH
RRKLMQIVYK TDTIFPRKCK DMFLKVCRIG CYLYASGDEF
TTPQQMMEDM KSLVYQPLTV DDMSAKELTS VRN

A nucleic acid encoding the Perovskia atriplicifolia miltiradiene synthase (PaTPS1) with SEQ ID NO:13 is shown below as SEQ ID NO:18.

ATGTCACTCA CTTTCAACGC TGGAGTCGTC CGTTTCTCCA
GCCACCGCGT TCGGAGCACG AAAGATTGCT TTACAGTTTA
CGGATTTCCG ATGATTGCAA ATAAGGCAGC TTTCGCAGTT
AAATGCAGCC TTACTCCAAC CGATTTGATG GGGAGAGTAG
AGGAGAAGTT CAAGGGCAAA AATGGTAATT CACTAGCAGC
CTCGACGACG GTTGAATCCG CGGATATACC CTCGAACCTG
TGTATAATCG ACACCCTCCA AAGATTGGGA GTCGACCGAT
ACTTTCAAAC TGAAATCAAT GCCATTCTAG AGGACACTTA
CAGATTATGG GAACGAAAAG ACAAAGACAT ATATTCCGAT
GCCACAACTC ACGCGATGGC GTTTAGGTTA CTACGAGTGA
AAGGATACGA AGTTTCATCA GAGGAACTGG CTCCTTACGC
TGATCAAGAG TGCGTGAACG TGCAAACGGC TGATGTGGCA
ACAGTTATCG AGCTTTACAG AGCAGCGCAG GTGAGAATAA
GCGAAGAAGA GAGCAGTCTT AAGAAGCTTC ATGCTTGGAC
CACCACCTTT CTCAAATATC AGTTGCAGAG TAACTCCATA
CCTGAAAAGA AACTGCACAA ACTGGTGGAA TATTACTTGA
AGAACTACCA TGGCATATTG GATAGAATGG GAGTTCGAAT
GGACCTCGAC TTATTCGACA TCAGCCATTA TCGAACTCTA
CAAGCTTCCG ATAGGTTCTC TAGTCTGCGT AACGAAGATT
TTCTAGAGTT TGCAAGGCAA GATTTCAATA TCTGCCAAGC
CAAGCACCAG AAAGAACTCC AACAACTGCA AAGGTGGTAT
GCAGATTGCA GGCTCGACAC CTTGAAGTTC GGGAGAGACG
TCGTACGCGT TGCTAATTTT CTGACTTCAG CAATCTTTGG
CGAACCCGAG CTATCCGATG CTCGTCTGAT CTTTGCCAAG
CATATCGTGC TCGTAACATG TATCGATGAA TTCTTCGATC
ATGGTGGGTC TAAAGAAGAG TCCTACAAGA TCCTTGAATT
AGTAGAAGAA TGGAAAGAGA AGCCAACTGG AGAATATGGG
TGTGAGGAGG TTGAGATCCT TTTCACAGCA GTGTACAGTA
CAGTGAATGA GTTGGCAGAG ATGGCTCATG TCGAACAAGG
ACGTAGTGTG AAAGAGTTTC TAGTTAAACT GTGGGTGCAG
ATACTGTCGA TTTTCAAGAT AGAACTGGAT ACATGGAGTG
ATGACACGGA ACTGACGTTG GACAGCTACT TGAACAACTC
GTGGGTGTCG ATCGCATGCA GAATCTGCAT TCTCATGTCG
ATGCAGTTCG CCGGTGTAAA ACTGTCCGAC GAAATGCTTC
TGAGTGAAGA GTGTGTTGAC TTGTGCAGGC ACGTCTCCAT
GGTCGATCGC CTCCTGAACG ATGTGCAAAC TTTCGAGAAG
GAACGCAAGG AAAATACAGG AAACAGTGTG AGCCTTCTGC
AAGCAGCAGC TGAGAGAGAA GGAAGACCCA TTACAGAAGA
GGAAGCTATT ACACAGATCA AAGAATTGGC TGAATACCAC
AGGAGAAAAC TGATGCAGAT TGTGTACAAA ACAGACACCA
TTTTCCCAAG AAAATGCAAA GATATGTTCT TGAAGGTGTG
CAGGATTGGG TGCTATCTGT ACGCAAGTGG AGACGAATTC
ACAACTCCAC AACAAATGAT GGAAGACATG AAATCATTGG
TTTATCAACC CCTAACAGTT GATGACATGA GTGCCAAAGA
ATTGACTTCT GTGAGAAACT AG

The Salvia officinalis miltiradiene synthase (SoTPS1) can have the amino acid sequence shown below (SEQ ID NO:19).

MSLAFNAAVA TFSGHRIRSR REILPGQGFP MITNKSSFAV
KCNLTTTDLM GKITEKFKGR DSNFSAATAV QPAADIPSNL
CIIDTLQRLG VDRYFQSEID TILEDTYRLW QRKEREIFSD
ITIHAMAFRL LRVKGYVVSS EELAPYADQE RINLQRIDVA
TVIELYRAAQ ERISEDESSL EKLHAWTATY LKQQLLTNSI
PDKKLNKLVE CYLKNYHGIL DRMGVRQNLD LYDISHYQTL
KAADRFSNLR NEDFLAFARQ DFNICQEQHQ KELQQLQRWY
ADCRLDTLKY GRDVVRVANF LTSAIIGDPE LSEVRLVFAK
HIVLVTRIDD FFDHGGSREE SYKILELLKE WKEKPAAEYG
SKEVEILFIA VYNTVNELAE MAHIEQGRSV KEFLIKLWVQ
IISIFKIELD TWSDETALTL DEYLSSSWVS IGCRICILMS
MQFIGIKLSD EMLLSEECID LCREVSMVDR LLNDVQTFEK
ERKENTGNSV SLLLAANKDD SAFTEEEAIT KAKEMAECNR
RQLMKIVYKT GTIFPRKCKD MFLKVCRIGC YLYASGDEFT
SPQQMMEDMK SLVYEPLTVD PLEAKNVSGK

A nucleic acid encoding the Salvia officinalis miltiradiene synthase (SoTPS1) with SEQ ID NO:19 is shown below as SEQ ID NO:20.

ATGTCCCTCG CCTTCAACGC AGCAGTTGCC ACTTTCTCCG
GCCACAGAAT TCGGAGCAGG AGAGAAATTC TTCCGGGGCA
AGGATTTCCG ATGATCACCA ACAAGTCGTC TTTCGCCGTG
AAATGTAACC TTACTACAAC AGATTTGATG GGCAAGATAA
CAGAGAAATT CAAGGGAAGA GACAGTAATT TTTCAGCAGC
AACCGCTGTT CAACCTGCGG CGGATATACC CTCTAACCTG
TGCATAATCG ACACCCTCCA AAGGTTGGGA GTCGACCGAT
ACTTCCAATC TGAAATCGAC ACTATTCTAG AGGACACATA
CAGGTTATGG CAAAGGAAAG AGAGAGAGAT ATTTTCGCAT
ATAACTATTC ATGCAATGGC ATTTAGACTT TTGCGAGTTA
AAGGATATGT AGTTTCATCA GAGGAACTGG CTCCGTATGC
TGACCAAGAG CGCATTAACC TGCAAAGGAT TGATGTAGCG
ACAGTTATCG AGCTTTACAG AGCAGCACAG GAGAGAATAA
GTGAAGACGA GAGCAGTCTT GAGAAACTAC ATGCTTGGAC
CGCCACCTAT CTCAAGCAGC AGCTGCTCAC TAACTCCATT
CCTGAGAAGA AATTGAACAA ACTGGTGGAA TGCTACTTGA
AGAACTATCA CGGGATATTA GATAGAATGG GAGTTAGACA
AAACCTCGAC CTCTACGACA TAAGCCACTA TCAAACTCTA
AAAGCTGCAG ATAGGTTCTC TAATCTACGT AATGAAGATT
TTCTAGCATT TGCGAGGCAA GATTTTAATA TTTGCCAAGA
ACAACACCAA AAAGAACTTC AGCAACTGCA AAGGTGGTAT
GCAGATTGTA GGTTGGACAC ATTGAAGTAT GGAAGAGATG
TCGTGCGGGT TGCTAATTTT CTAACATCAG CAATTATTGG
TGATCCTGAA TTGTCTGAAG TCCGTCTAGT CTTCGCCAAA
CATATTGTGC TTGTAACACG TATTGATGAT TTTTTCGATC
ATGGTGGATC TAGAGAAGAG TCCTACAAGA TCCTTGAATT
ACTAAAAGAA TGGAAAGAGA AGCCAGCTGC AGAATATGGT
TCCAAAGAAG TTGAAATTCT TTTCACAGCA GTATACAATA
CAGTAAACGA GTTGGCAGAG ATGGCTCACA TCGAACAAGG
ACGTAGTGTT AAAGAATTTC TAATAAAGCT GTGGGTTCAA
ATCATATCGA TTTTCAAGAT AGAATTAGAT ACATGGAGCG
ATGAGACAGC GCTGACCTTG GATGAGTACT TGTCTTCGTC
GTGGGTGTCA ATTGGGTGCA GAATCTGCAT TCTCATGTCG
ATGCAATTCA TTGGTATAAA ATTATCTGAT GAAATGCTTC
TGAGTGAAGA GTGTATTGAT TTGTGTCGGC ATGTCTCCAT
GGTTGACCGG CTGCTCAACG ACGTGCAGAC TTTCGAGAAG
GAACGCAAGG AAAATACAGG AAATAGCGTG AGCCTTCTGC
TAGCAGCTAA CAAAGACGAC AGCGCCTTTA CTGAAGAGGA
AGCTATTACA AAAGCAAAAG AAATGGCGGA ATGTAACAGG
AGACAACTGA TGAAGATTGT GTATAAAACA GGAACCATTT
TCCCAAGAAA ATGCAAAGAT ATGTTTCTGA AGGTATGCAG
GATTGGCTGT TACTTGTATG CAAGCGGCGA TGAATTCACA
TCTCCACAAC AAATGATGGA AGATATGAAA TCCTTGGTCT
ATGAACCCCT AACAGTTGAT CCTCTCGAGG CCAAAAATGT
GAGTGGCAAA TGA

Ajuga reptans (+)-copalyl diphosphate synthase (ArTPS1) is a (+)-copalyl diphosphate ((+)-CPP) [31] synthase, and compound 31 is shown below.

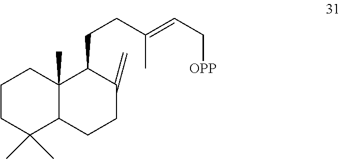

The Ajuga reptans(+)-copalyl diphosphate synthase (ArTPS1) can have the amino acid sequence shown below (SEQ ID NO:21).

MASLSTFHLY SSSLLHRKTL QSSPKLNLSS ECFSTRTWMN
SSKNLSLNYQ VNQKIGKLTG TRVATVDAPQ QLEHDDSTAK
GHDIVDIETQ DPIEYIRMLL NTTGDGRISV SPYDTAWIAL
IKDVEGRDFP QFPSSLEWIA NHQLADGSWG DEGFFCVYDR
LVNTIACVVA LRSWNVHHDK SQRGIQYIKE NVHQLKDGNA
EHMMCGFEVV FPALLQKAKN MGIDDLPYEA PVIQDIYHTR
EQKLKRIPLE MMHKVPTSLL FSLEGLENLD WDKLLKLQSA
DGSFLTSPSS TAFAFMQTKD EKCFQFIKNT VETFNGGAPH
TYPVDVFGRL WAVDRLQRLG ISRFFEAEIA DCLSHIHRYW
NDKGLFSGRE SDFVDIDDTS MGFRLLRMQG YDVSPNVLRN
FKNGDKFSCY GGQTIESSTP IYNLYRASQF RFPGEEILEE
ADKFAHEFLS EQLGNNQLLD KWVISDRLQE EISIGLGMPF
YATLPRVEAS YYIQHYAGAD DVWIGKTLYR MPEISNDTYL
ELARNDFKRC QAQHQFEWIY MQEWYESCNI EEFGISRKEL
LRVYFLACSS IFEVERTKER MAWAKSQIIS RMITSFENKQ
TTSSEEKETL LTEFRNINGL HKSNNTRDGD MNIVLATLHQ
FFAGFDRYTS HQLKNAWGVW LSKLQRGAVD GGADAELITT
TINVCAGHIA LKEDILSHDE YKTLTDLTSK ICQQLSHIQN
EKVVEIDGGI TAKSRLKNEE LQRDMQSLVK LVLEKSVGLN
RNIKQTFLTV AKTYYYRAYN AEETMDAHIF KVLFEPVA

A nucleic acid encoding the Ajuga reptans (+)-copalyl diphosphate synthase (ArTPS1) with SEQ ID NO:21 is shown below as SEQ ID NO:22.

ATCGCCTCTT TGTCCACTTT CCACCTCTAC TCTTCCTCAC
TCCTTCACCG CAAAACACTG CAATCTTCAC CAAAGCTTAA
CCTGTCTTCA GAATGCTTCT CCACCAGAAC TTGGATGAAC
AGCAGCAAAA ACTTGTCGTT AAATTACCAA GTTAATCAGA
AAATAGGAAA GCTGACAGGG ACTCGAGTTG CCACTGTGGA
TGCGCCACAA CAACTTGAAC ACGATGATTC AACTGCTAAA
GGCCATGATA TAGTCGATAT TGAAACTCAG GATCCAATTG
AATATATTAG AATGCTGTTG AACACAACAG GCGATGGCAG
AATCAGCGTT TCGCCTTACG ACACAGCATG GATTGCTCTT
ATTAAGGACG TGGAAGGACG TGATTTTCCT CAATTTCCAT
CCAGCCTTGA GTGGATCGCG AACCATCAAC TCGCTGATGG
TTCATGGGGA GACGAAGGAT TTTTCTGTGT GTATGATCGG
CTCGTAAATA CTATAGCATG TGTCGTAGCA TTGAGATCAT
CGAATGTCCA TCACGACAAG AGCCAAAGAG GAATACAATA
TATCAAGGAA AATGTGCATC AACTTAAGGA TGGAAATGCT
GAGCACATGA TGTGTGGTTT CGAAGTAGTG TTTCCTGCAC
TTCTTCAAAA AGCCAAAAAT ATGGGCATTG ATGATCTTCC
ATATGAGGCT CCTGTCATCC AGGATATTTA CCATACAAGG
GAGCAGAAAT TGAAAAGGAT ACCATTGGAG ATGATGCACA
AAGTGCCTAC TTCTCTGCTG TTTAGTTTGG AAGGACTGGA
GAATTTAGAT TGGGATAAAC TCCTTAAGTT GCAGTCAGCT
GATGGCTCTT TCCTCACTTC TCCCTCCTCT ACTGCTTTCG
CATTCATGCA AACAAAAGAC GAAAAATGCT TCCAGTTCAT
CAAGAACACT GTTGAAACCT TTAATGGAGG AGCACCACAT
ACTTATCCGG TCGATGTTTT TGGAAGACTT TGGGCGGTTG
ATAGGCTGCA GCGCCTCGGA ATTTCTCGAT TCTTTGAGGC
TGAGATTGCT GATTGCTTAA GTCACATTCA TAGATATTGG
AATGATAAGG GGCTTTTCAG TGGACGTGAA TCGGACTTTG
TCGATATTGA CGACACATCC ATGGGTTTCA GACTTCTAAG
AATGCAAGGC TATGATGTTA GTCCAAATGT ACTGAGGAAT
TTCAAGAATG GTGACAAGTT TTCATGTTAC GGAGGTCAAA
CGATCGAGTC ATCAACTCCA ATATACAATC TGTACAGACC
TTCTCAATTC CGGTTTCCAG GAGAAGAAAT TCTTGAAGAA
GCCGACAAGT TCGCCCATGA GTTCTTGTCC GAACAGCTTG
GCAACAACCA ATTGCTTGAT AAATGGGTTA TATCCGACCG
CTTGCAGGAA GAGATAAGTA TTGGATTGGG GATGCCATTT
TATGCCACCC TTCCCAGAGT TGAAGCAAGC TACTATATAC
AACATTACGC TGGTGCCGAC GACGTGTGGA TCGGCAAGAC
ACTCTACAGG ATGCCGGAAA TAAGTAATGA TACATACCTG
GAGCTAGCAA GAAATGATTT CAAGAGATGC CAAGCACAAC
ATCAGTTCGA GTGGATCTAC ATGCAAGAAT GGTATGAGAG
TTGCAACATT GAAGAATTCG GGATAAGCCG AAAGGAGCTC
CTTCGCGTTT ACTTTTTGGC TTGCTCTAGC ATCTTTGAGG
TCGAGAGGAC TAAAGAGAGA ATGGCATGGG CAAAATCTCA
AATTATTTCT AGAATGATCA CTTCTTTCTT TAATAAACAA
ACTACTTCAT CTGAGGAAAA AGAAACACTT TTAACCGAAT
TCAGAAACAT CAACGGTCTG CACAAATCAA ACAATACAAG
AGATGGAGAT ATGAACATTG TGCTTGCAAC CCTCCATCAA
TTCTTCGCTG GATTTGACAG ATATACTAGC CATCAACTGA
AAAATGCTTG GGGAGTATGG TTGACCAAGC TGCAACGAGG
AGCAGTAGAC GGTGGAGCAG ACGCAGAGCT GATAACAACC
ACCATAAACG TATGCGCCGG TCATATAGCT CTTAAGGAAG
ACATATTGTC CCACGATGAG TACAAGACTC TCACCGACCT
CACCAGCAAG ATTTGTCAGC AGCTTTCTCA TATTCAAAAC
GAAAAGGTTG TGGAAATTGA CGGTGGGATT ACAGCAAAAT
CTAGGTTGAA GAATGAGGAA CTGCAACGTG ACATGCAATC
ATTGGTGAAA TTAGTACTTG AGAAATCAGT TGGGCTCAAC
CGGAATATAA AGCAAACATT TCTAACGGTT GCAAAAACAT
ACTACTACAG AGCCTACAAT GCTGAGGAAA CTATGGATGC
CCATATATTC AAAGTTCTTT TCGAACCAGT TGCGTGA

Ajuga reptans cleroda-4(18),13E-dienyl diphosphate synthase (ArTPS2) was identified and isolated as described herein. ArTPS2 was identified as a (5R,8R,9S,10R) neo-cleroda-4(18),13E-dienyl diphosphate [38] synthase. In addition, the combination of ArTPS2 and SsSS enzymes generated neo-cleroda-4(18),14-dien-13-ol [37]. These compounds are shown below.

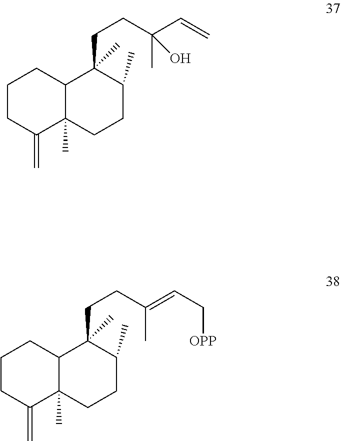

ArTPS2 is of particular interest for applications in agricultural biotechnology, for example, because it is useful for production of neo-clerodane diterpenoids. Neo-clerodane diterpenoids, particularly those with an epoxide moiety at the 4(18) position, have garnered significant attention for their ability to deter insect herbivores (Coll et al., Phytochem Rev 7(1):25 (2008); Klein Gebbinck et al. Phytochemistry 61(7):737-770 (2002); Li et al. Nat Prod Rep 33(10):1166-1226 (2016)). The 4(18)-desaturated products produced by ArTPS2 (e.g., compounds 37 and 38 with the ═CH₂ 4(18) desaturation projecting from the A ring) the can be used in biosynthetic or semisynthetic routes to yield potent insect antifeedants.

The Ajuga reptans cleroda-4(18),13E-dienyl diphosphate synthase (ArTPS2) can have the amino acid sequence shown below (SEQ ID NO:23).

MSFASQATSL LSSPNRLGHV PTPSSPARFA AGGAPFWKIL
FTARSNGQYK AISRARNQGN VEYIDEIQKG PQVVLEAENS
LEDDTQKDTD QIRELVENVR VKLQNIGGGG ISISAYDTAW
VALVEDINGS GQPQFPTSLD WISNHQFPDG SWGSSKFLYY
DRILCTLACI VALKTWNVHP DKYHKGLDFI RENIHKLADE
EEVHMPIGFE VAFPSIIETA KKVGIEIPED FPGEKEIYAK
RDLKLKKIPM DILHKMPTPL LFSIEGMEGL DWQKLFKFRD
DGSFLTSPSS TAYALQQTKD ELCLKYLTDL VKKDNGGVPN
AFPVDLFDRN YTVDRLRRLG ISRYFQPEIE ECMKYVYRFW
DKRGISWARN TNVQDLDDTA QGFRNLRMHG YEVTLDVFKQ
FEKCGEFFSF HGQSSDAVLG MFNLYRASQV LFPGEHMLAD
ARKYAANYLH KRRLNNRVVD KWIINKDLEG EVAYGLDVPF
YASLPRLEAR FYIEQYGGSD DVWIGKALYR MVNVSCDTYL
ELAKLDYNKC QSVHQNEWKS FQKWYKSCSL GEFGFSEGSL
LQAYYIAAST IFEPEKSGER LAWAKTAALM ETIQQLSSQQ
KREFVDEFKH KNILKNENGE RYRSSTSLVE TLISTVNQLS
SDILLEQGRD VHQELCHVWL KWLSTWEERG NLVEAEAELL
LRTLHLNSGL DESSFSHPKY QQLLEVSTKV CHLLRLFQKR
KVYDPEGCTT DIATGTTFQI EACMQELVKL VFSRSSEDLD
SLTKLRFLDV ARSFYYTABC DPQVVESHID KVLFEKVV

A nucleic acid encoding the Ajuga reptans cleroda-4(18),13E-dienyl diphosphate synthase (ArTPS2) with SEQ ID NO:23 is shown below as SEQ ID NO:24.

ATGTCATTTG CTTCCCAAGC CACCTCCCTC CTATCATCCC
CCAACCGTCT CGGCCATGTT CCGACGCCAA GCTCGCCGGC
TCGTTTCGCT GCCGGTGGTG CCCCATTTTG GAAGATATTA
TTTACAGCTA GGTCTAATGG GCAGTATAAA GCTATTTCAA
GAGCTCGTAA CCAAGGAAAT GTAGAGTACA TTGATGAGAT
TCAGAAAGGC CCGCAAGTCG TATTGGAGGC AGAAAACAGC
TTGGAAGATG ACACACAAAA AGATACTGAT CAGATAAGGG
AACTAGTGGA AAATGTCCGA GTAAAGCTGC AGAATATCCG
TGGTGGAGGG ATAAGCATAT CGGCGTACGA CACCGCATGG
GTGGCGCTGG TGGAGGACAT CAACGGCAGT GGCCAGCCAC
AGTTTCCGAC GAGCCTCGAT TGGATATCGA ACCATCAGTT
CCCTGATGGG TCATGGGGCA GCAGCAAGTT TTTGTATTAT
GATCGGATTC TATGCACATT AGCATGTATA GTTGCATTGA
AAACCTGGAA TGTGCATCCT GATAAGTACC ACAAAGGGTT
GGATTTCATC AGAGAGAACA TTCACAAGCT TGCGGACGAA
GAAGAAGTGC ACATGCCAAT TGGGTTCGAA GTGGCATTCC
CATCAATTAT TGAAACAGCT AAAAAAGTAG GAATCGAAAT
CCCTGAGGAT TTTCCTGGCA AGAAAGAAAT TTATGCAAAA
AGAGATTTAA AGCTAAAAAA AATACCAATG GATATACTGC
ATAAAATGCC CACACCATTG CTCTTCAGCA TAGAAGGAAT
GGAAGGCCTT GACTGGCAAA AGCTATTCAA ATTCCGCGAT
GATGGCTCGT TTCTTACGTC TCCGTCCTCA ACAGCCTATG
CACTCCAGCA AACAAAGGAT GAGCTATGCC TCAAGTATCT
AACAGATCTT GTCAAGAAAG ACAACGGAGG AGTTCCGAAT
GCATTTCCAG TAGACCTGTT TGATCGTAAC TATACAGTAG
ACCGCTTGCG AAGGCTAGGA ATTTCACGGT ACTTTCAACC
TGAAATTGAA GAATGCATGA AATATGTTTA CAGATTTTGG
GATAAAAGAG GAATTAGCTG GGCAAGAAAT ACCAATGTTC
AGGACCTTGA TGACACTGCA CAGGGATTCA GGAATTTAAG
GATGCATGGT TATGAAGTCA CTCTAGATGT TTTCAAACAA
TTTGAGAAAT GTGGAGAGTT TTTCAGTTTT CATGGGCAAT
CCAGCGATGC TGTTTTAGGA ATGTTCAACT TGTACCGGGC
TTCTCAGGTT TTATTTCCGG GAGAACACAT GCTTGCAGAT
GCGAGGAAGT ATGCAGCCAA CTATTTGCAT AAACGAAGAC
TTAATAATAG GGTGGTCGAC AAATGGATTA TCAACAAAGA
CCTTGAAGGC GAGGTGGCAT ATGGGCTAGA TGTTCCGTTC
TACGGCAGCC TACCTCGACT CGAAGCAAGG TTCTACATAG
AACAATATGG GGGTAGTGAT GATGTGTGGA TTGGAAAAGC
TTTATACAGA ATGGTAAATG TAAGCTGCGA CACTTACCTT
GAGCTAGCAA AATTAGACTA CAACAAATGC CAATCCGTGC
ATCAGAATGA GTGGAAAAGC TTTCAAAAAT GGTACAAAAG
TTGCAGTCTT GGGGAGTTTG GGTTCAGTGA AGGAAGCCTA
CTCCAAGCTT ACTACATAGC AGCCTCAACT ATATTCGAGC
CAGAGAAATC AGGAGAACGC CTAGCTTGGG CTAAAACAGC
AGCTCTAATG GAGACAATTC AACAACTTTC CAGCCAGCAA
AAACGTGAAT TTGTTGATGA ATTCAAACAT AAAAACATAC
TGAAGAATGA AAATGGAGAA AGGTATAGAT CAAGTACCAG
TTTGGTAGAG ACTCTGATAA GCACTGTAAA TCAGCTCTCA
TCAGACATAC TATTGGAGCA AGGCAGAGAC GTTCATCAAG
AATTATGTCA CGTGTGGCTA AAATGGCTGA GTACATGGGA
GGAAAGAGGA AACCTGGTGG AAGCGGAAGC CGAGCTTCTT
CTGCGAACCT TACATCTCAA CAGCGGATTG GATGAATCAT
CATTTTCCCA CCCTAAATAT CAACAGCTCT TGGAGGTGTC
TACCAAAGTT TGCCACCTCC TTCGCCTATT TCAGAAACGA
AAGGTGTATG ATCCCGAAGG GTGTACAACC GACATAGCAA
CAGGAACAAC GTTCCAGATA GAAGCATGCA TGCAAGAACT
AGTGAAATTA GTGTTCAGCA GATCCTCAGA AGATTTAGAT
TCTCTTACTA AGTTGAGATT TTTGGATGTT GCTAGAAGTT
TCTATTACAC TGCCCATTGT GATCCACAGG TGGTCGAGTC
CCACATCGAT AAAGTATTGT TTGAGAAGGT AGTCTAG

The Plectranthus barbatus (+)-Copalyl diphosphate synthase (CfTPS16) was identified and isolated using the methods described herein, and this CfTPS116 protein can have the amino acid sequence shown below (SEQ ID NO:25).

MQASMSSLNL NNAPAVCSSR SQLSAKLHPP EYSTVGAWLN
RGNKNQRLGY RIRPKQLSKL TECRVASADV SGEIGKVCQS
VRTPEEVNKK IEESIKYVKE LLMTSGDGRI SVAPYDTAIV
ALIKDLEGRD APEFPSCLEW IANNQKDDGS WGDDFFCIYD
RIVNTIASVV ALKSWNVHPD KIERGVSYIK ENAHKLKGGN
LEHMTSGFEF VVPGCFDRAK ALGIEGLPYD DPIIKEIYAT
KERRLSKVPK DMIYKVPTTL LFSLEGLGME DLDWQKILKL
QSGDGSFLTS PSSTAYAFMQ TGDEKCYKFL QNAVRNCNGG
APHTYPVDVF ARLWAVDRLQ RLGISRFFQP EIKFCLDHIK
NVWTKNGVFS GRDSEFVDID DTSMGIRLLK MHGYDVDPNA
LKHFKQEDGR FSCYGGQMIE SASPIYNLYR AAQLRFPGEE
ILEEATKFAY NFLQQKLANN QIQEKWVISE HLIDEIKMGL
KMPWYATLPR VEASYYLQYY AASGDVWIGK TFYRMPEISN
DTYKELALLD FNRCQAQHQF EWIYMQEWYQ SNNIKEFGIS
KKELLLAYFL AAATIFEPER SQERIVWAKT QVVSKMITSF
LSQENALSSX QKTALFIDFG HSINGLNQIT SVEKENGLAQ
TVLATFGQLL EEYDRYTRHQ LKNAWSQWFM KLQQGDDNGG
ADAELLANTL NICAGHIAFN EDILSHNEYT SLSSLTNKIC
QRLSQIRDNK ILEIEDGSIK DKELEQEMQA LVKLVLEETG
GIDRNIKQTF LSVFKMFYYR AYHDAEAIDX HIFKVMFEPV
V

A nucleic acid encoding the Plectranthus barbatus (+)-Copalyl diphosphate synthase (CfTPS16) with SEQ ID NO:25 is shown below as SEQ ID NO:26.

ATGCAGGCTT CTATCTCATC TCTGAACTTG AACAATGCAC
CGGCCGTCTG CAGCAGCAGG TCACAGCTAT CCGCTAAACT
TCACCCGCCG GAATATTCCA CCGTGGGTGC ATGGCTGAAT
CGTGGCAACA AAAACCAGCG GTTGGGCTAC CGGATTCGTC
CAAAGCAACT ATCAAAACTA ACTGAGTGTC GAGTAGCAAG
TGCAGATGTG TCACAAGAGA TTGGAAAAGT CGGCCAATCT
GTTCGGACTC CTGAAGAGGT AAATAAAAAG ATAGAGGAAT
CCATCAAGTA CGTGAAGGAG CTGCTGATGA CGTCGGGCGA
CGGGCGAATC AGTGTGGCGC CCTACGACAC GGCCATAGTT
GCCCTTATCA AGGACTTGGA AGGGCGCGAT GCCCCGGAGT
TTCCATCTTG CTTGGAGTGG ATTGCAAACA ATCAAAAAGA
CGATGGTTCT TGGGGGGATG ACTTCTTCTG CATCTATGAT
CGGATCGTTA ATACCATAGC ATCCGTCGTC GCCTTAAAAT
CATGGAATGT GCACCCAGAC AAGATTGAGA GAGGAGTATC
CTACATCAAG GAAAACGCGC ATAAACTAAA AGGTGGGAAT
CTCGAACACA TGACATCAGG GTTCGAGTTC GTGGTTCCCG
CGTGTTTTGA CAGAGCCAAA GCCTTGGGCA TCGAAGGCCT
TCCCTATGAT GATCCCATCA TCAAGGAGAT TTATGCTACA
AAAGAAAGGA CATTGAGCAA GGTACCGAAG GACATGATCT
ACAAAGTTCC GACAACTCTA TTGTTTAGTT TAGAGGGACT
GGGCATGGAG GATTTGGACT GGCAAAAGAT ACTGAAACTG
CAGTCGGGCG ACGGCTCATT CCTCACCTCT CCGTCGTCCA
CCGCCTACGC ATTCATGCAG ACCGGAGACG AAAAATGCTA
CAAATTCCTC CAGAACGCCG TCAGAAATTG CAACGGCGGA
GCGCCGCACA CTTATCCAGT CGACGTCTTT GCACGGCTCT
GGGCGGTCGA CCGACTTCAG CGACTCGGAA TTTCTCGCTT
CTTTCAGCCC GAGATCAAGT TTTGCCTAGA CCACATCAAA
AATGTGTGGA CTAAGAACGG AGTTTTCAGT GGACGGGATT
CAGAGTTTGT GGATATCGAC GACACATCCA TGGGCATCAG
GCTTCTGAAA ATGCACGGAT ACGATGTCGA CCCAAATGCA
CTGAAACATT TCAAGCAGGA GGATGGGAGG TTTTCATGCT
ACGGTGGTCA AATGATCGAG TCTGCATCTC CGATTTACAA
TCTCTACAGG GCTGCTCAGC TTCGTTTTCC AGGAGAAGAA
ATTCTTGAAG AAGCCACTAA ATTTGCCTAC AACTTCCTGC
AACAGAAGCT GGCCAACAAT CAAATTCAAG AAAAGTGGGT
CATATCCGAG CACCTAATTG ATGAGATAAA AATGGGATTG
AAGATGCCAT GGTACGCCAC CCTACCTAGA GTTGAGGCTT
CATACTATCT CCAATATTAT GCAGCTTCTG GCGACGTATG
GATTGGCAAG ACTTTTTACA GGATGCCAGA AATAAGTAAT
GACACGTACA AAGAGCTTGC ACTATTGGAT TTCAACCGAT
GCCAAGCACA ACATCAGTTC GAATGGATTT ACATGCAAGA
GTGGTATCAA AGCAACAACA TTAAAGAATT TGGGATAAGC
AAGAAAGAGC TTCTTCTTGC TTACTTCTTG GCTGCTGCAA
CCATTTTTGA ACCCGAACGA TCGCAAGAGC GGATCGTGTG
GGCTAAAACC CAAGTTGTTT CTAAGATGAT CACATCGTTT
CTGTCTCAAG AAAACGCTTT GTCATCGGAN CAAAAGACTG
CACTTTTCAT CGATTTTGGG CATAGTATCA ATGGCCTCAA
TCAAATAACT AGTGTTGAGA AAGAGAATGG GCTTGCTCAG
ACTGTCCTGG CAACCTTCGG ACAACTACTC GAGGAATTCG
ACAGATACAC AAGGCATCAA CTGAAAAATG CTTGGAGCCA
ATGGTTCATG AAACTGCAGC AAGGAGATGA CAATGGCGGG
GCAGACGCAG AGCTCCTAGC AAACACATTG AACATCTGCG
CTGGTCATAT TGCTTTTAAC GAAGACATAT TATCTCACAA
CGAATACACC TCTCTCTCCT CCCTCACAAA CAAAATCTGT
CAGCGGCTAA GTCAAATTCG AGATAATAAG ATACTGGAAA
TTGAGGATGG GAGCATAAAA GATAAGGAAC TAGAACAGGA
AATGCAGGCG CTGGTGAAGT TAGTCCTGGA AGAAACCGGT
GGCATCGACA GGAACATCAA GCAAACATTT TTGTCAGTTT
TCAAAATGTT TTACTACAGA GCCTACCACG ATGCTGAGGC
TATCGATGNC CATATTTTCA AAGTAATGTT TGAACCAGTC
GTATGA

Hyptis suaveolens labda-7,13E-dienyl diphosphate synthase (HsTPS1) was identified and isolated as described herein, and is a (55, 95, 105) labda-7,13E-dienyl diphosphate [21] synthase. When HsTPS1 was expressed in N. benthamiana, labda-7,13(16),14-triene [22] was formed. The combination of HsTPS1 with OmTPS3 produced labda-7,12E,14-triene [24].

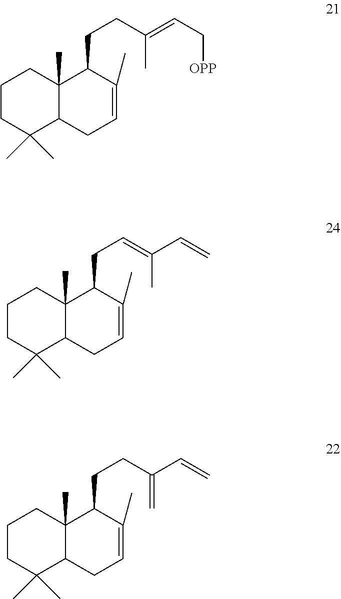

The Hyptis suaveolens labda-7,13E-dienyl diphosphate synthase (HsTPS1) can have the amino acid sequence shown below (SEQ ID NO:27).

MAYMISISNL NCSSLINTNL SAKIQLHQGL KGTWLKTSKR
MCMDQQVHGK QIAKVIESRV TDKDVSTAQD FEVLKVNRVE
DLISSIKSSL KTMEDGRISV SPYSTSWIAL IPSIDGRQTP
QFPSSLEWIV KHQLSDGSWG DALFFCVYDR LVNTIACIIA
LHTWKVHADK VKKGVSFVKE NIWKLEDANE VHMTSGFEVI
FPILLRRARD MGIDGLPSDD TPVVRMISAA RDHKLKKIPR
EVMHQVTTIL LYSLEGLEDL DWSRLFKLQS ADGSFLTSPS
STAFAFMQTN NHNCLRFITS VVQTFNGGAP DNYPIDIFAR
LWAVDRLQRL GISRFFEQEI NDCLSYVYRF WNANGVFSAG
ATNFCDLDDT SMAFRLLRLH GYDVDPNVLR KFKEGDRFCC
HSGEVAMSTS PTYALYRASQ IQFPGEEILD EAFSFTRDYL
QDWLARDQVL DKWIVSKDLP DEIKVGLEVP WYASLPRVEA
AYYMQRHYGG STDAWVAKTC YRMPDVSNDD YLELARLDFK
RCQAQHQSEL SYMQRWYDSC NVEEFGISRK ELLVAYFVAA
ATIFEPERAT ERIVWAKTEI VSKMIKAFFG EDSLDQKTML
LKEFRNSINN GSHRFMKSEH RIVNILLQAL QELLHGSDDC
RIGQLKNAWY EWLMKFEGGD EASLWGEGEL LVTTLNICTA
HFLQHHDLLL NHDYITLSEL TNRICLKLSQ IQVGEMNEMR
EDMQALTKLV IGESCIVNKN IKQTFLAVAK TFYYRAYFDA
DTVDLHIFKV LFEPIV

A nucleic acid encoding the Hyptis suaveolens labda-7,13E-dienyl diphosphate synthase (HsTPS1) with SEQ ID NO:27 is shown below as SEQ ID NO:28.

ATGGCGTATA TGATATCTAT TTCAAATCTC AACTGTTCCT
CGCTACTAAA CACCAATCTT TCAGCAAAGA TTCAGCTGCA
CCAAGGTCTC AAAGGAACAT GGCTAAAAAC CAGCAAACGC
ATGTGCATGG ATCAACAGGT TCATGGCAAG CAGATAGCAA
AAGTGATCGA GAGCCGAGTT ACTGATAAGG ATGTTTCCAC
TGCTCAGGAC TTTGAAGTGT TAAAGGTCAA TAGAGTGGAG
GATCTGATAT CAAGCATTAA GAGTTCATTG AAGACAATGG
AAGATGGAAG AATAAGCGTG TCGCCCTACA GCACATCATG
GATCGCACTC ATTCCAAGTA TTGATGGGCG CCAGACGCCC
CAGTTTCCAT CTTCACTGGA GTCGATCGTG AAGCATCAGC
TATCAGATGG TTCATGGGGT GATGCCCTTT TTTTCTGCGT
TTATGATCGT CTCGTAAATA CGATTGCATG CATCATTGCC
CTGCACACCT GGAAGGTTCA TGCAGACAAG GTTAAAAAAG
GAGTAAGTTT TGTGAAGGAA AATATATGGA AACTTGAAGA
CGCCAACGAG GTCCACATGA CTAGTGGTTT CGAAGTTATA
TTTCCCATCC TTCTTCGAAG AGCACGAGAC ATGGGAATTG
ATGGTCTTCC TTCTGATGAT ACTCCAGTTG TTAGGATGAT
TTCTGCTGCT AGGGATCACA AATTGAAAAA GATTCCGAGG
GAGGTGATGC ACCAAGTGAC AACAACTCTA TTATATAGTT
TGGAAGGGTT GGAAGATTTA GACTGGTCAA GGCTTTTCAA
ACTTCAGTCA GCTGATGGTT CATTCTTAAC TTCTCCATCT
TCAACTGCCT TCGCATTCAT GCAAACTAAT AACCACAATT
GCTTGAGATT CATCACTAGC GTTGTCCAAA CATTCAATGG
AGGAGCTCCA GATAACTATC CAATCGACAT CTTTGCGAGA
CTGTGGGCAG TTGACAGGTT ACAGCGGTTA GGGATTTCTC
GTTTCTTCGA GCAGGAGATA AATGATTGCC TAAGCTATGT
ATATAGATTT TGGAATGCAA ATGGAGTTTT CAGTGCAGGA
GCCACTAATT TTTGTGATCT TGACGACACA TCCATGGCTT
TCCGGCTACT ACGTTTGCAT GGATATGATG TCGACCCAAA
TGTTCTGAGG AAATTCAAAG AGGGAGACAG ATTCTGTTGC
CACAGTGGTG AAGTGGCGAT GTCGACATCG CCAACGTACG
CTCTCTACAG AGCTTCCCAA ATTCAGTTTC CAGGAGAAGA
AATTCTGGAT GAAGCCTTCA GCTTCACTCG CGACTATCTA
CAGGACTGGT TAGCAAGAGA TCAAGTTCTT GATAAGTGGA
TTGTATCCAA GGACCTTCCA GATGAGATTA AGGTAGGACT
AGAGGTGCCA TGGTATGCCA GCCTGCCACG GGTAGAGGCT
GCTTATTACA TGCAACGACA TTACGGCGGG TCTACTGATG
CGTGGGTGGC CAAGACTTGT TACAGGATGC CTGATGTGAG
CAACGATGAT TACCTGGAGC TTGCAAGATT GGATTTCAAG
AGATGTCAAG CCCAACATCA GACTGAATTG AGTTACATGC
AACGATGGTA TGACAGTTGC AATGTCGAAG AATTCGGAAT
AAGCAGAAAA GAGTTGCTTG TAGCTTATTT TGTGGCTGCT
GCAACTATTT TTGAACCTGA GAGAGCAACT GAGAGAATTG
TGTGGGCAAA AACTGAAATA GTTTCTAAGA TGATCAAAGC
ATTTTTTGGT GAAGACTCAT TAGACCAAAA AACTATGTTG
TTAAAAGAAT TCAGAAACAG CATCAATAAT GGCTCCCACA
GATTCATGAA GAGTGAGCAT AGAATCGTCA ACATTCTACT
ACAAGCCTTG CAGGAGCTAT TACATGGATC TGATGATTGT
CGTATTGGTC AACTCAAAAA TGCTTGGTAT GAGTGGCTGA
TGAAATTCGA GGGAGGAGAT GAAGCAAGTT TGTGGGGAGA
AGGAGAGCTT CTTGTCACCA CCTTAAACAT TTGCACAGCT
CATTTCCTTC AACACCATGA TTTACTGTTG AATCATGACT
ACATAACTCT TTCTGAGCTC ACAAACAAGA TCTGCCTCAA
GCTTTCTCAG ATTCAGGTAG GAGAAATGAA TGAAATGAGA
GAAGATATGC AGGCGTTGAC GAAATTAGTG ATTGGGGAAT
CATGCATCGT CAACAAAAAC ATTAAGCAAA CATTTCTTGC
AGTTGCAAAG ACTTTCTATT ACAGAGCCTA CTTCGATGCC
GACACCGTTG ATCTCCATAT ATTTAAAGTT CTATTTGAGC
CCATTGTCTG A

Leonotis leonurus peregrinol diphosphate synthase (LITPS1) was identified and isolated using the methods described herein. The LITPS1 enzyme was identified as a peregrinol diphosphate (PgPP) [5] synthase, where the peregrinol diphosphate (PgPP) [5] compound is shown below.

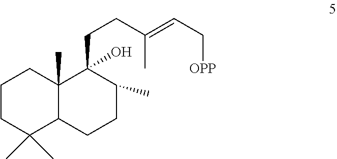

The Leonotis leonurus peregrinol diphosphate synthase (LITPS1) can have the amino acid sequence shown below (SEQ ID NO:29).

MASTASTLNL TINSTPFVST KTQAKVSLTA CLWMQDRSSS
RHVSLKHKFC RNQQLKCRAS LDVQQVRDEV FSTAQSPESV
DKKIEERKKW VKNLLSTMDD GRINWSAYDT AWISLIKEFE
GRDATQFPST LMRIAENQLA DGSWGDPDYD CSYDRIINTL
ACVVALTTWN AHPEHNKKGI KYIKENMYKL EETPVVLMTS
AFEVVFPALL NRAKNLGIQD LPYDMPIVKE ICKIGDEKLA
RIPKKMMEKE PTSLMYAAEG VENLDWEKLL KQRTPENGSF
LSSPAATAVA FMHTKDENCL RYIMYLLDKF NGGAPNVYPI
DLWSRLWATD RIQRLGISRF FKEEIKEILS YVYSYWTDIG
VYCTRDSKYA DIDDTSMGFR LLRMHGFKMD PNVFKYFQKD
DRFVCLGGQM NDSPTATYNL YRAAQYQFPG EKILEDARKF
SQEFLQHCID TNNLLDKWVI SPRFPEELKF GMEMTWYSCL
PRIEARYYVQ HYGATEDVWL GYTFFRMEEI SNENYKELAK
LDFSKCQAQH QTEWIHMQEW YESSNAKEFG ISRKDLLFAY
FLAAASIFET ERAKERILWA KSQIICKMVK SYLENQTASL
EHKIAFLTGF GDNNNGLHTI NKGSGPVNNV MRTLQQLLGE
FDGYISSQLE NAWAAWLTKL EQGEANDGEL LATTLNICSG
RIVYNEDTLS NKEYKAFADL TNKICQNLAQ IQNKKGDEIK
DPNEGEKDKE VEQGMQALAK LVFEESGLER SIKETFLAVV
RTYHYGAYVA DEKIDVHMFK VLFEPVE

A nucleic acid encoding the Leonotis leonurus peregrinol diphosphate synthase (LITPS1) with SEQ ID NO:29 is shown below as SEQ ID NO:30.

ATGGCCTCCA CTGCATCCAC TCTAAATTTG ACCATCAATA
GTACACCATT TGTAAGCACC AAAACGCAAG CAAAGGTTTC
CTTGCCCGCA TGTTTATGGA TGCAGGATAG AAGCAGCAGT
AGACACGTGT CGTTAAAACA CAAATTCTGT CGAAATCAAC
AACTTAAGTG TCGAGCAAGT CTGGATGTTC AGCAAGTACG
TGATGAAGTT TTTTCCACTG CTCAATCCCC TGAATCGGTG
GATAAAAAAA TAGAGGAACG TAAAAAATGG GTGAAGAATT
TGTTGAGTAC AATGGACGAT GGACGAATAA ATTGGTCAGC
CTATGACACG GCATGGATTT CACTTATTAA AGAATTTGAA
GGACGAGATG CTCCCCAGTT TCCGTCGACT CTCATGCGCA
TCGCGGAGAA CCAATTGGCC GACGGGTCAT GGGGCGATCC
AGATTACGAC TGCTCCTATG ATCGGATAAT AAACACACTA
GCGTGTGTTG TAGCCTTGAC AACATGGAAT GCTCATCCTG
AACACAATAA AAAAGGAATA AAATACATCA AGGAAAATAT
GTATAAACTA GAAGAGACGC CTGTTGTACT CATGACTAGT
GCATTTGAAG TTGTGTTTCC GGCGCTTCTT AACAGAGCTA
AAAACTTGGG CATTCAAGAT CTTCCCTATG ATATGCCCAT
CGTGAAGGAG ATTTGTAAAA TAGGGGATGA GAAGTTGGCA
AGGATACCAA AGAAAATGAT GGAGAAAGAG CCAACATCGC
TGATGTATGC CGCGGAAGGA GTCGAAAACT TGGACTGGGA
AAAGCTTCTG AAACAGCGGA CACCCGAGAA TGGCTCGTTC
CTCTCTTCCC CGGCCGCAAC TGCCGTTCCA TTTATGCACA
CAAAAGATGA AAATTGCTTA AGATACATCA TGTACCTTTT
GGACAAATTT AATGGAGGAG CACCAAATGT TTATCCGATC
GACCTCTGGT CAAGACTTTG GGCAACGGAC AGGATACAAC
GTCTGGGAAT TTCCCGCTTC TTTAAGGAAG AGATTAAGGA
AATCTTAAGT TATGTCTATA GCTATTGGAC AGACATTGGA
GTCTATTGTA CACGAGATTC CAAATATGCT GACATTGACG
ACACATCCAT GGGATTCAGG CTTCTGAGGA TGCACGGATT
TAAAATGGAC CCAAATGTAT TTAAATACTT CCAGAAAGAC
GACAGATTTG TTTGTCTAGG TGGTCAAATG AATGATTCTC
CAACTGCAAC ATACAATCTT TACAGGGCTG CTCAATACCA
ATTTCCAGGT GAAAAAATTC TAGAAGATGC TAGAAAGTTC
TCTCAAGAGT TTCTACAACA TTGTATAGAC ACCAATAACC
TTCTAGATAA ATGGGTGATA TCCCCGCGCT TTCCGGAAGA
GTTGAAATTT GGAATGGAGA TGACATGGTA TTCCTGCCTA
CCACGAATTG AGGCTAGATA CTACGTACAA CATTATGGTG
CTACAGAGGA CGTCTGGCTT GGAAAGACTT TTTTCAGGAT
GGAAGAAATC AGTAATGAGA ACTATAAGGA GCTTGCAAAA
CTTGATTTCA GTAAATGCCA AGCACAACAT CAGACAGAGT
GGATTCATAT GCAAGAGTGG TATGAAAGTA GCAATGCTAA
GGAATTTGGG ATAAGCAGAA AAGACCTACT TTTTGCTTAC
TTTTTGGCTG CAGCTTCCAT ATTTGAAACC GAAAGGGCAA
AAGAGAGAAT TCTGTGGGCA AAATCTCAAA TTATTTGCAA
GATGGTTAAG TCATATCTGG AAAACCAAAC GGCGTCGTTG
GAGCACAAAA TCGCCTTTTT AACTGGATTC GGAGATAACA
ACAATGGCCT GCACACAATT AATAAGGGGT CTGGACCTGT
TAACAATGTC ATGAGAACCC TCCAACAGCT CCTTGGAGAA
TTCGACGGAT ATATTAGTAG TCAATTGGAA AATGCTTGGG
CAGCATGGTT GACGAAACTC GAGCAAGGCG AGGCCAACGA
TGGCGAGCTC CTCGCAACCA CACTAAACAT TTGTTCTGGG
CGTATTGTGT ATAACGAGGA TACATTATCG AACAAGGAGT
ACAAGGCTTT CGCAGACCTC ACAAATAAAA TTTGTCAAAA
TCTTGCTCAA ATCCAAAATA AAAAGGGTGA CGAAATTAAG
GATCCGAATG AAGGCGAAAA GGACAAGGAA GTCGAGCAAG
GCATGCAGGC ATTGGCTAAG TTAGTTTTTG AGGAATCTGG
GCTTGAGAGG AGTATCAAAG AAACATTCTT AGCAGTGGTG
AGAACTTATC ACTATGGGGC CTATGTTGCT GATGAGAAGA
TTGATGTCCA CATGTTCAAG GTTTTGTTCG AACCAGTTGA
ATGA

Nepeta mussinii (+)-copalyl diphosphate synthase (NmTPS1) was identified and isolated. The NmTPS1 enzyme can synthesize compound 31, shown below.

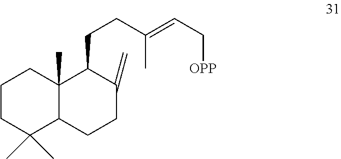

The Nepeta mussinii (+)-copalyl diphosphate synthase (NmTPS1) can have the amino acid sequence shown below SEQ ID NO:31).

MTSISSLNLS NAAAARRRLQ LPANVHLPEF HSVCAWLNSS
SKHDPFSCRI HRKQKSKVTE CRVASVDASP VSDHKMSSPV
QTQEEANKNM EESIEYIKNL LMTSGDGRIS VSAYDTSIVA
LIKDIEGRDA PQFPSCLEWI GQNQKADGSW GDDFFCIYDR
FVNTLACIVA LKSWNLHPHK IQKGVTYIKK NVHKLKDGRP
ELMTSGFEIC VPAILQRAKD LGIQDLPYDD PMIKQITDTK
ERRLKKIPKD FIYQLPTTLL FSLEGQENLD WEKILKLQSA
DGSFLTSPSS TAAVFMHTKD EKCLKFIENA VKNCDGGVPH
TYPVDVFARL WAVDRLQRLG ISRFFQPEIK YFLDHIQSVW
TENGVFSGRD SQFCDIDDTS MGIRLLKMHG YKIDPNALEH
FKQEDGKFSC YGGQMIESAS PIYNLYRAAQ LRFPGEEILE
EAIKFSYNFL QEKLAKDEIQ EKWVISEHLI DEIKTGLKMP
WYATLPRVEA AYYLDYYAGS GDVWIGKTFY RMPEISNDTY
KEMAILDFNR CQAQHQFEWI YMQEWYESSN VKEFGISKKE
LLVAYFLAAS TIFEPERAQE RIMWAKTKIV SKMIASSLNK
QTTLSLDQKT ALFTQLEHSL NGLDSDEKDN GVAETKNLVA
TFQQLLDGFD KYTRHQLKNA WSQWLKQVQQ GEATGGADAE
LEANTLNICA GHIAFNEQVL SHNEYTTLST LTNKICHRLT
QIQDKKTLEI IDGGIRYKEL EQEMQALVKL VVEENDGGGI
DRNIKQTFLS VFKNYYYSAY HDAHTTDVHI FKVLFGPVV

A nucleic acid encoding the Nepeta mussinii (+)-copalyl diphosphate synthase (NmTPS1) with SEQ ID NO:31 is shown below as SEQ ID NO:32.

ATGACTTCAA TATCCTCTCT AAATTTGAGC AATGCAGCAG
CTGCTCGCCG CAGGTTACAA CTACCAGCAA ACGTTCACCT
GCCGGAATTT CACTCCGTCT GTGCATGGCT GAATAGCAGC
AGCAAACACG ATCCCTTTAG TTGCCGAATT CATCGAAAGC
AAAAATCGAA AGTAACCGAG TGTCGAGTAG CAAGCGTGGA
TGCATCACCA GTGAGTGATC ATAAAATGAG TTCTCCTGTT
CAAACTCAAG AAGAGGCAAA TAAAAATATG GAGGAGTCAA
TCGAGTACAT AAAGAATTTG TTGATGACAT CTGGAGACGG
GCGAATAAGC GTGTCGGCAT ACGACACGTC AATAGTCGCC
CTAATTAAGG ACATAGAAGG ACGCCACGCC CCGCAATTTC
CATCATGCCT GGAGTGGATC GGGCAAAACC AAAAGGCCGA
TGGCTCGTGG GGGGACGACT TCTTCTGTAT TTACGACCGC
TTCGTAAATA CACTAGCATG TATCGTGGCC TTGAAATCAT
GGAACCTTCA CCCTCACAAG ATTCAAAAAG GAGTGACATA
CATCAAGAAA AACGTGCATA AGCTTAAAGA TGGGAGGCCT
GAGCTGATGA CGTCAGGGTT CGAAATTTGT GTTCCCGCCA
TTCTTCAAAG AGCCAAAGAC TTGGGCATCC AAGATCTTCC
CTATGATGAT CCCATGATTA AACAGATCAC TGATACGAAA
GAGCGACGAC TCAAAAAGAT ACCGAAGGAT TTTATATACC
AATTGCCGAC GACTTTACTC TTCAGTTTGG AAGGGCAGGA
GAATTTGGAC TGGGAAAAGA TACTCAAACT GCAGTCAGCT
CACGGCTCCT TCCTTACTTC GCCGTCCTCC ACCGCCGCCG
TCTTCATGCA TACCAAAGAT GAAAAATGCT TGAAGTTCAT
AGAGAACGCC GTCAAAAATT GCGACGGCGG AGTGCCCCAT
ACCTACCCAG TAGACGTGTT TGCAAGACTT TGGGCAGTTG
ACAGACTACA ACGCCTAGGG ATTTCTCGCT TTTTTCAGCC
TGAGATTAAA TATTTCTTAG ATCACATACA AAGCGTTTGG
ACTGAGAACG GAGTTTTCAG TGGACGAGAT TCACAATTTT
GCGACATTGA TGATACGTCC ATGGGGATAA GGCTTCTGAA
AATGCATGGA TACAAAATCG ACCCAAATGC ACTTGAGCAT
TTCAAGCAGG AGGATGGTAA ATTTTCGTGC TACGGTGGTC
AAATGATCGA GTCTGCATCA CCGATATACA ATCTGTACCG
AGCTGCTCAA CTCCGATTTC CAGGAGAAGA AATTCTTGAA
GAGGCCATTA AATTTTCCTA TAACTTTTTG CAAGAAAAGC
TAGCCAAGGA TGAAATTCAA GAAAAATGGG TCATATCGGA
GCACTTAATT GATGAGATTA AGATCGGGCT AAAGATGCCA
TGGTACGCCA CTCTACCCCG AGTTGAAGCT GCATATTACC
TGGACTATTA TGCAGGATCC GGCGATGTGT GGATTGGCAA
GACTTTCTAC AGGATGCCAG AAATCAGTAA TGATACATAC
AAAGAAATGG CCATTTTGGA TTTCAACCGA TGCCAAGCAC
AACATCAGTT TGAATGGATT TACATGCAAG AGTGGTATGA
AAGTAGCAAC GTAAAGGAAT TTGGGATAAG CAAAAAAGAG
CTACTTGTTG CTTATTTCTT GGCTGCATCA ACCATATTTG
AACCGGAAAG AGCACAAGAG AGGATTATGT GGGCAAAAAC
AAAAATTGTT TCCAAAATGA TCGCATCATC TCTTAACAAA
CAAACCACTC TATCGTTAGA CCAAAAGACT GCACTTTTTA
CCCAACTCGA ACATAGTCTC AATGGCCTCG ACAGTGATGA
GAAAGATAAT GGAGTAGCTG AGACGAAAAA TCTAGTGGCA
ACCTTCCAGC AGCTGCTAGA TGGATTCGAC AAATACACTC
GCCATCAATT GAAAAATGCT TGGAGCCAGT GGTTGAAGCA
AGTGCAGCAA GGAGAGGCGA CCGGGGGCGC AGACGCGGAG
CTGGAAGCAA ACACGTTGAA CATCTGTGCC GGTCATATCG
CATTCAACGA ACAAGTATTA TCGCACAACG AATACACAAC
TCTCTCCACA CTCACAAACA AGATCTGCCA CCGGCTTACC
CAAATTCAAG ACAAAAAGAC GCTTGAGATA ATCGACGGCG
GCATAAGATA TAAGGAGCTG GAGCAGGAGA TGCAGGCGTT
GGTGAAATTA GTTGTTGAAG AAAACGACGG CGGCGGCATA
GACAGGAATA TTAAACAAAC ATTTTTATCA GTTTTCAAGA
ATTATTACTA CAGTGCCTAC CACGATGCTC ACACAACCGA
TGTTCATATT TTCAAAGTAT TATTTGGACC GGTCGTCTGA

Origanum majorana (+)-copalyl diphosphate synthase (OmTPS1) was identified and isolated as describe herein. The OmTPS1 enzyme can synthesize compound 31. OmTPS1 can also synthesize palustradiene [29] (shown below), when combined with OmTPS5.

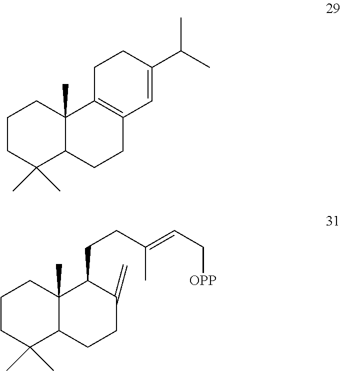

The Origanum majorana (+)-copalyl diphosphate synthase (OmTPS1) can have the amino acid sequence shown below (SEQ ID NO:33).

MTDVSSLRLS NAPAAGGRLP LPGKVHLPEF RTVCAWLNNG
CKYEPLTCRI SRRKISECRV ASLNSSQLIE KVGSPAQSLE
EANKKIEDSI EYIKNLLMTS GDGRISVSAY DTSLVALIKD
VKGRDAPQFP SCLEWIAQNQ MADGSWGDEF FCIYDRIVNT
LACLVALKSW NLHPDKIEKG VTYINENVHK LKDGSTEHMT
SGFEIVVPAT LERAKVLGIQ GLPYDHPFIK EIINTKERRL
SKIPKDLIYK LPTTLLFSLE GQGELDWEKI LKLQSSDGSF
LTSPSSTASV FMRTKDEKCL KFIENAVKNC GGGAPHTYPV
DVFARLWAVD RLQRLGISRF FQHEIKYFLD HINSVWTENG
VFSGRDSQFC DIDDTSMGVR LLKMHGYNVD PNALKHFKQE
DGNFSCYPGQ MIESASPIYN LYRAAQLRFP GEEILEEASR
FAFNFLQEKI ANHEIQEKWV ISEHLIDEIK LGLKMPWYAT
LPRVEAAYYL EYYAGSGDVW IGKTFYRMPE ISNDTYKEVA
ILDFNTCQAQ HQFEWIYMQE WYESSKVKDF GISKKDLLVA
YFLAASTIFE PERTQERIIW AKTLILSRMI TSFMNKQATL
SSQQKNAILT QLGESVDGLD KIYSGEKDSG LAETLLATFQ
QLLDGFDRYT RHQLKNAWGQ WLMKVQQGEA NGGADAELIA
NTLNICAGLI AFNEDVLLHS EYTTLSSLTN KICQRLSQIE
DEKTLEVIEG GIKDKELEED IQALVKLALE ENGGCGVDRR
IKQSFLSVFK TFYYRAYHDA ETTDLHIFKV LFGPVM

A nucleic acid encoding the Origanum majorana (+)-copalyl diphosphate synthase (OmTPS1) with SEQ ID NO:33 is shown below as SEQ ID NO:34.

ATGACCGATG TATCCTCTCT TCGTITGAGC AATGCACCAG
CTGCCGGCGG CAGGTTGCCG CTGCCGGGAA AGGTTCACCT
GCCTGAATTT CGCACCGTTT GTGCATGGTT GAACAATGGC
TGCAAATACG AGCCCTTGAC TTGTCGAATT AGTCGACGGA
AGATATCTGA ATGTCGAGTA GCAAGTCTGA ATTCGTCGCA
AGTAATTGAA AAGGTCGGTT CTCCTGCTCA ATCTCTAGAA
GAGGCAAACA AAAAGATCGA GGACTCCATC GAGTACATTA
AGAATCTATT GATGACATCT GGCGACGGGC GGATAAGTGT
GTCGGCTTAC GACACGTCGC TAGTCGCCCT AATAAAGGAC
GTGAAAGGAC GAGATGCCCC TCAGTTCCCG TCGTGCCTGG
AGTGGATAGC GCAAAACCAA ATGGCCGACG GGTCGTGGGG
GGATGAGTTC TTCTGTATTT ACGACCGGAT CGTGAATACA
TTAGCATGCC TCGTTGCCTT GAAATCATGG AACCTTCACC
CCGACAAGAT CGAAAAAGGA GTGACGTACA TCAACGAAAA
TGTGCACAAA CTGAAAGACG GGAGCACCGA GCACATGACG
TCAGGGTTCG AAATCGTGGT CCCCGCCACT CTAGAAAGAG
CCAAAGTCTT GGGCATCCAA GGCCTCCCTT ATGATCATCC
CTTCATTAAG GAGATTATTA ATACTAAGGA GCGAAGATTA
AGCAAAATAC CCAAGGATTT GATATACAAA CTGCCAACGA
CGCTGCTGTT CAGTTTAGAA GGGCAGGGAG AATTAGATTG
GGAAAAGATA CTGAAACTGC AGTCAAGCGA TGGCTCCTTC
CTTACTTCGC CCTCGTCGAC CGCCTCCGTC TTCATGCGGA
CGAAAGACGA GAAATGCCTC AAGTTCATTG AGAACGCCGT
TAAGAATTGC GGCGGGGGAG CGCCGCATAC TTACCCAGTG
GATGTGTTTG CAAGACTTTG GGCAGTTGAC AGACTACAGC
GATTAGGGAT TTCTCGATTC TTCCAACACG AGATTAAATA
CTTCTTAGAT CACATTAAGA GTGTATGGAC CGAGAATGGA
GTTTTCAGTG GACGAGATTC ACAATTTTGT GATATCGACG
ACACTTCTAT GGGAGTTAGG CTTCTAAAAA TGCATGGATA
CAATGTTGAT CCAAATGCGC TCAAGCATTT CAAGCAGGAG
GATGGCAAAT TCTCTTGCTA CCCTGGCCAA ATGATCGAGT
CTGCATCTCC GATATACAAT CTCTACCGAG CCGCTCAACT
CCGGTTCCCC GGAGAAGAAA TTCTCGAAGA AGCAAGTCGA
TTCGCCTTCA ACTTTCTGCA GGAAAAGATA GCCAACCATG
AAATTCAAGA AAAATGGGTC ATATCTGAGC ACTTAATTGA
TGAGATAAAG TTGGGACTGA AGATGCCATG GTACGCGACT
CTGCCCCGAG TTGAGGCCGC TTATTATCTA GAGTATTATG
CTGGCTCAGG CGACGTATGG ATTGGAAAGA CTTTCTACCG
GATGCCGGAA ATCAGTAACG ATACGTATAA AGAGGTGGCC
ATTTTGGATT TCAACACATG CCAAGCTCAA CACCAGTTTG
AATGGATTTA CATGCAAGAG TGGTACGAAA GTAGCAAGGT
TAAAGATTTC GGGATAAGCA AAAAGGACCT ACTTGTTGCT
TACTTTCTGG CGGCATCGAC TATATTTGAA CCCGAAAGAA
CACAAGAGAG GATTATTTGG GCAAAAACCC TAATTCTTTC
TAGGATGATC ACATCATTTC TCAACAAACA AGCTACACTT
TCATCCCAAC AAAAGAATGC CATCTTAACA CAACTTGGAG
AGAGTGTCGA TGGCCTCGAT AAAATATATA GTGGTGAGAA
AGATTCTGGG CTGGCTGAGA CTCTGCTGGC TACCTTCCAG
CAACTGCTCG ACGGATTCGA TAGATACACT CGCCATCAAC
TGAGAAATGC TTGGGGGCAA TGGTTGATGA AAGTGCAGCA
AGGAGAGGCC AACGGTGGCG CCGACGCTGA GCTCATAGCA
AACACACTCA ATATCTGCGC CGGCCTTATC GCCTTCAACG
AAGACGTATT GTTGCACAGC GAATACACGA CTCTCTCCTC
CCTCACCAAC AAAATATGCC ACCGCCTTAG CCAGATTGAA
GATGAAAAGA CGCTTGAAGT GATTGAAGGG GGCATAAAAG
ATAAGGAACT GGAGGAGGAT ATTCAGGCGT TGGTGAAGCT
AGCCCTCGAA GAAAACGGCG GCTGCGGCGT CGACAGAAGA
ATCAAGCAGT CATTCTTATC AGTATTCAAG ACTTTTTACT
ACAGAGCCTA CCATGATGCT GAGACCACCG ATCTTCATAT
TTTCAAAGTA CTGTTGGGGC CGGGTATGTG A

A Perovskia atriplicifolia (+)-Copalyl diphosphate synthase (PaTPS1) enzyme was identified and isolated as described herein. This Perovskia atriplicifolia (+)-Copalyl diphosphate synthase (PaTPS1) enzyme was identified to be a (+)-copalyl diphosphate ((+)-CPP) synthase that can synthesize compound 31. The Perovskia atriplicifolia (+)-Copalyl diphosphate synthase (PaTPS1) can have the amino acid sequence shown below (SEQ ID NO:35).

MTSMSSLNLS RAPATTHRLQ LQAKVHVPEF YAVCAWLNSS
SKQAPLSCQI RCKQLSRVTE CRVASLDASQ VSEKDTSHVQ
TPDEVNKKIE DYIEYVKNLL MTSGDGRISV SPYDTSIVAL
IKDSKGRNIP QFPSCLEWIA QHQMADGSWG DQFFCIYDRI
LNTLACVVAL KSWNVHGDMI EKGVTYVKEN VHKLKDGNIE
HMTSGFEIVV PALVQRAKDL GIQGLPYDDP LIKEIADTKE
RRLKKIPKDM IYQTPTTLLF SLEGQGDLEW EKILKLQSGD
GSFLTSPSST AHVFVQTKDE KCLKFIENAV KNCSGGAPHT
YPVDVFARLW AIDRLQRLGI SRFFQPEIKY FIDHINSVWT
ENGVFSGRDS EFCDIDDTSM GIRLLKMHGY KVDPNALNHF
KQQDGKFSCY GGQMIESASP IYNLYRAAQL RFPGEEILEE
ASKFAFNFLQ EKIANDQFQE KWVISDHLID EVKLGLKMPW
YATLPRVEAA YYLQYYAGSG DVWIGKVFYR MPEISNDTYK
ELAILDFNRC QAQHQFEWIY MQEWYHRSSV SEFG1SKKEL
LRTYFLAAAT IFEPERTQER LVWAKTQIVS RMITSFVNNG
TTLSLDQMTA LATQIGHNFD GLDQIISAMK DHGLAGTLLT
TFQQLLDGFD RYTRHQLKNA WSQWFMKLQQ GEANGGEDAE
LLANTLNICA GFIAFNEDVL SHDEYTTLST LTNKICKRLS
QIQDKKALEV VDGSIKDKEL EQDMQALVKL VLEENGGGVD
RNIKQTFLSV FKTFYYTAYH DDETTDVHIF KVLFGPVV

A nucleic acid encoding the Perovskia atriplicifolia (+)-Copalyl diphosphate synthase (PaTPS1) enzyme with SEQ ID NO:35 is shown below as SEQ ID NO:36.

ATGACCTCTA TGTCCTCTCT AAATTTGAGC AGAGCACCAG
CTACCACCCA CCGGTTACAG CTACAGGCAA AGGTTCACGT
GCCGGAATTT TATGCCGTGT GTGCATGGCT GAATAGCAGC
AGCAAACAGG CACCCTTGAG TTGCCAAATT CGCTGCAAGC
AACTATCAAG AGTAACTGAA TGTCGGGTAG CAAGTCTGGA
TGCGTCGCAA GTGAGTGAAA AAGACACTTC TCATGTCCAA
ACTCCCGATG AGGTGAACAA AAAGATCGAG GACTATATCG
AGTACGTCAA GAATCTGTTG ATGACGTCGG GCGACGGGCG
AATAAGCGTG TCGCCCTACG ACACGTCAAT AGTCGCCCTT
ATTAAGGACT CGAAAGGGCG CAACATCCCG CAGTTTCCGT
CGTGCCTCGA GTGGATAGCG CAGCACCAAA TGGCGGATGG
CTCATGGGGG GATCAATTCT TCTGCATTTA CGACCGGATT
CTAAATACAT TAGCATGTGT CGTAGCTTTG AAATCCTGGA
ACGTTCACGG TGACATGATC GAAAAAGGAG TGACGTACGT
CAAGGAAAAT GTGCATAAGC TTAAAGATGG GAATATTGAG
CACATGACGT CGGGGTTCGA AATTGTGGTT CCCGCCCTTG
TTCAAAGAGC CAAAGACTTG GGCATCCAAG GCCTGCCCTA
TGATGATCCC CTCATCAAGG AGATTGCTGA TACAAAAGAA
AGAAGATTGA AAAAGATACC CAAGGATATG ATTTACCAAA
CGCCAACGAC ATTACTATTC AGTTTAGAAG GGCAGGGAGA
TTTGGAGTGG GAAAAGATAC TGAAACTGCA GTCAGGCGAT
GGCTCCTTCC TCACTTCGCC GTCATCCACC GCCCACGTGT
TCGTGCAGAC CAAAGATGAA AAATGCTTGA AATTCATCGA
GAACGCCGTC AAGAATTGCA GTGGAGGAGC GCCGCATACT
TATCCAGTCG ATGTCTTCGC AAGACTTTGG GCAATTGACA
GACTACAACG CCTAGGAATT TCTCGTTTCT TCCAGCCGGA
AATTAAGTAT TTCATAGACC ACATCAACAG CGTTTGGACA
GAGAACGGAG TTTTCAGTGG GCGAGATTCG GAATTTTGCG
ATATTGATGA CACGTCCATG GGCATCAGGC TTCTCAAAAT
GCACGGATAC AAAGTCGACC CAAATGCACT CAATCATTTC
AAGCAGCAAG ATGGTAAATT TTCTTGCTAC GGTGGTCAAA
TGATCGAGTC TGCATCTCCA ATATACAATC TCTACAGGGC
TGCTCAGCTA CGATTTCCAG GAGAAGAAAT TCTTGAAGAA
GCCAGTAAAT TTGCCTTTAA CTTTTTGCAA GAAAAAATAG
CCAACGATCA ATTTCAAGAA AAATGGGTGA TATCCGACCA
CTTAATCGAT GAGGTGAAGC TCGGGCTGAA GATGCCATGG
TACGCCACTC TACCCCGGGT TGAGGCTGCA TATTATCTAC
AATACTATGC TGGTTCTGGC GACGTATGGA TTGGCAAGGT
TTTCTACAGG ATGCCGGAAA TCAGCAATGA TACATACAAA
GAGCTGGCCA TATTGCATTT CAACAGATGC CAAGCACAGC
ATCAGTTCGA ATGGATTTAT ATGCAAGAGT GGTATCACAG
AAGCAGCGTT AGTGAATTCG GGATAAGCAA AAAAGAGCTG
CTTCGTACTT ACTTTCTGGC TGCAGCAACC ATATTCGAAC
CCGAGAGAAC ACAAGAGAGG CTTGTGTGGG CAAAAACCCA
AATTGTCTCT AGGATGATCA CATCATTTGT TAACAATGGA
ACTACACTAT CTTTGGACCA AATGACTGCA CTTGCAACAC
AAATCGGCCA TAATTTCGAT GGCCTCGATC AAATAATTAG
TGGTATGAAA GATCATGGAC TGGCTGGGAC TCTGCTGACA
ACCTTCCAGC AACTTCTAGA TGGATTCGAC AGATACACTC
GCCATCAACT CAAAAATGCT TGGAGCCAAT GGTTCATGAA
ACTCCACCAA GGGGAGGCGA ACGGCGGGGA AGACGCGGAG
CTCCTAGCAA ACACGCTCAA CATCTGCGCG GGTTTCATTG
CTTTCAACGA AGACGTATTG TCGCACGATG AATACACGAC
TCTCTCCACC CTTACAAACA AAATCTGCAA GCGCCTTAGC
CAAATTCAAG ATAAAAAGGC GCTGGAAGTT GTCGACGGGA
GCATAAAGGA TAAGGAGCTC GAACAGGATA TGCAGGCGTT
GGTGAAGTTG GTCCTTGAAG AAAATGGCGG CGGCGTCGAC
AGGAACATCA AACAGACATT TTTGTCCGTT TTCAAGACTT
TTTACTACAC CGCCTACCAC GATGATGAGA CCACTGATGT
TCATATTTTC AAAGTACTGT TTGGACCGGT CGTATGA

Pogostemon cablin (10R)-labda-8,13E-dienyl diphosphate synthase (PcTPS1) was identified and isolated as described herein. This Pogostemon cablin (10R)-labda-8,13E-dienyl diphosphate synthase (PcTPS1) enzyme was identified to be a (10R)-labda-8,13E-dienyl diphosphate synthase, which can synthesize compound 25.

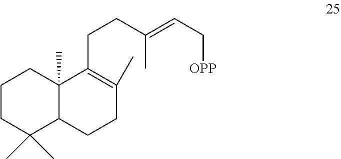

The combination of PcTPS1 and SsSS, both in-vitro, and in N. benthamiana expression produced (10R)-labda-8,14-en-13-ol [26], shown below.

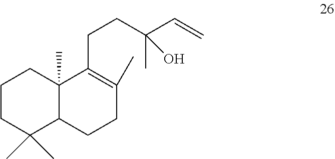 [SMILES: C=CC(C)(O)CCC1=C(C)CCC2C(C)(C)CCC[C@@]12C]

This Pogostemon cablin (10R)-labda-8,13E-dienyl diphosphate synthase (PcTPS1) can have the amino acid sequence shown below (SEQ ID NO:37).

MSFASQSHVA FVLRRPSAVA PPPPTRIPTT AALSPLKPGD
FSHGRSSFMP TSIKCNAIST SRVEEYKYTD DHNQSGLLEH
DGLISDKINE LVTKIQLMLQ NMDDGEISIS PYDTAWVSLV
EDVGGNDRPQ FPTSLEWISN NQLPDGSWGD PNAFLVHDRI
LNTLACVVAL KSWKMHPHKC NRGVSFVREN IYRMDDEKEE
HMPNGFEVVF PALLQKAKTL NIDIPYEFPG IQKFYAKRDL
KFARIPMDIL HSVPTTLLFS LEGVRCGLDL DWGKLLELQA
ADGSFLYSPS STAFALEQTK DQNCLKYLSK LVRKFDGGVP
NVYPVDLFEH NWAVDRLQRL GISRYFTPEI NQCLDYSYRY
WSNSKGMYSA SNSQIQDVDD TAMGFRLLRL NGYDVSTQGF
RQFEAGGDFF CFAGQSSQAV TGMYNLYRAS QVMFPGEKLL
EDAKKFSTNF LQQKRANNQL TDKWVIAKDV PAEVGYALDI
PWYASLPRLE ARFFIQQYGG DDDVWIGKTL YRMGYVNNNT
YLELAKLDYN TCQRLHQHEW ITIQRWYEIN LKITSVGLSK
RGVLLSYYLA AANLFEPQNS THRIAWAKTS ILVSAIQLSP
LQKRDFINQF HRSTANNGYE TSNVLVKSVI KGVHETSMDA
MLTHNKDIHR QLFNAWRKWM SVWEEGGDGE AELLLSTLNT
CDGVDESTFS DPKYEHLLEI TVRVTHQLHL IQNAETKRVG
DREEIDLSMQ QLVKLVFTKS SSDLDSCIKQ RFFAIARSFY
YVAHCDPEMV DSHIAKVLFE RVM

A nucleic acid encoding the Pogostemon cablin (10R)-labda-8,13E-dienyl diphosphate synthase (PcTPS1) enzyme with SEQ ID NO:35 is shown below as SEQ ID NO:38.

ATGTCATTTG CTTCTCAATC ACATGTCGCC TTTGTACTCC
GACGGCCATC TGCCGTTGCT CCGCCACCAC CGACTAGAAT
TCCGACAACA GCCGCTCTTT CTCCTCTCAA ACCAGGTGAT
TTTTCCCATG GCAGATCATC ATTTATGCCC ACTTCCATTA
AATGTAATGC AATTTCCACA TCTCGCGTCG AAGAATACAA
GTACACGGAT GATCATAATC AGAGTGGTTT ATTGGAGCAT
GATGGTTTGA TATCAGACAA GATAAATGAA TTGGTGACCA
AGATACAATT GATGCTACAA AACATGGATG ACGGAGAGAT
AAGCATCTCC CCATATGACA CCGCATGGGT GTCGTTGGTG
GAGGATGTGG GCGGCAACGA CCGCCCACAG TTTCCTACGA
GCCTGGAGTG GATATCGAAT AACCAGCTCC CCGACGGCTC
GTGGGGCGAC CCGAATGCCT TTTTGGTGCA CGACCGTATC
CTCAACACAT TGGCATGCGT CGTTGCACTC AAATCCTGGA
AAATGGACCC CCACAAATGC AATAGAGGAG TTAGTTTCGT
CACAGAAAAT ATATACAGAA TGGATGATGA AAAAGAGGAA
CACATGCCAA ATGGATTCGA AGTGGTATTT CCAGCACTCC
TTCAAAAAGC GAAAACCCTA AACATTGATA TCCCGTACGA
GTTTCCAGGA ATACAAAAAT TTTATGCCAA AAGAGATTTA
AAATTCGCCA GGATTCCAAT GGATATATTG CATAGCGTTC
CGACAACATT ACTGTTCAGC TTAGAAGGTG TAAGATGTGG
TCTTGATCTG GATTGGGGGA AGCTTCTAGA ATTGCAAGCT
GCTGATGGCT CATTTCTCTA CTCTCCATCC TCTACTGCCT
TTGCACTAGA ACAAACCAAG GATCAAAACT GCCTCAAATA
TCTATCTAAA CTTGTTCGAA AATTCGATGG CGGAGTACCC
AACGTGTACC CGGTGGACTT GTTCGAACAT AATTGGGCAG
TTGATCGTCT CCAAAGGCTC GGAATTTCTC GTTATTTTAC
GCCTGAAATC AACCAATGTC TTGATTATTC TTACAGATAT
TGGTCAAATA GTAAAGGGAT GTACTCGGCA AGCAATTCCC
AGATTCAGCA CGTTGATGAC ACCGCCATGG GATTCAGGCT
TTTGAGACTC AACGGCTACG ATGTCTCTAC ACAAGGGTTT
AGGCAATTCG AGGCAGGGGG GGACTTCTTC TGCTTCGCGG
GGCAGTCGAG CCAAGGTGTA ACCGGAATGT ACAACCTCTA
CAGAGCTTCC CAAGTGATGT TCCCTGGAGA GAAGCTACTG
GAAGATGCCA AGAAATTCTC CACCAACTTC TTGCAACAAA
AACGAGCCAA TAACCAGCTC ACTGACAAGT GGGTTATTGC
CAAAGATGTT CCAGCTGAGG TGGGATATGC CTTGGATATT
CCCTGGTATG CCAGTCTGCC CCGACTGGAA GCAAGATTTT
TCATACAACA ATACGGTGGA GACGACGACG TTTGGATCGG
CAAAACCTTG TATAGAATGG GATATGTGAA CAACAACACT
TATCTGGAAC TCGCAAAGCT AGACTACAAC ACCTGCCAAA
GGTTGCATCA GCATGAGTGG ATAACCATTC AACGATGGTA
CGAAATTAAT TTAAAAATTA CTAGTGTTGG GTTGAGCAAA
AGAGGGGTCC TGTTGAGTTA TTACTTAGCC GCAGCCAATC
TGTTTGAGCC TCAAAACTCA ACACACCGCA TCGCTTGGGC
CAAAACTTCG ATTTTAGTAA GCGCTATTCA ACTTTCTCCC
CTCCAAAAGC GCGACTTTAT TAACCAATTC CACCGCTCCA
CCGCAAATAA TGGGTATGAA ACAAGTAATG TGTTGGTGAA
GAGTGTAATC AAGGGTGTGC ATGAGCTCTC CATGGACGCT
ATGTTGACGC ACAATAAAGA CATACATCGC CAACTTTTTA
ATGCTTGGCG AAAGTGGATG TCAGTGTGGG AAGAGGGAGG
TGATGGAGAA GCGGAGCTGT TATTGTCGAC GCTTAAGACG
TGCGACGGAG TAGATGAATC CACATTCAGC GATCCCAAAT
ACGAGCACCT CTTAGAGATC ACCGTCAGAG TCACCCACCA
GCTTCATCTC ATTCAGAATG CAGAGACGAA GCGTGTGGGT
GACCGTGAGG AAATAGATTT GAGCATGCAA CAACTTGTTA
AGTTGGTGTT CACTAAATCA TCATCGGATC TGGATTCTTG
TATCAAGCAA AGATTTTTTG CGATTGCCAG AAGTTTCTAT
TACGTGGCTC ATTGTGATCC GGAGATGGTG GACTCCCACA
TAGCCAAAGT ATTGTTTGAG AGGGTGATGT AG

Prunella vulgaris 11-hydroxy vulgarisane synthase (PvHVS) was identified and isolated as described herein. The Prunella vulgaris 11-hydroxy vulgarisane synthase (PvHVS) enzyme catalyzes the first Committed step and forms the scaffold found in all Vulgarisins, a class of diterpenes with pharmaceutical applications (e.g., gout, cancer). For example, PvH-VS can synthesize 11-hydroxy vulgarisane (shown below).

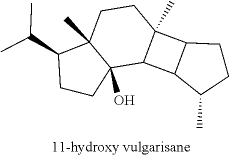 [SMILES: CC(C)[C@@H]1CC[C@@]2(O)C3C4C(CC[C@@H]4C)[C@]3(C)CC[C@@]12C]

An example of a formula for several Vulgarisin diterpenes is shown below.

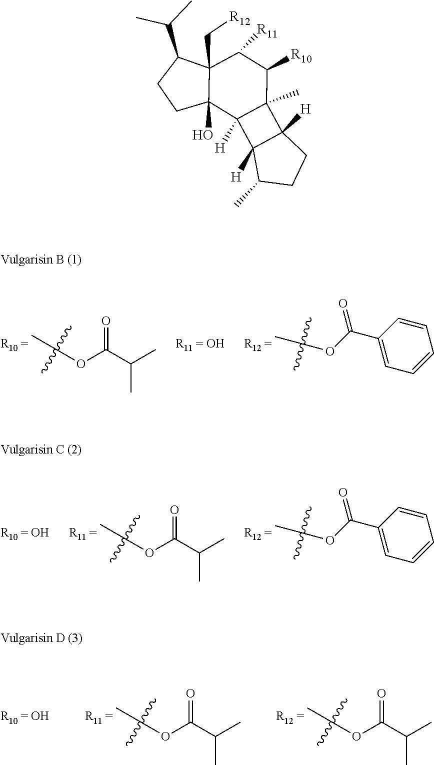

Vulgarisins B (1) and C (2) exhibit modest cytotoxicity activity against human lung carcinoma A549 cell line (Lou et al. Tetrahedron Letters 58: 401-404 (2017)).

The Prunella vulgaris 11-hydroxy vulgarisane synthase (PvHVS) can have the amino acid sequence shown below (SEQ ID NO:39).

MSSLSIPFSS AICTSSIPKI STGHHRRTAR MPAHDTSRLV
FRPSAVMVEG SPMTTSSNGK EVQRLITTEK PSMWKDIFST
FSFDNQVQEK YLKEIEELKK EVRSTLMSAT HRKLFDLIDN
LERMGIAYHF ETEIEDKLKQ AHASLEEEDD YDLFTTALRF
RLLRQHRYHV SCDPFAKFVD QDNKLKESLS SDVEGLLSLF
EASHLRIHNE DVLDEAlVFT THHLNRMKPQ LESPLKEEVK
HALRYPLHKC LGILSLRFHI DRYENDKSRD EVVLRLGQVN
FNYMQNIYMN ELYEITTWWN KLQMTSKVPY FRDRLVECYM
WGLAYHFEPE YAPVRVLITK YYMTATTVDD TYDNYATLEE
IELFTQAIDR WSEDEIDQLP DEYLKIVYKG LMNFTEEFRR
DAEERGKGYV IPYFIEETKR ATQGYANEQR WIMKREMPSF
EEYMVNSRVT SLMYVTYVAV VAVIESATKE TVDWALSDSD
IFVYTNDIGR LIDDLATHRR ERKDGTMLTS MDYYMKEYGG
TMEEGEAAFR KLMEEKWKLL NAAWVDTING KESKEIVVQV
LDLARICGTL YGDEEDGFTY PEKNFAPLVA ALLMNPIHI

A nucleic acid encoding the Prunella vulgaris 11-hydroxy vulgarisane synthase (PvHVS) enzyme with SEQ ID NO:39 is shown below as SEQ ID NO:40.

ATGAGCTCTC TCTCAATTCC CTTTTCTTCC GCCATTTGCA
CTTCATCAAT CCCAAAGATC AGTACTGGGC ATCATCGCCG
CACCGCGAGG ATGCCCGCGC ACGACACATC GCGTCTCGTC
TTTCGCCCTT CAGCTGTGAT GGTGGAAGGA AGTCCGATGA
CTACTTCAAG CAACGGGAAG GAAGTCCAAC GACTTATAAC
CACTTTCAAG CCTAGCATGT GGAAAGATAT TTTTTCTACC
TTCTCTTTCG ATAATCAGGT GCAAGAAAAG TATTTGAAAG
AAATTGAGGA ATTGAAGAAA GAAGTAAGAA GCACACTAAT
GAGTGCTACG CATAGGAAAT TGTTTGACTT GATCGACAAT
CTCGAGCGTA TGGGAATCGC CTATCATTTC GAGACAGAAA
TCGAAGACAA GCTCAAACAA GCTCATGCTT CTCTAGAGGA
GGAAGATGAC TACGACTTGT TCACTACTGC ACTTCGCTTT
CGTCTGCTCA GACAACATCG CTATCATGTT TCTTGCGATC
CCTTTGCGAA ATTTGTTGAC CAAGACAACA AATTGAAAGA
GAGTCTTAGT AGCGACGTCG AGGGGCTATT AAGCTTGTTC
GAGGCATCCC ATCTTCGGAT CCACAACGAG GATGTTCTAG
ATGAAGCTAT AGTGTTCACA ACCCATCACT TGAATCGAAT
GATGCCACAA TTGGAATCGC CCCTTAAAGA AGAAGTGAAG
CATGCTCTTC GATACCCCCT TCACAAGTGT CTTGGAATCC
TTAGCCTTCG TTTTCATATC GACAGATATG AGAATGATAA
GTCGAGGGAT GAAGTTGTTC TCAGACTAGG CCAAGTTAAT
TTCAATTACA TGCAGAACAT TTACATGAAC GAGCTCTATG
AAATCACCAC GTGGTGGAAC AAGTTGCAGA TGACTTCAAA
AGTACCTTAC TTTAGAGATA GATTGGTAGA GTGCTATATG
TGGGGTTTGG CATATCATTT CGAACCAGAA TACGCTCCCG
TTCGAGTCCT CATTACCAAG TACTATATGA CCGCCACAAC
TGTCGACGAT ACCTATGATA ATTATGCTAC ACTCGAAGAA
ATCGAACTCT TCACTCAGGC CATTGACAGG TGGAGCGAGG
ATGAGATTGA TCAGCTACCT GATGAATACC TAAAAATAGT
GTACAAAGGT CTAATGAACT TCACTGAAGA GTTTAGACGT
GACGCAGAAG AGCGAGCGAA AGGCTATGTG ATTCCTTACT
TTATTGAAGA AACGAAGAGA GCAACACAGG GTTATGCAAA
CGAGCAGAGG TGGATAATGA AGAGAGAAAT GCCGAGTTTT
GAAGAGTATA TGGTGAACTC AAGGGTAACA TCACTTATGT
ATGTGACCTA CGTTGCTGTT GTGGCAGTCA TAGAATCAGC
TACCAAAGAA ACCGTAGATT GGGCGCTAAG TGACTCCGAT
ATCTTTGTCT ACACTAACGA TATCGGCCGA CTTATCGACG
ACCTTGCCAC TCATCGACGC GAGAGGAAAG ACGGGACAAT
GCTTACATCG ATGGATTATT ACATGAAGGA ATATGGCGGT
ACGATGGAAG AGGGGGAAGC TGCATTTAGG AAATTGATGG
AGGAGAAATG GAAACTTTTG AATGCAGCAT GGGTAGATAC
TATTAATGGA AAAGAGTCGA AGGAAATAGT TGTGCAAGTT
CTCGACCTCG CCAGGATATG CGGAACGCTC TATCGGGACG
AAGAAGATGG CTTCACCTAC CCAGAGAAGA ATTTTGCACC
ACTCGTTGCT GCTCTATTGA TGAATCCTAT ACATATTTGA

A Chiococca alba ent-CPP synthase (CaTPS1) was identified and isolated. This CaTPS1 enzyme was identified that converts GGPP to ent-CPP [16].

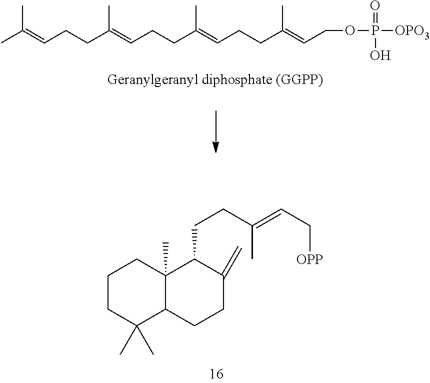

The Chiococca alba ent-CPP synthase (CaTPS1) has the amino acid sequence shown below (SEQ ID NO:41).

| | |
|---|---|
| 1 | MSSSTSAAAT LLGLSPASRR FVSFPPANGP IETITGIWSP |
| 41 | GKALHHFNFR LRCSTVSSPR TQELGQVSQN GMSGIKWHDI |
| 81 | VEEGVTEKGT LEANTSSWIK ESIEAIRWML RTMDDGDISI |
| 121 | SAYDTAWVAL VEDINGSGGP QFPSSLEWIA NNQLPDGSWG |
| 161 | DSDIFSAHDR ILNTLGCVVA LKSWNMHPEK SEKGLLYLRD |
| 201 | NIHKLEDENV EHMPIGFEVA FPSLIEIAKK LSIDIPDDSA |
| 241 | ILQEIYARRN LKLTRIPKDI MHTVPTTLLH SLEGMPELDW |
| 281 | KRLISLKCED GSFLFSPSST AFALTQTKDA DCLRYLIKTV |
| 321 | QKENGGVPNV YPVDLFEHIW AVDRLQRLGI SRYFQSEIRE |
| 361 | CIDYVHRYWT DKGICWARNT HVYDIDDTAM GFRLLRLHGY |
| 401 | DVSADVFRYY EKDGEFVCFA GQSNQAVTGM YNLYRASQVM |
| 441 | FPGENILSDA ERFSSEFLHD KRANNELLDK WIITKDLPGE |
| 481 | VAYALDVPWY ASLPRLETRL YLEQYGGEDD VWIGKTLYRM |
| 521 | QKVNNNIYLE LGKLDYNNCQ ALHQLEWRSI QKWYNECGLG |
| 561 | EYGLSERSLL LSYYLAAASI FEPERSKERL AWAKTTMLIR |
| 601 | TIESYLSSEQ MVEDHNGAFV SEFQYYCSNL DYVNGGRHKP |
| 641 | TQRLVRTLLG TLNQISLDAV LVHGRDIHQY LRQAWEKWLI |
| 681 | ALQEGDDSDM GQEEAELLVR TLNLCAGRYA SEELLLSHPK |
| 721 | YQQLLHITTR VCNQIRHFQH KKVQDGENGR ANMGDGITSI |
| 761 | SSIESDMQEL TKLVVGNTQN DLDADTKQTF LTVAKSFYYT |
| 801 | AHCNPGTINC HIAKVLFERV L |

A nucleic acid encoding the Chiococca alba ent-CPP synthase (CaTPS1) with SEQ ID NO:41 is shown below as SEQ ID NO:42.

| | |
|---|---|
| 1 | ATGTCTTCTT CTACCTCAGC AGCAGCAACC CTTCTCGGAT |
| 41 | TATCGCCGGC AAGCCGCCGG TTTGTATCAT TTCCTCCGGC |
| 81 | AAATGGACCT ATAGAAACTA TTACCGGTAT TTGGTCGCCC |
| 121 | GGCAAAGCTC TTCATCACTT TAATTTCCGT CTGCGTTGTA |
| 161 | GCACGGTGTC CAGTCCTCGC ACCCAAGAAT TGGGCCAGGT |
| 201 | GTCACAAAAT GGCATGTCTG GTATAAAGTG GCATGACATA |
| 241 | GTGGAAGAAG GAGTCACAGA AAAAGGAACT CTTGAGGCGA |
| 281 | ACACATCAAG CTGGATAAAA GAAAGCATAG AAGCCATTCG |
| 321 | TTGGATGCTG CGTACCATGG ATGACGGGGA TATCAGCATA |
| 361 | TCTGCTTATG ATACTGCATG GGTTGCCCTT GTGGAAGATA |
| 401 | TCAACGGAAG TGGCGGTCCT CAATTTCCTT CAAGCCTCGA |
| 441 | GTGGATTGCC AACAATCAGC TTCCTGATGG TTCATGGGGC |
| 481 | GACAGCGACA TCTTTTCAGC TCACGATCCG ATTCTCAACA |
| 521 | CTTTGGGATG CGTTGTTGCA TTAAAATCTT GGAACATGCA |
| 561 | CCCTGAAAAG AGTGAAAAAG GATTATTATA TTTAAGGGAT |
| 601 | AACATTCACA AGCTTGAGGA TGAAAATGTC GAGCACATGC |
| 641 | CTATCGGTTT TGAAGTGGCA TTTCCTTCAC TAATTGAGAT |
| 681 | AGCCAAAAAG TTGAGCATTG ATATTCCGGA TGATTCTGCA |
| 721 | ATCTTGCAGG AGATATATGC CAGAAGAAAT CTAAAGCTAA |
| 761 | CAAGGATACC GAAGGACATT ATGCACACAG TGCCCACAAC |
| 801 | ATTGCTCCAC AGCTTGGAAG GCATGCCAGA ACTAGACTGG |
| 841 | AAAAGGCTAA TATCTCTAAA GTGTCAGGAT GGTTCCTTTC |
| 881 | TGTTTTCTCC ATCCTCCACT GCTTTTGCCC TCACGCAAAC |
| 921 | TAAAGATGCT GATTGCCTCA GATATTTAAC TAAAACCGTA |
| 961 | CAAAAATTCA ATGGAGGAGT TCCCAATGTT TACCCCGTGG |
| 1001 | ACTTATTCGA ACACATCTGG GCTGTTGATC GACTTCAAAG |
| 1041 | ACTAGGAATT TCTCGATACT TCCAGTCAGA AATCCGCGAG |
| 1081 | TGCATCGATT ATGTTCACCG ATATTGGACG GATAAAGGTA |
| 1121 | TCTGTTGGGC TAGAAATACC CACGTTTATG ACATTGATGA |
| 1161 | TACAGCTATG GGTTTTAGAC TTCTAAGGTT GCATGGCTAC |
| 1201 | GATGTTTCTG CAGATGTTTT CAGATACTAT GAGAAGGATG |
| 1241 | GCGAATTCGT TTGCTTTGCC GGACAGTCAA ACCAGGCGGT |
| 1281 | GACCGGAATG TATAACCTGT ATAGAGCTTC TCAAGTGATG |
| 1321 | TTTCCAGGGG AGAATATACT TTCGGATGCT AGGAAATTCT |
| 1361 | CGTCCGAATT CTTGCATGAT AAGCGAGCCA ACAATGAGCT |
| 1401 | CCTAGATAAA TGGATCATAA CCAAAGATTT GCCTGGGGAG |
| 1441 | GTAGCATATG CTTTAGATGT TCCATGGTAT GCCAGTTTAC |
| 1481 | CTCGTTTAGA AACCAGATTG TATTTGGAAC AATATGGCGG |
| 1521 | CGAAGATGAT GTCTGGATTG GCAAGACATT GTACAGGATG |
| 1561 | CAAAAAGTTA ACAACAACAT CTATCTTGAA CTTGGCAAAT |
| 1601 | TAGATTACAA CAACTGTCAG GCATTGCATC AGCTTGAGTG |
| 1641 | GAGAAGCATC CAAAAATGGT ACAATGAATG CGGTCTTGGA |
| 1681 | GAGTACGGAT TAAGCGAGAG AAGCCTCCTT CTTTCGTATT |
| 1721 | ATTTGGCCGC AGCCAGTATA TTTGAAGCGG AGAGGTCAAA |
| 1761 | GGAACGGCTT GCCTGGGCCA AAACTACTAT GCTAATCCGC |
| 1801 | ACAATTGAAT CTTATTTGAG TAGTGAACAA ATGGTTGAGG |
| 1841 | ATCACAATGG AGCCTTTGTT AGCGAGTTCC AATACTATTG |
| 1881 | CAGTAACCTT GACTACGTAA ATGGTGGAAG GCATAAGCCA |
| 1921 | ACACAAAGGC TAGTGAGGAC TCTACTCGGA ACTTTAAATC |
| 1961 | AGATTTCTTT GGACGCAGTG TTAGTCCACG GCAGAGATAT |
| 2001 | CCATCAATAT TTGCGTCAAG CCTGGGAAAA GTGGTTGATA |
| 2041 | GCTTTGCAAG AGGGAGATGA TAGTGACATG GGTCAAGAGG |
| 2081 | AAGCAGAACT TTTAGTGCGC ACACTAAACC TATGCGCCGG |
| 2121 | TCGCTACGCA TCGGAGGAGC TATTGTTGTC CCATCCCAAG |
| 2161 | TATCAACAAC TTTTGCACAT CACTACTAGA GTCTGTAACC |
| 2201 | AAATTCGTCA TTTCCAACAC AAAAAGGTGC AAGATGGGGA |
| 2241 | AAATGGAAGA GCAAACATGG GTGATGGCAT CACAAGCATC |
| 2281 | AGCTCAATAG AGTCGGACAT GCAAGAACTA AGGAAATTAG |
| 2321 | TTGTCGGCAA TACCCAAAAC GATCTAGATG CTGATACGAA |
| 2361 | GCAAACATTT CTCACGGTGG CAAAAAGCTT CTACTACACC |
| 2401 | GCCCACTGCA ATCCCGGAAC AATCAATTGC CATATTGCTA |
| 2441 | AAGTATTATT TGAGAGAGTA CTTTGA |

A Chiococca alba (5R,8S,9S,10S)-labda-13-en-8-ol diphosphate (ent-8-LPP) synthase (CaTPS2) was identified and isolated as described herein. This CaTPS2 enzyme was identified as an 5R,8S,9S,10S)-labda-13-en-8-ol diphosphate (ent-8-LPP) synthase, which converts GGPP to 5R,8S,9S,10S)-labda-13-en-8-ol diphosphate (ent-8-LPP, [7]).

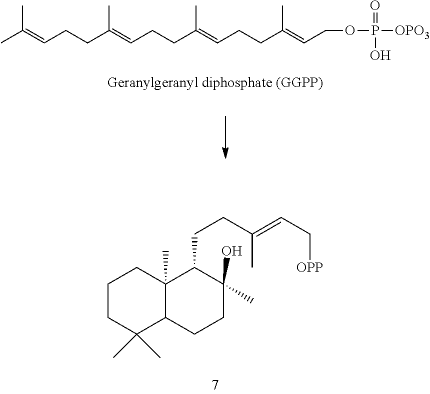

The Chiococca alba (5R,8S,9S,10S)-labda-13-en-8-ol diphosphate (ent-8-LPP) synthase (CaTPS2) has the amino acid sequence shown below (SEQ ID NO:43).

| | |
|---|---|
| 1 | MPVIKSHEFI EEVGPEKGTL KLSRSSRINE LVESIQTMLQ |
| 41 | SMDDGEISMS AYDTAWVALV EDINGSSYPQ FPMSLEWIAN |
| 81 | NQLPDGSWGD GSIFSVHDRI ISTLCCVLAL KSWNMHPDKS |
| 121 | EKGLLFIRDN IHKVGDESAE HMPIGFEVVF PSLIERAKNL |
| 161 | DIDIPDISAI LQEIYARRNL KLARIPKDIL YTVPTTLLHS |
| 201 | LEGMPELDWQ KLLPLKCEDG SFLFSPSCTA FALMQTKDGD |
| 241 | CLRYLTNTIE KFNGGVPGVY PVDLFEHIWA VDRLQRLGIS |
| 281 | RYFQTEIEEC MSYVYRYWTD KGICWARNSK VEDIDDTAMG |
| 321 | FRLLRLHGYM VSADVFAQFE KGGEFVCFAG QSNQALTGMF |
| 361 | NLYRASQVMF PGEKILADAK KFSSNFLHEK RANNELLDKW |
| 401 | IITKDLPGEV TYALDVPWYA SLPRVETRLY LEQYGGEDDV |
| 441 | WIAKTLYRMR KVNNKIYLEL GILDYNNCQA LHQLEWRSIQ |
| 481 | KWYKDSGLEE YGLSERNLLL AYYLATACIF EPERLVERLS |
| 521 | WAKTTALIYT TKSYFRTECN SGEQRKAFLH EFQQYCNDLD |
| 561 | YVSGARHKPT IRLIEALLGT LEQVSLDAIL DHGRYIHQDL |
| 601 | RNAWEKWLIA LQEGVDMDQE EAELTVLTLH LCAGSYTSEE |
| 641 | LLLSHPKYQQ LLNITSRVCH QIRQFQREKA QDTDNGRENL |
| 681 | VAITSIKAIE SDMQELAKLV LTKSTGDLAA KIKQTFLIVA |
| 721 | KSFYYTAHCL PGIISTHIAK VLFEKVF |

A nucleic acid encoding the Chiococca alba (5R,8S,9S,10S)-labda-13-en-8-ol diphosphate (ent-8-LPP) synthase (CaTPS2) with SEQ ID NO:43 is shown below as SEQ ID NO:44.

| | |
|---|---|
| 1 | ATGCCAGTAA TAAAGTCGCA TGAGTTTATT GAAGAGGTCG |
| 41 | GCCCGGAAAA AGGAACTCTG AAGCTGAGCA GATCAAGTAG |
| 81 | GATAAACGAA CTTGTAGAAT CAATTCAAAC GATGCTTCAA |
| 121 | TCGATGGATG ATGGGGAAAT AAGCATGTCT GCTTATGACA |
| 161 | CCGCGTGGGT TGCCCTTGTG GAAGATATTA ATGGAAGCAG |
| 201 | CTACCCTCAA TTCCCTATGA GCCTCGAGTG GATTGCCAAC |
| 241 | AATCAGCTTC CTGATGGTTC ATGGGGTGAC GGCAGTATCT |
| 281 | TTTCGGTTCA TGATCGGATA ATCAGCACAT TAGGATGTGT |
| 321 | TCTTGCATTA AAATCATGGA ACATGCACCC GGACAAAAGC |
| 361 | GAAAAAGGAC TGTTATTTAT AAGGGACAAT ATTCACAAGG |
| 401 | TTGGAGATGA CAGCGCTGAG CACATGCCTA TTGGTTTTGA |
| 441 | GGTGGTATTT CCTTCGCTTA TTGAGAGAGC CAAAAACTTG |
| 481 | GACATTGATA TTCCAGATAT TTCTGCTATC TTGCAAGAGA |
| 521 | TTTATGCACG AAGAAATCTA AAGCTCGCAA GGATTCCAAA |
| 561 | GGATATACTG TATACCGTGC CCACGACATT ACTTCATAGC |
| 601 | TTAGAAGGAA TGCCAGAACT GGACTGGCAA AAGCTACTGC |
| 641 | CATTAAAATG TGAGGATGGT TCATTTCTAT TTTCTCCATC |
| 681 | GTGCACTGCT TTTGCCCTCA TGCAGACTAA GGATGGTGAT |
| 721 | TGCCTCAGAT ATCTAACTAA TACCATAGAA AAATTCAATG |
| 761 | GGGGAGTTCC CGGTGTATAC CCTGTGGACT TGTTCGAACA |
| 801 | CATTTGGGCT GTTGATCGCT TGCAAAGACT AGGAATTTCC |
| 841 | CGGTATTTTC AGACAGAAAT TGAAGAATGT ATGAGTTATG |
| 881 | TTTACCGATA TTGGACGGAT AAAGGTATCT GTTGGGCTAG |
| 921 | AAACTCCAAA GTTGAAGACA TCGATGACAC AGCCATGGGT |
| 961 | TTTAGACTTC TAAGGTTGCA TGGTTACATG GTTTCTGCAG |
| 1001 | ATGTGTTTGC ACAGTTTGAG AAAGGGGGTG AATTCGTTTG |
| 1041 | CTTTGCTGGA CAGTCGAACC AGGCGCTGAC TGGAATGTTT |
| 1081 | AACCTGTATA GAGCTTCTCA AGTAATGTTT CCAGGGGAGA |
| 1121 | AGATACTTGC TGATGCCAAG AAATTCTCAT CGAACTTCTT |
| 1161 | ACATGAAAAG CGTGCAAACA ACGAGCTTCT AGATAAATGG |
| 1201 | ATCATAACTA AAGATTTGCC TGGAGAGGTG ACGTATGCGC |
| 1241 | TAGATGTTCC ATGGTACGCC AGTTTACCTC GTGTAGAAAC |
| 1281 | GAGATTATAT CTGGAACAAT ATGGAGGAGA GGATGATGTC |
| 1321 | TGGATTGCCA AGACATTGTA CAGGATGAGA AAAGTTAACA |
| 1361 | ACAAAATTTA CCTTGAACTT GGCATATTAG ATTACAATAA |
| 1401 | CTGTCAAGCA TTGCATCAGC TGGAGTGGAG AAGCATCCAA |
| 1441 | AAATGGTATA AGGATTCTGG CCTTGAAGAG TACGGGTTGA |
| 1481 | GCGAGAGGAA CCTTCTCCTG GCATATTATC TGGCCACAGC |
| 1521 | TTGTATATTT GAACCCGAAA GGTTGGTGGA GCGCCTTTCC |
| 1561 | TGGGCGAAAA CAACCGCCTT AATCTACACA ACAAAATCTT |
| 1601 | ATTTCAGAAC TGAATGCAAC TCTGGGGAAC AGAGAAAAGC |
| 1641 | TTTTCTTCAT GAGTTCCAAC AGTACTGCAA TGACCTGGAC |
| 1681 | TACGTTAGTG GCGCAAGGCA CAAGCCAACA ATAAGATTGA |
| 1721 | TCGAAGCTCT ACTTGGAACC CTAGAGCAGG TCTCTTTGGA |
| 1761 | TGCAATATTA GATCATGGCC GATATATCCA TCAAGATTTG |
| 1801 | CGTAATGCTT GGGAGAAATG GTTGATAGCT TTGCAAGAGG |
| 1841 | GAGTTGACAT GGACCAAGAA GAAGCAGAAC TTACAGTGCT |
| 1881 | CACACTACAC CTGTGTGCCG GCAGCTACAC ATCGGAGGAG |
| 1921 | TTACTGTTAT CTCATCCCAA GTATCAACAA CTTTTAAATA |
| 1961 | TCACTAGTAG AGTCTGCCAC CAAATTCGTC AATTCCAGCG |
| 2001 | CGAAAAGGCA CAGGATACGG ATAATGGAAG AGAAAACTTG |
| 2041 | CTTGCCATCA CAAGCATCAA GGCGATAGAA TCAGACATGC |
| 2081 | AAGAACTTGC GAAATTAGTT CTGACCAAAT CCACTGGCGA |
| 2121 | TTTAGCTGCT AAAATCAAGC AAACATTTCT TATAGTGGCA |
| 2161 | AAGAGCTTCT ACTACACCGC ACATTGCCTT CCTGGAATTA |
| 2201 | TCAGTACCCA CATTGCCAAA GTACTATTTG AGAAAGTTTT |
| 2241 | CTGA |

A Chiococca alba CaTPS3 and CaTPS4 were identified and isolated. CaTPS3 and CaTPS4 were identified as an ent-kaurene synthase, converting ent-CPP [16] into ent-kaurene [19].

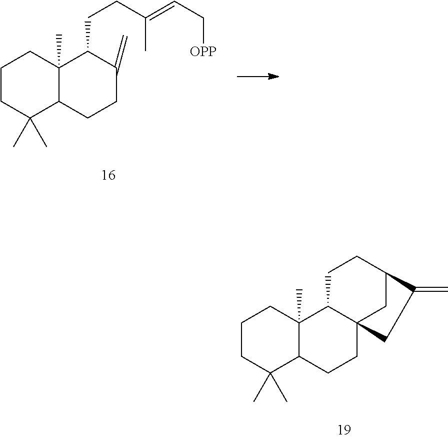

The Chiococca alba ent-kaurene synthase (CaTPS3) has the amino acid sequence shown below (SEQ ID NO:45).

| | |
|---|---|
| 1 | MMMMMVVMNT APAHSYHPFP FAGPKSSATL FSNYYCSSRK |
| 41 | KSSPPRISAS VSLLTGVEST TAINSSDPEI KERIRKLFHD |
| 81 | VDISLSSYDT AWVAMVPAPH SSQSPLFPQC INWLLDNQLP |
| 121 | DGSWSLPPPH HHPLLLKDAL SSTLACVLAL RRWGIGQEQV |
| 161 | DKGIRFVELN FASASDQNQH LPVGFDIIFP GMLEYARDLN |
| 201 | LNLQLESATV NALLLKRDQE LTRFFKSYSD ESKAYLAYVS |
| 241 | EGIVKLQNWD TVMKFQRKNG SLFNSPSATA AAVMHVHNPG |
| 281 | CLDYLHSVLE KHGNAVPTVY PLDIYPRLCL VDNLERLGIC |
| 321 | GHFRKEILSV LDDTYRCWMQ GDEEIFAEKS TCAIAFTLLR |
| 361 | KHGYNISADP LTPFLKEECF SNSLGGCLKD TSAVLELYRA |
| 401 | LEMIISQNES ALVKKSLWSR SFLKEHISGG CDLKGFSNQI |
| 441 | SILVDDILNF PSHATLQRVA NRRSIEQYNL DSTKILKTSY |
| 481 | CSSNFSNKDL LILAVKDFNH CQLIHREELK ELERWVTDNR |
| 521 | LDKLKFARQK SAYCYFSAAA TIFSPELSDA RMSWAKNGVL |
| 561 | ATLVDDFFDV GGSLEELKKL IELVEKWDIN VSDGCCSEPV |
| 601 | QILFSALHST IQEIGDkAFK WQARSVTNHI FKIWLDLLNS |
| 641 | MLREAEWARN ATVPTVEEYM TNGYVSFALG PIILPALYLV |
| 681 | GPKLSEEVVK DSEFHSLFKL VSTCGRLLND VHSFERESKS |
| 721 | GQLNALSLRL IHGGVGITEA AAVAEMKSSI ENLRRELLRL |
| 761 | VLRKEGSVVP RACKDLFWNM SKVLHQFYNK DDGFTSEEMI |
| 801 | QLVKSIIYEP IAVNEFLNSC HT |

A nucleic acid encoding the Chiococca alba ent-kaurene synthase (CaTPS3) with SEQ ID NO:45 is shown below as SEQ ID NO:46.

| | |
|---|---|
| 1 | ATGATGATCA TGATCGTGGT GATGAACACA GCTCCCGCCC |
| 41 | ACTCTTACCA TCCTTTCCCC TTTGCCGGCC CAAANTCCTC |
| 81 | AGCCACACTT TTTTCCAATT ATTATTGTTC CAGTAGGAAG |
| 121 | AAATCATCGC CACCTCGCAT CTCTGCCTCA GTTTCTTTGC |
| 241 | TAACTGGAGT TGAAAGCACA ACTGCAATTA ATTCTTCAGA |
| 281 | CCCGGAGATC AAAGAAAGAA TAAGGAAACT ATTTCATGAT |
| 321 | GTTGATATCT CGCTTTCTTC ATATGACACT GCATGGGTGG |
| 361 | CAATGGTCCC TGCTCCACAT TCTTCCCAGT CTCCCCTTTT |
| 401 | TCCCCAGTGC ATTAATTGGT TATTGGACAA TCAGCTTCCT |
| 441 | GATGGCTCAT GGAGTCTTCC TCCTCCTCAT CATCATCCTC |
| 481 | TATTACTTAA AGATGCATTA TCCTCTACCC TTGCATGTGT |
| 521 | TCTTGCGCTC AGGAGATGGG GAATTGGTCA AGAACAAGTT |
| 561 | GACAAGGGTA TTCGTTTTGT TGAGTTAAAT TTTGCTTCAG |
| 601 | CATCTGACCA GAACCAGCAT TTGCCACTTG GATTTGACAT |
| 641 | TATATTCCCT GGCATGCTCG AATATGCTAG AGATTTAAAT |
| 681 | TTAAATCTTC AACTAGAATC TGCAACAGTA AATGCCTTAC |
| 721 | TTCTTAAAAG AGATCAGGAG CTTACAAGAT TCTTTAAAAG |
| 761 | CTACTCAGAC GAGAGTAAAG CATACCTTGC ATATGTATCA |
| 801 | GAAGGTATAG TAAAGTTACA GAACTGGGAT ACAGTTATGA |
| 841 | AGTTCCAAAG AAAGAACGGG TCACTATTCA ATTCACCTTC |
| 881 | AGCTACAGCA GCTGCTGTTA TGCATGTCCA CAATCCTGGT |
| 921 | TGCCTCGATT ACCTTCACTC AGTGTTGGAG AAGCATGGAA |
| 961 | ATGCTGTTCC AACAGTTTAC CCTTTGGATA TATATCCACG |
| 1001 | CCTCTGCTTG GTTGACAACC TTGAGAGACT GGGTATTTGT |
| 1041 | GGTCATTTTA GGAAGGAAAT TCTGAGTGTA TTGGATGATA |
| 1081 | CATACAGATG CTGGATGCAG GGGGATGAAG AGATATTTGC |
| 1121 | AGAAAAATCA ACTTGTGCCA TAGCATTTAC ATTATTGCGA |
| 1161 | AAGCATGGGT ACAACATCTC TGCAGATCCA TTGACCCCAT |
| 1201 | TCTTAAAGGA AGAGTGTTTT TCCAATTCTT TGGGTGGATG |
| 1241 | TTTGAAAGAT ACTAGTGCTG TACTTGAATT ATACCGGGCA |
| 1281 | TTAGAGATGA TTATTAGCCA GAATGAATCA GCTCTGGTGA |
| 1321 | AAAAAAGCTT GTGGTCCAGA AGCTTCCTGA AAGAGCATAT |
| 1361 | TTCTGGTGGT TGTGATTTAA AGGGATTCAG CAATCAAATT |
| 1401 | TCCATACTGG TGGATGATAT CCTCAACTTT CCATCGCATG |
| 1481 | CTACTTTGCA ACGGGTTGCT AACAGGAGAA GCATAGAGCA |
| 1521 | ATACAACTTA GACAGTACAA AAATTTTAAA AACTTCATAT |
| 1561 | TGCTCGTCGA ATTTTAGCAA CAAAGATTTA TTGATCCTGG |
| 1601 | CAGTCAAAGA TTTTAATCAT TGCCAACTCA TACACCGTGA |
| 1641 | AGAACTGAAA GAACTAGAAA GGTGGGTCAC AGACAATAGA |
| 1681 | TTGGACAAGT TAAAGTTTGC TAGGCAGAAG TCTGCATACT |
| 1721 | GTTACTTTTC TGCTGCAGCA ACCATATTCT CACCTGAACT |
| 1761 | TTCTGATGCC CGCATGTCAT GGGCCAAGAA TGGTGTACTT |
| 1801 | GCTACTTTGG TTGATGACTT CTTTGACGTG GGAGGTTCTC |
| 1841 | TAGAGGAATT AAAGAAACTG ATTGACTTGG TTGAAAAGTG |
| 1881 | GGATATAAAT GTCAGTGATG GTTGTTGCTC TGAACCAGTG |
| 1921 | CAAATCCTCT TCTCAGCACT ACATAGTACA ATCCAGGAGA |
| 1961 | TTGGAGATAA AGCATTCAAA TGGCAAGCAC GCAGTGTAAC |
| 2001 | AAACCACATA TTTAAGATAT GGTTAGATTT GCTTAATTCT |
| 2041 | ATGTTGAGGG AAGCTGAGTG GGCTAGAAAT GCAACAGTGC |
| 2081 | CTACAGTTGA AGAATATATG ACAAATGGTT ATGTATCATT |
| 2121 | THCTTTGGGG CCAATTATCC TCCCTGCTCT TTATCTTGTT |
| 2161 | GGACCTAAGC TGTCAGAGGA AGTAGTTAAG GATTCTGAAT |
| 2201 | TCCACTCCCT TTTTAAGCTA GTGAGTACCT GTGGGCGGCT |
| 2241 | TCTGAATGAT GTCCACAGCT TCGAGAGGGA ATCAAAGTCC |
| 2281 | GGCCAACTAA ATGCTCTGTC TCTGCGCCTG ATTCATGGTG |
| 2321 | GTGTTGGCAT TACTGAAGCA GCTGCTGTTG CAGAGATGAA |
| 2361 | GAGTTCAATT GAGAATCTAA GGAGAGAACT GCTGAGACTA |
| 2401 | GTCTTGCGCA AAGAGGGTAG TGTAGTTCCA AGAGCTTGCA |
| 2441 | AGGATTTGTT TTGGAATATG AGTAAAGTGC TACATCAATT |
| 2481 | TTACAACAAA GATGATGGAT TTACTTCAGA GGAGATGATT |
| 2521 | CAGCTTGTGA AGTCGATCAT TTATGAGCCA ATTGCGGTCA |
| 2561 | ATGAATTTTT GAATAGTTGC CATACATGA |

The Chiococca alba ent-kaurene synthase (CaTPS4) has the amino acid sequence shown below (SEQ ID NO:47).

| | |
|---|---|
| 1 | MMIMVMNTAP VHAYHALPIP TQKSSTTLFP NYNCSSRKKS |
| 41 | SPPRISAASV SLQTGVERTT AIHSSDLEIK ERIRKLFHDV |
| 81 | DISLSSYDTA WVAKVPAPHS SQSPLFPQCI NWLLDNQLPD |
| 121 | GSWSLPPHHH HHHPLLLKDA LSSTLACVLA LRRWGIGQEQ |
| 161 | VDKGIRFVEL NFASASDQNQ HLPVGFDIIF PGMLEYARDL |
| 201 | NLNLQLESAT VDALLLKRDQ ELIRFFKSYS DESKAYLAYV |
| 241 | SEGIIKLQNW DTVMKFQRKN GSLFNSPSAT AAAVMHVHNP |
| 281 | GCLDYLHSVL EKHGNAVPTV YPLDIYPRLC LVDNLERLGI |
| 321 | CGHFRKEILS VLDDTYRCWM QGDEEIFAEK STCAIAFTLL |
| 361 | RKHGYNISAD PLTPFLKEEC FSNSLGGCLK DTSAVLELYR |
| 401 | ALEMIISQNE SALVKKSLWS RSFLKEHISG GCDLKGFSNQ |
| 441 | ISKQVDDILN FPSHATLQRV ANRRSIEQYN LDSTKILKTS |
| 481 | YCSSNFSNKD LLILAVKDFN HCQLIHREEL KELERWVADN |
| 521 | RLDKLKFARQ KSAYCYFSAA ATIFSPELSD ARISWAKNGV |
| 561 | LTTLVDDFFD VGGSLEELKK LIELVEKWDI NVSDGCCSEP |
| 601 | VQILFSALHS TIQEIGDKAF KWQARSVINH IIKIWLDLLN |
| 641 | SMLREAEWAR NATVPTVEEY MINGYVSFAL GPIILPALYL |
| 681 | VGPKLSEELV KDSEFHSLFK LVSTCGRLLN DVHSFERESK |
| 721 | AGQLNALSLR LIHGGVGITE AAAVAEMKSS IEKQRRELLR |
| 761 | LVLRKEGSVV PRACKDLFWN MSRVLHQFYV KDDGFTSEEM |
| 801 | IELVKSIIYE PIAVNEF |

A nucleic acid encoding the Chiococca alba ent-kaurene synthase (CaTPS4) with SEQ ID NO:47 is shown below as SEQ ID NO:48.

| | |
|---|---|
| 1 | ATGATGATAA TGGTGATGAA CACAGCTCCC GTCCACGCTT |
| 41 | ACCACGCTTT ACCCATTCCC ACCCAAAAAT CCTCAACCAC |
| 81 | ACTTTTTCCC AATTATAACT GTTCCAGTAG GAAGAAATCA |
| 121 | TCGCCACCTC GCATCTCTGC CGCCTCAGTT TCTTTGCAAA |
| 161 | CTGGAGTTGA AAGAACGACG GCAATTCATT CTTCAGACCT |
| 201 | AGAGATCAAA GAAAGAATAA GGAAACTATT TCATGATGTT |
| 241 | GATATCTCGC TTTCTTCATA TGACACTGCA TGGGTGGCAA |
| 281 | TGGTCCCTGC TCCACATTCT TCCCAGTCTC CCCTTTTTCC |
| 321 | CCAGTGCATT AATTGGTTAT TGGACAATCA GCTTCCTGAT |
| 361 | GGCTCATGGA GTCTTCCTCC TCATCATCAT CATCATCATC |
| 401 | CCCTATTACT TAAAGATGCA TTATCCTCTA CGCTTGCATG |
| 441 | TGTTCTTGCG CTCAGGAGAT GGGGAATTGG TCAAGAACAA |
| 481 | GTTGACAAGG GTATTCGTTT TGTTGAGTTA AATTTTGCTT |
| 521 | CTGCATCTGA CCAGAACCAG CATTTGCCAG TTGGATTTGA |
| 561 | CATTATATTC CCTGGCATGC TCGAATATGC TAGAGATTTA |
| 601 | AATTTAAATC TTCAACTAGA ATCCGCAACT GTAGATGCCT |
| 641 | TACTTCTCAA AAGAGATCAG GAGCTTATAA GATTCTTTAA |
| 681 | AAGCTACTCA GACGAGAGTA AAGCATACCT TGCATATGTA |
| 721 | TCAGAAGGTA TCATAAAGTT ACAGAACTGG GATACAGTTA |
| 761 | TGAAGTTCCA AAGAAAGAAC GGGTCACTGT TCAATTCACC |
| 801 | TTCAGCTACA GCAGCTGCTG TTATGCATGT CCACAATCCT |
| 841 | GGCTGCCTCG ATTACCTTCA CTCAGTGTTG GAGAAGCATG |
| 881 | GCAATGCTGT TCCAACAGTT TACCCTTTGG ATATATATCC |
| 921 | ACGCCTCTGC TTGGTTGACA ACCTTGAGAG ACTGGGTATT |
| 961 | TGTGGTCATT TTAGGAAGGA AATTCTGAGT GTATTGGATG |
| 1001 | ATACATACAG ATGCTGGATG CAGGGGGATG AAGAGATATT |
| 1041 | TGCAGAAAAA TCAACTTGTG CCATAGCATT TACATTATTG |
| 1081 | CGAAAGCATG GGTACAACAT CTCTGCAGAT CCATTGACCC |
| 1121 | CATTCTTAAA GGAAGAGTGT TTTTCCAATT CTTTGGGTGG |
| 1161 | ATGTTTGAAA GATACTAGTG CTGTACTTGA ATTATACCGG |
| 1201 | GCATTAGAGA TGATTATTAG CCAGAATGAA TCAGCTCTGG |
| 1241 | TGAAAAAAAG CTTGTGGTCC AGAAGCTTCC TGAAAGAGCA |
| 1281 | TATTTCTGGT GGTTGTGATT TAAAGGGATT CAGCAATCAA |
| 1321 | ATTTCCAAAC AGGTGGATGA TATCCTCAAC TTTCCATCGC |
| 1361 | ATGCTACTTT GCAACGGGTT GCTAACAGGA GAAGCATAGA |
| 1401 | GCAATACAAC TTAGACAGTA CAAAAATTTT AAAAACTTCA |
| 1441 | TATTGCTCGT CGAATTTTAG TAACAAAGAT TTATTGATCC |
| 1481 | TGGCAGTCAA AGATTTTAAT CATTGCCAAC TCATACACCG |
| 1521 | TGAAGAACTG AAAGAACTAG AAAGGTGGGT CGCAGACAAT |
| 1561 | AGATTGGACA AGTTAAAGTT TGCTAGGCAG AAGTCTGCAT |
| 1601 | ACTGTTACTT TTCTGCTGCA GCAACCATAT TCTCACCTGA |
| 1641 | ACTTTCTGAT GCCCGCATCT CATGGGCCAA AAATGGTGTA |
| 1681 | CTTACTACTT TGGTTGATGA CTTCTTTGAC GTGGGAGGTT |
| 1721 | CTCTAGAGGA ATTAAAGAAA CTGATTGAGT TGGTTGAAAA |
| 1761 | GTGGGATATA AATGTCAGTG ATGGTTGTTG CTCTGAACCA |
| 1801 | GTGCAAATCC TCTTCTCAGC ACTACATAGT ACAATCCAGG |
| 1841 | AGATTGGAGA TAAAGCATTC AAATGGCAAG CACGCAGTGT |
| 1881 | AACAAACCAC ATAATTAAGA TATGGTTAGA TTTGCTTAAT |
| 1921 | TCTATGTTGA GGGAAGCTGA GTGGGCTAGA AATGCAACAG |
| 1961 | TGCCTACAGT TGAAGAATAT ATGACAAATG GTTATGTATC |
| 2001 | ATTTGCCTTG GGGCCAATTA TCCTCCCTGC TCTTTATCTT |
| 2041 | GTTGGACCTA AGCTCTCAGA GGAATTAGTT AAGGATTCTG |
| 2081 | AATTCCACTC CCTTTTTAAG CTAGTGAGTA CCTGTGGGCG |
| 2121 | GCTTCTGAAT GATGTCCACA GCTTCGAGAG GGAATCAAAG |
| 2161 | GCCGGCCAAC TAAATGCTCT TTCTCTGCGC CTGATTCATG |
| 2201 | GTGGAGTTGG CATTACTGAA GCAGCTGCTG TTGCAGAGAT |
| 2241 | GAAGAGTTCA ATTGAGAAGC AAAGGAGAGA ACTGCTGAGA |
| 2281 | CTAGTCTTGC GCAAAGAGGG TAGTGTAGTT CCAAGAGCTT |
| 2321 | GCAAGGATTT GTTTTGGAAT ATGAGTAGGG TGCTACATCA |
| 2361 | ATTTTACCTC AAAGATGATG GATTTACTTC AGAGGAGATG |
| 2401 | ATTGAGCTTG TGAACTCGAT CATTTATGAG CCAATTGCCG |
| 2441 | TCAATGAATT TTGA |

A Chiococca alba 13(R)-epi-dolabradiene synthase (CaTPS5) was identified and isolated. This CaTPS5 enzyme was identified as an 13(R)-epi-dolabradiene synthase, which converts ent-CPP [16] to 13(R)-epi-dolabradiene.

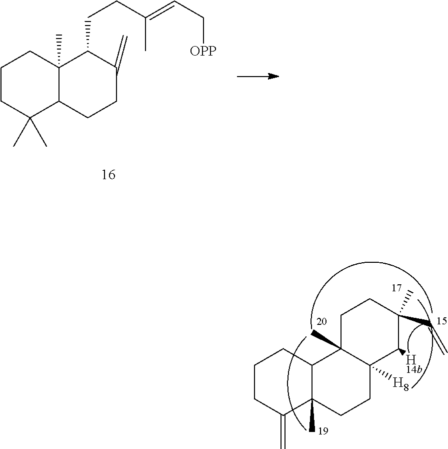

The Chiococca alba 13(R)-epi-dolabradiene synthase (CaTPS5) has the amino acid sequence shown below (SEQ ID NO:49).

| | |
|---|---|
| 1 | MIHTLPHGGQ AHFISHKTQP YYSSRPRFSS AASLDTRVRR |
| 41 | TSPSNSSVLD FNETKERITK LFHNVDYSIS SYDTAWVAMV |
| 81 | PDPHSSQAPL FPECINWLLD NQFHDGSWSL PHHNSLLLKD |
| 121 | VLSSTLACVL ALKRWGIGGR QIDKGVRFIE MNFGSASDNC |
| 161 | QHTPIGFDII FPGMLENARD LDLNLRLEPR IVIDMQRKRD |
| 201 | MQLTRLHESD LKGDQAYLAY VSEGMQKLQN WDLAMKFQRK |
| 241 | NGSLFNSPSA TAAAVMHVQN PASLNYLHSV VDKFGHAVPA |
| 281 | VYPLDLYARL CLVDNLERLG ICRHFTNEIE IVMEDTYRCW |
| 321 | LQDDEDIFAE ISTCALAFRL LRKHGYVVSP DPLTKIIEEE |
| 401 | DVSNSSGNGY WNDIHAVMEV HRASEVVIHE NESDLKNQNT |
| 441 | ISKHLLRHHL FNGSDVKPFP NPIYKQVDYA LKFPTPLILQ |
| 481 | RVENKTLIQN YDVDSTRLLK TSYRSSNFCN EDLLRLAVKD |
| 521 | FNDCQLLHRK ELKELERWSA DNRLHELKFA RQKAIYCSFS |
| 561 | AAATIFIPEW YEARMSLAKN SVLATVVDDF FDVGGSMEEL |
| 601 | KKLIEFVEKW DIDITKESCS EPLKIIFSAL HSTISEIGEQ |
| 641 | AVKWQGRNVT SHIIEIWLDL LNSMLRESEW TTDVHMPTLD |
| 681 | EYMEAAYVSF AMGPIIIPAL YFVGPKLSDE IVRDPEIRSL |
| 721 | HKLVSICGRL LNDMQGFERE KKAGKPNAVS IRISQNGDGI |
| 761 | TESAAFEEVK MELEDARREL LRLVVQKDGS VVPRACKDAF |
| 801 | WSVSRMLHHF YFNNDGYTSE VEMVELVNSI IHEPLK |

A nucleic acid encoding the Chiococca alba 13(R)-epi-dolabradiene synthase (CaTPS5) with SEQ ID NO:49 is shown below as SEQ ID NO:50.

| | |
|---|---|
| 1 | ATGATTCATA CTCTCCCTCA TGGCGGCCAG GCTCACTTCA |
| 41 | TTTCCCACAA AACACACCCT TATTATTCCA GTAGACCTCG |
| 81 | CTTTTCTTCA GCAGCTTCTT TGGACACACG AGTCCGGAGA |
| 121 | ACATCGCCCT CTAATTCCTC TGTCCTAGAC TTCAAGGAGA |
| 161 | CCAAAGAAAG AATCACAAAA TTATTTCATA ATGTTGATTA |
| 201 | TTCAATTTCT TCATATGATA CAGCATGGGT TGCTATGGTC |
| 241 | CCGGACCCAC ATTCTTCTCA GGCTCCCCTT TTCCCAGAGT |
| 281 | GCATAAATTG GTTGCTAGAT AATCAATTTC ATGATGGCTC |
| 321 | CTGGAGTCTT CCTCATCACA ATTCTCTATT GCTTAAGGAT |
| 361 | GTTTTATCCT CTACGCTTGC GTGTGTTCTT GCTCTTAAGA |
| 401 | GATGGGGAAT AGGAGGAAGG CAGATTGACA AAGGTGTTCG |
| 441 | CTTTATTGAG ATGAATTTTG GCTCAGCATC TGACAATTGC |
| 481 | CAGCATACTC CAATAGGATT TGACATAATA TTTCCAGGAA |
| 521 | TGCTTGAAAA TGCCAGAGAT TTGGATCTAA ATCTTAGACT |
| 561 | ACAACCCAGA ATTGTAACTG ACATGCAACG TAAAAGAGAC |
| 601 | ATGCAGCTTA CAAGACTCCA TGAAAGCGAT CTAAAGGGGG |
| 641 | ACCAAGCATA CTTGGCATAT GTATCCGAAG GGATGCAAAA |
| 681 | GTIACAGAAT TGGGATTTGG CGATGAAGTT TCAAAGGAAG |
| 721 | AATGGATCGC TCTTCAACTC ACCATCAGCT ACAGCAGCCG |
| 801 | CTGTTATGCA TGTCCAAAAT CCTGCTTCCC TCAATTATCT |
| 841 | TCATTCAGTC GTCGACAAAT TCGGCCATGC AGTTCCGGCT |
| 881 | GTTTACCCTT TGGATCTCTA TGCGCGCCTT TGCTTGGTTG |
| 921 | ACAATCTTGA GAGGCTGGGT ATCTGTCGAC ATTTTACTAA |
| 961 | TGAAATTGAA ATTGTAATGG AGGACACGTA CAGGTGCTGG |
| 1001 | CTGCAGGATG ATGAAGATAT ATTTGCCGAA ATATCAACTT |
| 1041 | GTGCCTTAGC TTTTCGGTTA TTGAGAAAAC ATGGCTATGT |
| 1081 | TGTCTCCCCA GATCCACTGA CAAAAATCAT AGAAGAAGAA |
| 1121 | GATGTTTCCA ATTCTTCTGG TAATGGATAT TGGAATGATA |
| 1161 | TACATGCTGT AATGGAAGTG CATCGGGCAT CAGAGGTGGT |
| 1201 | TATACATGAA AATGAATCAG ATTTAAAGAA TCAAAATACC |
| 1241 | ATATCAAAAC ACCTTCTCAG ACACCATCTT TTCAATGGTT |
| 1281 | CTGATGTGAA GCCCTTTCCT AATCCAATAT ACAAGCAGGT |
| 1321 | GGACTATGCT CTCAAGTTTC CAACCCCCTT AATTCTACAA |
| 1361 | CGTGTTGAAA ACAAGACCCT CATACAGAAC TACGACGTAG |
| 1401 | ACAGTACAAG ACTTCTTAAA ACTTCATATC GATCATCAAA |
| 1441 | TTTCTGCAAT GAAGATTTAC TGAGGTTAGC AGTGAAAGAT |
| 1481 | TTTAATGACT GTCAACTCCT GCACCGGAAA GAACTAAAAG |
| 1521 | AACTAGAAAG ATGGTCCGCA GATAACAGAC TGCACGAACT |
| 1601 | AAAAITTGCT CGGCAGAAAG CTATATACTG CTCCTTTTCT |
| 1641 | GCTGCAGCAA CGATTTTCAT ACCTGAATGG TACGAAGCCC |
| 1681 | GCATGTCATT GGCCAAAAAT AGTGTACTTG CTACTGTGGT |
| 1721 | TGATGACTTC TTTGATGTGG GTGGTTCGAT GGAGGAATTA |
| 1761 | AAGAAGCTAA TTGAATTTGT TGAAAAGTGG GATATTGACA |
| 1801 | TCACCAAGGA ATCCTGCTCT GAGCCACTCA AAATCATATT |
| 1841 | TTCAGCACTG CACAGTACAA TCTCTGAGAT TGGAGAGCAA |
| 1881 | GCAGTTAAAT GGCAAGGACG CAATGTAACA AGCCACATAA |
| 1921 | TTGAGATCTG GTTGGATTTG CTCAATTCGA TGTTGAGGGA |
| 1961 | GTCTCAATGG ACTACAGATG TGCACATGCC AACATTGGAT |
| 2001 | GAATATATGG AAGCTGCTTA TGTATCATTC GCCATGGGGC |
| 2041 | CAATTATCAT CCCTGCTCTG TATTTTGTTG GGCCTAAGCT |
| 2081 | ATCTGATGAA ATTGTTCGGG ATCCTGAAAT ACGATCCCTC |
| 2121 | CATAAGCTTG TGAGCATTTG TGGGCGGCTT CTAAATGATA |
| 2161 | TGCAAGGGTT CGAGAGGGAA AAGAAGGCTG GTAAACCAAA |
| 2201 | TGCCGTGTCT ATACGCATTA GTCAAAATGG TGATGGCATT |
| 2241 | ACCGAATCAG CAGCTTTCGA AGAAGTGAAG ATGGAATTAG |
| 2281 | AGGATGCAAG GAGAGAATTG CTAAGATTAG TTGTGCAAAA |
| 2321 | AGATGGTAGT GTAGTTCCAA GAGCTTGCAA GGATGCGTTT |
| 2361 | TGGAGCGTAA GCAGAATGTT GCATCATTTC TACTTCAATA |
| 2401 | ATGATGGATA CACGTCAGAG GTGGAGATGG TTGAGCTCGT |
| 2441 | GAATTCAATT ATTCATGAAC CACTAAAATA A |

A Salvia hispanica (−)-kolavenyl diphosphate synthase (ShTPS1) was identified and isolated. This ShTPS1 enzyme was identified as an (−)-kolavenyl diphosphate synthase, which converts GGPP to (−)-kolavenyl diphosphate [36].

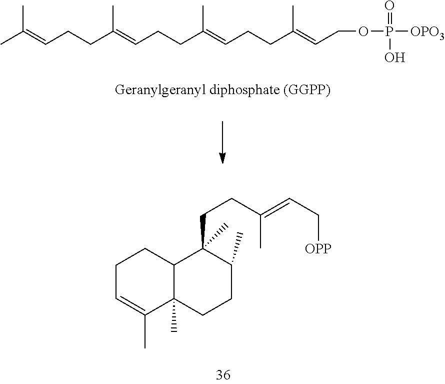

The Salvia hispanica (−)-kolavenyl diphosphate synthase (ShTPS1) has, for example, an amino acid sequence shown below (SEQ ID NO:51).

| | |
|---|---|
| 1 | MSIQANMSFA TSLHRSTTPG VGLPLKPCIS PSPSLSFSPN |
| 41 | FGTFNNTSLR LKPEAGSKSY EGIRRSHQLA ASTILEGQTP |
| 81 | ITPEVESEKT RLIERIRSML QDMDNDGQIS VSPYDTAWVA |
| 121 | LVEDIGGSGG PQFPTSLEWI SNHQYDDGSW GDRKFVLYDR |
| 161 | ILNTLACVVA LTNWKMHPNK CEKGLRFIHE NIKKLADEDE |
| 201 | ELMPVGFEIA LPSVIDLAKR LGIEIPENSA SIKRIYELRD |
| 241 | SKLKKIPMDL VHKRPTSLLF SLEGMEGLNW DKLMNFLAEG |
| 281 | SFLSSPSSTA YALQHTKNEL CLEYLLKAVK RFNGGVPNAY |
| 321 | PVDMFEHLWS VDRLQRLGIS RYFQAEIEEN MAYAYRYWTN |
| 361 | KGITWARNMV VQDSDDSAQG FRLLRLYGYD IPIDVFKHFE |
| 401 | QGGQFCSIPG QMTHAITGMY NLYRASELLF PGEHILSDAR |
| 441 | KYTGNFLHQR RITNTVVDKW IITKDLHGEV AYALDVPFYA |
| 481 | SLPRLEARFF IEQYGGDEDV WIGKTLYRMF KVNSDTYLEM |
| 521 | AKLDYKQCQS VHQLEWNSMQ RLYRDCNLGE FGLSERSLLL |
| 561 | AYYIAASTTF EPEKSSERLA WAITTILVEI IASQKLSDEQ |
| 601 | KREFVDEFVK GSIVNNQNGG RHKPGNRLVE VLINNITLMA |
| 641 | EGRGTYQQLS NAWKKWLKTW EEGGDLGEAE ARLLLHTIHL |
| 681 | SSGLDDSSFS HPKYQQLLEA TSKVCHQLRV FQSVKVYDDQ |
| 721 | ESTSQLVTRT TFQIEAGMQE LVKLVFTKTL EDLPSTTKQS |
| 761 | FFSVARSFYY TACIHADTID SHINKVLFEK IV |

A nucleic acid encoding the Salvia hispanica (−)-kolavenyl diphosphate synthase (ShTPS1) with SEQ ID NO:51 is shown below as SEQ ID NO:52.

| | |
|---|---|
| 1 | ATGAGTATTC AAGCAAACAT GTCATTTGCC ACCTCCCTCC |
| 41 | ACCGATCAAC CACCCCCGGA GTTGGCCTTC CGCTAAAACC |
| 81 | ATGTATCTCT CCCTCTCCCT CTCTTTCCTT TTCCCCAAAC |
| 121 | TTTGGCACTT TTAACAACAC AAGTTTGAGA CTCAAACCAG |
| 161 | AGGCTGGGAG CAAAAGTTAT GAGGGGATTC GAAGAAGTCA |
| 201 | TCAATTAGCA GCATCAACAA TTTTGGAGGG TCAAACTCCG |
| 241 | ATTACTCCGG AGGTTGAATC GGAGAAAACA CGCCTGATTG |
| 281 | AAAGGATTCG TTCGATGTTA CAAGACATGG ACAACGATGG |
| 321 | CCAGATAAGT GTGTCACCAT ACGACACAGC ATGGGTGGCG |
| 361 | CTCGTGGAAG ATATTGGTGG CAGCGGAGGG CCACAGTTTC |
| 401 | CAACGAGCCT AGAGTGGATT TCTAACCACC AGTACGACGA |
| 441 | TGGATCGTGG GGGGATCGCA AATTTGTTCT CTATGACCGG |
| 481 | ATACTCAATA CATTAGCATG TGTTGTCGCA CTCACGAATT |
| 521 | GGAAAATGCA TCCTAACAAA TGCGAAAAAG GGTTGAGGTT |
| 561 | TATTCATGAG AATATTAAGA AACTCGCGGA TGAAGATGAA |
| 601 | GAGCTCATGC CCGTAGGATT CGAAATCGCA CTGCCATCAG |
| 641 | TCATTGATTT AGCTAAAAGA CTGGGTATAG AAATCCCAGA |
| 681 | AAATTCTGCA AGCATAAAAA GAATTTATGA ATTGAGAGAT |
| 721 | TCAAAACTTA AAAAAATACC AATGGATTTA GTGCACAAAA |
| 761 | GGCCCACATC ACTACTCTTC AGCTTGGAAG GCATGGAAGG |
| 301 | CCTTAACTGG GACAAACTAA TGAATTTTCT AGCCGAGGGT |
| 841 | TCGTTTCTTT CATCGCCATC GTCCACTGCC TACGCTCTCC |
| 881 | AACACACCAA GAATGAGTTA TGCCTAGAGT ATTTACTCAA |
| 921 | GGCAGTCAAG AGATTCAATG GTGGAGTTCC AAATGCATAC |
| 961 | CCTGTCGACA TGTTTGAGCA TCTGTGGTCC GTGGATCGCT |
| 1001 | TACAGAGATT AGGAATTTCT CGGTATTTTC AAGCTGAAAT |
| 1041 | TGAAGAAAAC ATGGCCTATG CTTACAGATA CTGGACAAAT |
| 1081 | AAAGGAATCA CCTGGGCAAG AAATATGGTT GTCCAAGACA |
| 1121 | GTGACGACAG CGCACAGGGA TTCAGGCTCT TAAGGTTGTA |
| 1161 | CGGATACGAT ATTCCTATAG ATGTTTTCAA ACATTTCGAG |
| 1201 | CAAGGTGGAC AATTCTGCAG CATACCAGGA CAGATGACAC |
| 1241 | ACGCTATTAC AGGAATGTAC AACTTGTATA GAGCTTCTGA |
| 1281 | ACTTCTGTTC CCTGGAGAAC ACATACTTTC TGATGCTAGA |
| 1321 | AAATACACAG GTAACTTCTT GCATCAAAGA AGAATTACTA |
| 1361 | ACACGGTAGT AGACAAGTGG ATCATTACCA AAGACCTTCA |
| 1401 | CGGCGAGGTG GCTTATGCAT TGGATGTGCC ATTCTACGCC |
| 1441 | AGTCTGCCAC GACTGGAAGC ACGATTCTTC ATAGAACAAT |
| 1481 | ATGGGGGTGA TGAAGATGTT TGGATTGGGA AAACATTGTA |
| 1521 | CAGGATGTTT AAAGTAAACT CCGACACATA CCTTGAGATG |
| 1561 | GCAAAATTAG ATTACAAACA ATGCCAGTCT GTGCATCAGT |
| 1601 | TAGAGTGGAA TAGCATGCAA AGATTGTATA GAGATTGCAA |
| 1641 | TCTAGGAGAG TTTGGGTTGA GCGAAAGAAG CCTTCTCCTA |
| 1681 | GCTTACTACA TAGCAGCCTC AACTACATTT GAGCCGGAAA |
| 1721 | AATCAAGTGA AAGACTGGCT TGGGCTATAA CAACAATTTT |
| 1761 | AGTCGAAATA ATCGCATCCC AAAAACTCTC TGATGAGCAA |
| 1801 | AAGAGAGAGT TTGTTGATGA ATTTGTAAAA GGAAGCATCG |
| 1841 | TCAATAACCA AAATGGAGGA AGACATAAAC CGGGAAACAG |
| 1881 | ATTGGTTGAA GTTTTGATCA ACAATATAAC ACTGATGGCA |
| 1921 | GAAGGCAGAG GCACATATCA GCAGTTGTCT AATGCGTGGA |
| 1961 | AAAAATGGCT AAAGACATGG GAAGAGGGAG GTGACCTGGG |
| 2001 | GGAAGCACAA GCACGGCTTC TCCTGCACAC GATACATTTG |
| 2041 | AGCTCCGGAT TGGATGATTC ATCATTTTCC CATCCAAAAT |
| 2081 | ATCAGCAGCT CTTGGAGGCA ACCAGCAAAG TCTGCCACCA |
| 2121 | ACTTCGCGTA TTCCAGAGTG TAAAGGTGTA TGATGACCAA |
| 2161 | GAGTCTACAA GCCAACTGGT AACTAGGACA ACTTTCCAAA |
| 2201 | TAGAAGCAGG CATGCAAGAA CTAGTGAAAT TAGTTTTCAC |
| 2241 | AAAAACCTTG GAAGATTTGC CTTCTACTAC CAAGCAAAGC |
| 2281 | TTTTTTAGTG TTGCTAGAAG TTTCTATTAC ACTGCCTGTA |
| 2321 | TTCATGCAGA CACTATAGAC TCCCACATAA ACAAAGTATT |
| 2361 | GTTTGAAAAA ATTGTCTAG |

A Teucrium canadense cleroda-4(18),13E-dienyl diphosphate synthase (TcTPS1) was identified and isolated as described herein. This TcTPS1 enzyme was identified as a cleroda-4(18),13E-dienyl diphosphate synthase, which converts GGPP to cleroda-4(18),13E-dienyl diphosphate [38]. In addition, the combination of TcTPS1 and SsSS enzymes generated neo-cleroda-4(18),14-dien-13-ol [37]. These compounds are shown below.

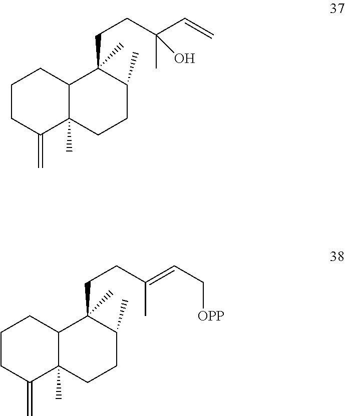

The Teucrium canadense cleroda-4(18),13E-dienyl diphosphate synthase (TcTPS1) amino acid sequence is shown below as SEQ ID NO:53.

| | |
|---|---|
| 1 | MSFASQATSL LLSSHNATAL PPLSAARLPP LTAGAAPFGR |
| 41 | ISFTTTSLRQ YKLVSRAQSQ EVDEIEKVTQ VVLEAEKDID |
| 81 | QEAKVRELVE NVRVKLQNIG EGGISISPYD TAWVALVEDV |
| 121 | GGSGRPQFPE SLDWISNHQF PDGSWGSHKF LYYDRVLCTL |
| 161 | ACIVALKTWN LHPHKFDKGL KFVRENIGKL ADEEDVHMPI |
| 201 | GFEVAFPSLI ETAKRKGIDI PEDFPGKKEI YAKRDLKLKK |
| 241 | IPMDILHKIP TPLLFSIEGI EGLDWQKLFK FRDHGSFLTS |
| 281 | PSSTAHALQQ TKDELCLKYL TNLVKKNNGG VPNAFPVDLF |
| 321 | DRNYTVDRLR RLGILRYFQP EIEECMKYVY REWDKRCISW |
| 361 | ARNTHVQDLD DTVQGFRNLR MHGYDVTLDV FKQFERCGEF |
| 401 | FSFHGQSSDA VLCMFNLYRA SQVLFPGEDM LADARKYAAN |
| 441 | YLHKRRVSNR VVDKWIINKD LPGEVAYGLD VPFYASLPRL |
| 481 | EARFYVEQYG GNDDVWIGKA LYRMLNVSCD TYLELAKLDY |
| 521 | NICQAVHQKE WKSFQKWHRD GEFGLDEKSL LLAYYIAAST |
| 561 | VFEPEKSLER LAWAKTAVLM EAILSQQLPS TKKHELVDEF |
| 601 | KHASILNNQN GGSYKTRTPL VETLVNAISE LSTTILLEQD |
| 641 | RDIHLQLSNA WLKWLSRWEA RGNLVEAEAE LLLQTLHLSN |
| 681 | GLEESSFSHP KYQQLLQVIS KVCHLLRLFQ KRKVHDPEGC |
| 721 | TTDIATGTTF QIEACMQQVV KLVFTKSSHD LDSVVKQRFL |
| 761 | DVARSFYYTA HCDPQVIQSH INKVLFEKVV |

A nucleic acid encoding the Teucrium canadense Cleroda-4(18),13E-dienyl diphosphate synthase (TcTPS1) has with SEQ ID NO:53 is shown below as SEQ ID NO:54.

| | |
|---|---|
| 1 | ATGTCATTTG CTTCCCAAGC CACCTCCCTC CTCCTTTCTT |
| 41 | CCCACAACGC CACCGCTCTT CCGCCTCTCT CTGCCGCCCG |
| 81 | CCTTCCGCCT CTCACTGCCG GTGCTGCTCC ATTCGGAAGA |
| 121 | ATATCATTTA CTACTACCTC TCTTCGGCAG TATAAACTGG |
| 161 | TGTCAAGAGC TCAAAGCCAA GAGGTGGATG AGATTGAAAA |
| 201 | AGTGACACAA GTGGTATTGG AGGCAGAAAA AGACATCGAT |
| 241 | CAAGAGGCGA AGGTAAGGGA GCTGGTGGAA AATGTCCGAG |
| 281 | TGAAGCTGCA AAATATCGGG GAAGGAGGGA TAAGCATATC |
| 321 | GCCGTACGAC ACCGCATGGG TGGCGCTGGT GGAGGATGTC |
| 361 | GGCGGCAGCG GCAGACCGCA GTTCCCGGAG AGCCTGGATT |
| 401 | GGATATCAAA CCACCAGTTC CCGGACGGGT CGTGGGGCAG |
| 441 | CCACAAATTC TTGTACTATG ACCGGGTTTT GTGCACGTTA |
| 481 | GCATGTATAG TTGCATTGAA AACTTGGAAT CTGCATCCTC |
| 521 | ACAAATTCGA CAAAGGGTTG AAATTCGTCA GAGAGAACAT |
| 561 | TGGAAAGCTC GCGGATGAAG AAGACGTGCA CATGCCGATT |
| 601 | GGGTTCGAAG TGGCATTCCC ATCACTTATA GAGACTGCAA |
| 641 | AGAGAAAAGG AATTGACATC CCGGAAGATT TCCCTGGCAA |
| 681 | GAAAGAAATC TATGCAAAAA GAGACCTAAA GCTGAAAAAG |
| 721 | ATACCTATGG ATATACTGCA CAAAATCCCC ACACCATTAC |
| 761 | TGTTCAGCAT AGAAGGGATA GAAGGCCTTG ATTGGCAGAA |
| 801 | GCTATTCAAA TTCCGCGATC ACGGCTCCTT CCTCACGTCC |
| 841 | CCGTCCTCAA CGGCCCACGC TCTCCAGCAA ACAAAGGACG |
| 881 | AGTTATGCCT CAAATATCTG ACCAATCTTG TCAAAAAGAA |
| 921 | CAATGGGGGA GTTCCAAATG CATTTCCGGT GGACCTATTT |
| 961 | GATCGTAACT ATACAGTAGA TCGCCTGAGG AGGCTGGGAA |
| 1001 | TTTTGCGCTA TTTTCAACCT GAAATCGAGG AATGCATGAA |
| 1041 | ATATGTATAC AGATICTGGG ATAAAAGAGG AATCAGCTGG |
| 1081 | GCAAGAAATA CCCATGTTCA GGACCTTGAT GATACCGTAC |
| 1121 | AGGGATTCAG GAACTTAAGG ATGCATGGTT ATGATGTCAC |
| 1161 | CTTAGATGTT TTCAAACAGT TCGAGAGATG TGGAGAATTC |
| 1201 | TTTAGCTTCC ACGGGCAATC AAGTGATGCT GTCTTAGGAA |
| 1241 | TGTTCAACTT GTACCGAGCT TCTCAGGTTC TGTTTCCAGG |
| 1281 | AGAAGACATG CTTGCAGATG CAAGGAAGTA CGCGGCCAAC |
| 1321 | TATTTGCATA AAAGAAGAGT TAGTAATAGG GTCGTGGACA |
| 1401 | AATGGATTAT TAACAAAGAT CTTCCAGGCG AGGTGGCGTA |
| 1441 | TGGGCTAGAT GTTCCGTTCT ACGCCAGTCT ACCTCGACTG |
| 1481 | GAAGCAAGAT TCTACGTCGA ACAATATGGG GGTAACGATG |
| 1521 | ATGTCTGGAT TGGAAAAGCT TTATATAGAA TGTTGAATGT |
| 1601 | GAGCTGTGAT ACTTACCTTG AGCTAGCAAA ATTAGACTAC |
| 1641 | AATATTTGCC AGGCTGTGCA TCAGAAAGAG TGGAAAAGCT |
| 1681 | TTCAAAAATG GCACAGGGAT GGGGAGTTTG GATTGGATGA |
| 1721 | AAAAAGCTTA CTTTTAGCTT ACTACATAGC AGCCTCGACT |
| 1761 | GTTTTCGAGC CTGAAAAATC TCTAGAGCGA CTGGCTTGGG |
| 1801 | CTAAAACCGC AGTTCTAATG GAGGCAATTT TGTCCCAACA |
| 1841 | ACTTCCTAGC ACAAAAAAAC ATGAGCTTGT TGACGAATTT |
| 1881 | AAACATGCAA GCATCCTCAA CAACCAAAAT GGAGGAAGCT |
| 1921 | ATAAAACAAG AACTCCTTTG GTAGAGACTC TAGTAAACGC |
| 1961 | CATAAGTGAG CTCTCAACTA CCATACTATT GGAGCAAGAC |
| 2001 | AGAGACATTC ATCTGCAATT ATCTAATGCG TGGCTGAAGT |
| 2041 | GGCTAAGTAG ATGGGAGGCA AGAGGCAACC TAGTGGAAGC |
| 2081 | AGAAGCAGAG CTTCTTCTGC AAACCTTACA TCTGAGCAAT |
| 2121 | GGATTAGAAG AATCATCATT TTCTCATCCA AAATATCAAC |
| 2161 | AACTCTTACA GGTTACCAGC AAAGTCTGTC ACCTACTTCG |
| 2201 | GCTATTCCAG AAACGAAAGG TGCATGATCC GGAAGGGTGT |
| 2241 | ACAACAGACA TTGCAACAGG GACAACTTTC CAAATAGAAG |
| 2281 | CATGCATGCA ACAAGTAGTG AAATTAGTGT TCACCAAATC |
| 2321 | CTCACATGAT TTAGATTCTG TTGTTAAGCA GAGATTTTTG |
| 2361 | GATGTTGCCA GAAGTTTCTA TTACACAGCC CACTGTGATC |
| 2401 | CACAAGTGAT CCAGTCCCAC ATTAATAAAG TGTTGTTTGA |
| 2441 | AAAAGTAGTC TAG |

Salvia officinalis (SoTPS2), Scutellaria baicalensis SbTPS1, and SbTPS2 enzymes were identified and isolated. These SoTPS2, SbTPS1, SbTPS2, CfTPS18a and CfTPS18b enzymes were all identified as ent-CPP synthases, which convert GGPP to ent-CPP.

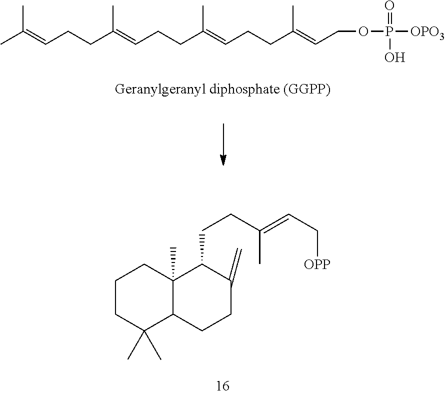

The Salvia officinalis (SoTPS2) enzyme can have the amino acid sequence shown below (SEQ ID NO:55).

| | |
|---|---|
| 1 | MSFASTTSLL RPSVTGFGVS PRVTSTSILS RSYGQILKGK |
| 41 | TKYITDNRRN RQLAVKFEGQ IALDLEDGVA KQTNQEAESE |
| 81 | KIRQLKGKIR WILQNMEDGE MSVSPYDTAW VALVEDISGG |
| 121 | GGPQFPTSLE WISKNQLADG SWGDPNYFLL YDRILNTLAC |
| 161 | VVALTTWNMH PHKCDQGLRF IRDNIEKLED EDEELILVGF |
| 201 | EIALPSLIDY AQNLGIQIQY DSPFIKKICA KRDLKLRKIP |
| 241 | MDLMHRKPTS LLYSLEGMEG LEWEKLMNLR SEGSFLSSPS |
| 281 | STAYALQHTK DELCLDYLVK AVNKFNGGVP NVYPVDMYEH |
| 321 | LWCVDRLQRL GISRYFQLEI QQCLDYVYRY WTNEGISWAR |
| 361 | YTNIRDSDDT AMGFRLLRLY GYDVSIDAFK PFEESGEFYS |
| 401 | MAGQMNHAVT GMYNLYRASQ LMFPQEHILS DARNFSAKFL |
| 441 | HQKRRTNALV DKWIITKDLP GEVGYALDVP FYASLPRLEA |
| 481 | RFFLEQYGGD DDVWIGYTLY RMPYVNSNTY LELAKVDYKN |
| 521 | CQSVHQLEWK SMQKWYRECN IGEFGLSERS LLLAYYIAAS |
| 561 | TTFEPEKSGE RLAWATTAIL IETIASQQLS DEQKREFVDE |
| 601 | FENSIIIKNQ NGGRYKARNR LVKVLINTVT LVAEGRGINQ |
| 641 | QLFNAWQKWL KTWEEGGDMG EAEAQLLLRT LHLSSGFDQS |
| 681 | SFSHPKYEQL LEATSKVCHQ LRLFQNRKVD DGQGCISRLV |
| 721 | IGTTSQIEAG MQEVVKLVFT KTSQDLTSAT KQSFFNIARS |
| 761 | FYYTAYFHAD TIDSHIYKVL FQTIV |

A nucleic acid encoding the Salvia officinalis (SoTPS2) has with SEQ ID NO:55 is shown below as SEQ ID NO:56.

| | |
|---|---|
| 1 | ATGTCATTTG CTTCCACCAC CTCCCTCCTC CGACCAAGCG |
| 41 | TCACTGGGTT CGGTGTTTCT CCAAGGGTTA CTTCCACCTC |
| 81 | CATTCTTAGC CGAAGTTATG GTCAAATATT AAAAGGAAAA |
| 121 | ACAAAATACA TAACTGATAA CCGTAGAAAT CGACAATTGG |
| 161 | CGGTAAAATT TGAGGGCCAA ATTGCTTTGG ATTTGGAGGA |
| 201 | TGGCGTAGCA AAGCAGACGA ATCAAGAGGC GGAATCTGAG |
| 241 | AAGATAAGGC AACTGAAGGG AAAGATCCGA TGGATTCTGC |
| 281 | AAAACATGGA GGACGGCGAG ATGAGCGTGT CGCCGTACGA |
| 321 | CACCGCATGG GTGGCGCTGG TGGAAGATAT CAGCGGCGGC |
| 361 | GGCGGGCCGC AGTTCCCGAC GAGCCTCGAG TGGATTTCCA |
| 401 | AGAATCAGTT GGCGGATGGG TCATGGGGGG ATCCTAATTA |
| 441 | TTTCCTTCTC TACGACAGAA TACTCAATAC TTTAGCATGT |
| 481 | GTAGTCGCAC TCACGACTTG GAATATGCAT CCTCACAAAT |
| 521 | GCGATCAAGG GTTGAGGTTT ATAAGAGACA ACATTGAGAA |
| 561 | ACTTGAGGAT GAAGATGAGG AGCTAATTCT CGTAGGATTC |
| 601 | GAGATCGCAC TGCCTTCACT CATTGATTAT GCTCAAAACC |
| 641 | TTGGGATACA AATCCAATAT GATTCTCCAT TCATTAAAAA |
| 681 | AATTTGTGCA AAGAGAGATC TAAAACTCAG AAAAATACCA |
| 721 | ATGGATTTAA TGCACAGAAA GCCAACATCA TTGCTCTACA |
| 761 | GCTTGGAAGG CATGGAAGGC CTTGAGTGGG AAAAGCTAAT |
| 801 | GAATTTGCGA TCGGAGGGTT CGTTTCTGTC ATGGCCGTCG |
| 841 | TCCACGGCCT ACGCTCTCCA ACACACCAAG GATGAGTTAT |
| 881 | GCCTTGACTA TCTGGTCAAG GCGGTCAACA AATTCAATGG |
| 921 | TGGAGTTCCC AACGTGTACC CTGTCGACAT GTATGAGCAT |
| 961 | CTATGGTGCG TAGACCGCTT GCAGAGGTTG GGAATTTCTC |
| 1001 | GCTATTTTCA ACTTGAAATT CAACAATGCC TCGACTATGT |
| 1041 | TTACAGATAC TGGACAAATG AAGGAATTTC GTGGGCAAGA |
| 1081 | TATACTAATA TCCGGGATAG TGACGACACC GCAATGGGAT |
| 1121 | TCAGGCTTCT AAGGTTGTAC GGCTATGATG TCTCTATAGA |
| 1161 | TGCTITTAAA CCATTCGAGG AAAGCGGAGA ATTCTATAGC |
| 1201 | ATGGCAGGGC AGATGAACCA CGCTGTTACA GGAATGTACA |
| 1241 | ACTTGTACAG AGCTTCTCAA CTTATGTTCC CTCAAGAACA |
| 1281 | CATACTTTCC GATGCCAGAA ACTTCTCTGC CAAATTCTTG |
| 1321 | CATCAAAAGA GGCGTACTAA TGCACTAGTA GACAAGTGGA |
| 1361 | TCATTACCAA AGACCTTCCC GGCGAGGTTG GATATGCATT |
| 1401 | GGATGTGCCG TTCTACGCCA GTCTGCCTCG ACTGGAAGCA |
| 1441 | CGATTCTTCT TAGAACAATA TGGGGGTGAT GATGATGTTT |
| 1481 | GGATTGGAAA AACTTTGTAC AGGATGCCAT ATGTGAACTC |
| 1521 | CAACACATAC CTTGAGCTTG CAAAAGTAGA CTACAAAAAC |
| 1561 | TGCCAGTCCG TGCATCAGTT GGAGTGGAAG AGCATGCAAA |
| 1601 | AATGGTACAG AGAATGCAAT ATAGGTGAGT TTGGGTTGAG |
| 1641 | CGAAAGAAGC CTTCTCCTAG CTTACTACAT AGCAGCCTCA |
| 1681 | ACTACATTCG AGCCAGAAAA ATCAGGTGAG CGGCTCGCTT |
| 1721 | GGGCTACAAC AGCAATTTTA ATCGAGACAA TCGCGTCCCA |
| 1761 | ACAACTCTCC GATGAACAAA AGAGAGAGTT CGTTGATGAA |
| 1801 | TTTGAAAACA GCATCATTAT CAAGAATCAA AATGGAGGGA |
| 1841 | GATATAAAGC AAGAAACAGA TTGGTCAAGG TTTTGATCAA |
| 1381 | CACTGTAACA CTGGTAGCAG AAGGCAGAGG CATAAATCAG |
| 1921 | CAGTTGTTTA ATGCGTGGCA AAAATGGCTA AAGACATGGG |
| 1961 | AAGAAGGAGG TGACATGGGG GAAGCAGAAG CCCAGCTTCT |
| 2001 | TCTGCGCACG CTACATTTGA GCTCCGGATT CGATCAATCA |
| 2041 | TCATTTTCCC ATCCAAAATA TGAGCAGCTC TTGGAGGCGA |
| 2081 | CCAGCAAAGT TTGCCACCAA CTTCGCCTAT TCCAGAATCG |
| 2121 | AAAGGTGGAT GATGGCCAAG GGTGTATAAG TCGATTGGTA |
| 2161 | ATTGGGACAA CTTCCCAAAT AGAAGCAGGC ATGCAAGAAG |
| 2201 | TAGTGAAATT AGTTTTCACC AAAACCTCAC AAGACTTGAC |
| 2241 | TTCTGCTACC AAGCAAAGCT TTTTCAATAT TGCTAGAAGT |
| 2281 | TTCTATTATA CTGCCTACTT TCATGCAGAC ACTATAGACT |
| 2321 | CCCACATATA CAAAGTATTG TTTCAAACAA TAGTATAG |

A Scutellaria baicalensis SbTPS1 amino acid sequence shown below (SEQ ID NO:57).

| | |
|---|---|
| 1 | MPFLLPSSAT SSPAFYTPAA PLAGHHVFPS FKPLIISRSS |
| 41 | LQCNAISRPR TQEYIDVIQN GLPVIKWHEA VEEDETDKDS |
| 81 | LNKEATSDKI RELVNLIRSM LQSMGDGEIS SSPYDAAWVA |
| 121 | LVPDVGGSGG PQFPSSLEWI SKNQLPDGSW GDTCTFSIYD |
| 161 | RIINTLACVV ALKSWNIHPH KTYQGISFIK ANMDKLEDEN |
| 201 | EEHMPIGFEV ALPSLIEIAK RLDIDISSDS RGLQEIYTRR |
| 241 | EVKLKRIPKE IMHQVPTTLL HSLEGMAELT WHKLLKLQCQ |
| 281 | DGSFLFSPSS TAFALHQTKD HNCLHYLTKY VHKFHGGVPN |
| 321 | VYPVDLFEHL WAVDRIQRLG ISRHFKPQVD ECIAYVYRYW |
| 361 | TDKGICWARN SVVQDLDDTA MGFRLLRLHG YDVSADVFKH |
| 401 | FENGGEFFCF KGQSTQAVTG MYNLYRASQL MFPGESILED |
| 441 | AKTESSKFLQ RKRANNELLD KWIITKDLPG EVGYALDVPW |
| 481 | YASLPRVETR FYLEQYGGED DVWIGKTLYR MPYVNNNKYL |
| 521 | ELAKLDYSNC QSLHQQEWKN IQKWYESCNL GEFGLSERRV |
| 561 | LLAYYVAAAC IYEPEKSNQR LAWAKTVILM ETITSYFEHQ |
| 601 | QLSAEQRRAF VNEFEHGSIL KYANGGRYKR RSVLGTLLKT |
| 641 | LNQLSLDILL THGRNVHQPF KNAWHKWLKT WEEGGDIEEG |
| 681 | EAEVLVRTLN LSGEGRHDSY VLEQSLLSQP IYEQLLKATM |
| 721 | SVCKKLRLFQ HRKDENGCMT KMRGITTLEI ESEMQELVKL |
| 761 | VFTKSSDDLD CEIKQNFFTI ARSFYYVAYC NQGTINYHIA |
| 801 | KVLFERVL |

A nucleic acid encoding the Scutellaria baicalensis SbTPS1 with SEQ ID NO:57 is shown below as SEQ ID NO:58.

| | |
|---|---|
| 1 | ATGCCTTTCC TCCTCCCTTC CTCCGCCACC AGCTCCCCCG |
| 41 | CGTTCTATAC TCCGGCCGCG CCTCTCGCCG GTCATCATGT |
| 31 | TTTTCCATCT TTCAAGCCAC TCATTATTTC CCGTTCTTCA |
| 121 | CTCCAATGCA ATGCAATCTC TCGACCTCGT ACCCAAGAAT |
| 161 | ACATAGATGT GATTCAGAAT GGATTGCCAG TAATAAAGTG |
| 201 | GCACGAAGCT GTGGAAGAAG ATGAGACAGA TAAAGATTCT |
| 241 | CTTAATAAGG AGGCCACGTC AGACAAGATA AGAGAGTTGG |
| 281 | TAAATCTGAT CCGTTCGATG CTCCAATCAA TGGGCGACGC |
| 521 | AGAGATAAGC TCGTCGCCGT ACGACGCCGC ATGGGTGGCG |
| 561 | CTGGTGCCGG ACGTCGGCGG CTCCGGCGGG CCCCAGTTCC |
| 601 | CCTCCAGCCT CGAATGGATA TCCAAAAACC AACTCCCCGA |
| 641 | CGGCTCCTGG GGCGACACGT GTACCTTTTC CATTTATGAT |
| 681 | CGAATCATCA ACACACTGGC TTGCGTTGTT GCTTTGAAAT |
| 721 | CTTGGAACAT ACATCCCCAC AAAACTTATC AAGGGATTTC |
| 761 | ATTCATAAAG GCAAATATGG ACAAACTTGA AGACGAGAAC |
| 801 | GAGGAGCACA TGCCGATCGG ATTTGAAGTG GCACTCCCGT |
| 841 | CGCTAATCGA GATAGCGAAA AGGCTCGATA TCGATATTTC |
| 881 | CAGCGATTCG AGAGGGCTGC AAGAGATATA CACGAGGAGG |
| 921 | GAGGTAAAGC TGAAAAGGAT ACCGAAAGAG ATAATGCACC |
| 961 | AAGTGCCCAC AACACTGCTT CATAGCTTGG AGGGTATGGC |
| 1041 | CGAGCTGACG TGGCACAAGC TTTTGAAATT ACAGTGCCAA |
| 1081 | GATGGCTCCT TTCTTTTCTC TCCATCTTCA ACTGCCTTTG |
| 1121 | CTCTTCACCA AACTAAGGAC CATAATTGTC TCCATTATTT |
| 1161 | GACCAAATAT GTTCACAAAT TTCATGGTGG AGTGCCAAAT |
| 1201 | GTGTATCCGG TGGACTTGTT CGAGCATCTA TGGGCAGTTG |
| 1241 | ATCGGATCCA ACGGCTGGGG ATTTCCCGGC ATTTCAAGCC |
| 1281 | CCAAGTTGAT GAATGTATTG CCTATGTTTA TAGATATTGG |
| 1321 | ACAGATAAAG GAATATGCTG GGCAAGAAAT TCAGTAGTTC |
| 1361 | AAGATCTTGA TGACACAGCC ATGGGATTCA GGCTTCTTAG |
| 1401 | GTTGCATGGC TACGATGTTT CAGCAGATGT TTTCAAACAT |
| 1441 | TTTGAAAATG GTGGAGAGTT CTTCTGCTTC AAAGGGCAAA |
| 1481 | GCACGCAGGC AGTGACTGGA ATGTACAATC TGTACAGAGC |
| 1521 | TTCTCAGTTG ATGTTTCCTG GAGAAAGCAT ACTGGAAGAT |
| 1601 | GCTAAGACCT TCTCATCTAA GTTTTTGCAA CGAAAACGAG |
| 1641 | CCAATAACGA GTTGTTAGAT AAGTGGATTA TTACCAAGGA |
| 1681 | TCTTCCTGGA GAGGTGGGAT ATGCTCTAGA TGTACCATGG |
| 1721 | TATGCTAGCT TACCTAGAGT TGAAACTAGA TTCTACTTGG |
| 1801 | AACAATATGG TGGTGAAGAT GATGTTTGGA TTGGCAAAAC |
| 1841 | TTTATACAGG ATGCCATATG TTAACAATAA TAAATATCTA |
| 1881 | GAACTGGCAA AATTAGACTA TAGTAACTGC CAGTCATTAC |
| 1921 | ATCAACAAGA GTGGAAAAAC ATTCAAAAAT GGTATGAGAG |
| 1961 | TTGCAATCTG GGAGAATTTG GTITGAGTGA AAGAAGGGTT |
| 2001 | CTACTAGCCT ACTACGTAGC TGCTGCCTGT ATATATGAGC |
| 2041 | CCGAAAAGTC AAACCAGCGC TTGGCTTGGG CCAAAACCGT |
| 2081 | AATTTTAATG GAGACTATTA CTTCCTATTT TGAGCACCAA |
| 2121 | CAACTCTCCG CAGAACAGAG ACGCGCCTTT GTTAATGAAT |
| 2161 | TTGAACATGG GAGTATCCTC AAATATGCAA ATGGAGGAAG |
| 2201 | ATACAAAAGG AGGAGTGTTT TGGGGACTTT GCTCAAAACA |
| 2241 | CTAAATCAGC TTTCATTGGA TATATTATTG ACACACGGTC |
| 2281 | GAAACGTCCA TCAGCCTTTC AAAAATGCGT GGCACAAGTG |
| 2321 | GCTAAAAACG TGGGAAGAAG GAGGTGACAT TGAAGAAGGC |
| 2361 | GAAGCAGAGG TATTGGTCCG AACCCTAAAC CTAAGCGGCG |
| 2401 | AAGGGAGGCA CGACTCCTAT GTATTGGAGC AATCATTATT |
| 2441 | GTCAGAACCT ATATATGAAC AACTTTTGAA AGCCACCATG |
| 2481 | AGTGTTTGCA AGAAGCTTCG ATTGTTCCAA CATCGAAAGG |
| 2521 | ATGAGAATGG ATGTATGACG AAGATGAGAG GCATTACAAC |
| 2561 | GTTAGAGATA GAATCGGAGA TGCAAGAATT AGTGAAATTA |
| 2601 | GTATTTACTA AATCCTCAGA TGATTTAGAT TGTGAAATTA |
| 2641 | AACAAAACTT TTTTACAATT CGTAGGAGTT TCTATTATGT |
| 2681 | GGCTTATTGT AACCAAGGAA CTATCAACTT TCACATTGCT |
| 2721 | AAGGTGCTCT TTGAAAGAGT TCTTTAG |

A Scutellaria baicalensis SbTPS2 amino acid sequence is shown below (SEQ ID NO:59).

| | |
|---|---|
| 1 | MASLSTLSLN FSPAIHRKIQ QSSAKLQFQG HCFTISSCMN |
| 41 | NSKRLSLNHQ SNHKRTSNVS ELQVATLDAP QIREKEDYST |
| 81 | AQGYEKVDEV EDPIEYIRML LNTTGDGRIS VSPYDTAWIA |
| 121 | LIKDVEGRDA PQFPSSLEWI ANNQLSDGSW GDEKFFCVYD |
| 161 | RLVNTLACVV ALRSWNIDAE KSEKGIRYIK ENVDKLKDGN |
| 201 | PEHMTCGFEV VEPSLLQRAQ SMGIHDLPYD APVIQDIYNT |
| 241 | RESKLKRIPM EVMHKVPTSL LFSLEGLENL EWDKLLKLQS |
| 281 | SDGSFLTSPS STAYAFMHTK DPKCFEFIKN TVETFNGGAP |
| 321 | HTYPVDVFGR LWAIDRLQRL GISRFFESEI ADCLDHIYKY |
| 361 | WTDKGVFSGR ESDFVDVDDT SMGVRLLRMH GYQVDPNVLR |
| 401 | NFKQGDKFSC YGGQMIESSS PIYNLYRASQ LRFPGEDILE |
| 441 | DANKFAYEFL QEQLSNNQLL DKWVISKHLP DEIKLGLQMP |
| 481 | WYATLPRVEA KYYLQYYAGA DDVWIGKTLY RMPEISNDTY |
| 521 | LELARMDFKR CQAQHQFEWI SMQEWYESCN IEEFGISRKE |
| 561 | LLQAYFLACS SVFELERTTE RIGWAKSQII SRMIASFFNN |
| 601 | ETTTADEKDA LLTRFRNING PNRTKSGQRE SEAVNMLVAT |
| 641 | LQQYLAGFDR YTRHQLKDAW SVWFRKVQEE EAIYGAEAEL |
| 681 | LTTTLNICAG HIAFDENIMA NYDYTTLSSL TSKICQKLSE |
| 721 | IRNEKVEEME SGIKAKSSIK DKEVEHDMQS LVKLVLERCE |
| 761 | GINNRKLKQT FLSVAKTYYY RAYNADETMD IHMFKVLFEP |
| 801 | VM |

A nucleic acid encoding the Scutellaria baicalensis SbTPS2 with SEQ ID NO:59 is shown below as SEQ ID NO:60.

| | |
|---|---|
| 1 | ATGGCCTCTC TATCAACTCT GAGCCTCAAC TTTTCCCCAG |
| 41 | CAATTCACCG CAAAATACAG CAATCATCTG CAAAACTTCA |
| 81 | GTTCCAGGGA CATTGTTTCA CCATAAGTTC ATGCATGAAC |
| 121 | AACAGTAAAA GACTGTCTTT GAACCACCAA TCTAATCACA |
| 161 | AAAGAACGTC AAACGTATCT GAGCTGCAAG TTGCCACTTT |
| 201 | GGATGCGCCC CAAATACGTG AAAAAGAAGA CTACTCCACT |
| 241 | GCTCAAGGCT ATGAGAAGGT GGATGAAGTA GAGGATCCTA |
| 281 | TCGAATATAT TAGAATGCTG TTGAACACAA CAGGTGATGG |
| 321 | GCGAATAAGT GTGTCGCCAT ACGACACAGC CTGGATCGCT |
| 361 | CTTATTAAAG ACGTGGAAGG ACGTGATGCT CCCCAGTTCC |
| 401 | CATCTAGTCT CGAATGGATT GCCAATAATC AACTGAGTGA |
| 441 | TGGGTCGTGG GGCGATGAGA AGTTTTTCTG TGTGTATGAT |
| 481 | CGCCTTGTTA ATACACTTGC ATGTGTCGTG GCATTGAGAT |
| 521 | CATGGAATAT TGATGCTGAA AAGAGCGAGA AAGGAATAAG |
| 561 | ATACATAAAA GAAAACGTGG ATAAACTGAA AGATGGGAAT |
| 601 | CCAGAGCACA TGACCTGTGG TTTTGAGGTG GTGTTTCCTT |
| 641 | CCCTTCTTCA GAGAGCCCAA AGTATGGGAA TTCATGATCT |
| 681 | TCCCTATGAT GCTCCTGTCA TCCAAGACAT TTACAATACC |
| 721 | AGGGAGAGTA AATTGAAAAG CATTCCAATG GAGGTTATCC |
| 761 | ACAAGGTGCC AACATCTCTA TTGTTCAGCT TGGAAGGATT |
| 801 | GGAGAATTTG GAGTGGGATA AGCTCCTCAA ACTTCAGTCT |
| 841 | TCTGATGGTT CATTCCTCAC TTCTCCATCC TCAACTGCCT |
| 881 | ATGCTTTCAT GCACACTAAG GACCCTAAAT GCTTCGAATT |
| 921 | CATCAAAAAC ACCGTCGAAA CATTTAATGG AGGAGCACCT |
| 961 | CATACTTATC CGGTGGATGT TTTTGGAAGA CTGTGGGCCA |
| 1001 | TTGACAGGCT GCAGCGCCTC GGAATCTCTC GCTTCTTTGA |
| 1041 | GTCCGAGATT GCTGATTGCT TAGATCACAT CTATAAATAT |
| 1081 | TGGACAGACA AAGGAGTGTT CAGTGGAAGA GAATCAGATT |
| 1121 | TTGTGGATGT GGATGACACA TCCATGGGTG TTAGGCTTCT |
| 1161 | AAGGATGCAC GGATATCAAG TTGATCCAAA TGTATTGAGG |
| 1201 | AACTTCAAGC AGGGTGACAA ATTTTCATGC TATGGTGGTC |
| 1241 | AAATGATAGA GTCATCATCT CCGATATACA ATCTCTATAG |
| 1281 | GGCTTCTCAA CTCCGATTTC CAGGAGAAGA CATTCTTCAA |
| 1321 | GATGCCAACA AATTCGCATA CGAGTTCTTG CAAGAACAGC |
| 1361 | TATCCAACAA TCAACTTTTG GACAAATGGG TTATATCCAA |
| 1401 | GCACTTGCCT GATGAGATAA AGCTTGGATT GCAGATGCCA |
| 1441 | TGGTATGCCA CCCTACCCCG AGTGGAGGCT AAATACTACC |
| 1481 | TACAGTATTA TGCTGGTGCT GATGATGTCT GGATCGGCAA |
| 1521 | GACTCTCTAC AGAATGCCAG AAATCAGTAA TGATACATAT |
| 1561 | CTGGAGTTAG CAAGAATGGA TTTCAAGAGA TGCCAAGCAC |
| 1601 | AGCATCAATT TGAGTGGATT TCCATGCAAG AATGGTATGA |
| 1641 | AAGTTGCAAC ATTGAAGAAT TTGGGATAAG CAGLAAAGAG |
| 1681 | CTTCTTCAGG CTTACTTTTT GGCCTGCTCA AGTGTATTTG |
| 1721 | AACTCGAGAG GACAACAGAG AGAATAGGAT GGGCCAAATC |
| 1761 | CCAAATTATT TCAAGGATGA TAGCTTCTTT CTTCAACAAT |
| 1801 | GAAACTACAA CAGCCGATGA AAAAGATGCA CTTTTAACCA |
| 1841 | GATTCAGAAA CATCAATGGC CCAAACAAAA CAAAAAGTGG |
| 1881 | TCAGAGAGAG AGTGAAGCTG TGAACATGTT GGTAGCAACG |
| 1921 | CTCCAACAAT ACCTGGCAGG ATTTGATAGA TATACCAGAC |
| 1961 | ATCAATTGAA AGATGCTTGG AGTGTGTGGT TCAGAAAAGT |
| 2001 | GCAAGAAGAA GAGGCCATCT ACGGGGCAGA AGCGGAGCTT |
| 2041 | CTAACAACCA CCTTAAACAT CTGTGCTGGT CATATTGCTT |
| 2081 | TCGACGAAAA CATAATGGCC AACAAAGATT ACACCACTCT |
| 2121 | TTCCAGCCTT ACAAGCAAAA TTTGCCAGAA GCTTTCTGAA |
| 2161 | ATTCGAAATG AAAAGGTTGA GGAAATGGAG AGTGGAATTA |
| 2201 | AAGCAAAATC AAGCATCAAA GACAAGGAAG TGGAACATGA |
| 2241 | TATGCAGTCA CTGGTGAAAT TAGTCCTGGA GAGATGTGAA |
| 2281 | GGCATAAACA ACAGAAAACT GAAGCAAACA TTTCTATCGG |
| 2321 | TTGCAAAAAC ATATTACTAC AGAGCCTATA ATGCTGATGA |
| 2361 | AACCATGGAC ATCCATATGT TCAAAGTACT TTTCGAACCA |
| 2401 | GTCATGTGA |

An example of a Salvia sclarea sclareol synthase amino acid sequence is shown below (SEQ ID NO:176, NCBI accession no. AET21246.1).

| | |
|---|---|
| 1 | MSLAFNVGVT PFSGQRVGSR KEKFPVQGFP VTTPNRSRLI |
| 41 | VNCSLTTIDF MAKMKENFKR EDDKFPTTTT LRSEDIPSNL |
| 81 | CIIDTLQRLG VDQFFQYEIN TILDNTFRLW QEKHKVIYGN |
| 121 | VTTHAMAFRL LRVKGYEVSS EELAPYGNQE AVSQQTNDLP |
| 161 | MIIELYRAAN ERIYEEERSL EKILAWTTIF LNKQVQDNSI |
| 201 | PDKKLHKLVE FYLRNYKGIT IRLGARRNLE LYDMTYYQAL |
| 241 | KSTNRESNLC NEDFLVFARQ DFDIHEAQNQ KGLQQLQRWY |
| 281 | ADCRLDTLNF GRDVVIIANY LASLIIGDHA FDYVRLAFAK |
| 321 | TSVLVTIMDD FFDCHGSSQE CDKIIELVKE WKENPDAEYG |
| 361 | SEELEILFMA LYNTVNELAE RARVEQGRSV KEFLVKLWVE |
| 401 | ILSAFKIELD TWSNGTQQSF DEYISSSWLS NGSRLTGLLT |
| 441 | MQFVGVKLSD EMLMSEECTD LARHVCMVGR LLNDVCSSER |
| 481 | EREENIAGKS YSILLATEKD GRKVSEDEAI AEINEMVEYH |
| 521 | WRKVLQIVYK KESILPRRCK DVFLEMAKGT FYAYGINDEL |
| 561 | TSPQQSKEDM KSFVF |

A nucleic acid encoding the Salvia sclarea sclareol synthase with SEQ ID NO:176 is shown below as SEQ ID NO:177.

| | |
|---|---|
| 1 | ATGTCGCTCG CCTTCAACGT CGGAGTTACG CCTTTCTCCG |
| 41 | GCCAAAGAGT TGGGAGCAGG AAAGAAAAAT TTCCAGTCCA |
| 81 | AGGATTTCCT GTGACCACCC CCAATAGGTC ACGTCTCATC |
| 121 | GTTAACTGCA GCCTTACTAC AATAGATTTC ATGGCGAAAA |
| 161 | TGAAAGAGAA TTTCAAGAGG GAAGACGATA AATTTCCAAC |
| 201 | GACAACGACT CTTCGATCCG AAGATATACC CTCTAATTTG |
| 241 | TGTATAATCG ACACCCTTCA AAGGTTGGGG GTCGATCAAT |
| 231 | TCTTCCAATA TGAAATCAAC ACTATTCTAG ATAACACATT |
| 321 | CAGGTTGTGG CAAGAAAAAC ACAAAGTTAT ATATGGCAAT |
| 361 | GTTACTACTC ATGCAATGGC ATTTAGGCTT TTGCGAGTGA |
| 401 | AAGGATACGA AGTTTCATCA GAGGAGTTGG CTCCATATGG |
| 441 | TAACCAAGAG GCTGTTAGGC AGCAAACAAA TGACCTGCCG |
| 481 | ATGATTATTG AGCTTTATAG AGCAGCAAAT GAGAGAATAT |
| 521 | ATGAAGAAGA GAGGAGTCTT GAAAAAATTC TTGCTTGGAC |
| 561 | TACCATCTTT CTCAATAAGC AAGTGCAAGA TAACTCAATT |
| 601 | CCCGACAAAA AACTGCACAA ACTGGTGGAA TTCTACTTGA |
| 641 | GGAATTACAA AGGCATAACC ATAAGATTGG GAGCTAGACG |
| 681 | AAACCTCGAG CTATATGACA TGACCTACTA TCAAGCTCTG |
| 721 | AAATCTACAA ACAGGTTCTC TAATTTATGC AACGAAGATT |
| 761 | TTCTAGTTTT CGCAAAGGAA GATTTCGATA TACATGAAGC |
| 801 | CCAGAACCAG AAAGGACTTC AACAACTGCA AAGGTGGTAT |
| 841 | GCAGATTGTA GGTTGGACAC CTTAAACTTT GGAAGAGATG |
| 831 | TAGTTATTAT TGCTAATTAT TTGGCTTCAT TAATTATTGG |
| 921 | TGATCATGCG TTTGACTATG TTCGTCTCGC ATTTGCCAAA |
| 961 | ACATCTGTGC TTGTAACAAT TATGGATGAT TTTTTCGACT |
| 1001 | GTCATGGCTC TAGTCAAGAG TGTGAGAAGA TCATTGAATT |
| 1041 | AGTAAAAGAA TGGAAGGAGA ATCCGGATGC AGAGTACGGA |
| 1081 | TCTGAGGAGC TTGAGATCCT TTTTATGGCG TTGTACAATA |
| 1121 | CAGTAAATGA GTTGGCGGAG AGGGCTCGTG TTGAACAGGG |
| 1161 | GCGTAGTGTC AAAGAGTTTC TAGTCAAACT GTGGGTTGAA |
| 1201 | ATACTCTCAG CTTTCAAGAT AGAATTAGAT ACATGGAGCA |
| 1241 | ATGGCACGCA GCAAAGCTTC GATGAATACA TTTCTTCGTC |
| 1281 | GTGGTTGTCG AACGGTTCCC GGCTGACAGG TCTCCTGACG |
| 1321 | ATGCAATTCG TCGGAGTAAA ATTGTCCGAT GAAATGCTTA |
| 1361 | TGAGTGAAGA GTGCACTGAT TTGGCTAGGC ATGTCTGTAT |
| 1401 | GGTCGGCCGG CTGCTCAACG ACGTGTGCAG TTCTGAGAGG |
| 1441 | GAGCGCGAGG AAAATATTGC AGGAAAAAGT TATAGCATTC |
| 1431 | TACTAGCAAC TGAGAAAGAT GGAAGAAAAG TTAGTGAAGA |
| 1521 | TGAAGCCATT GCAGAGATCA ATGAAATGGT TGAATATCAC |
| 1561 | TGGAGAAAAG TGTTGCAGAT TGTGTATAAA AAAGAAAGCA |
| 1601 | TTTTGCCAAG AAGATGCAAA GATGTATTTT TGGAGATGGC |
| 1641 | TAAGGGTACG TTTTATGCTT ATGGGATCAA CGATGAATTG |
| 1681 | ACTTCTCCTC AGCAATCCAA GGAAGATATG AAATCCTTTG |
| 1721 | TCTTTTGA |

Enzymes described herein can have one or more deletions, insertions, replacements, or substitutions in a part of the enzyme. The enzyme(s) described herein can have, for example, at least 60%, or at least 70%, or at least 80%, or at least 90%, or at least 93%, or at least 95%, or at least 96%, or at least 97%, or at least 98%, or at least 99% sequence identity to a sequence described herein.

In some cases, enzymes can have conservative changes such as one or more deletions, insertions, replacements, or substitutions that have no significant effect on the activities of the enzymes. Examples of conservative substitutions are provided below in Table 1A.

TABLE 1A
Conservative Substitutions

| Type of Amino Acid | Substitutable Amino Acids |
|---|---|
| Hydrophilic | Ala, Pro, Gly, Glu, Asp, Gln, Asn, Ser, Thr |
| Sulfhydryl | Cys |
| Aliphatic | Val, Ile, Leu, Met |
| Basic | Lys, Arg, His |
| Aromatic | Phe, Tyr, Trp |

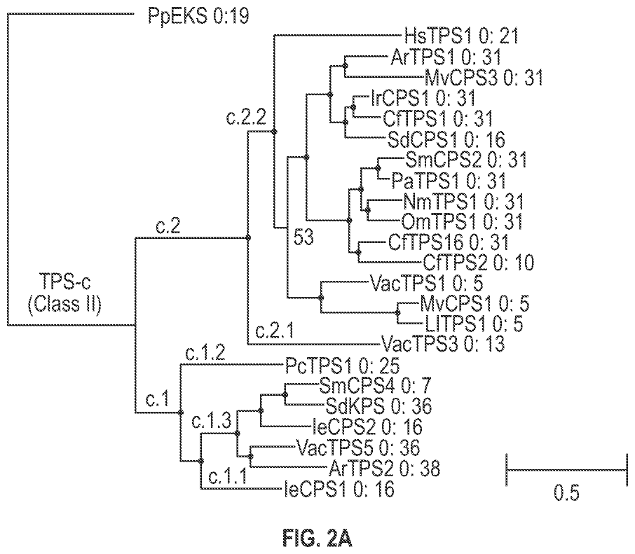

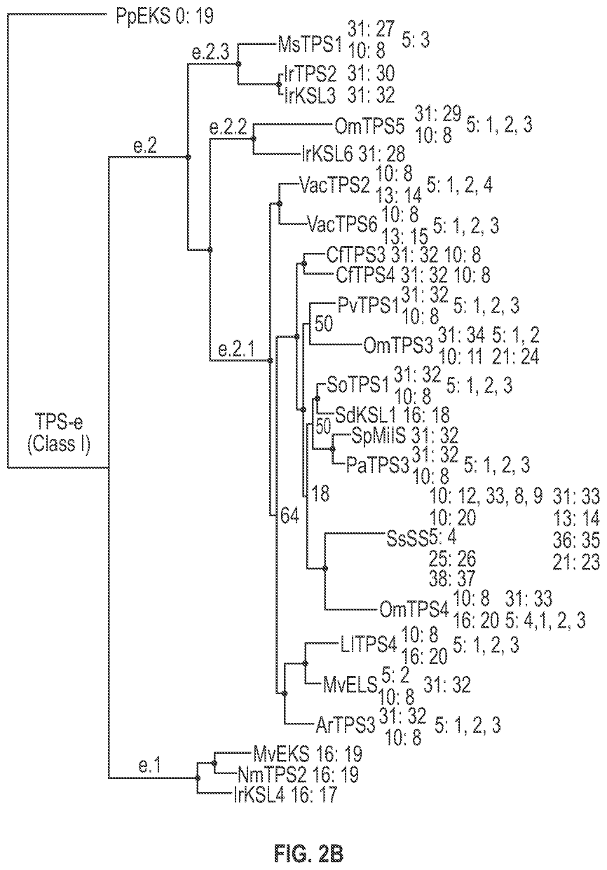

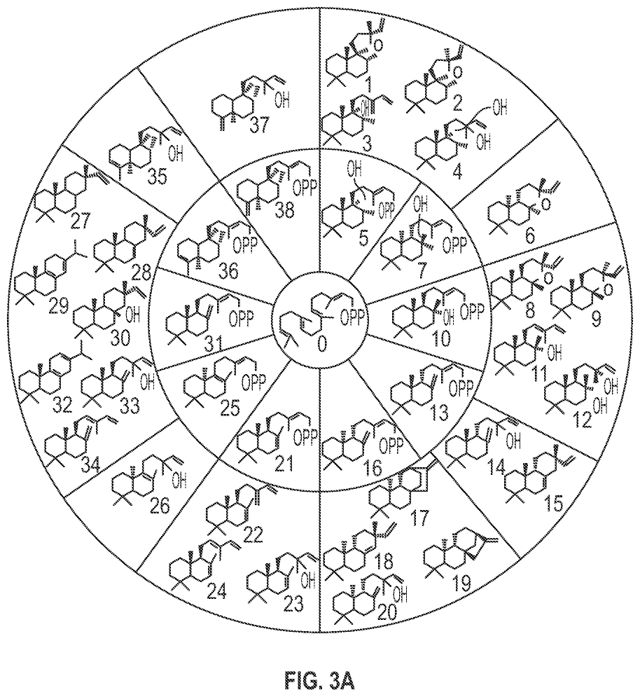

Due to an increase in resolution at the taxonomic level and consistent clustering of enzymes with identical, or related function, the inventors propose a hierarchical scheme for classifying TPS genes in Lamiaceae from the TPS-e and TPS-c subfamilies. TPS-c genes (class II diTPSs) from Lamiaceae fall broadly into two clades (FIG. 2A), which are referred to herein as c.1 and c.2. These c.1 and c.2 clades are further divided into three, and two subclades, respectively. The characterized genes from c.1.1 are all ent-CPP [16] synthases, presumably involved in primary metabolism. The taxonomic organization among c.1.1 sequences closely resembles the consensus phylogeny generated from 520 genes from each species (19), together with the short branch lengths compared to other TPS-c clades suggests that diTPSs in c.1.1 are more conserved and evolve more slowly.

The remaining TPS-c clades contain genes involved in specialized metabolism. The only characterized gene from clade c.1.2 is PcTPS1 which makes an ent-labda-8-ene diphosphate product [25]. Enzymes from clade c.1.3 catalyze the production of a variety of products, including ent-CPP [16], ent-8-LPP [7], kolavenyl-PP [36], and 38. 36 and 38 are the only products without the labdane (Sk4) skeleton produced by Lamiaceae class II diTPSs. Compounds apparently derived from 36 are widespread among Lamiaceae (Table 6), so the inventors hypothesize that the progenitor of c.1.3 was a kolavenyl-PP synthase present in an early common ancestor. The labdane compounds produced by enzymes in c.1 are all in the ent-configuration. With two exceptions, the known enzymes from clade c.2 all make products with the labdane skeleton in the normal configuration, suggesting that the founder of that clade may have been a normal configuration labdadiene diphosphate synthase. The exceptions are VacTPS3, the only characterized member of c.2.1, which produces syn-CPP [13], and the curious case of SdCPS1, which produces ent-CPP.

Among TPS-e (class I) genes, all but one of the characterized enzymes from e.1 are ent-kaurene [19] synthases, presumably involved in gibberellin biosynthesis. As with the c.1.1 clade, e.1 reflects the taxonomic distribution among the species. Notable in this clade are IrKSL4, which is an ent-atiserene synthase, and SmKSL2, which, in addition to ent-kaurene synthase activity, can convert ent-8-LPP 7 into ent-13-epi-manoyl oxide [6]. Andersen-Ranberg et al. (Angew Chem Int Ed 55(6):2142-2146 (2016)) have tested four of four ent-kaurene synthases and have data indicating that one was from Lamiaceae, which had the ability to convert 7 to 6, so it is likely that this is a general characteristic of enzymes in the e.1 group.

Most of the specialized class I diTPSs in Lamiaceae fall into clade e.2. Enzymes in e.2 have lost the γ domain, present in many diTPSs, and located on the opposite end of the peptide from the class I active site. Characteristic of enzymes in e.2 is their ability to act on multiple substrates. The extreme example is SsSS (Caniard et al. M C Plant Biology 12:119 (2012)) which so far has been able to catalyze the dephosphorylation and minor rearrangement of all class II enzyme products that it has been tested. The range of substrates accepted by other enzymes in this group has not been tested systematically, but among the e.2 enzymes characterized in this study, only one (OmTPS4) accepted ent-CPP, and all accepted (+)-CPP [31], (+)-8-LPP [10], and PgPP [5]. There is great diversity the products of e.2 enzymes, with over 20 distinct compounds represented. Most of the enzymes in e.2 convert (+)-CPP to miltiradiene [32], and (+)-8-LPP to 13R-(+)-manoyl oxide [8], with other activities arising sporadically across the clade. Both characterized enzymes in the Nepetoideae specific e.2.2 have unusual activities: IrKSL6 converts (+)-CPP to isopimara-7,15-diene [28], and OmTPS5 converts (+)-CPP to palustradiene [29]. Most of the enzymes in e.2 fall into the e.2.1 clade which also accounts for most of the known products. Enzymes that we characterized from e.2.1 lent support to emerging functionally consistent subclades. OmTPS4 activity, for three out of four substrates tested, mimics that of its nearest homolog (SsSS), notably accepting ent-CPP as a substrate to produce ent-manool [20]. LITPS4 likewise has activities most similar to its closest homolog, MvELS, converting PgPP into 9,13(S)-epoxy-labd-14-ene [2] with greater specificity than other enzymes tested, although the products from (+)-CPP are different. From the remaining clade, e.2.3, the three characterized enzymes all come from Nepetoideae, and convert (+)-CPP into different products: IrKSL3 produces miltiradiene, IrTPS2 produces nezukol [30], and MsTPS1 produces sandaracopimaradiene [27].

The existence of two strongly supported subclades of specialized diTPSs within c.1, together with the presence of an ent-atiserene synthase in e.1, indicate that the emergence of specialized diTPSs from ent-CPP and ent-kaurene synthases is an ongoing process that has occurred multiple times in the Lamiaceae lineage. While it is evident that candidates selected from anywhere in the phylogenetic tree may have novel activities, clades that seem particularly promising and underexplored are c.2.1, c.1.2, and e.2.3. So far, including this work and previous work, diTPSs have been characterized from only four of the twelve major Lamiaceae clades: Ajugoideae, Lamioideae, Nepetoideae, and Viticoideae. Further expanding to the remaining eight Lamiaceae clades may also be a promising strategy for finding new enzyme activities.

### Expression of Enzymes

Also described herein are expression systems that include at least one expression cassette (e.g., expression vectors or transgenes) that encode one or more of the enzyme(s) described herein. The expression systems can also include one or more expression cassettes encoding an enzyme that can synthesize terpene building blocks. For example, the expression systems can include one or more expression cassettes encoding terpene synthases that facilitate production of terpene precursors or building blocks such as those involved in the synthesis of isopentenyl diphosphate (IPP) or dimethylallyl diphosphate (DMAPP).

Cells containing such expression systems are further described herein. The cells containing such expression systems can be used to manufacture the enzymes (e.g., for in vitro use) and/or one or more terpenes, diterpenes, or terpenoids produced by the enzymes. Methods of using the enzymes or cells containing expression cassettes encoding such enzymes to make products such as terpenes, diterpenes, terpenoids, and combinations thereof are also described herein.

Nucleic acids encoding the enzymes can have sequence modifications. For example, nucleic acid sequences described herein can be modified to express enzymes that have modifications. Most amino acids can be encoded by more than one codon. When an amino acid is encoded by more than one codon, the codons are referred to as degenerate codons. A listing of degenerate codons is provided in Table 1B below.

TABLE 1B
Degenerate Amino Acid Codons

| Amino Acid | Three Nucleotide Codon |
|---|---|
| Ala/A | GCT, GCC, GCA, GCG |
| Arg/R | CGT, CGC, CGA, CGG, AGA, AGG |
| Asn/N | AAT, AAC |
| Asp/D | GAT, GAC |
| Cys/C | TGT, TGC |
| Gln/Q | CAA, CAG |
| Glu/E | GAA, GAG |
| Gly/G | GGT, GGC, GGA, GGG |
| His/H | CAT, CAC |
| Ile/I | ATT, ATC, ATA |
| Leu/L | TTA, TTG, CTT, CTC, CTA, CTG |
| Lys/K | AAA, AAG |
| Met/M | ATG |
| Phe/F | TTT, TTC |
| Pro/P | CCT, CCC, CCA, CCG |
| Ser/S | TCT, TCC, TCA, TCG, AGT, AGC |
| Thr/T | ACT, ACC, ACA, ACG |
| Trp/W | TGG |
| Tyr/Y | TAT, TAC |
| Val/V | GTT, GTC, GTA, GTG |
| START | ATG |
| STOP | TAG, TGA, TAA |

Different organisms may translate different codons more or less efficiently (e.g., because they have different ratios of tRNAs) than other organisms. Hence, when some amino acids can be encoded by several codons, a nucleic acid segment can be designed to optimize the efficiency of expression of an enzyme by using codons that are preferred by an organism of interest. For example, the nucleotide coding regions of the enzymes described herein can be codon optimized for expression in various plant species. For example, many of the enzymes described herein were originally isolated from the mint family (Lamiaceae), however such enzymes can be expressed in a variety of host cells, including for example, as Nicotiana benthamiana, Nicotiana tabacum, Nicotiana rustica, Nicotiana excelsior, and Nicotiana excelsiana.

An optimized nucleic acid can have less than 98%, less than 97%, less than 95%, or less than 94%, or less than 93%, or less than 92%, or less than 91%, or less than 90%, or less than 89%, or less than 88%, or less than 85%, or less than 83%, or less than 80%, or less than 75% nucleic acid sequence identity to a corresponding non-optimized (e.g., a non-optimized parental or wild type enzyme nucleic acid) sequence.

The enzymes described herein can be expressed from an expression cassette and/or an expression vector. Such an expression cassette can include a nucleic acid segment that encodes an enzyme operably linked to a promoter to drive expression of the enzyme. Convenient vectors, or expression systems can be used to express such enzymes. In some instances, the nucleic acid segment encoding an enzyme is operably linked to a promoter and/or a transcription termination sequence. The promoter and/or the termination sequence can be heterologous to the nucleic acid segment that encodes an enzyme. Expression cassettes can have a promoter operably linked to a heterologous open reading frame encoding an enzyme. The invention therefore provides expression cassettes or vectors useful for expressing one or more enzyme(s).

Constructs, e.g., expression cassettes, and vectors comprising the isolated nucleic acid molecule, e.g., with optimized nucleic acid sequence, as well as kits comprising the isolated nucleic acid molecule, construct or vector are also provided.

The nucleic acids described herein can also be modified to improve or alter the functional properties of the encoded enzymes. Deletions, insertions, or substitutions can be generated by a variety of methods such as, but not limited to, random mutagenesis and/or site-specific recombination-mediated methods. The mutations can range in size from one or two nucleotides to hundreds of nucleotides (or any value there between). Deletions, insertions, and/or substitutions are created at a desired location in a nucleic acid encoding the enzyme(s).

Nucleic acids encoding one or more enzyme(s) can have one or more nucleotide deletions, insertions, replacements, or substitutions. For example, the nucleic acids encoding one or more enzyme(s) can, for example, have less than 95%, or less than 94.8%, or less than 94.5%, or less than 94%, or less than 93.8%, or less than 94.50% nucleic acid sequence identity to a corresponding parental or wild-type sequence. In some cases, the nucleic acids encoding one or more enzyme(s) can have, for example, at least 50%, or at least 55%, or at least 60%, or at least 65%, or at least 70%, or at least 75%, or at least 80%, or at least 85%, or at 90% sequence identity to a corresponding parental or wild-type sequence. Examples of parental or wild type nucleic acid sequences for unmodified enzyme(s) with amino acid sequences SEQ ID NOs:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176 include nucleic acid sequences SEQ ID NOs:2, 4, 6, 8, 10, 12, 14, 16, 18, 20, 22, 24, 26, 28, 30, 32, 34, 36, 38, 40, 42, 44, 46, 48, 50, 52, 54, 56, 58, 60, or 177 respectively. Any of these nuclei acid or amino acid sequences can, for example, encode or have enzyme sequences with less than 99%, less than 98%, less than 97%, less than 96%, less than 95%, less than 94.8%, less than 94.5%, less than 94%, less than 93.8%, less than 93.5%, less than 93%, less than 92%, less than 91%, or less than 90% sequence identity to a corresponding parental or wild-type sequence.

Also provided are nucleic acid molecules (polynucleotide molecules) that can include a nucleic acid segment encoding an enzyme with a sequence that is optimized for expression in at least one selected host organism or host cell. Optimized sequences include sequences which are codon optimized, i.e., codons which are employed more frequently in one organism relative to another organism. In some cases, the balance of codon usage is such that the most frequently used codon is not used to exhaustion. Other modifications can include addition or modification of Kozak sequences and/or introns, and/or to remove undesirable sequences, for instance, potential transcription factor binding sites.

An enzyme useful for synthesis of terpenes, diterpenes, and terpenoids may be expressed on the surface of, or within, a prokaryotic or eukaryotic cell. In some cases, expressed enzyme(s) can be secreted by that cell.

Techniques of molecular biology, microbiology, and recombinant DNA technology which are within the skill of the art can be employed to make and use the enzymes, expression systems, and terpene products described herein. Such techniques available in the literature. See, e.g., Sambrook, Fritsch & Maniatis, Molecular Cloning: A Laboratory Manual, Second Edition (1989); DNA Cloning, Vols. I and II (D. N. Glover ed. 1985); Oligonucleotide Synthesis (M. J. Gait ed. 1984); Nucleic Acid Hybridization (B. D. Hames & S. J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series Methods In Enzymology (S. Colowick and N. Kaplan eds., Academic Press, Inc.); Current Protocols In Molecular Biology (John Wiley & Sons, Inc), Current Protocols In Protein Science (John Wiley & Sons, Inc), Current Protocols In Microbiology (John Wiley & Sons, Inc), Current Protocols In Nucleic Acid Chemistry (John Wiley & Sons, Inc), and Handbook of Experimental Immunology, Vols. I-IV (D. M. Weir and C. C. Blackwell eds., 1986, Blackwell Scientific Publications).

Modified plants that contain nucleic acids encoding enzymes within their somatic and/or germ cells are described herein. Such genetic modification can be accomplished by available procedures. For example, one of skill in the art can prepare an expression cassette or expression vector that can express one or more encoded enzymes. Plant cells can be transformed by the expression cassette or expression vector, and whole plants (and their seeds) can be generated from the plant cells that were successfully transformed with the enzyme nucleic acids. Some procedures for making such genetically modified plants and their seeds are described below.

Promoters: The nucleic acids encoding enzymes can be operably linked to a promoter, which provides for expression of mRNA from the nucleic acids encoding the enzymes. The promoter is typically a promoter functional in plants and can be a promoter functional during plant growth and development. A nucleic acid segment encoding an enzyme is operably linked to the promoter when it is located downstream from the promoter. The combination of a coding region for an enzyme operably linked to a promoter forms an expression cassette, which can optionally include other elements as well.

Promoter regions are typically found in the flanking DNA upstream from the coding sequence in both the prokaryotic and eukaryotic cells. A promoter sequence provides for regulation of transcription of the downstream gene sequence and typically includes from about 50 to about 2,000 nucleotide base pairs. Promoter sequences also contain regulatory sequences such as enhancer sequences that can influence the level of gene expression. Some isolated promoter sequences can provide for gene expression of heterologous DNAs, that is a DNA different from the native or homologous DNA.

Promoter sequences are also known to be strong or weak, or inducible. A strong promoter provides for a high level of gene expression, whereas a weak promoter provides for a very low level of gene expression. An inducible promoter is a promoter that provides for the turning gene expression on and off in response to an exogenously added agent, or to an environmental or developmental stimulus. For example, a bacterial promoter such as the P_{tac} promoter can be induced to varying levels of gene expression depending on the level of isopropyl-beta-D-thiogalactoside added to the transformed cells. Promoters can also provide for tissue specific or developmental regulation. An isolated promoter sequence that is a strong promoter for heterologous DNAs is advantageous because it provides for a sufficient level of gene expression for easy detection and selection of transformed cells and provides for a high level of gene expression when desired.

Expression cassettes generally include, but are not limited to, examples of plant promoters such as the CaMV 35S promoter (Odell et al., Nature. 313:810-812 (1985)), or others such as CaMV 19S (Lawton et al., Plant Molecular Biology. 9:315-324 (1987)), nos (Ebert et al., Proc. Natl. Acad. Sci. USA. 84:5745-5749 (1987)), Adh1 (Walker et al. Proc. Natl. Acad. Sci. USA. 84:6624-6628 (1987)), sucrose synthase (Yang et al., Proc. Natl. Acad. Sci. USA. 87:4144-4148 (1990)), α-tubulin, ubiquitin, actin (Wang et al, Mol. Cell. Biol. 12:3399 (1992)), cab (Sullivan et al., Mol. Gen. Genet. 215:431 (1989)), PEPCase (Hudspeth et al., Plant Molecular Biology. 12:579-589 (1989)) or those associated with the R gene complex (Chandler et al, The Plant Cell. 1:1175-1183 (1989)). Further suitable promoters include a CYP71D16 trichome-specific promoter and the CBTS (cembratrienol synthase) promotor, cauliflower mosaic virus promoter, the Z10 promoter from a gene encoding a 10 kD zein protein, a Z27 promoter from a gene encoding a 27 kD zein protein, the plastid rRNA-operon (rrn) promoter, inducible promoters, such as the light inducible promoter derived from the pea rbcS gene (Coruzzi et al., EMBO J. 3:1671 (1971)), RUBISCO-SSU light inducible promoter (SSU) from tobacco and the actin promoter from rice (McElroy et al., The Plant Cell. 2:163-171 (1990)). Other promoters that are useful can also be employed.

Alternatively, novel tissue specific promoter sequences may be employed. cDNA clones from a particular tissue can be isolated and those clones which are expressed specifically in that tissue can be identified, for example, using Northern blotting. Preferably, the gene isolated is not present in a high copy number but is relatively abundant in specific tissues. The promoter and control elements of corresponding genomic clones can then be localized using techniques well known to those of skill in the art.

A nucleic acid encoding an enzyme can be combined with the promoter by standard methods to yield an expression cassette, for example, as described in Sambrook et al. (MOLECULAR CLONING: A LABORATORY MANUAL. Second Edition (Cold Spring Harbor, NY: Cold Spring Harbor Press (1989); MOLECULAR CLONING: A LABORATORY MANUAL. Third Edition (Cold Spring Harbor, NY: Cold Spring Harbor Press (2000)). Briefly, a plasmid containing a promoter such as the 35S CaMV promoter or the CYP71D16 trichome-specific promoter can be constructed as described in Jefferson (Plant Molecular Biology Reporter 5:387-405 (1987)) or obtained from Clontech Lab in Palo Alto. California (e.g., pBI121 or pBI221). Typically, these plasmids are constructed to have multiple cloning sites having specificity for different restriction enzymes downstream from the promoter.

The nucleic acid sequence encoding for the enzyme(s) can be subcloned downstream from the promoter using restriction enzymes and positioned to ensure that the DNA is inserted in proper orientation with respect to the promoter so that the DNA can be expressed as sense RNA. Once the nucleic acid segment encoding the enzyme is operably linked to a promoter, the expression cassette so formed can be subcloned into a plasmid or other vector (e.g., an expression vector).

In some embodiments, a cDNA clone encoding an enzyme is isolated from a mint species, for example, from leaf, trichome, or root tissue. In other embodiments, cDNA clones from other species (that encode an enzyme) are isolated from selected plant tissues, or a nucleic acid encoding a wild type, mutant or modified enzyme is prepared by available methods or as described herein. For example, the nucleic acid encoding the enzyme can be any nucleic acid with a coding region that hybridizes to SEQ ID NOs: 4, 6, 8, 10, 12, 14, 16, 18, 20, 22, 24, 26, 28, 30, 32, 34, 36, 38, 40, 42, 44, 46, 48, 50, 52, 54, 56, 58, 60, or 177, and that has enzyme activity. Using restriction endonucleases, the entire coding sequence for the enzyme is subcloned downstream of the promoter in a 5′ to 3′ sense orientation.

Targeting Sequences: Additionally, expression cassettes can be constructed and employed to target the nucleic acids encoding an enzyme to an intracellular compartment within plant cells or to direct an encoded protein to the extracellular environment. This can generally be achieved by joining a DNA sequence encoding a transit or signal peptide sequence to the coding sequence of the nucleic acid encoding the enzyme. The resultant transit, or signal, peptide can transport the protein to a particular intracellular, or extracellular, destination and can then be co-translationally or post-translationally removed. Transit peptides act by facilitating the transport of proteins through intracellular membranes, e.g., vacuole, vesicle, plastid and mitochondrial membranes, whereas signal peptides direct proteins through the extracellular membrane. By facilitating transport of the protein into compartments inside or outside the cell, these sequences can increase the accumulation of a particular gene product within a particular location. For example, see U.S. Pat. No. 5,258,300.

For example, in some cases it may be desirable to localize the enzymes to the plastidic compartment and/or within plant cell trichomes. The best compliment of transit peptides/secretion peptide/signal peptides can be empirically ascertained. The choices can range from using the native secretion signals akin to the enzyme candidates to be transgenically expressed, to transit peptides from proteins known to be localized into plant organelles such as trichome plastids in general. For example, transit peptides can be selected from proteins that have a relative high titer in the trichomes. Examples include, but not limited to, transit peptides form a terpenoid cyclase (e.g. cembratrieneol cyclase), the LTP1 protein, the Chlorophyll a-b binding protein 40, Phylloplanin, Glycine-rich Protein (GRP), Cytochrome P450 (CYP71D16); all from Nicotiana sp. alongside RUBISCO (Ribulose bisphosphate carboxylase) small unit protein from both Arabidopsis and Nicotiana sp.

3′ Sequences: When the expression cassette is to be introduced into a plant cell, the expression cassette can also optionally include 3′ untranslated plant regulatory DNA sequences that act as a signal to terminate transcription and allow for the polyadenylation of the resultant mRNA. The 3′ untranslated regulatory DNA sequence can include from about 300 to 1,000 nucleotide base pairs and can contain plant transcriptional and translational termination sequences. For example, 3′ elements that can be used include those derived from the nopaline synthase gene of Agrobacterium tumefaciens (Bevan et al., Nucleic Acid Research. 11:369-385 (1983)), or the terminator sequences for the T7 transcript from the octopine synthase gene of Agrobacterium tumefaciens, and/or the 3′ end of the protease inhibitor I or 11 genes from potato or tomato. Other 3′ elements known to those of skill in the art can also be employed. These 3′ untranslated regulatory sequences can be obtained as described in An (Methods in Enzymology. 153:292 (1987)). Many such 3′ untranslated regulatory sequences are already present in plasmids available from commercial sources such as Clontech, Palo Alto, California. The 3′ untranslated regulatory sequences can be operably linked to the 3′ terminus of the nucleic acids encoding the enzyme.

Selectable and Screenable Marker Sequences: To improve identification of transformants, a selectable or screenable marker gene can be employed with the expressible nucleic acids encoding the enzyme(s). “Marker genes” are genes that impart a distinct phenotype to cells expressing the marker gene and thus allow such transformed cells to be distinguished from cells that do not have the marker. Such genes may encode either a selectable or a screenable marker, depending on whether the marker confers a trait which one can ‘select’ for by chemical means, i.e., through the use of a selective agent (e.g., a herbicide, antibiotic, or the like), or whether it is simply a trait that one can identify through observation or testing, i.e., by ‘screening’ (e.g., the R-locus trait). Of course, many examples of suitable marker genes are available can be employed in the practice of the invention.

Included within the terms ‘selectable or screenable marker genes’ are also genes which encode a “secretable marker” whose secretion can be detected as a means of identifying or selecting for transformed cells. Examples include markers which encode a secretable antigen that can be identified by antibody interaction, or secretable enzymes that can be detected by their catalytic activity. Secretable proteins fall into a number of classes, including small, diffusible proteins detectable, e.g., by ELISA; and proteins that are inserted or trapped in the cell wall (e.g., proteins that include a leader sequence such as that found in the expression unit of extensin or tobacco PR-S).

With regard to selectable secretable markers, the use of an expression system that encodes a polypeptide that becomes sequestered in the cell wall, where the polypeptide includes a unique epitope may be advantageous. Such a cell wall antigen can employ an epitope sequence that would provide low background in plant tissue, a promoter-leader sequence that imparts efficient expression and targeting across the plasma membrane, and that can produce protein that is bound in the cell wall and yet is accessible to antibodies. A normally secreted cell wall protein modified to include a unique epitope would satisfy such requirements.

Example of protein markers suitable for modification in this manner include extensin or hydroxyproline rich glycoprotein (HPRG). For example, the maize HPRG (Stiefel et al., The Plant Cell. 2:785-793 (1990)) is well characterized in terms of molecular biology, expression, and protein structure and therefore can readily be employed. However, any one of a variety of extensins and/or glycine-rich cell wall proteins (Keller et al., EMBO J. 8:1309-1314 (1989)) could be modified by the addition of an antigenic site to create a screenable marker.

Selectable markers for use in connection with the present invention can include, but are not limited to, a neo gene (Potrykus et al., Mol. Gen. Genet. 199:183-188 (1985)) which codes for kanamycin resistance and can be selected for using kanamycin, G418; a bar gene which codes for bialaphos resistance; a gene which encodes an altered EPSP synthase protein (Hinchee et al., Bio/Technology 6:915-922 (1988)) thus conferring glyphosate resistance; a nitrilase gene such as bxn from Klebsiella ozaenae which confers resistance to bromoxynil (Stalker et al., Science. 242:419-423 (1988)); a mutant acetolactate synthase gene (ALS) which confers resistance to imidazolinone, sulfonylurea or other ALS-inhibiting chemicals (European Patent Application 154,204 (1985)); a methotrexate-resistant DHFR gene (Thillet et al, J. Biol. Chem. 263:12500-12508 (1988)); a dalapon dehalogenase gene that confers resistance to the herbicide dalapon; or a mutated anthranilate synthase gene that confers resistance to 5-methyl tryptophan. Where a mutant EPSP synthase gene is employed, additional benefit may be realized through the incorporation of a suitable chloroplast transit peptide. CTP (European Patent Application 0 218 571 (1987)).

An illustrative embodiment of a selectable marker gene capable of being used in systems to select transformants is the gene that encode the enzyme phosphinothricin acetyltransferase, such as the bar gene from Streptomyces hygroscopicus or the pat gene from Streptomyces viridochromogenes (U.S. Pat. No. 5,550,318). The enzyme phosphinothricin acetyl transferase (PAT) inactivates the active ingredient in the herbicide bialaphos, phosphinothricin (PPT). PPT inhibits glutamine synthetase, (Murakami et al. Mol. Gen. Genet. 205:42-50 (1986); Twell et al., Plant Physiol. 91:1270-1274 (1989)) causing rapid accumulation of ammonia and cell death. Screenable markers that may be employed include, but are not limited to, a β-glucuronidase or uidA gene (GUS) that encodes an enzyme for which various chromogenic substrates are known; an R-locus gene, which encodes a product that regulates the production of anthocyanin pigments (red color) in plant tissues (Dellaporta et al., In: Chromosome Structure and Function: Impact of New Concepts, 18^{th} Stadler Genetics Symposium, J. P. Gustafson and R. Appels, eds. (New York: Plenum Press) pp. 263-282 (1988)); a β-lactamase gene (Sutcliffe, Proc. Natl. Acad. Sci. USA. 75:3737-3741 (1978)), which encodes an enzyme for which various chromogenic substrates are known (e.g., PADAC, a chromogenic cephalosporin); a xyIE gene (Zukowsky et al., Proc. Natl. Acad. Sci. USA 80:1101 (1983)) which encodes a catechol dioxygenase that can convert chromogenic catechols; an α-amylase gene (Ikuta et al, Bio/technology 8:241-242 (1990)); a tyrosinase gene (Katz et al., J Gen. Microbiol. 129:2703-2714 (1983)) which encodes an enzyme capable of oxidizing tyrosine to DOPA and dopaquinone which in turn condenses to form the easily detectable compound melanin; a β-galactosidase gene, which encodes an enzyme for which there are chromogenic substrates; a luciferase (lux) gene (Ow et al., Science. 234:856-859.1986), which allows for bioluminescence detection; or an aequorin gene (Prasher et al., Biochem. Biophys. Res. Comm. 126:1259-1268 (1985)), which may be employed in calcium-sensitive bioluminescence detection, or a green or yellow fluorescent protein gene (Niedz et al., Plant Cell Reports. 14:403 (1995)).

Another screenable marker contemplated for use is firefly luciferase, encoded by the lux gene. The presence of the lux gene in transformed cells may be detected using, for example, X-ray film, scintillation counting, fluorescent spectrophotometry, low-light video cameras, photon counting cameras or multiwell luminometry. It is also envisioned that this system may be developed for population screening for bioluminescence, such as on tissue culture plates, or even for whole plant screening.

Other Optional Sequences: An expression cassette of the invention can also include plasmid DNA. Plasmid vectors include additional DNA sequences that provide for easy selection, amplification, and transformation of the expression cassette in prokaryotic and eukaryotic cells, e.g., pUC-derived vectors such as pUC8, pUC9, pUC18, pUC19, pUC23, pUC119, and pUC120, pSK-derived vectors, pGEM-derived vectors, pSP-derived vectors, or pBS-derived vectors. The additional DNA sequences can include origins of replication to provide for autonomous replication of the vector, additional selectable marker genes, for example, encoding antibiotic or herbicide resistance, unique multiple cloning sites providing for multiple sites to insert DNA sequences or genes encoded in the expression cassette and sequences that enhance transformation of prokaryotic and eukaryotic cells.

Another vector that is useful for expression in both plant and prokaryotic cells is the binary Ti plasmid (as disclosed in Schilperoort et al., U.S. Pat. No. 4,940,838) as exemplified by vector pGA582. This binary Ti plasmid vector has been previously characterized by An (Methods In Enzymology. 153:292 (1987)) and is available from Dr. An. This binary Ti vector can be replicated in prokaryotic bacteria such as E. coli and Agrobacterium. The Agrobacterium plasmid vectors can be used to transfer the expression cassette to dicot plant cells, and under certain conditions to monocot cells, such as rice cells. The binary Ti vectors can include the nopaline T DNA right and left borders to provide for efficient plant cell transformation, a selectable marker gene, unique multiple cloning sites in the T border regions, the colE1 replication of origin and a wide host range replicon. The binary Ti vectors carrying an expression cassette of the invention can be used to transform both prokaryotic and eukaryotic cells but is usually used to transform dicot plant cells.

DNA Delivery of the DNA Molecules into Host Cells: Methods described herein can include introducing nucleic acids encoding enzymes, such as a preselected cDNA encoding the selected enzyme, into a recipient cell to create a transformed cell. In some instances, the frequency of occurrence of cells taking up exogenous (foreign) DNA may be low. Moreover, it is most likely that not all recipient cells receiving DNA segments or sequences will result in a transformed cell wherein the DNA is stably integrated into the plant genome and/or expressed. Some recipient cells may show only initial and transient gene expression. However, certain cells from virtually any dicot or monocot species may be stably transformed, and these cells regenerated into transgenic plants, through the application of the techniques disclosed herein.

Another aspect of the invention is a plant that can produce terpenes, diterpenes and terpenoids, wherein the plant has introduced nucleic acid sequence(s) encoding one or more enzymes. The plant can be a monocotyledon or a dicotyledon. Another aspect of the invention includes plant cells (e.g., embryonic cells or other cell lines) that can regenerate fertile transgenic plants and/or seeds. The cells can be derived from either monocotyledons or dicotyledons. In some embodiments, the plant or cell is a monocotyledon plant or cell. In some embodiments, the plant or cell is a dicotyledon plant or cell. For example, the plant or cell can be a tobacco plant or cell. The cell(s) may be in a suspension cell culture or may be in an intact plant part, such as an immature embryo, or in a specialized plant tissue, such as callus, such as Type I or Type II callus.

Transformation of plant cells can be conducted by any one of a number of methods available in the art. Examples are: Transformation by direct DNA transfer into plant cells by electroporation (U.S. Pat. Nos. 5,384,253 and 5,472,869, Dekeyser et al., The Plant Cell. 2:591-602 (1990)); direct DNA transfer to plant cells by PEG precipitation (Hayashimoto et al., Plant Physiol. 93:857-863 (1990)); direct DNA transfer to plant cells by microprojectile bombardment (McCabe et al., Bio/Technology. 6:923-926 (1988); Gordon-Kamm et al., The Plant Cell. 2:603-618 (1990); U.S. Pat. Nos. 5,489,520; 5,538,877; and 5,538,880) and DNA transfer to plant cells via infection with Agrobacterium. Methods such as microprojectile bombardment or electroporation can be carried out with “naked” DNA where the expression cassette may be simply carried on any E. coli-derived plasmid cloning vector. In the case of viral vectors, it is desirable that the system retain replication functions, but lack the functions for disease induction.

One method for dicot transformation, for example, involves infection of plant cells with Agrobacterium tumefaciens using the leaf-disk protocol (Horsch et al., Science 227:1229-1231 (1985). Methods for transformation of monocotyledonous plants utilizing Agrobacterium tumefaciens have been described by Hiei et al. (European Patent 0 604 662, 1994) and Saito et al. (European Patent 0 672 752, 1995).

Monocot cells such as various grasses or dicot cells such as tobacco can be transformed via microprojectile bombardment of embryogenic callus tissue or immature embryos, or by electroporation following partial enzymatic degradation of the cell wall with a pectinase-containing enzyme (U.S. Pat. Nos. 5,384,253; and 5,472,869). For example, embryogenic cell lines derived from immature embryos can be transformed by accelerated particle treatment as described by Gordon-Kamm et al. (The Plant Cell. 2:603-618 (1990)) or U.S. Pat. Nos. 5,489,520; 5,538,877 and 5,538,880, cited above. Excised immature embryos can also be used as the target for transformation prior to tissue culture induction, selection and regeneration as described in U.S. application Ser. No. 08/112,245 and PCT publication WO 95/06128.

The choice of plant tissue source for transformation may depend on the nature of the host plant and the transformation protocol. Useful tissue sources include callus, suspensions culture cells, protoplasts, leaf segments, stem segments, tassels, pollen, embryos, hypocotyls, tuber segments, meristematic regions, and the like. The tissue source is selected and transformed so that it retains the ability to regenerate whole, fertile plants following transformation, i.e., contains totipotent cells.

The transformation is carried out under conditions directed to the plant tissue of choice. The plant cells or tissue are exposed to the DNA or RNA encoding enzymes for an effective period of time. This may range from a less than one second pulse of electricity for electroporation to a 2-day to 3-day co-cultivation in the presence of plasmid-bearing Agrobacterium cells. Buffers and media used will also vary with the plant tissue source and transformation protocol. Many transformation protocols employ a feeder layer of suspended culture cells (tobacco, for example) on the surface of solid media plates, separated by a sterile filter paper disk from the plant cells or tissues being transformed.

Electroporation: Where one wishes to introduce DNA by means of electroporation, it is contemplated that the method of Krzyzek et al. (U.S. Pat. No. 5,384,253) may be advantageous. In this method, certain cell wall-degrading enzymes, such as pectin-degrading enzymes, are employed to render the target recipient cells more susceptible to transformation by electroporation than untreated cells. Alternatively, recipient cells can be made more susceptible to transformation, by mechanical wounding.

To effect transformation by electroporation, one may employ either friable tissues such as a suspension cell cultures, or embryogenic callus, or alternatively, one may transform immature embryos or other organized tissues directly. The cell walls of the preselected cells or organs can be partially degraded by exposing them to pectin-degrading enzymes (pectinases or pectolyases) or mechanically wounding them in a controlled manner. Such cells would then be receptive to DNA uptake by electroporation, which may be carried out at this stage, and transformed cells then identified by a suitable selection or screening protocol dependent on the nature of the newly incorporated DNA.

Microprojectile Bombardment: A further advantageous method for delivering transforming DNA segments to plant cells is microprojectile bombardment. In this method, microparticles may be coated with DNA and delivered into cells by a propelling force. Exemplary particles include those comprised of tungsten, gold, platinum, and the like.

It is contemplated that in some instances DNA precipitation onto metal particles would not be necessary for DNA delivery to a recipient cell using microprojectile bombardment. In an illustrative embodiment, non-embryogenic BMS cells were bombarded with intact cells of the bacteria E. coli or Agrobacterium tumefaciens containing plasmids with either the β-glucoronidase or bar gene engineered for expression in selected plant cells. Bacteria were inactivated by ethanol dehydration prior to bombardment. A low level of transient expression of the β-glucoronidase gene was observed 24-48 hours following DNA delivery. In addition, stable transformants containing the bar gene were recovered following bombardment with either E. coli or Agrobacterium tumefaciens cells. It is contemplated that particles may contain DNA rather than be coated with DNA. Hence it is proposed that particles may increase the level of DNA delivery but are not, in and of themselves, necessary to introduce DNA into plant cells.

An advantage of microprojectile bombardment, in addition to being an effective means of reproducibly stably transforming monocots, microprojectile bombardment does not require the isolation of protoplasts (Christou et al., PNAS 84:3962-3966 (1987)), the formation of partially degraded cells, and no susceptibility to Agrobacterium infection is required. An illustrative embodiment of a method for delivering DNA into maize cells by acceleration is a Biolistics Particle Delivery System, which can be used to propel particles coated with DNA or cells through a screen, such as a stainless steel or Nytex screen, onto a filter surface covered with maize cells cultured in suspension (Gordon-Kamm et al., The Plant Cell 2:603-618 (1990)). The screen disperses the particles so that they are not delivered to the recipient cells in large aggregates. It is believed that a screen intervening between the projectile apparatus and the cells to be bombarded reduces the size of projectile aggregate and may contribute to a higher frequency of transformation, by reducing the damage inflicted on recipient cells by an aggregated projectile.

For bombardment, cells in suspension are preferably concentrated on filters or solid culture medium. Alternatively, immature embryos or other target cells may be arranged on solid culture medium. The cells to be bombarded are positioned at an appropriate distance below the microprojectile stopping plate. If desired, one or more screens are also positioned between the acceleration device and the cells to be bombarded. Through the use of techniques set forth herein, one may obtain up to 1000 or more foci of cells transiently expressing a marker gene. The number of cells in a focus which express the exogenous gene product 48 hours post-bombardment often range from about 1 to 10 and average about 1 to 3.

In bombardment transformation, one may optimize the prebombardment culturing conditions and the bombardment parameters to yield the maximum numbers of stable transformants. Both the physical and biological parameters for bombardment can influence transformation frequency. Physical factors are those that involve manipulating the DNA/microprojectile precipitate or those that affect the path and velocity of either the macro- or microprojectiles. Biological factors include all steps involved in manipulation of cells before and immediately after bombardment, the osmotic adjustment of target cells to help alleviate the trauma associated with the bombardment, and also the nature of the transforming DNA, such as linearized DNA or intact supercoiled plasmid DNA.

One may wish to adjust various bombardment parameters in small scale studies to fully optimize the conditions and/or to adjust physical parameters such as gap distance, flight distance, tissue distance, and helium pressure. One may also minimize the trauma reduction factors (TRFs) by modifying conditions which influence the physiological state of the recipient cells and which may therefore, influence transformation and integration efficiencies. For example, the osmotic state, tissue hydration and the subculture stage or cell cycle of the recipient cells may be adjusted for optimum transformation. Execution of such routine adjustments will be known to those of skill in the art.

Selection: An exemplary embodiment of methods for identifying transformed cells involves exposing the bombarded cultures to a selective agent, such as a metabolic inhibitor, an antibiotic, or the like. Cells which have been transformed and have stably integrated a marker gene conferring resistance to the selective agent used, will grow and divide in culture. Sensitive cells will not be amenable to further culturing.

To use the bar-bialaphos or the EPSPS-glyphosate selective system, bombarded tissue is cultured for about 0-28 days on nonselective medium and subsequently transferred to medium containing from about 1-3 mg/l bialaphos or about 1-3 mM glyphosate, as appropriate. While ranges of about 1-3 mg/l bialaphos or about 1-3 mM glyphosate can be employed, it is proposed that ranges of at least about 0.1-50 mg/l bialaphos or at least about 0.1-50 mM glyphosate will find utility in the practice of the invention. Tissue can be placed on any porous, inert, solid or semi-solid support for bombardment, including but not limited to filters and solid culture medium. Bialaphos and glyphosate are provided as examples of agents suitable for selection of transformants, but the technique of this invention is not limited to them.

The enzyme luciferase is also useful as a screenable marker in the context of the present invention. In the presence of the substrate luciferin, cells expressing luciferase emit light which can be detected on photographic or X-ray film, in a luminometer (or liquid scintillation counter), by devices that enhance night vision, or by a highly light sensitive video camera, such as a photon counting camera. All of these assays are nondestructive and transformed cells may be cultured further following identification. The photon counting camera is especially valuable as it allows one to identify specific cells or groups of cells which are expressing luciferase and manipulate those in real time.

It is further contemplated that combinations of screenable and selectable markers may be useful for identification of transformed cells. For example, selection with a growth inhibiting compound, such as bialaphos or glyphosate at concentrations that provide 100% inhibition followed by screening of growing tissue for expression of a screenable marker gene such as luciferase would allow one to recover transformants from cell or tissue types that are not amenable to selection alone.

Regeneration and Seed Production: Cells that survive the exposure to the selective agent, or cells that have been scored positive in a screening assay, are cultured in media that supports regeneration of plants. One example of a growth regulator that can be used for such purposes is dicamba or 2,4-D. However, other growth regulators may be employed, including NAA, NAA+2,4-D or perhaps even picloram. Media improvement in these and like ways can facilitate the growth of cells at specific developmental stages. Tissue can be maintained on a basic media with growth regulators until sufficient tissue is available to begin plant regeneration efforts, or following repeated rounds of manual selection, until the morphology of the tissue is suitable for regeneration, at least two weeks, then transferred to media conducive to maturation of embryoids. Cultures are typically transferred every two weeks on this medium. Shoot development signals the time to transfer to medium lacking growth regulators.

The transformed cells, identified by selection or screening and cultured in an appropriate medium that supports regeneration, can then be allowed to mature into plants. Developing plantlets are transferred to soilless plant growth mix, and hardened, e.g., in an environmentally controlled chamber at about 85% relative humidity, about 600 ppm CO₂, and at about 25-250 microeinsteins/sec·m² of light. Plants can be matured either in a growth chamber or greenhouse. Plants are regenerated from about 6 weeks to 10 months after a transformant is identified, depending on the initial tissue. During regeneration, cells are grown on solid media in tissue culture vessels. Illustrative embodiments of such vessels are petri dishes and Plant Con™. Regenerating plants can be grown at about 19° C. to 28° C. After the regenerating plants have reached the stage of shoot and root development, they may be transferred to a greenhouse for further growth and testing.

Mature plants are then obtained from cell lines that are known to express the trait. In some embodiments, the regenerated plants are self-pollinated. In addition, pollen obtained from the regenerated plants can be crossed to seed grown plants of agronomically important inbred lines. In some cases, pollen from plants of these inbred lines is used to pollinate regenerated plants. The trait is genetically characterized by evaluating the segregation of the trait in first and later generation progeny. The heritability and expression in plants of traits selected in tissue culture are of particular importance if the traits are to be commercially useful.

Regenerated plants can be repeatedly crossed to inbred plants to introgress the nucleic acids encoding an enzyme into the genome of the inbred plants. This process is referred to as backcross conversion. When a sufficient number of crosses to the recurrent inbred parent have been completed in order to produce a product of the backcross conversion process that is substantially isogenic with the recurrent inbred parent except for the presence of the introduced nucleic acids, the plant is self-pollinated at least once in order to produce a homozygous backcross converted inbred containing the nucleic acids encoding the enzyme(s). Progeny of these plants are true breeding.

Alternatively, seed from transformed plants regenerated from transformed tissue cultures is grown in the field and self-pollinated to generate true breeding plants.

Seed from the fertile transgenic plants can then be evaluated for the presence and/or expression of the enzyme(s). Transgenic plant and/or seed tissue can be analyzed for enzyme expression using methods such as SDS polyacrylamide gel electrophoresis, Western blot, liquid chromatography (e.g., HPLC) or other means of detecting an enzyme product (e.g., a terpene, diterpene, terpenoid, or a combination thereof).

Once a transgenic seed expressing the enzyme(s) and producing one or more terpenes, diterpenes, and/or terpenoids in the plant is identified, the seed can be used to develop true breeding plants. The true breeding plants are used to develop a line of plants expressing terpenes, diterpenes, and/or terpenoids in various plant tissues (e.g., in leaves, bracts, and/or trichomes) while still maintaining other desirable functional agronomic traits. Adding the trait of terpene, diterpene, and/or terpenoid production can be accomplished by back-crossing with selected desirable functional agronomic trait(s) and with plants that do not exhibit such traits and studying the pattern of inheritance in segregating generations. Those plants expressing the target trait(s) in a dominant fashion are preferably selected. Back-crossing is carried out by crossing the original fertile transgenic plants with a plant from an inbred line exhibiting desirable functional agronomic characteristics while not necessarily expressing the trait of terpene, diterpene, and/or terpenoid production in the plant. The resulting progeny can then be crossed back to the parent that expresses the terpenes, diterpenes, and/or terpenoids. The progeny from this cross will also segregate so that some of the progeny carry the trait and some do not. This back-crossing is repeated until the goal of acquiring an inbred line with the desirable functional agronomic traits, and with production of terpenes, diterpenes, and/or terpenoids within various tissues of the plant is achieved. The enzymes can be expressed in a dominant fashion.

Subsequent to back-crossing, the new transgenic plants can be evaluated for synthesis of terpenes, diterpenes, and/or terpenoids in selected plant lines. This can be done, for example, by gas chromatography, mass spectroscopy, or NMR analysis of whole plant cell walls (Kim, H., and Ralph, J. Solution-state 2D NMR of ball-milled plant cell wall gels in DMSO-d₆/pyridine-d₅. (2010) Org. Biomol. Chem. 8(3), 576-591; Yelle, D. J., Ralph, J., and Frihart, C. R. Characterization of non-derivatized plant cell walls using high-resolution solution-state NMR spectroscopy. (2008) Magn. Reson. Chem. 46(6), 508-517; Kim, H., Ralph, J., and Akiyama, T. Solution-state 2D NMR of Ball-milled Plant Cell Wall Gels in DMSO-d₆. (2008) BioEnergy Research 1(1), 56-66; Lu, F., and Ralph, J. Non-degradative dissolution and acetylation of ball-milled plant cell walls; high-resolution solution-state NMR. (2003) Plant J. 35(4), 535-544). The new transgenic plants can also be evaluated for a battery of functional agronomic characteristics such as lodging, yield, resistance to disease, resistance to insect pests, drought resistance, and/or herbicide resistance.

Determination of Stably Transformed Plant Tissues: To confirm the presence of the nucleic acids encoding terpene synthesizing enzymes in the regenerating plants, or seeds or progeny derived from the regenerated plant, a variety of assays may be performed. Such assays include, for example, molecular biological assays, such as Southern and Northern blotting and PCR; biochemical assays, such as detecting the presence of enzyme products, for example, by enzyme assays, by immunological assays (ELISAs and Western blots). Various plant parts can be assayed, such as trichomes, leaves, bracts, seeds or roots. In some cases, the phenotype of the whole regenerated plant can be analyzed.

Whereas DNA analysis techniques may be conducted using DNA isolated from any part of a plant, RNA may only be expressed in particular cells or tissue types and so RNA for analysis can be obtained from those tissues. PCR techniques may also be used for detection and quantification of RNA produced from introduced nucleic acids. PCR can also be used to reverse transcribe RNA into DNA, using enzymes such as reverse transcriptase, and then this DNA can be amplified through the use of conventional PCR techniques. Further information about the nature of the RNA product may be obtained by Northern blotting. This technique will demonstrate the presence of an RNA species and give information about the integrity of that RNA. The presence or absence of an RNA species can also be determined using dot or slot blot Northern hybridizations. These techniques are modifications of Northern blotting and also demonstrate the presence or absence of an RNA species.

While Southern blotting may be used to detect the nucleic acid encoding the enzyme(s) in question, it may not provide information as to whether the preselected DNA segment is being expressed. Expression may be evaluated by specifically identifying the protein products of the introduced nucleic acids or evaluating the phenotypic changes brought about by their expression.

Assays for the production and identification of specific proteins may make use of physical-chemical, structural, functional, or other properties of the proteins. Unique physical-chemical or structural properties allow the proteins to be separated and identified by electrophoretic procedures, such as, native or denaturing gel electrophoresis or isoelectric focusing, or by chromatographic techniques such as ion exchange, liquid chromatography or gel exclusion chromatography. The unique structures of individual proteins offer opportunities for use of specific antibodies to detect their presence in formats such as an ELISA assay. Combinations of approaches may be employed with even greater specificity such as Western blotting in which antibodies are used to locate individual gene products that have been separated by electrophoretic techniques. Additional techniques may be employed to absolutely confirm the identity of the enzyme such as evaluation by amino acid sequencing following purification. Other procedures may be additionally used.

The expression of a gene product can also be determined by evaluating the phenotypic results of its expression. These assays also may take many forms including but not limited to analyzing changes in the chemical composition, morphology, or physiological properties of the plant. Chemical composition may be altered by expression of preselected DNA segments encoding storage proteins which change amino acid composition and may be detected by amino acid analysis.

### Hosts

Terpenes, including diterpenes and terpenoids, can be made in a variety of host organisms either in vitro or in vivo. In some cases, the enzymes described herein can be made in host cells, and those enzymes can be extracted from the host cells for use in vitro. As used herein, a “host” means a cell, tissue or organism capable of replication. The host can have an expression cassette or expression vector that can include a nucleic acid segment encoding an enzyme that is involved in the biosynthesis of terpenes.

The term “host cell”, as used herein, refers to any prokaryotic or eukaryotic cell that can be transformed with an expression cassettes or vector carrying the nucleic acid segment encoding an enzyme that is involved in the biosynthesis of one or more terpenes. The host cells can, for example, be a plant, bacterial, insect, or yeast cell. Expression cassettes encoding biosynthetic enzymes can be incorporated or transferred into a host cell to facilitate manufacture of the enzymes described herein or the terpene, diterpene, or terpenoid products of those enzymes. The host cells can be present in an organism. For example, the host cells can be present in a host such as a plant.

For example, the enzymes, terpenes, diterpenes, and terpenoids can be made in a variety of plants or plant cells. Although some of the enzymes described herein are from species of the mint family, the enzymes, terpenes, diterpenes, and terpenoids can be made in species other than in mint plants or mint plant cells. The terpenes, diterpenes, and terpenoids can, for example, be made and extracted from whole plants, plant parts, plant cells, or a combination thereof. Enzymes can conveniently, for example, be produced in bacterial, insect, plant, or fungal (e.g., yeast) cells.

Examples of host cells, host tissues, host seeds and plants that may be used for producing terpenes and terpenoids (e.g., by incorporation of nucleic acids and expression systems described herein) include but are not limited to those useful for production of oils such as oilseeds, camelina, canola, castor bean, corn, flax, lupins, peanut, potatoes, safflower, soybean, sunflower, cottonseed, oil firewood trees, rapeseed, rutabaga, sorghum, walnut, and various nut species. Other types host cells, host tissues, host seeds and plants that can be used include fiber-containing plants, trees, flax, grains (maize, wheat, barley, oats, rice, sorghum, millet and rye), grasses (switchgrass, prairie grass, wheat grass, sudangrass, sorghum, straw-producing plants), softwood, hardwood and other woody plants (e.g., poplar, pine, and eucalyptus), oil (oilseeds, camelina, canola, castor bean, lupins, potatoes, soybean, sunflower, cottonseed, oil firewood trees, rapeseed, rutabaga, sorghum), starch plants (wheat, potatoes, lupins, sunflower and cottonseed), and forage plants (alfalfa, clover and fescue). In some embodiments the plant is a gymnosperm. Examples of plants useful for pulp and paper production include most pine species such as loblolly pine, Jack pine, Southern pine, Radiata pine, spruce, Douglas fir and others. Hardwoods that can be modified as described herein include aspen, poplar, eucalyptus, and others. Plants useful for making biofuels and ethanol include corn, grasses (e.g., miscanthus, switchgrass, and the like), as well as trees such as poplar, aspen, pine, oak, maple, walnut, rubber tree, willow, and the like. Plants useful for generating forage include legumes such as alfalfa, as well as forage grasses such as bromegrass, and bluestem. In some cases, the plant is a Brassicaceae or other Solanaceae species. In some embodiments, the plant is not a species of Arabidopsis, for example, in some embodiments, the plant is not Arabidopsis thaliana.

Additional examples of hosts cells and host organisms include, without limitation, tobacco cells such as Nicotiana benthamiana, Nicotiana tabacum, Nicotiana rustica, Nicotiana excelsior, and Nicotiana excelsiana cells; cells of the genus Escherichia such as the species Escherichia coli, cells of the genus Clostridium such as the species Clostridium ljungdahlii, Clostridium autoethanogenum or Clostridium kluyveri, cells of the genus Corynebacterium such as the species Corynebacterium glutamicum, cells of the genus Cupriavidus such as the species Cupriavidus necator or Cupriavidus metallidurans; cells of the genus Pseudomonas such as the species Pseudomonas fluorescens Pseudomonas putida or Pseudomonas oleovorans; cells of the genus Delftia such as the species Delftia acidovorans; cells of the genus Bacillus such as the species Bacillus subtilis, cells of the genus Lactobacillus such as the species Lactobacillus delbrueckii, or cells of the genus Lactococcus such as the species Lactococcus lactis.

“Host cells” can further include, without limitation, those from yeast and other fungi, as well as, for example, insect cells. Examples of suitable eukaryotic host cells include yeasts and fungi from the genus Aspergillus such as Aspergillus niger, from the genus Saccharomyces such as Saccharomyces cerevisae, from the genus Candida such as C. tropicalis, C. albicans, C. cloacae, C. guilliermondii, C. intermedia, C. maltosa, C. parapsilosis, and C. zeylenoides; from the genus Pichia (or Komagataella) such as Pichia pastoris; from the genus Yarrowia such as Yarrowia lipolytica; from the genus Issatchenkia such as Issatchenkia orientalis, from the genus Debaryomyces such as Debaryomyces hansenii, from the genus Arxula such as Arxula adeninivorans, or from the genus Kluyveromyces such as Kluyveromyces lactis or from the genera Exophiala, Mucor, Trichoderma, Cladosporium, Phanerochaete, Cladophialophora, Paecilomyces, Scedosporium, and Ophiostoma.

In some cases, the host cells can have organelles that facilitate manufacture or storage of the terpenes, diterpenes, and terpenoids. Such organelles can include lipid droplets, smooth endoplasmic reticulum, plastids, trichomes, vacuoles, vesicles, plastids, and cellular membranes. During and after production of the terpenes, diterpenes, and terpenoids these organelles can be isolated as a semi-pure source of the of the terpenes, diterpenes, and terpenoids.

The Diterpene Skeletons of Lamiaceae and how to Make them

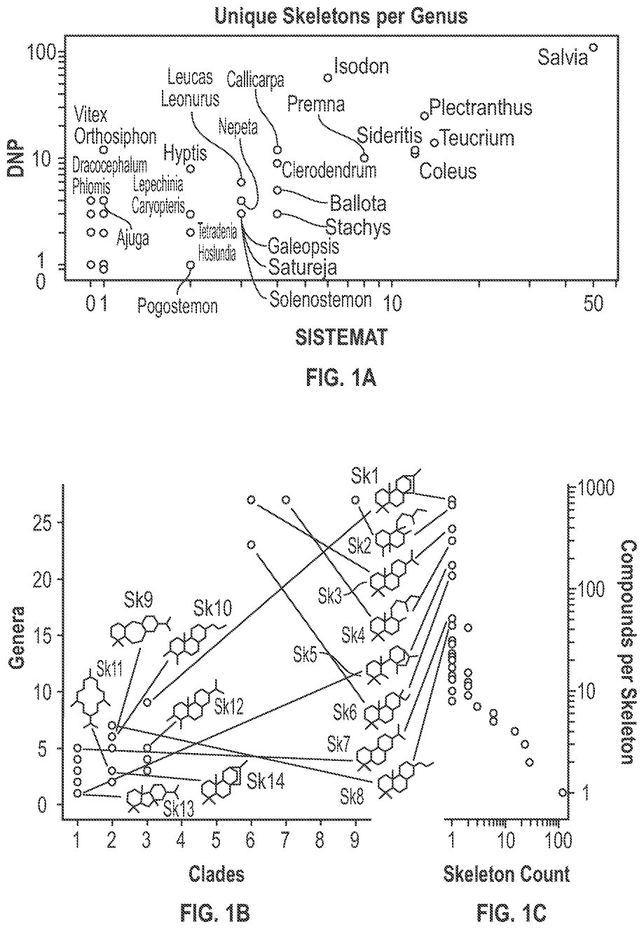

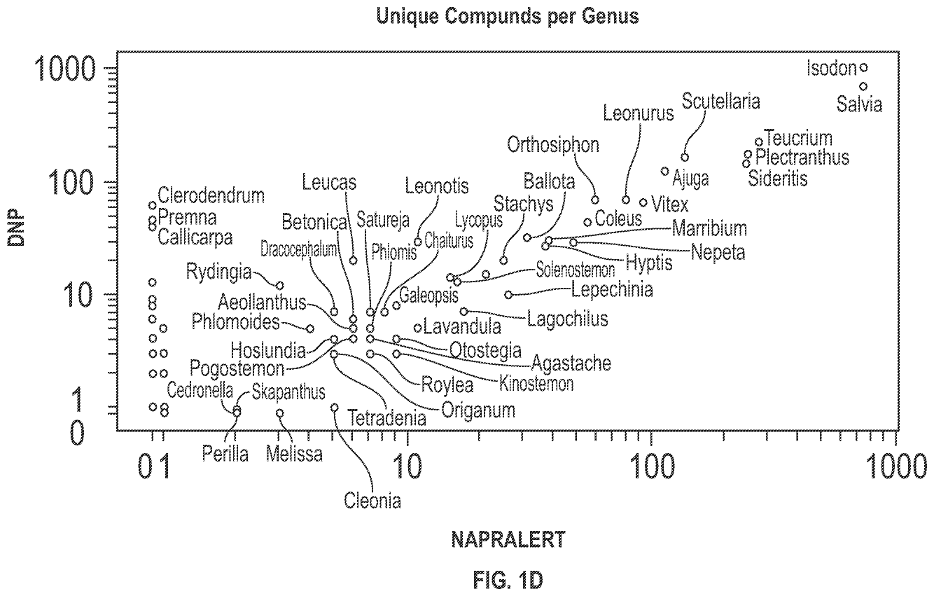

Enzymes responsible for all new skeletons were not specifically located, but considering the known skeletons and diTPS activities, the inventors have deduced how diverse skeletons arise and what strategies may be used for finding the enzymes responsible. All of the six diterpene skeletons with a known biosynthetic route in Lamiaceae contain a decalin core: Sk2, and Sk4 (FIG. 1B-1C) are skeletons of the direct products of TPS-c enzymes, while Sk1, Sk3, Sk6, and Sk14 are skeletons of the products a TPS-e enzyme acting on a labdadiene diphosphate (Sk4) precursor.

Many diterpene skeletons with an intact decalin core can be made by as-yet undiscovered diTPSs from the TPS-c and TPS-e subfamilies, for example through methyl shifts during cyclization. Examples of diTPSs that catalyze methyl shifts are the TPS-c enzymes SdKPS and ArTPS2 which produce the clerodane skeleton (Sk2), and the TPS-e enzyme OmTPS5 which has a product with the abietane skeleton (Sk3). The same mechanisms may form skeletons such as Sk8 and Sk12. Other decalin-containing skeletons, for example the nor-diterpenes (missing one or more methyl side chains, e.g. Sk7) are can be made by oxidative decarboxylation occurring after the TPS steps. Ring rearrangements catalyzed by TPS-e enzymes also have precedent, for example the generation of ent-kaurene (with skeleton Sk1) or ent-atiserene (with skeleton Sk14) from ent-CPP (with skeleton Sk4), but always preserve the decaline core structure.

Diterpenoids lacking a decalin core are taxonomically restricted within Lamiaceae, with no single skeleton being reported in more than two clades (FIG. 1B). Many can be explained as modifications occurring after the TPS steps to decalin-containing skeletons. Cytochrome P450 driven ring contraction, akin to that in the gibberellin pathway, can play a role in the formation of skeletons such as Sk13. Ring opening and ring expansion may also occur, for example in pathways to compounds with the 6,7-seco-kaurane (Sk5), and icetaxane (Sk9) skeletons, respectively. Skeletons such as cembrane (Sk11), lacking any apparent biosynthetic connection to a decalin core can arise from diTPSs outside the TPS-c and TPS-e subfamilies. In Euphorbiaceae and Solanaceae, where cembranoid compounds are common, the relevant TPSs come from the TPS-a subfamily. Elucidation of pathways to the remaining diterpene skeletons in Lamiaceae will depend on broadening the search to new genera and species and new TPS subfamilies, eventually moving beyond TPSs to look at cytochromes P450 and other enzyme families.

### Implications for Biotechnology

Arrays of compounds can be produced by combining class II diTPSs with different class I diTPSs. Particularly prolific enzymes for combinatorial biosynthesis have been Cyc2 from the bacterium Streptomyces griseolosporeus (Hamano et al. J Biol Chem 277(40):37098-37104 (2002); Dairi et 1. J Bacteriol 183(20):6085-4094 (2001)), which generates alkene moieties on prenyl-diphosphate substrates, and SsSS, which installs an alcohol at the 13 position and a double bond at the 14 position; both of these enzymes have demonstrated activity on 12 different class II enzyme products. The inventors have found that SsSS is also active on the products of PcTPS1 and ArTPS2. In addition, the inventors have found class I enzymes that provide routes to products that previously were biosynthetically inaccessible or poorly accessible. OmTPS3 is active on class II products with a labdane skeleton and normal absolute configuration, typically generating a trans-methyl-pentadiene moiety, as in 11, 34, and 24. An enzyme with similar activity, producing 24 and 34, was recently reported from the bacterium Streptomyces cyslabdanicus (Yamada et al. The Journal of Antibiotics 69(7):515-523 (2016); Ikeda et al. J Ind Microbiol Biotechnol 43(2-3):325-342 (2016)) but was not tested against additional substrates. LITPS4 produces sandaracopimaradiene [27] from 31, with greater specificity than the earlier enzyme, Euphorbia peplus TPS8 (Andersen-Ranberg et al. Angew Chem Int Ed 55(6):2142-2146 (2016)). Finally, OmTPS5 enables efficient and specific production of palustradiene 1291 from 31. The other known biosynthetic route to 29 is as a minor spontaneous degradation product of 13-hydroxy-8(14)-abietane from Picea abies levopimaradiene/abietadiene synthase and related enzymes.

ArTPS2 is of particular interest for applications in agricultural biotechnology. Neo-clerodane diterpenoids, particularly those with an epoxide moiety at the 4(18)-position, have garnered significant attention for their ability to deter insect herbivores. The 4(18)-desaturated product of ArTPS2 could be used in biosynthetic or semisynthetic routes to potent insect antifeedants.

### Definitions

As used herein, the singular forms “a,” “an,” and “the” are intended to include the plural forms as well, unless the context clearly indicates otherwise. Also, as used herein, “and/or” refers to, and encompasses, any and all possible combinations of one or more of the associated listed items. Unless otherwise defined, all terms, including technical and scientific terms used in the description, have the same meaning as commonly understood by one of ordinary skill in the art to which this invention pertains.

The term “about”, as used herein, can allow for a degree of variability in a value or range, for example, within 10%, within 5%, or within 1% of a stated value or of a stated limit of a range.

The term “enzyme” or “enzymes”, as used herein, refers to a protein catalyst capable of catalyzing a reaction. Herein, the term does not mean only an isolated enzyme, but also includes a host cell expressing that enzyme. Accordingly, the conversion of A to B by enzyme C should also be construed to encompass the conversion of A to B by a host cell expressing enzyme C.

The term “heterologous” when used in reference to a nucleic acid refers to a nucleic acid that has been manipulated in some way. For example, a heterologous nucleic acid includes a nucleic acid from one species introduced into another species. A heterologous nucleic acid also includes a nucleic acid native to an organism that has been altered in some way (e.g., mutated, added in multiple copies, linked to a non-native promoter or enhancer sequence, etc.). Heterologous nucleic acids can include cDNA forms of a nucleic acid; the cDNA may be expressed in either a sense (to produce mRNA) or anti-sense orientation (to produce an anti-sense RNA transcript that is complementary to the mRNA transcript). For example, heterologous nucleic acids can be distinguished from endogenous plant nucleic acids in that the heterologous nucleic acids are typically joined to nucleic acids comprising regulatory elements such as promoters that are not found naturally associated with the natural gene for the protein encoded by the heterologous gene. Heterologous nucleic acids can also be distinguished from endogenous plant nucleic acids in that the heterologous nucleic acids are in an unnatural chromosomal location or are associated with portions of the chromosome not found in nature (e.g., the heterologous nucleic acids are expressed in tissues where the gene is not normally expressed).

The terms “identical” or percent “identity”, as used herein, in the context of two or more nucleic acids or polypeptide sequences, refer to two or more sequences or subsequences that are the same or have a specified percentage of amino acid residues or nucleotides that are the same (e.g., 75% identity, 80% identity, 85% identity, 90% identity, 95% identity, 97% identity, 98% identity, 99% identity, or 100% identity in pairwise comparison). Sequence identity can be determined by comparison and/or alignment of sequences for maximum correspondence over a comparison window, or over a designated region as measured using a sequence comparison algorithm, or by manual alignment and visual inspection. The percentage is calculated by determining the number of positions at which the identical nucleic acid base or amino acid residue occurs in both sequences to yield the number of matched positions, dividing the number of matched positions by the total number of positions in the window of comparison and multiplying the results by 100 to yield the percentage of sequence identity. A “reference sequence” is a defined sequence used as a basis for a sequence comparison; a reference sequence may be a subset of a larger sequence.

As used herein, a “native” nucleic acid or polypeptide means a DNA, RNA or amino acid sequence or segment that has not been manipulated in vitro, i.e., has not been isolated, purified, amplified and/or modified.

As used herein, the term “plant” is used in its broadest sense. It includes, but is not limited to, any species of grass (fodder, ornamental or decorative), crop or cereal, fodder or forage, fruit or vegetable, fruit plant or vegetable plant, herb plant, woody plant, flower plant or tree. It is not meant to limit a plant to any particular structure. It also refers to a unicellular plant (e.g. microalga) and a plurality of plant cells that are largely differentiated into a colony (e.g. volvox) or a structure that is present at any stage of a plant's development. Such structures include, but are not limited to, a seed, a tiller, a sprig, a stolen, a plug, a rhizome, a shoot, a stem, a leaf, a flower petal, a fruit, et cetera.

The term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture.

As used herein, the term “plant part” as used herein refers to a plant structure or a plant tissue, for example, pollen, an ovule, a tissue, a pod, a seed, a leaf and a cell. Plant parts may comprise one or more of a tiller, plug, rhizome, sprig, stolen, meristem, crown, and the like. In some instances, the plant part can include vegetative tissues of the plant.

The terms “in operable combination,” “in operable order,” and “operably linked” refer to the linkage of nucleic acid sequences in such a manner that a nucleic acid molecule capable of directing the transcription of a coding region (e.g., gene) and/or the synthesis of a desired protein molecule is produced. The term also refers to the linkage of amino acid sequences in such a manner so that a functional protein is produced.

As used herein the term “terpene” includes any type of terpene or terpenoid, including for example any monoterpene, diterpene, sesquiterpene, sesterterpene, triterpene, tetraterpene, polyterpene, and any mixture thereof.

The term “transgenic” when used in reference to a plant or leaf or vegetative tissue or seed for example a “transgenic plant,” transgenic leaf,” “transgenic vegetative tissue,” “transgenic seed,” or a “transgenic host cell” refers to a plant or leaf or tissue or seed that contains at least one heterologous or foreign gene in one or more of its cells. The term “transgenic plant material” refers broadly to a plant, a plant structure, a plant tissue, a plant seed or a plant cell that contains at least one heterologous gene in one or more of its cells.

As used herein, the term “wild-type” when made in reference to a gene refers to a functional gene common throughout an outbred population. As used herein, the term “wild-type” when made in reference to a gene product refers to a functional gene product common throughout an outbred population. A functional wild-type gene is that which is most frequently observed in a population and is thus arbitrarily designated the “normal” or “wild-type” form of the gene.

The following non-limiting Examples describe some procedures that can be performed to facilitate making and using the invention.

### Example 1: Materials and Methods

This Example illustrates some of the materials and methods used in the development of the invention.

### Data Mining

A subset of the NAPRALERT database including all the occurrences of diterpenoids in mint species was obtained. NAPRALERT reports chemical names, but not structures. For Lamiaceae, the species reported in NAPRALERT largely overlap with those from the Dictionary of Natural Products (DNP), which does include structures. A simplifying assumption was therefore made that each unique name represents a unique compound, and structures for the 3080 Lamiaceae diterpenes in NAPRALERT were not all located due to the deficiencies of the NAPRALERT database.

For SISTEMAT, structure files were obtained by redrawing the structures from the publication by Alvarenga et al. (2001) into MarvinSketch (ChemAxon, Budapest. Hungary). The occurrence counts were obtained by transcribing the association table into a spreadsheet. A publicly available digital version of SISTEMAT, called SISTAMATX exists (see website at sistematx.ufpb.br/), but there is no option for bulk downloads, limiting assessment of its completeness or the ability to cross-reference it with other data. For the present work, the proprietary DNP therefore appeared to be one of the only viable option for many analyses.

Lamiaceae diterpene structures were obtained from the DNP by searching for them through the DNP web interface. Additional compounds were found by searching for individual species names for which transcriptome data was available. This additional search step was used because some species have been reclassified between families, or their family is not correctly annotated in the DNP. Records for all the Lamiaceae diterpenes were downloaded and converted into a spreadsheet using a Python script. Species names were extracted from the Biological Source field in a semi-automated method. The DNP contains structural information in the form of IUPAC International Chemical Identifier (InChI) strings (Heller et al. J Cheminform 7 (2015)). In most cases, the DNP InChIs do not include stereochemical information, so for consistency, all stereochemical information was ignored. Skeletons were extracted from the structures using the RDKit (see website at rdkit.org) Python interface. Briefly, all bonds were converted into single bonds, bonds involving at least one non-carbon atom were broken, and the fragment with a carbon-count closest to 35 was retained as the skeleton. The resulting skeletons were then manually examined to correct those where the algorithm chose the wrong fragment, for example, a small number of diterpenoids are attached to acyl chains of more than 20 carbons, in which case the algorithm would incorrectly select the acyl chain as the skeleton; the diterpenoid was therefore selected instead. There are a few cases where sesquiterpenes or other terpenes seemed to have been misannotated in DNP as diterpenes, and those sesquiterpenes or other terpenes were left in the dataset, but their presence or absence does not significantly change any of the analyses.

For all three databases, genus and species names were cross-referenced to TaxIDs from the NCBI Taxonomy database (Federhen Nucleic Acids Res 40(D1): D136-D143 (2012)), first by automated text comparisons, then by manual inspection of un-matched names. Genus level TaxID assignments were possible for every entry in NAPRALERT and the DNP, but in some cases, species-level TaxID assignments were not possible, so species-level analyses were avoided.

### Phylogenetic Trees

Peptide sequences were aligned using Clustal Omega (v. 1.2.1) (Sievers et al., Molecular Systems Biology 7:539 (2011)) and maximum likelihood trees were generated using RAxML (v. 8.2.11) (Stamatakis Bioinformatics 30(9):1312-1313 (2014)) using automatic model selection and 1000 bootstrap iterations. Tree visualizations were generated using ETE3 (Huerta-Cepas Mol Biol Evol 33(6):1635-1638 (2016)).

Plant Material, RNA Isolation and cDNA Synthesis

The following types of plants were obtained from different commercial nurseries or botanical gardens: Ajuga reptans L., Hyptis suaveolens (L.) Poit., Leonotis leonurus (L.) R.Br., Mentha spicata L., Nepeta mussinii Spreng. ex Henckel, Origanum majorana L., Perovskia atriplicifolia Benth., Plectranthus barbatus, Pogostemon cablin (Blanco) Benth., Prunella vulgaris L., and Salvia officinalis L. The plants were grown in a greenhouse under ambient photoperiod and 24° C. day/17° C. night temperatures. Nicotiana benthamiana were grown in a greenhouse under 16 h light (24° C.) and 8 h dark (17° C.) regime.

Total RNA from leaf tissues of A. reptans, N. mussinii, L. leonurus, P. atriplicifolia, and S. officinalis was extracted using methods described by Hamberger et al. (Plant Physiology 157(4):1677-1695 (2011)). Total RNA from leaves of P. vulgaris, M. spicata, P. cablin, H. Suaveolens, O. majorana was extracted using the Spectrum Plant Total RNA Kit (Sigma-Aldrich, St. Louis, MO, USA). RNA extraction was followed by DNase I digestion using DNA-Free™ DNA Removal Kit (Thermo Fisher Scientific, Waltham, MA, USA). First-strand cDNAs were synthesized from 5 μg of total RNA, with oligo(dT) primer, using the RevertAid First Strand cDNA Synthesis Kit (Thermo Fisher Scientific, Waltham, MA, USA). cDNA was diluted 5-fold and used as template for cloning of full length cDNAs. See Table 2 for primers and other oligonucleotides.

Characterization of diTPS Genes by Transient Expression in N. benthamiana

Full length coding sequences of diTPSs were cloned into pEAQ-HT vector (Sainsbury et al., 2009: kindly provided by Prof. G. Lomonossoff, John Innes Centre, UK) using In-Fusion® HD Cloning Plus (Takara Bio, California, USA). pEAQ-HT vector contains a copy of anti-post transcriptional gene silencing protein p19 that suppresses the silencing of transgenes (Voinnet et al. The Plant Journal 33(5):949-956). Expression vectors carrying full length coding sequence of candidate diTPS genes were transformed into the LBA4404 A. tumefaciens strain by electroporation. DXS and GGPPS are known to be the rate limiting enzymes in GGPP biosynthesis and have been shown to substantially increase the production of diterpenes in N. benthamiana system. Therefore, the Plectranthus barbatus 1-deoxy-D-xylulose 5-phosphate synthase (CfDXS) (genbank accession: KP889115) and geranylgeranyl diphosphate synthase (CfGGPPS) (genbank accession: KP889114) coding regions were cloned, and a chimeric polyprotein was created with a LP4-2A hybrid linker peptide between CfDXS and CfGGPPS. LP4/2A contains the first nine amino acids of LP4 (a linker peptide originating from a natural polyprotein occurring in seeds of Impatiens balsamina) and 20 amino acids of the self-processing FMDV 2A (2A is a peptide from the foot-and-mouth disease virus).

The transformed A. tumefaciens were subsequently transferred to 1 mL SOC media and grown for 1 hour at 28° C. 100 μL cultures were transferred to LB-agar solid media containing 50.0 μg/mL rifampicin and 50.0 μg/mL kanamycin and grown for 2 days. A single colony PCR positive clone was transferred to 10 mL LB media in a falcon tube containing 50.0 μg/mL rifampicin and 50.0 μg/mL kanamycin and grown at 28° C. over-night (at 225 rpm). About 1% of the primary culture was transferred to 25 mL of fresh LB media and grown overnight. Cells were pelleted by centrifugation at 4000×g for 15 min and resuspended in 10 mL water containing 200 μM acetosyringone. Cells were diluted with water-acetosyringone solution to a final OD₆₀₀ of 1.0 and incubated at 28° C. for 2-3 hours to increase the infectivity. Equal volumes of culture containing the plasmids with cDNA encoding different diTPS genes were mixed. Each combination of A. tumefaciens culture mixture was infiltrated into independent 4-5 weeks old N. benthamiana plants. Plants were grown for 5-7 days in the greenhouse before metabolite extraction. Leaf discs of 2 cm diameter (approximately 0.1 g fresh weight) were cut from the infiltrated leaves. Diterpenes were extracted in 1 mL n-hexane with 1 mg/L 1-eicosene as internal standard (IS) at room temperature overnight in an orbital shaker at 200 rpm. Plant material was collected by centrifugation and the organic phase transferred to GC vials for analysis.

### In-Vitro Enzyme Activity Assays

To confirm the biosynthetic products obtained in N. benthamiana, diTPS combinations were tested in in vitro assays as described by Pateraki et al. (Plant Physiol 164(3):1222-1236 (2014)). TargetP (Emanuelsson et al. Journal of Molecular Biology 300(4):1005-1016 (2000)) was used for prediction of the plastidial target sequence. Pseudo mature variants versions of HsTPS1, ArTPS2, PcTPS1, OmTPS3, OmTPS5, SsSS, CfTPS1, CfTPS2 and codon optimized CfTPS3 (IDT, USA), lacking the predicted plastidial targeting sequences were cloned in pET-28b(+) (EMD Millipore, Burlington, MA), then expressed and purified from E. coli. The pET_diTPS constructs were transformed into chemically competent OverExpress™ C41(DE3) cells (Lucigen, Middleton, WI, USA), the cells were inoculated in a starter culture with terrific broth medium and 50 μg mL⁻¹ kanamycin, then grown overnight. About 1% of the starter culture was used to inoculate 50 mL terrific broth medium having 50 μg mL⁻¹ kanamycin, and the culture was grown at 37° C. with mixing at 200 rpm until the OD₆₀₀ reached 0.4. Cultures were grown at 16° C. until an OD₆₀₀ of approximately 0.6-0.8 was achieved at which point cultures were induced by 0.2 mM IPTG. Expression was allowed to proceed overnight, and cells were harvested by centrifugation at 5000 g/4° C. for 15 minutes. Cell pellets were resuspended in lysis buffer containing 20 mM HEPES, pH 7.5, 0.5 M NaCl, 25 mM Imidazole, 5% [v/v] glycerol, using one protease inhibitor cocktail tablet per 100 mL (Sigma Aldrich, St. Louis, MO, USA). Lysozyme (0.1 mg per liter) was added to the cell pellet, and the mixture was gently shaken for 30 min, then lysed by sonication. Cell lysate was centrifuged for 25 min at 14000 g, and the supernatant was subsequently used for purification of the recombinant proteins. Proteins were purified on 1-mL His SpinTrap columns (GE Healthcare Life Sciences, Piscataway, NJ, USA) using elution buffer (HEPES, pH 7.5, 0.5 M NaCl, 5% [v/v] glycerol, 350 mM Imidazole and 5 mM dithiothreitol [DTT]) and desalted on PD MiniTrap 0-25 columns (GE Healthcare, Life Sciences, Piscataway, NJ, USA) with a desalting buffer (20 mM HEPES, pH 7.2, 350 mM NaCl, 5 mM DTT, 1 mM MgCl₂, 5% [v/v] glycerol). In-vitro diTPS assays were performed by adding 15 μM GGPP and 50-100 μg purified enzymes in 400 μL enzyme assay buffer (50 mM HEPES, pH 7.2, 7.5 mM MgCl₂, 5% [v/v] glycerol, 5 mM DTT). 500 mL n-hexane (Fluka GC-MS grade) containing 1 ng/ml 1-eicosene as internal standard was gently added as an overlay onto the reaction mix. Assays were incubated for 60-120 min at 30° C. with mixing at approximately 75 rpm, and the hexane overlay was subsequently removed by centrifugation at 1500×g for 15 min before proceeding for GC-MS analysis.

Metabolite Analysis of O. majorana

Fresh leaf, stem, root, and flowers (20 to 50 mg) of O. majorana were harvested. Flowers were further separated with forceps into two parts, the green part (“calyx”), and the rest of the flower (“corolla”). Tissues were extracted overnight in 500 μL of methyl tert-butyl ether. Extracts were concentrated to 100 μL and subjected to GC-MS analysis.

### Compound Purification

For bulk production of diterpenes for structural determination, 15-30 N. benthamiana plants were vacuum infiltrated with diTPS combinations as well as CfGGPPS and CfDXS (46). After 5 days, 100-200 g (fresh weight) of leaves were subjected to two rounds of overnight extractions in 500 mL hexane, which was then concentrated using a rotary evaporator. Compounds were purified on silica gel columns using a mobile phase of hexane with 0-20% ethyl-acetate. In some cases, additional rounds of column purification, or preparative TLC using a hexane/ethyl-acetate or chloroform/methanol mobile phase, were necessary to obtain compounds of sufficient purity for structural determination by NMR.

### GC-MS

All GC-MS analyses were performed on an Agilent 7890A GC with an Agilent VF-5 ms column (30 m×250 μm×0.25 μm, with 10m EZ-Guard) and an Agilent 5975C detector. For N. benthamiana and in-vitro assays, the inlet was set to 250° C. splitless injection, using helium carrier gas with column flow of 1 mL/min. The oven program was 45° C. hold 1 min, 40° C./min to 230° C., 7° C./min to 320° C. hold 3 min. The detector was activated after a four-minute solvent delay. For analysis of O. majorana extracts, conditions were the same, except that the solvent cutoff was set to six minutes to allow monoterpenes to pass, and the oven program was a 45° C. hold for 1 min., 40° C./min to 200° C. 5° C./min to 260° C., 40° C./min to 320° C. with a hold for 3 min.

### NMR and Optical Rotation

The NMR spectra for trans-biformene (Yamada et al. The Journal of Antibiotics 69(7):515-523 (2016)) were measured on a Bruker AVANCE 900 MHz spectrometer. All other spectra were measured on an Agilent DirectDrive2 500 MHz spectrometer. All NMR was done in CDCl₃ solvent. The CDCl₃ peaks were referenced to 7.24 ppm and 77.23 ppm for ¹H and ¹³C spectra, respectively. To aid in the interpretation of NMR spectra, the NAPROC-13 (Lopez-Perez et al. Bioinformatics 23(23):3256-3257 (2007)), and Spektraris (Fischedick et al., Phytochemistry 113:87-95 (2015)) databases were used. Reconstruction of ¹³C spectra from the literature was performed with MestReNova (Mestrelab Research, Santiago de Compostela, Spain). Optical rotation was measured in chloroform at ambient temperature using a Perkin Elmer Polarimeter 341 instrument.

TABLE 2
List of synthetic oligonucleotides

| | |
|---|---|
| Primer Name | |
| (gene) | Sequence |

Amplification of full length genes from
cDNA synthesized from plant tissues total RNA

| | |
|---|---|
| ZmAN2-F | ATGGTTCTTTCATCGTCTTGCACA |
| (ZmAN2) | (SEQ ID NO: 61) |
| ZmAN2-R | TTATTTTGCGGCGGAAACAGGTTCA |
| (ZmAN2) | (SEQ ID NO: 62) |
| CfTPS2-F | AGATTGAGGATTCCATTGAGTACGTGAAGG |
| (CfTPS2) | (SEQ ID NO: 63) |
| CfTPS2-R | GAAGTTTAATATCCTTCATTCTTTATTACA |
| (CfTPS2) | (SEQ ID NO: 64) |
| CfTPS3-F | AGCTCCATTCAACTAGAGTCATGTCGT |
| (CfTPS3) | (SEQ ID NO: 65) |
| CfTPS3-R | TTCATCTGGCTTAACTAGTTGCTGACAC |
| (CfTPS3) | (SEQ ID NO: 66) |
| CfTPS16-F | TTAAAGTACTCTCTCAAAGAGTACTTTGG |
| (CfTPS16) | (SEQ ID NO: 67) |
| CfTPS16-R | GCGACCAACCATCATACGACT |
| (CfTPS16) | (SEQ ID NO: 68) |
| LITPS1-F | AATGGCCTCCACTGCATCCACTCTA |
| (LITPS1) | (SEQ ID NO: 69) |
| LITPS1-R | CCATACTCATTCAACTGGTTCGAACA |
| (LITPS1) | (SEQ ID NO: 70) |
| LITPS4-F | AGCCTGTGTACTCGAAATGTC |
| (LITPS4) | (SEQ ID NO: 71) |
| LITPS4-R | CAAGAGGATGATTCATGTACCAAC |
| (LITPS4) | (SEQ ID NO: 72) |
| SoTPS1-F | TCTCTTTCAAGAATATCCCCTCTC |
| (SoTPS1) | (SEQ ID NO: 73) |
| SoTPS1-R | GGCATTCAATGATTTTGAGTCG |
| (SoTPS1) | (SEQ ID NO: 74) |
| ArTPS1-F | AAATGGCCTCTTTGTCCACTCTC |
| (ArTPS1) | (SEQ ID NO: 75) |
| ArTPS1-R | TTACGCAACTGGTTCGAAAAGCA |
| (ArTPS1) | (SEQ ID NO: 76) |
| ArTPS2-F | TAATGTCATTTGCTTCCCAAGCCA |
| (ArTPS2) | (SEQ ID NO: 77) |
| ArTPS2-R | GGCCTAGACTATACCTTCTCAAACAA |
| (ArTPS2) | (SEQ ID NO: 78) |
| ArTPS3-F | AATGTCACTCTCGTTCACCATCAA |
| (ArTPS3) | (SEQ ID NO: 79) |
| ArTPS3-R | ACTTCAAGAGGATGAAGTGTTTAGG |
| (ArTPS3) | (SEQ ID NO: 80) |
| PaTPS1-F | CTCCAAAACTCGGGCCGGTAAAT |
| (PaTPS1) | (SEQ ID NO: 81) |
| PaTPS1-R | TACGTATTTCCTCACAATCGAGCA |
| (PaTPSI) | (SEQ ID NO: 82) |
| PaTPS3-F | CTAGAAATGTTACTTGCGTTCAAC |
| (PaTPS3) | (SEQ ID NO: 83) |
| PaTPS3-R | GGGTAAGAGTTGAATTTAGATGTCT |
| (PaTPS3) | (SEQ ID NO: 84) |
| NmTPS1-F | ATGACTTCAATATCCTCTCTAAATTTGAGC |
| (NmTPS1) | (SEQ ID NO: 85) |
| NmTPS1-R | GAATATAGTAATCAGACGACCGGTCCA |
| (NmTPS1) | (SEQ ID NO: 86) |
| NmTPS2-F | GCCATATCATGTCTCTTCCGCTCT |
| (NmTPS2) | (SEQ ID NO: 87) |
| NmTPS2-R | TTATTCATGCACCTTAAAATCCTTGAGAG |
| (NmTPS2) | (SEQ ID NO: 88) |
| OmTPS1-F | ATGACCGATGTATCCTCTCTTCGT |
| (OmTPS1) | (SEQ ID NO: 89) |
| OmTPS1-R | AAACACTCACATAACCGGCCCAA |
| (OmTPS1) | (SEQ ID NO: 90) |
| OmTPS3-F | GTCCTTGCTTTCGGAATACT |
| (OmTPS3) | (SEQ ID NO: 91) |
| OmTPS3-R | GAAGTGATCTACAAGGATTCATAAA |
| (OmTPS3) | (SEQ ID NO: 92) |
| OmTPS4-F | TCATTGATTTGCCCTGCATCCAC |
| (OmTPS4) | (SEQ ID NO: 93) |
| OmTPS4-R | CAAAGCTAGTGCTGCTTCTGATT |
| (0mTPS4) | (SEQ ID NO: 94) |
| OmTPS5-F | ATGGTATCTGCATGTCTAAAACTCAA |
| (0mTPS5) | (SEQ ID NO: 95) |
| OmTPS5-R | CTTTCTCTCTCTTGTGCATCTTAGT |
| (OmTPS5) | (SEQ ID NO: 96) |
| MsTPS1-F | ACGTTCATCTTCAATGAGTTCCA |
| (MsTPS1) | (SEQ ID NO: 97) |
| MsTPS1-R | TACGTGTATGTCGATCTGTTCCAAT |
| (MsTPS1) | (SEQ ID NO: 98) |
| PcTPS1-F | CATGTCATTTGCTTCTCAATCAC |
| (PcTPS1) | (SEQ ID NO: 99) |
| PcTPS1-R | CCCATTATCTAAAAGTCTACATCACC |
| (PcTPS1) | (SEQ ID NO: 100) |
| HsTPS1-F | TCCTCATAAAGCAATGGCGTATA |
| (HsTPS1) | (SEQ ID NO: 101) |
| HsTPS1-R | CTAAGATTCAGACAATGGGCTCA |
| (HsTPS1) | (SEQ ID NO: 102) |
| EpTPS8-F | GCAGACGCCAATCTTTCTTGGT |
| (EpTPS8) | (SEQ ID NO: 103) |
| EpTPS8-R | TTATGAAGTTAAAAGGAGTGGTTCGTTGAC |
| (EpTPS8) | (SEQ ID NO: 104) |
| PVTPS1-F | GGAACGAGAAATGTCACTCAC |
| (PVTPS1) | (SEQ ID NO: 105) |
| PVTPS1-R | TTCTAGTTTCTCACAGAAGTCAA |
| (PVTPS1) | (SEQ ID NO: 106) |
| LP4-2A Ver.1 | TCAAATGCAGCAGACGAAGTTGCTACT |
| sequence | CAACTTTTGAATTTTGACTTGCTGAAGTT |
| | GGCTGGTGATGTTGAGTCAAACCCTGGACCT |
| | (SEQ ID NO: 107) |

Cloning of full length diTPS genes into pEAQ-HT
for transient expression in N. benthamiana

| | |
|---|---|
| pEAQ_Infusion | TTCTGCCCAAATTCGATGGGGTCTCTATC |
| _CfTPS1-F | CACTATGA |
| (CfTPS1) | (SEQ ID NO: 108) |
| pEAQ_Infustion | AGTTAAAGGCCTCGATCAGGCGACTGGTTCG |
| _CfTPS1-R | AA |
| (CfTPS1) | AAGTA (SEQ ID NO: 109) |
| pEAQ_Infusion | TTCTGCCCAAATTCGATGTCGCTCGCCTT |
| _SsSCS-F | CAAC |
| (SsSS) | (SEQ ID NO: 110) |
| pEAQ_Infusion | AGTTAAAGGCCTCGATCAAAAGACAAAGGAT |
| _SsSCS-R | T |
| (SsSS) | TCATA (SEQ ID NO: 111) |
| pEAQ_Infusion | TTCTGCCCAAATTCGATGGTTCTTTCATCG |
| _ZrnAN2-F | TCTT |
| (ZmAN2) | GCAC (SEQ ID No: l12) |
| pEAQ_Infusion | AGTTAAAGGCCTCGATTATTTTGCGGCGGAA |
| _ZmAN2-R | AC |
| (ZmAN2) | AGGT (SEQ ID NO: 113) |
| pEAQ_Infusion | TTCTGCCCAAATTCGATGAAAATGTTGATG |
| _CfTPS2-F | ATCA |
| (CfTPS2) | AAAGT (SEQ ID NO: 114) |
| pEAQ_Infusion_ | AGTTAAAGGCCTCGATCAGACCACTGGTT |
| CfTPS2-R | CAAA |
| (CfTPS2) | TAGTA (SEQ ID NO: 115) |
| pEAQ_Infusion_ | TTCTGCCCAAATTCGATGTCGTCCCTCGCC |
| CfTPS3-F | GGC |
| (CfTPS3) | AACCT (SEQ ID NO: 116) |
| pEAQ_Infusion | AGTTAAAGGCCTCGACTAGTTGCTGACACAA |
| _CfTPS3-R | CT |
| (CfTPS3) | CATT (SEQ ID NO: 117) |
| pEAQ_Infusion | TTCTGCCCAAATTCGATGCAGGCTTCTATGTC |
| _CfTPS16-F | ATCT |
| (CfTPS16) | (SEQ ID NO: 118) |
| pEAQ_infusion | AGTTAAAGGCCTCGATCATACGACTGGTTCA |
| _CfTPS16-R | AA |
| (CfTPS16) | CATT (SEQ ID NO: 119) |
| pEAQ_Infusion | TTCTGCCCAAATTCGATGGCCTCCACTGCATC |
| _LITPS1-F | C |
| (LITPS1) | (SEQ ID NO: 120) |
| pEAQ_Infusion | AGTTAAAGGCCTCGATCATTCAACTGGTTCGA |
| _LITPS1-R | ACAA |
| (LITPS1) | (SEQ ID NO: 121) |
| pEAQ_Infusion | TTCTGCCCAAATTCGATGATTCCTAATCCCGA |
| _LITPS2-F | AA |
| (LITPS2) | (SEQ ID NO: 122) |
| pEAQ_Infusion | AGTTAAAGGCCTCGATTACATTGGCAATCCG |
| _LITPS2-R | ATGAA |
| (LITPS2) | (SEQ ID NO: 123) |
| pEAQ_Infusion | TTCTGCCCAAATTCGATGTCGGTGGCGTTCAA |
| _LITPS4-F | CCT |
| (LITPS4) | (SEQ ID NO: 124) |
| pEAQ_Infusion | AGTTAAAGGCCTCGATCAAGAGGATGATTCA |
| _LITPS4-R | TG |
| (LITPS4) | TACC (SEQ ID NO: 125) |
| pEAQ_Infusion | TTCTGCCCAAATTCGATGTCCCTCGCCTTCAA |
| _SoTPS1-F | CG |
| (SoTPS1) | (SEQ ID NO: 126) |
| pEAQ_/Infusion | AGTTAAAGGCCTCGATCATTTGCCACTCACAT |
| _SoTPS1-R | TT |
| (SoTPS1) | (SEQ ID NO: 127) |
| pEAQ_infusion | TTCTGCCCAAATTCGATGGCCTCTTTGTCCAC |
| _ArTPS1-F | TTTCC |
| (ArTPS1) | (SEQ ID NO: 128) |
| pEAQ_/Infusion | AGTTAAAGGCCTCGATCACGCAACTGGTTCG |
| _ArTPS1-R | AAA |
| (ArTPS1) | AGA (SEQ ID NO: 129) |
| pEAQ_Infusion | TTCTGCCCAAATTCGATGTCATTTGCTTCCCA |
| _ArTPS2-F | AG |
| (ArTPS2) | CCAC (SEQ ID NO: 130) |
| pEAQ_Infusion | AGTTAAAGGCCTCGACTAGACTACCTTCTCAA |
| _ArTPS2-R | ACA |
| (ArTPS2) | ATAC (SEQ ID NO: 131) |
| pEAQ_Infusion | TTCTGCCCAAATTCGATGTCACTCTCGTTCAC |
| _ArTPS3-F | CATCA |
| (ArTPS3) | (SEQ ID NO: 132) |
| pEAQ_Infusion | AGTTAAAGGCCTCGATCAAGAGGATGAAGTG |
| _ArTPS -R | TTTAG |
| (ArTPS3) | (SEQ ID NO: 133) |
| pEAQ_Infusion | TTCTGCCCAAATTCGATGACCTCTATGTCCTC |
| _PaTPS1-F | TCTAA |
| (PaTPS1) | (SEQ ID NO: 134) |
| pEAQ_Infusion | AGTTAAAGGCCTCGATCATACGACCGGTCCA |
| _PaTPS1-R | AAC |
| (PaTPS1) | AGT (SEQ ID NO: 135) |
| pEAQ_Infusion | TTCTGCCCAAATTCGATGTTACTTGCGTTCAA |
| _PaTPS3-F | CATA |
| (PaTPS3) | AGC (SEQ ID NO: 136) |
| pEAQ_Infusion | AGTTAAAGGCCTCGATTAATTAGGTAGGTAG |
| _PaTPS3-R | AGGG |
| (PaTPS3) | GTT (SEQ ID NO: 137) |
| pEAQ_Infusion | ATATTCTGCCCAAATTCGATGACTTCAATATC |
| __NmTPS1-F | CTCT |
| (NmTPS1) | CTAAATTTGAGCAATG (SEQ ID NO: 138) |
| pEAQ_Infusion | CAGAGTTAAAGGCCTCGATCAGACGACCGGT |
| _NmTPS1-R | CCAA |
| (NmTPS1) | (SEQ ID NO: 139) |
| pEAQ_Infusion | TTCTGCCCAAATTCGATGTCTCTTCCGCTCTC |
| _NmTPS2-F | CTCT |
| (NmTPS2) | (SEQ ID NO: 140) |
| pEAQ_Infusion | GATAAGTTAAAGGCCTCGATTATTCATGCACC |
| _NmTPS2-R | TTA |
| (NmTPS2) | AAATCCTTGAGAGC (SEQ ID NO: 141) |
| pEAQ_Infusion | TTCTGCCCAAATTCGATGACCGATGTATCCTC |
| _OmTPS1-F | TCTTC |
| (OmTPS1) | (SEQ ID NO: 142) |
| pEAQ_Infusion | AGTTAAAGGCCTCGATCACATAACCGGCCCA |
| _OmTPS1-R | AACA |
| (OmTPS1) | (SEQ ID NO: 143) |
| pEAQ_Infusion | TTCTGCCCAAATTCGATGGCGTCGCTCGCGTT |
| _OmTPS3-F | CAC |
| (OmTPS3) | (SEQ ID NO: 144) |
| pEAQ_Infusion | AGTTAAAGGCCTCGACTACAAGGATTCATAA |
| _OmTPS3-R | ATT |
| (OmTPS3) | AAGGA (SEQ ID NO: 145) |
| pEAQ_Infusion | TTCTGCCCAAATTCGCGAATGTCACTCGCCTT |
| _OmTPS4-F | CAGC |
| (OmTPS4) | (SEQ ID NO: 146) |
| pEAQ_Infusion | AGTTAAAGGCCTCGAGCTAGGAGCTTAGGGT |
| _OmTPS4-R | TT |
| (0mTPS4) | TCAT (SEQ ID NO: 147) |
| pEAQ_Infusion | TTCTGCCCAAATTCGATGGTATCTGCATGTCT |
| _OmTPS5-F | AAA |
| (0mTPS5) | (SEQ ID NO: 148) |
| pEAQ_Infusion | AGTTAAAGGCCTCGATCATGAAGGAATTGAA |
| _OmTPS5-R | GGAA |
| (OmTPS5) | (SEQ ID NO: 149) |
| pEAQ_Infusion | TTCTGCCCAAATTCGATGAGTTCCATTCGAAA |
| _MsTPS1-F | TTT |
| (MsTPS1) | AAGT (SEQ ID NO: 150) |
| pEAQ_Infusion | AGTTAAAGGCCTCGATCACTTGAGAGGCTCA |
| _MsTPS1-R | AAC |
| (MsTPS1) | ATCAT (SEQ ID NO: 151) |
| pEAQ_Infusion | TTCTGCCCAAATTCGATGTCATTTGCTTCTCA |
| _PcTPS1-F | AT |
| (PCTPS1) | CAC (SEQ ID NO: 152) |
| pEAQ_Infusion | AGTTAAAGGCCTCGACTACATCACCCTCTCAA |
| _PcTPS1-R | ACA |
| (PcTPS1) | ATAC (SEQ ID NO: 153) |
| pEAQ_Infusion | TTCTGCCCAAATTCGATGGCGTATATGATATC |
| _HsTPS1-F | TAT |
| (HsTPS1) | TTCAAATCTC (SEQ ID NO: 154) |
| pEAQ_/Infusion | AGTTAAAGGCCTCGATCAGACAATGGGCTCA |
| _HsTPS1-R | AAT |
| (HsTPS1) | AGAAC (SEQ ID NO: 155) |
| pEAQ_Infusion | TTCTGCCCAAATTCGATGCAAGTCTCTCTCTC |
| _EpTPS8-F | C |
| (EpTPS8) | CTCA (SEQ ID NO: 156) |
| pEAQ_Infusion | AGTTAAAGGCCTCGATTATGAAGTTAAAAGG |
| __EpTPS8-R | AG |
| (EpTPS8) | TGGTT (SEQ ID NO: 157) |
| pEAQ_Infusion | TTCTGCCCAAATTCGCGAATGTCACTCACTTT |
| _PVTPS1-F | CA |
| (PVTPS1) | ACG (SEQ ID NO: 158) |
| pEAQ_Infusion | AGTTAAAGGCCTCGAGCTAGTTTCTCACAGA |
| _PVTPS1-R | AG |
| (PVTPS1) | TCAA (SEQ ID NO: 159) |

Cloning of diTPS genes into pET-28 b
(+) for E. coli expression

| | |
|---|---|
| pET28_CfTPS1-F | AGGAGATATACCATGGCCGAGATTCGAGTG |
| (CfTPS1) | CCAC |
| | (SEQ ID NO: 160) |
| pET28_CfTPS1-R | GGTGGTGGTGCTCGAAGGCGACTGGTTCGAA |
| (CfTPS1) | AAG |
| | TAC (SEQ ID NO: 161) |
| pET28_SsSS-F | AGGAGATATACCATGGATTTCATGGCGAAAA |
| (SsSS) | TGAA |
| | AGAGA (SEQ ID NO: 162) |
| pET28_SsSS-R | GGTGGTGGTGCTCGAAAAAGACANAGGATTT |
| (SsSS) | CATAT |
| | (SEQ ID NO: 163) |
| pET28_CfTPS2-F | AGGAGATATACCATGCAAATTCGTGGAAAGC |
| (cfTPS2) | AAAG |
| | ATCAC (SEQ ID NO: 164) |
| pET28_CfTPS2-R | GGTGGTGGTGCTCGAAGACCACTGGTTCAAA |
| (CfTPS2) | TAG |
| | AACT (SEQ ID NO: 165) |
| pET28_CfTPS3-F | AGGAGATATACCATGTCTAAATCATCTGCAG |
| (CfTPS3) | CTGT |
| | (SEQ ID NO: 166) |
| pET28_CfTPS3-R | GGTGGTGGTGCTCGAAGTTGCTGACACAACT |
| (CfTPS3) | CATT |
| | (SEQ ID NO: 167) |
| pET28_OmTPS3-F | AGGAGATATACCATGACCGTCAAATGCTAC |
| (OmTPS3) | (SEQ ID NO: 168) |
| pET28_OmTPS3-R | GGTGGTGGTGCTCGAACAAGGATTCATAAAT |
| (OmTPS3) | TAAG |
| | (SEQ ID NO: 169) |
| pET28_OmTPS5-F | AGGAGATATACCATGACTGTCAAGTGCAGC |
| (OmTPS5) | (SEQ ID NO: 170) |
| pET28_OmTPS5-R | GGTGGTGGTGCTCGAATGAAGGAATTGAAG |
| (OmTPS5) | (SEQ ID NO: 171) |
| pET28_PcTPS1-F | AGGAGATATACCATGTTTATGCCCACTTCCAT |
| (pcTPS1) | TAA |
| | ATGTA (SEQ ID NO: 172) |
| pET28_PcTPS1-R | GGTGGTGGTGCTCGAACATCACCCTCTCAAA |
| (PcTPS1) | CAA |
| | TACTTTGG (SEQ ID NO: 173) |
| pET28_HsTPS1-F | AGGAGATATACCATGGTAGCAAAAGTGATCG |
| (HsTPS1) | AGAG |
| | CCGAGTTA (SEQ ID NO: 174) |
| pET28_HsTPS1-R | GGTGGTGGTGCTCGAAGACAATGGGCTCAAA |
| (HsTPS1) | TAGA |
| | ACTTTAAAT (SEQ. ID NO: 175) |

### Example 2: Diversity of Diterpenoids in Lamiaceae

To help determine the most promising species in which to find previously unknown but useful diterpene synthase (diTPS) activities, a dataset of diterpene occurrences in Lamiaceae species and a dataset of functionally characterized diTPS genes from Lamiaceae were generated. Information about diterpene occurrence was collected from three sources, SISTEMAT, DNP, and NAPRALERT.SISTEMAT (Vestri et al. Phytochemistry 56(6):583-595 (2001)) contains Lamiaceae diterpenes reported up to 1997, including 91 unique carbon skeletons (the core alkanes, disregarding all desaturation, acyl-side chains, heteroatoms, and stereochemistry) from 295 species and 51 genera. An electronic copy of SISTEMAT was not available, so it was reconstructed based on the figures and tables in the paper.

The Dictionary of Natural Products (DNP; see website at dnp.chemnetbase.com, accessed Jan. 11, 2018) includes a wealth of information on diterpenes from Lamiaceae, including full structures and the species where those structures have been reported. NAPRALERT (Loub et al., J Chem Inf Comput Sci 25(2):99-103 (1985)) identifies compounds by their common name rather than their structure or skeleton, but it does associate the compounds to genus and species names, and gives various other information, such as the tissue where the compound was found.

To enable comparison among the databases, and cross-referencing with transcriptome and enzyme data, all genus and species names were converted into TaxIDs from the NCBI Taxonomy database (Federhen Nucleic Acids Res 40(D1): D136-D143 (2012)). To put structure occurrences into clearer evolutionary context, each genus was annotated as a member of one of the 12 monophyletic clades that form the backbone of Lamiaceae, as delineated by Li and colleagues (Li et al. Scientific Reports 6:34343 (2016)).

In the context of diTPSs, examination of skeletons can be helpful because the skeleton often resembles the diterpene synthase product more obviously than a highly decorated downstream product would. Therefore, the skeletons were extracted from the DNP structures. An example of such skeleton extraction is shown below, where Table 3A provides an example of which class I diTPS generate which products when using a N. benthamiana transient expression. Bold numbers refer to assigned compound numbers; “np” indicates that the combination was tested but no product was detected: “-” indicates that the combination was not tested. The following are newly identified enzymes: LITPS1, HsPS1, PcTPS1, ArTPS2, OmTPS1, ArTPS3, LITPS4, MsTPS1, NmTPS2, OmTPS3, OmTPS4, OmTPS5, PaTPS3, PvTPS1, and SoTPS1.

TABLE 3A
Index of Enzyme Types and Products Observed in Transient Expression Assays

| | CfTPS1 | CfTPS2 | LlTPS1 | ZmAN2 | HsPS1 | PcTPS1 | ArTPS2 | OmTPS1 |
|---|---|---|---|---|---|---|---|---|
| Enzyme | [31] | [10] | [5] | [16] | [21] | [25] | [38] | [31] |
| ArTPS3 | 32 | 8 | 1, 2, 3 | np | — | — | np | — |
| LlTPS4 | 27 | 8 | 1, 2, 3 | np | — | — | — | — |
| MsTPS1 | 27 | 8 | 3 | np | — | — | np | — |
| NmTPS2 | np | np | np | 19 | — | — | np | — |
| OmTPS3 | 34 | 11 | 1, 2 | np | 24 | — | np | 34 |
| OmTPS4 | 33 | 8 | 1, 2, 3, 4 | 20 | — | — | — | 33 |
| OmTPS5 | 29 | 8 | 1, 2, 3 | np | — | — | np | 29 |
| PaTPS3 | 32 | 8 | 1, 2, 3 | np | — | — | — | — |
| PvTPS1 | 32 | 8 | 1, 2, 3 | np | — | — | — | — |
| SoTPS1 | 32 | 8 | 1, 2, 3 | np | — | — | — | — |
| CfTPS3 | 32 | 8 | 1, 2, 3 | np | 22 | np | np | 32 |
| SsSS | 33 | — | 4 | 20 | 23 | 26 | 37 | — |

Table 3B provides an example of an index of new class 11 diTPS enzymes and the products identified by functional assays of these enzymes using the N. benthamiana transient expression assay. The products were identified by GC-MS chromatography of hexane extracts from N. benthamiana transient expression assays that expressed new (+)-CPP synthases or new class II diTPSs along with reference combinations.

TABLE 3B
Products Identified for New Class II diTPS Enzymes

| Enzyme | Product |
|---|---|
| ArTPS1 | Copalyl-PP [31] |
| CfTPS16 | Copalyl-PP [31] |
| NmTPS1 | Copalyl-PP [31] |
| OmTPS1 | Copalyl-PP [31] |
| PaTPS1 | Copalyl-PP [31] |
| ArTPS2 | Neo-cleroda-4(18), 13E-dienyl-PP [38] |
| HsTPS1 | Labda-7,13E-dienyl-PP [21] |
| LlTPS1 | Peregrinol-PP [7] |
| PcTPS1 | Ent-labda-8,13E-dienyl-PP [25] |

Using data like that obtained in Tables 3A and 3B, a labdane skeleton was extracted from the forskolin structure shown below by deleting all heteroatoms, desaturations, and stereochemistry.

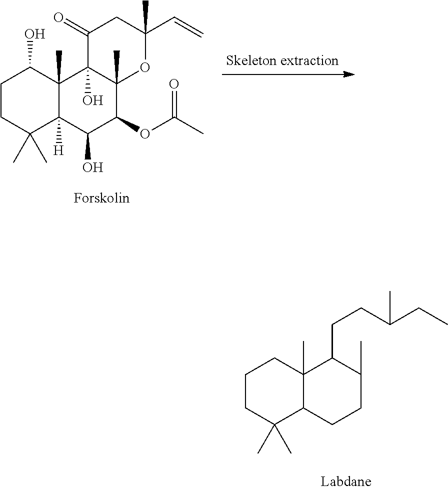

A tabulation of the skeletons from SISTEMAT and DNP was therefore generated.

The three databases were relatively consistent in their estimations of the diversity and distribution of diterpenes and diterpene skeletons, as illustrated in Table 4 and FIG. 1B, 1D.

TABLE 4
Comparison of different sources for data
about Lamiaceae diterpene chemotaxonomy

| | DNP | NAPRALERT | SISTEMAT |
|---|---|---|---|
| Genera | 67 | 60 | 44 |
| Species | 342 | 378 | — |
| Diterpene | 3336 | 3080 | — |
| names | | | |
| Diterpene | 3268 | — | — |
| structures | | | |
| Diterpene | 229 | — | 91 |
| skeletons | | | |

A total of 239 skeletons are represented, with five, the kaurane (Sk1), clerodane (Sk2), abietane (Sk3), labdane (Sk4), and pimarane (Sk6) being, by far, the most widely distributed and accounting for most of the total structures (Table 4, FIG. 1B-1C). The clerodane skeleton, for example, has the widest distribution, having been reported in 27 genera representing 9 of the 12 backbone clades, absent only in Tectona and two clades from which no diterpenes have yet been reported. The large number of less common, taxonomically restricted skeletons, including over 100 skeletons with only one associated compound (FIG. 1C), indicted to the inventors that searching across many species and genera would be a good strategy for finding diterpene synthases with new activities.

### Example 3: Identifying Candidate Diterpene Synthase Genes

Through a comprehensive literature search, a reference set was built of known Lamiaceae diTPSs and their activities. Fifty-four functional diTPSs have been reported in this family, which correspond to thirty class II and 24 class I enzymes. Combinations of these diterpene synthases account for twenty-seven distinct products represented by six different skeletons, the five widely distributed skeletons, Sk1-4 and Sk6, as well as the less common atisane (Sk14) skeleton. This leaves 233 skeletons for which the biosynthetic route remains unknown. Further, a single skeleton can correspond to multiple distinct diTPS products, so there is also a possibility of finding new diTPS activities for skeletons already accounted for by known enzymes.

BLAST homology searches (Camacho et al. BMC Bioinformatics 10: 421 (2009)) were performed to the list of Lamiaceae diTPSs to mine 48 leaf transcriptomes made available by the Mint Genome Project (Boachon et al. Molecular Plant. (2018)) for candidate diTPSs. The number of diTPS candidates was cross-referenced to the number of diterpenes and diterpene skeletons reported from each species and genus (Table 5). Table 5 shows species from which diTPSs were selected for cloning, the total number of diTPS candidate sequences, and the number of unique diterpene structures and skeletons for those species, based on DNP.

TABLE 5
Species from which diTPSs were Isolated

| | | diTPS | | |
|---|---|---|---|---|
| Full name | Code | hits | Diterpenes | Skeletons |
| Ajuga reptans | Ar | 5 | 13 | 2 |
| Hyptis suaveolens | Hs | 7 | 4 | 1 |
| Leonotis leonurus | Ll | 5 | 14 | 2 |
| Mentha spicata | Ms | 5 | 0 | 0 |
| Nepeta mussinii | Nm | 3 | 0 | 0 |
| Origanum majorana | Om | 5 | 0 | 0 |
| Perovskia | Pa | 5 | 2 | 2 |
| atriplicifolia | | | | |
| Plectranthus | Cf | 5 | 50 | 10 |
| barbatus | | | | |
| Pogostemon cablin | Pc | 2 | 0 | 0 |
| Pruneila vulgaris | Pv | 1 | 1 | 1 |
| Salvia officinalis | So | 5 | 13 | 5 |

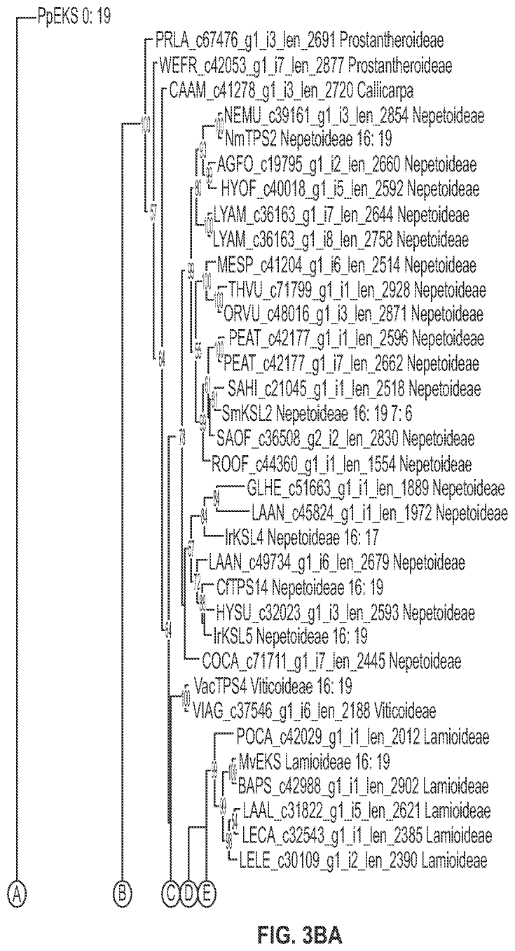

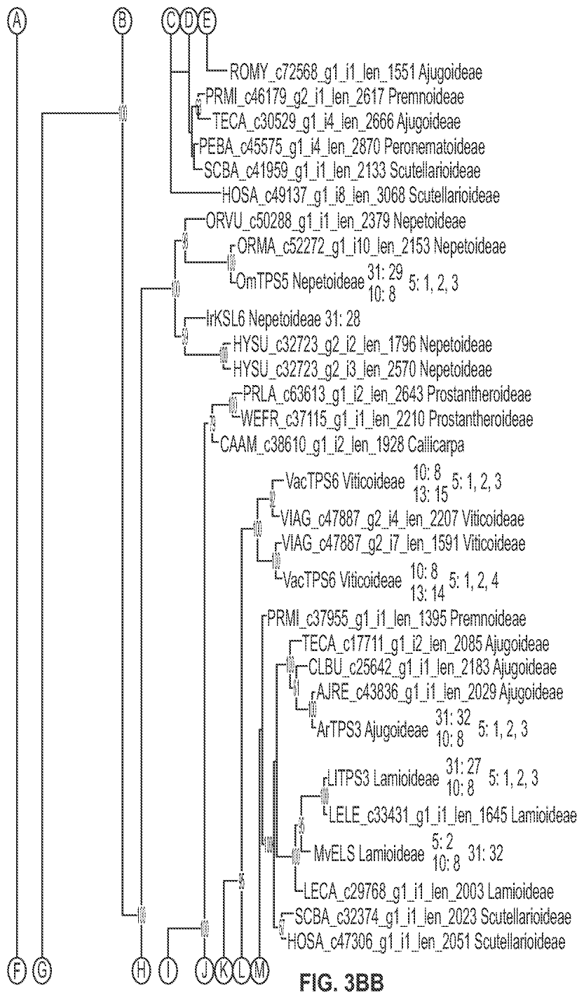

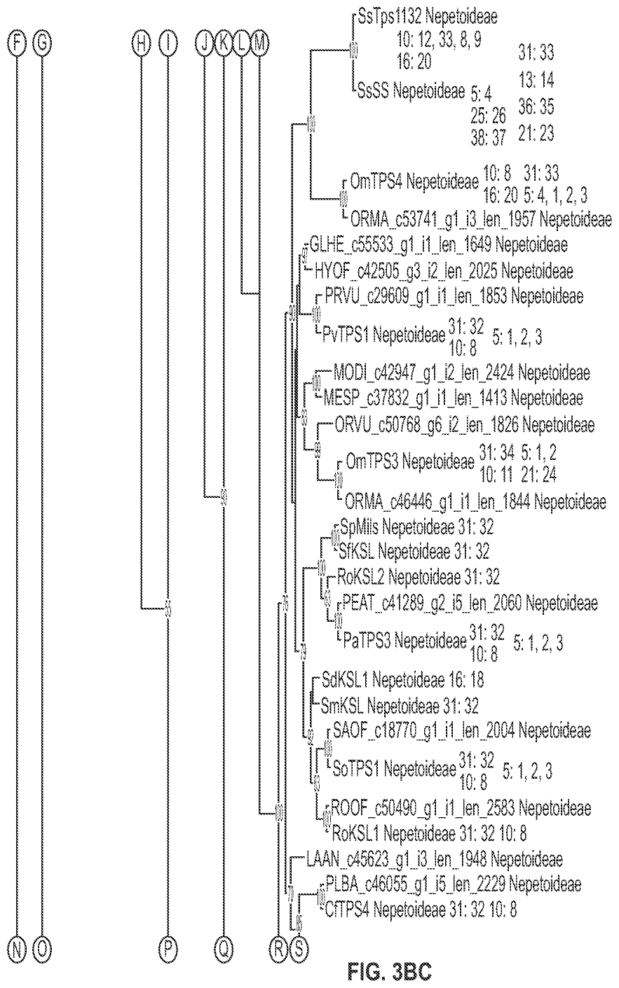

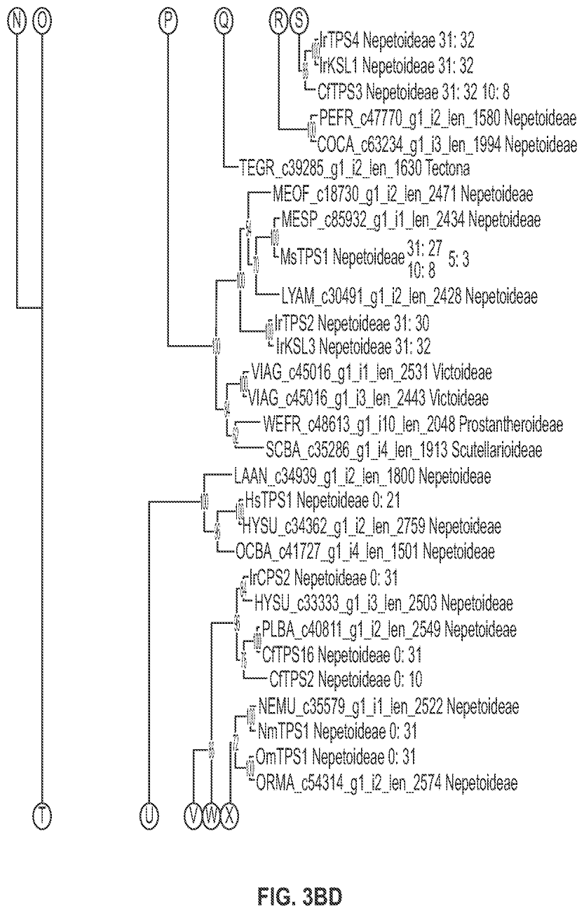

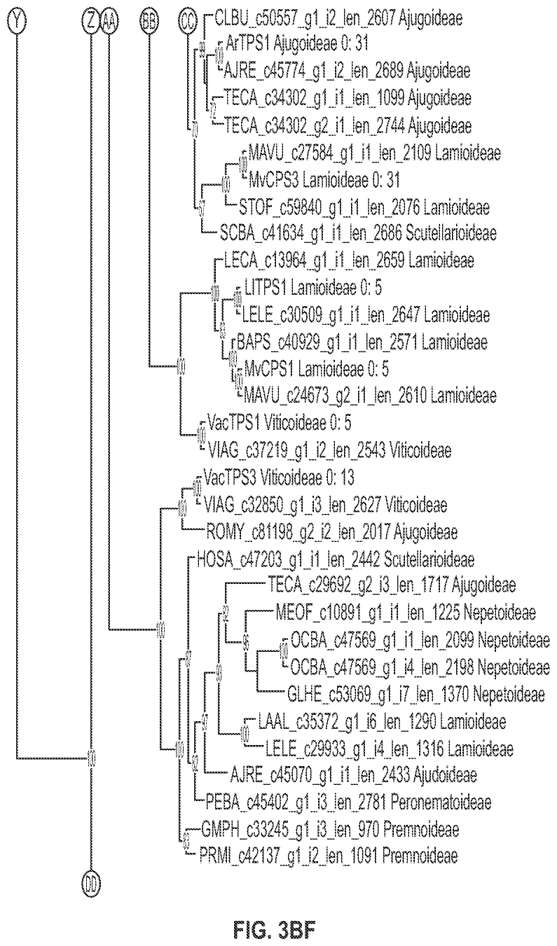

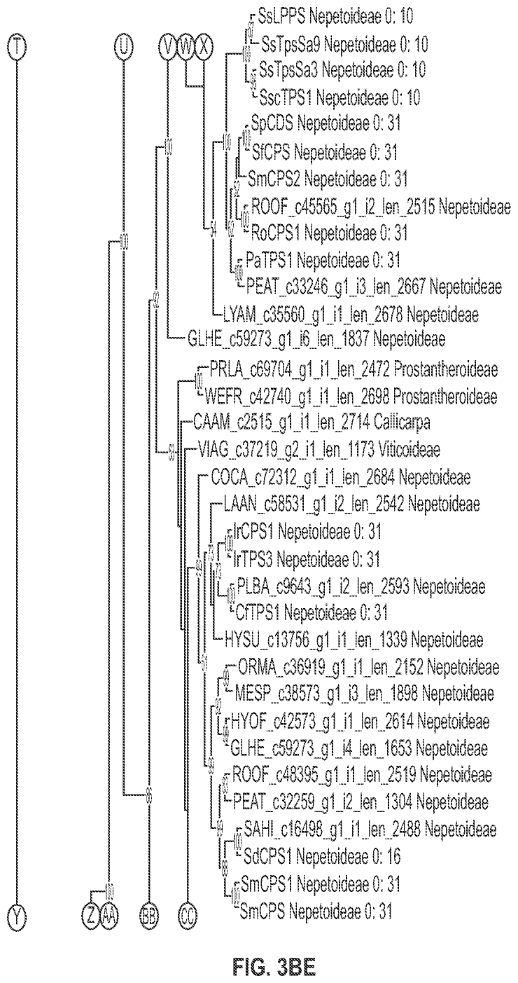

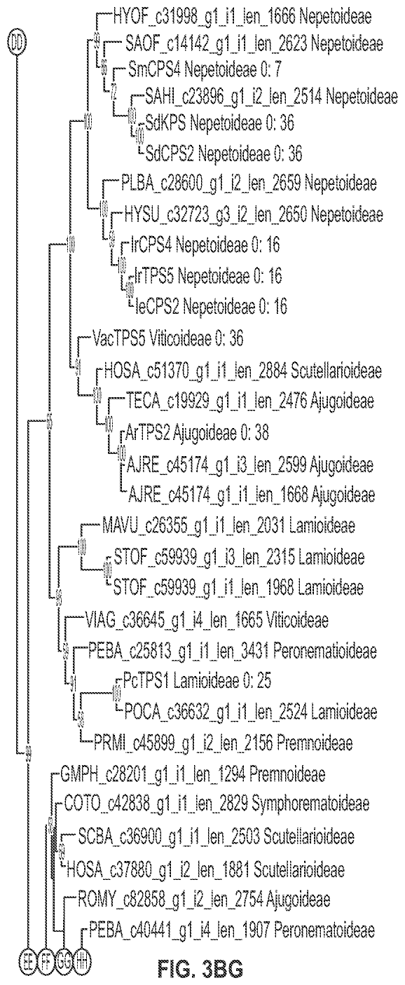

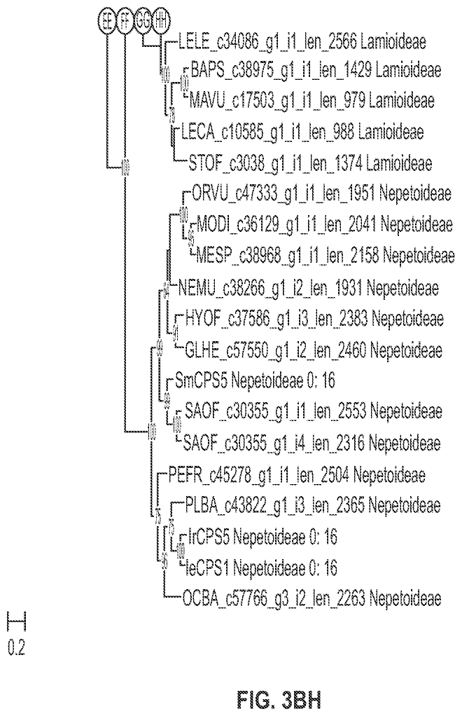

A phylogenetic tree was generated from the peptide sequences from the reference set, alongside those from the new transcriptome data, including established substrates and products for each enzyme (FIG. 3A, 3B-1 to 3B-4). Candidate genes were selected from species such as Mentha x spicata and Origanum majorana, where the transcriptome data showed multiple candidate diTPSs likely existed but where few or no diterpene product structures have been reported. Genes were also selected that had relatively low homology to known enzymes. In this way, the inventors attempted to evenly cover of the sequence homology space. A few candidates from Plectranthus and Salvia were also selected based on the great diversity of diterpenes that have been reported from these genera.

### Example 4: Characterization of Class H dITPSs

FIG. 3A presents a summary of Lamiaceae diTPS structures and activities reported from previous work, together with the newly characterized diTPS activities identified as described herein. Class II activities were established based on the activities of extracts from Nicotiana benthamiana that transiently expressed the new genes, compared with the activities of known diTPS (or combinations) that were similarly expressed.

Class II diTPS products retained the diphosphate group from the GGPP substrate. When expressed in-vivo, whether in E. coli or N. benthamiana, without a compatible class I diTPS, a diphosphate product degrades to the corresponding alcohol, presumably by the action of non-specific endogenous phosphatases. Due to difficulties in purifying and structurally characterizing diphosphate class II products it is customary in the field to instead characterize the alcohol derivatives (Heskes et al. Plant J 93(5):943-958 (2018): Pelot et al. Plant J 89(5):885-897 (2017)), which is the approach taken in this study. For clarity, the alcohol has been indicated by appending an “a” to the compound number, for example, 16a refers to ent-copalol.

ArTPS1, PaTPS1, NmTPS1, OmTPS1, and CfTPS1 were identified as (+)-copalyl diphosphate ((+)-CPP) [31] synthases by comparison to products of Plectranthus barbatus (synonym Coleus forskohli) CfTPS1, and the reference combination of CfTPS1 combined with CfTPS3, yielding miltiradiene (Pateraki et al. Plant Physiol 164(3):1222-1236 (2014)). LITPS1 was identified as a peregrinol diphosphate (PgPP) [5] synthase based on a comparison of products with Marrubium vulgare MvCPS1 (Zerbe et al. Plant J 79(6):914-927 (2014)), and MvCPS1 combined with M. vulgare 9,13-epoxylabdene synthase (MvELS), and Salvia sclarea sclareol synthase (SsSS) (Jia et al. Metabolic Engineering 37:24-34 (2016)).

Table 6 illustrates the distribution among selected Lamiaceae clades of diterpenes with various structural patterns. Blue enzyme names are placed according to the pattern they install and the clade of the species they were cloned from. A solid line indicates that only compounds with the bond-type shown at that position are counted. A dashed line indicates that all types of bonds and substituents are counted at that position. Based on data from the DNP.

TABLE 6A
Lamiaceae clades of diterpenes with various structural patterns.

| | Clerodane | Cleroda-4(18)-ene | 4(18)-epoxy-Clerodane |
|---|---|---|---|
| | 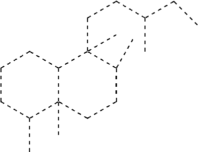 | 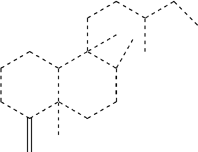 | 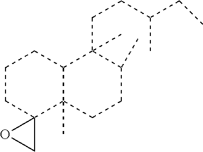 |
| Ajugoideae | 317 | (ArTPS2) 6 | 206 |
| Lamioideae | 32 | 3 | 1 |
| Nepetoideae | 132 | 1 | 1 |
| Scutellarioideae | 160 | 19 | 78 |
| Viticoideae | 1 | 0 | 0 |
| All clades | 668 | 31 | 289 |

TABLE 6B
Lamiaceae clades of diterpenes with various structural patterns.

| | Clerodane-3-ene | Labdane |
|---|---|---|
| | 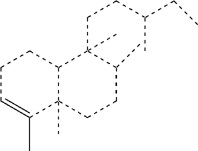 | 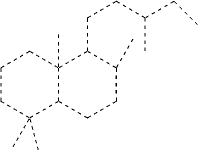 |
| Ajugoideae | 23 | 3 |
| Lamioideae | 25 | 201 |
| Nepetoideae | 84 | 60 |
| Seutellarioideae | 44 | 0 |
| Viticoideae | 0 | 37 |
| All clades | 189 | 300 |

TABLE 6C
Lamiaceae clades of diterpenes with various structural patterns.

| | Labda-8-ene | Labda-7-ene |
|---|---|---|
| | 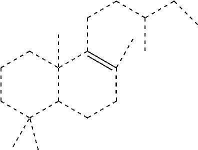 | 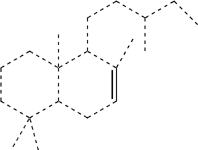 |
| Ajugoideae | 2 | 0 |
| Lamioideae | (PcTPS1)27 | 5 |
| Nepetoideae | 1 | (HsTPS1) 1 |
| Scutellarioideae | 0 | 0 |
| Viticoideae | 2 | 2 |
| AU clades | 33 | 9 |

HsTPS1 was identified as a (5S,9S,10S) labda-7,13E-dienyl diphosphate [21] synthase based on comparison to the product of an enzyme from Grindelia robusta, GrTPS2 (Zerbe et al. The Plant Journal 83(5):783-793 (2015)), and by NMR of the alcohol derivative [21a]. Normal absolute stereochemistry was assigned to the HsTPS1 product based on the optical rotation of 21a, [α]_{D} +8.3° (c. 0.0007, CHCl₃) (c.f. lit. [α]_{D}+5°, c. 1.0, CHCl₃ (Urones et al. Phytochemistry 35(3):713-719 (1994)); [α]_{D}²⁵+12°, c. 0.69, CHCl₃ (Suzuki et al. Phytochemistry 22(5):1294-1295 (1983)). When HsTPS1 was expressed in N. benthamiana, labda-7,13(16),14-triene [22] was formed, which seemed to be enhanced by co-expression with CfTPS3. The combination of HsTPS1 with OmTPS3 produced labda-7,12E,14-triene [24] (Roengsumran et al. Phytochemistry 50(3):449-453 (1999)), which has previously been accessible only by combinations of bacterial enzymes (Yamada et al. The Journal of Antibiotics 69(7):515-523 (2016)). Labdanes with a double bond at the 7-position have not been reported in H. suaveolens, and such labdanes do not seem to be common in Lamiaceae. Of nine compounds with the labdane skeleton and a double bond at position-7 (Table 6) only one was from the same clade as H. suaveolens. (13E)-ent-labda-7,13-dien-15-oic acid, from Isodon scoparius (Xiang et al. Helvetica Chimica Acta 87(11):2860-2865 (2004)), has the opposite absolute stereochemistry to the HsTPS1 product, likely not deriving from a paralog of HsTPS1 because absolute stereochemistry of a skeleton is not known to change after the diTPS steps.

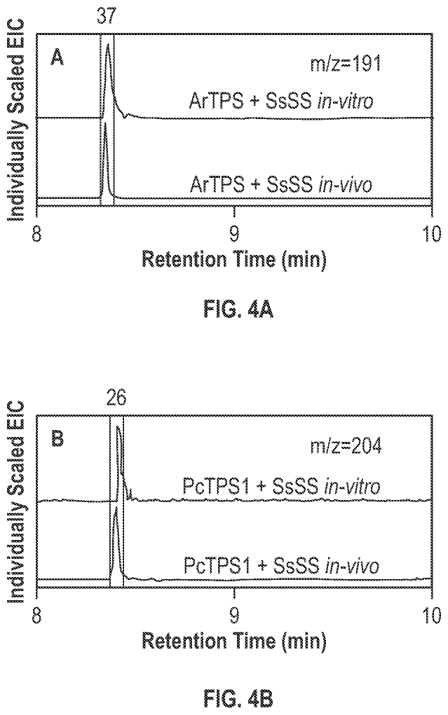

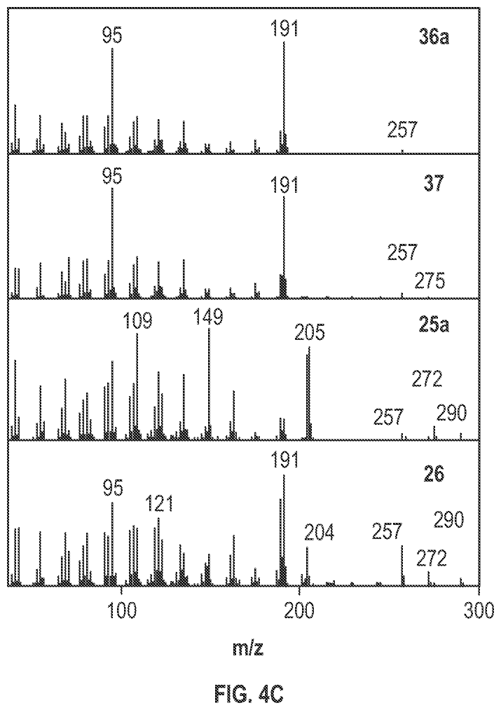

ArTPS2 was identified as a (5R,8R,9S,10R) neo-cleroda-4(18),13E-dienyl diphosphate [38] synthase. The combination of ArTPS2 and SsSS generated neo-cleroda-4(18),14-dien-13-ol [37] (FIG. 4A). The structures of compounds 37 and 38a were determined by NMR. The analysis included a comparison of compound 37 to chelodane (Rudi et al. J Nat Prod 55(10):1408-1414 (1992)), which based on small differences in ¹³C shifts, may be a stereoisomer of compound 37 at the 13 position, and a comparison of the NMR results for compound 38a with the NMR of its enantiomer (Ohaski et al. Bioorganic & Medicinal Chemistry Letters 4(24):2889-2892 (1994)). There were 20 to 19, and 20 to 17 NOE interactions in the NMR spectra of 37 and 38a, which closely resembled those reported for (−)-kolavelol [36a] (Pelot et al. Plant J 89(5):885-897 (2017)), indicating that the stereochemistry may be 5R,8R,9S,10R. The “neo” absolute configuration was established through optical rotation of 38a, [α]_{D}+30° (c. 0.0025, CHCl₃) (c.f. lit. [α]_{D} +20.9°, c. 0.7, CHCl₃) (Monaco et al. Rendiconto della Academia delle scienze fisiche e matematiche 48:465-470 (1982)).

Previously reported clerodane diTPSs from Lamiaceae produce kolavenyl diphosphate [36] (Heskes et al. Plant J 93(5):943-958 (2018); Chen et al. J Exp Bot 68(5):1109-1122 (2017): Pelot et al. Plant J 89(5):885-897 (2017)), and kolavenyl diphosphate [36] has a double bond at the 3-position. Clerodanes with desaturation at position-3 are spread throughout multiple clades but are most common in Nepetoideae (Table 6A-6C), which includes Salvia divinorum. Clerodanes with a double bond at the 4(18)-position are rare by comparison, but those with a 4(18)-epoxy moiety, make up nearly half of the clerodanes reported in Lamiaceae, including two-thirds of those reported from the Ajugoideae clade (Table 6A-6C), one of which is clerodin (Barton et al. J Chem Soc: 5061-5073 (1961)) and from which the clerodane skeleton gets its name. Neo-cleroda-4(18),13E-dienyl diphosphate is a logical biosynthetic precursor for the 4(18)-epoxy clerodanes. It is unclear if any of the previously described diTPSs directly produce an epoxide moiety.

PcTPS1 was identified as a (10R)-labda-8,13E-dienyl diphosphate [25] synthase. The structure was established by comparison of ¹³C NMR of compound 25a to previously reported spectra (Suzuki et al. Phytochemistry 22(5):1294-1295 (1983)). The 10R (ent-) absolute stereochemistry was established by optical rotation of compound 25a [α]_{D} −64° (c. 0.0008, CHCl₃), (c.f. lit. [α]_{D}²⁵ −71.2°, c. 1.11, CHCl₃) (Arima et al. Tetrahedron: Asymmetry 18(14):1701-1711 (2007)). The combination of PcTPS1 and SsSS, both in-vitro, and in N. benthamiana expression produced (10R)-labda-8,14-en-13-ol [26] (FIG. 4B), the structure of which was determined by comparison of ¹³C NMR to a published spectrum (Wu & Lin Phytochemistry 44(1):101-105 (1997)). The double bond between positions 8 and 9 is present in 33 distinct compounds isolated from Lamiaceae (Table 6A-6C), most of which occur in the Lamioideae clade, which includes Pogostemon cablin, the source of PcTPS1. Absolute stereochemistries of the reported compounds are mixed, with some in the normal configuration (Boalino et al. J Nat Prod 67(4):714-717 (2004)), and others in the ent-configuration (Gray et al. Phytochemistry 63(4):409-413 (2003)). As normal configuration 9-hydroxy labdanes are also abundant in Lamioideae, it is possible that the normal configuration 8(9) desaturated labdanes arise from dehydratase activities downstream of a PgPP synthase (MvCPS1 and its paralogs), while those in the ent-configuration arise from paralogs of PcTPS1. Another possibility is that some of the 8(9) desaturated labdanes reported as having normal absolute stereochemistry are actually ent-labdanes that were mis-assigned, as has occurred in at least one documented case (Gray et al. Phytochemistry 63(4):409-413 (2003)).

### Example 5: Characterization of Class I dITPSs

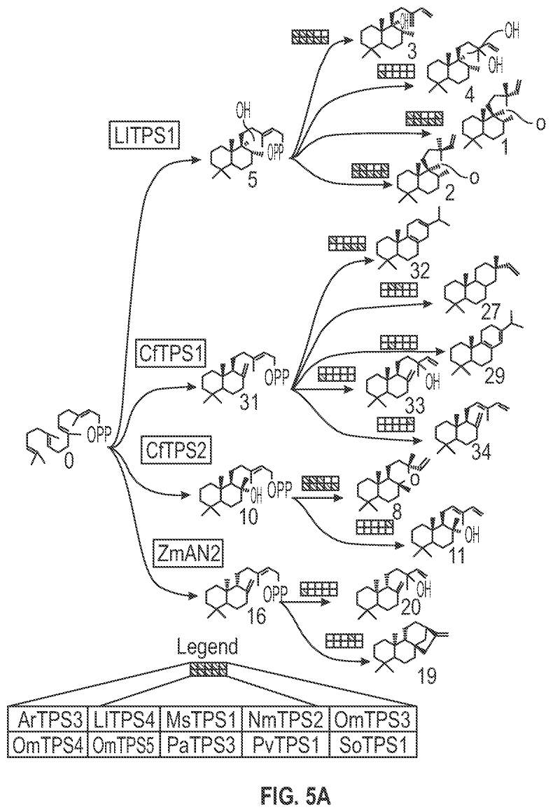

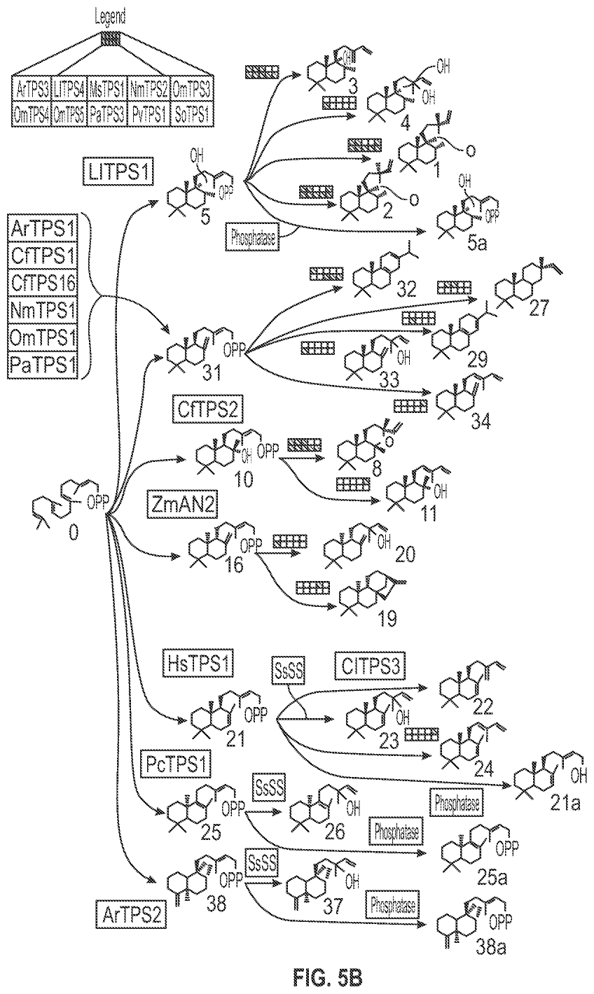

Class I diTPS candidates were characterized by transient expression in N. benthamiana in combination with four class II enzymes:
CfTPS1, a (+)-CPP [31] synthase:
CfTPS2, a labda-13-en-8-ol diphosphate ((+)-8-LPP) [10] synthase (Pateraki et al. Plant Physiol 164(3):1222-1236 (2014);
LITPS1, a PgPP 151 synthase; or
Zea mays ZmAN2, an ent-copalyl diphosphate (ent-CPP) [16] synthase (Harris et al. Plant Mol Biol 59(6):881-894 (2005)).
   Substrates accepted by each enzyme and the products are indicated in FIG. 2B and FIG. 5. NmTPS2 was identified as an ent-kaurene [19] synthase, converting ent-CPP into ent-kaurene (identified using Physcomitrella patens extract as a standard (Zhan et al. Plant Physiology and Biochemistry 96:110-114 (2015))), but not showing activity with any other substrate. The only other enzyme to show activity with ent-CPP was OmTPS4, which produced ent-manool [20], just as SsSS produces from ent-CPP.

PaTPS3, PvTPS1, SoTPS1, ArTPS3, OmTPS4, LITPS4, OmTPS5, and MsTPS1 converted (+)-8-LPP to 13R-(+)-manoyl oxide [8], verified by comparison to the product of CfTPS2 and CfTPS3 (Pateraki et al. Plant Physiol 164(3):1222-1236 (2014)). OmTPS3 produced trans-abienol [11]. The trans-abienol structure was determined by NMR, with the stereochemistry of the 12(13)-double bond supported by comparison of the NOESY spectrum to that of a commercial standard for cis-abienol (Toronto Research Chemicals. Toronto Canada). The trans-abienol showed clear NOE correlation between positions 16 and 11, while the cis-abienol standard showed correlations between 14 and 11.

PaTPS3, PvTPS1, SoTPS1, and ArTPS3, LITPS4, and OmTPS5 converted PgPP to a combination of 1, 2, and 3, with some variation in the ratios between the products. Because perigrinol [5a] spontaneously degrades into 1, 2, and 3 under GC conditions (Zerbe et al. Plant J 79(6):914-927 (2014)), it was difficult to distinguish whether these enzymes have low activity, but specific products, or moderate activity with a mix of products. Nevertheless, differences in relative amounts of the products observed between LITPS1 alone and in combination with these class 1 enzymes suggest that they do have some activity on PgPP. OmTPS4 produced 1, 2, 3, and 4. MsTPS1 produced only 3, and OmTPS3 produced only 1, and 2. PgPP products were established by comparison to MvCPS1, MvCPS1 with MvELS (Zerbe et al. Plant J 79(6):914-927 (2014)), and MvCPS1 with SsSS (Jia et al. Metabolic Engineering 37:24-34 (2016)).

PaTPS3, PvTPS1, SoTPS1, and ArTPS3 converted (+)-CPP to miltiradiene [32], similarly to CfTPS3. OmTPS4 produced manool [33], as compared to SsSS. LITPS4 and MsTPS1 produced sadaracopimaradiene [27], by comparison to a product from Euphorbia peplus EpTPS8 (Andersen-Ranberg et al. Angew Chem Int Ed 55(6):2142-2146 (2016)). OmTPS5 produced palustradiene [29], as compared to a minor product from Abies grandis abietadiene synthase (Vogel et al. J Biol Chem 271(38):23262-23268 (1996)). OmTPS3 produced trans-biformene [34], as established by comparison of ³C-NMR of compounds described by Bohlmann & Czerson, Phytochemistry 18(1):115-118 (1979)), with a trans configuration further supported by clear NOE correlations between 16 and 11, and the absence of NOE correlations between 14 and 11.

### Example 6: Origanum majorana Enzymes can Make Palustradiene and Other Diterpenoids

The class I enzymes from Origanum majorana, OmTPS3, OmTPS4, and OmTPS5 all produced different products from (+)-CPP, which itself is the product of OmTPS1 from the same species. Despite the apparent richness of activities of enzymes from O. majorana, no reports of diterpenes were located from that species either in database searches, or in a subsequent literature search.

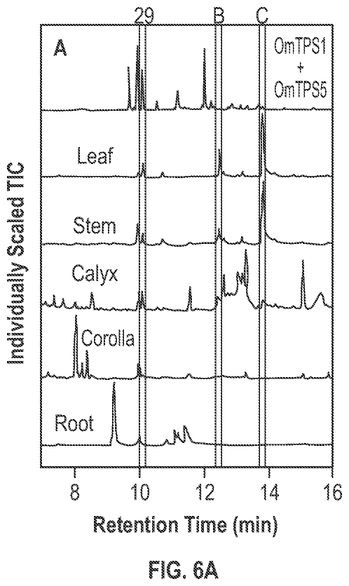

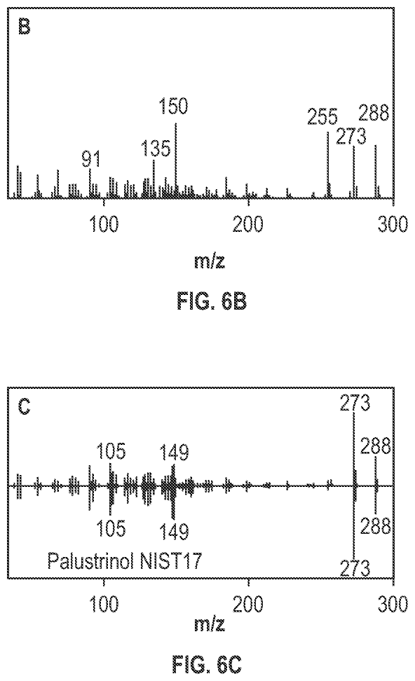

To determine whether diterpene synthases are active in O. majorana, the products of enzyme combinations with extracts from O. majorana leaf, stem, calyx, corolla, and root were evaluated. Palustradiene [29], the product of OmTPS1 and OmTPS5, was detected in all tissues except roots (FIG. 6). In addition, two diterpene alcohols were detected in the stem, leaf, and calyx. One diterpene alcohol, could not be identified, but the other was a close match to palustrinol, the 19-hydroxy derivative of palustradiene, in the NIST17 spectral library. The structures of the palustrinol, and the 19-hydroxy derivative of palustradiene are shown below.

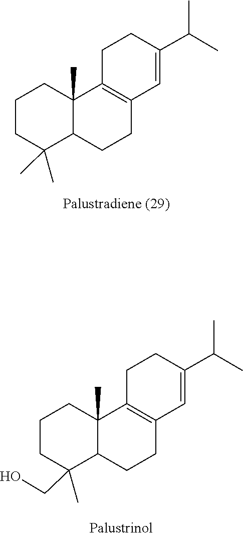

### Example 7: Chiococca alba Enzymes can Make 13(R)-Epi-Dolabradiene and Other Compounds

This Example illustrates that enzymes from Chiococca alba can produce products such as ent-kaurene, ent-dolabradiene (13-epi-dolabradiene), and (13R)-ent-manoyl oxide.

Enzyme assays were prepared as described herein that separately or in combination contained the following enzymes and substrates:
class I terpene synthase enzyme from Chiococca alba (CaTPS1) with SoTPS2, SbTPS1, and SbTPS2 and the substrate ent-copalyl diphosphate.
class II terpene synthase enzyme from Chiococca alba (CaTPS2) with substrate ent-labda-13-en-8-ol diphosphate
class III and class IV terpene synthase enzymes from Chiococca alba (CaTPS3 and CaTPS4) with substrate ent-kaurene
class V terpene synthase enzyme from Chiococca alba (CaTPS5) with substrate ent-dolabradiene
class I (−)-kolavenyl diphosphate synthase enzyme from Salvia hispanica (ShTPS1) with substrate (−)-kolavenyl diphosphate
class I cleroda-4(18),13E-dienyl diphosphate synthase enzyme from Teucrium canadense (TcTPS1) with substrate clerodadienyl diphosphate
class I sclareol synthase enzyme from Salvia sclarea (SsSCS) with substrate neo-clerodadienol.

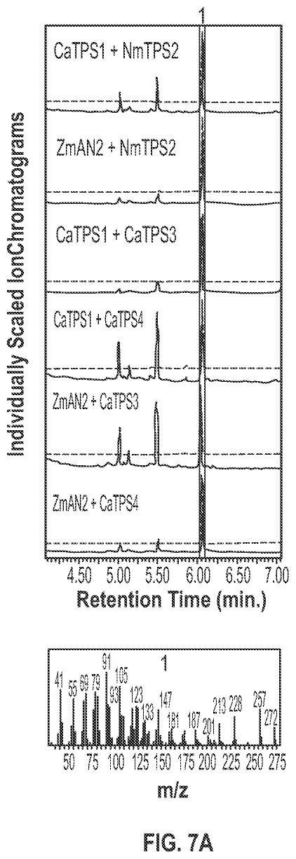

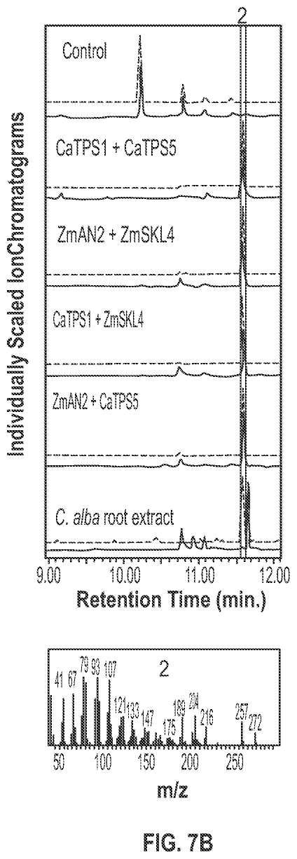

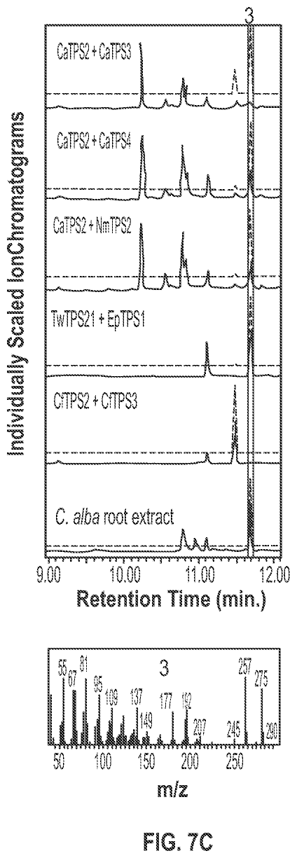

FIG. 7 illustrates the activities of the newly obtained Chiococca alba terpene synthases CaTPS1-5. FIGS. 7A-7C show GC-MS-total ion and extracted ion chromatograms from in vivo assays within N. benthamiana that transiently expressed various combinations of enzymes. Mass spectra are shown below the chromatograms of FIG. 7A-7C for peaks (1) to (3) containing the following products of the enzymatic conversion: (1) ent-kaurene; (2) ent-dolabradiene (13-epi-dolabradiene); (3) (13R)-ent-manoyl oxide. The ent-dolabradiene was identified through extensive structural studies with NMR and the stereochemistry at C-13 was unequivocally corroborated by optical rotation. The ent-kaurene and (13R)-ent-manoyl oxide were identified through direct comparison with biosynthesized authentic standards with reference enzymes.

Compounds ent-dolabradiene (13-epi-dolabradiene) and (13R)-ent-manoyl oxide are plausible intermediates in the biosynthetic routes to the structurally unusual merilactone and ribenone, that have demonstrated activity against Leishmanina and potential anti-cancer activity (Piozzi, F., Bruno, M. Diterpenoids from Roots and Aerial Parts of the Genus Stachys Rec. Nat. Prod. 5, 1-11, (2011)).

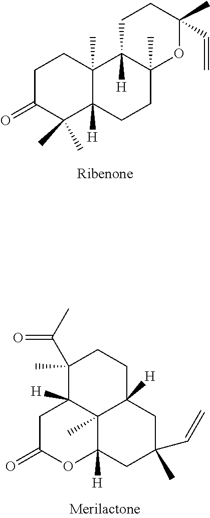

Both merilactone and ribenone are detected in the root extract of C. alba.

### REFERENCES

1. Dictionary of Natural Products 26.2 Available at: http://dnp.chemnetbase.com [Accessed Jan. 11, 2018].
2. Peters R J (2010) Two rings in them all: The labdane-related diterpenoids. Natural product reports 27(11):1521.
3. Chen F, Tholl D, Bohlmann J, Pichersky E (2011) The family of terpene synthases in plants: a mid-size family of genes for specialized metabolism that is highly diversified throughout the kingdom. The Plant Journal 66(1):212-229.
4. Zerbe P, Bohlmann J (2015) Plant diterpene synthases: exploring modularity and metabolic diversity for bioengineering. Trends In Biotechnology 33(7):419-428.
5. Hamberger B, Bak S (2013) Plant P450s as versatile drivers for evolution of species-specific chemical diversity. Philosophical Transactions of the Royal Society of London B: Biological Sciences 368(1612):20120426.
6. Banerjee A, Hamberger B (2018) P450s controlling metabolic bifurcations in plant terpene specialized metabolism. Phytochem Rev 17(1):81-111.
7. Pateraki I, et al. (2017) Total biosynthesis of the cyclic AMP booster forskolin from Coleus forskohlii. elife 6:e23001.
8. Ondari M E, Walker K D (2008) The Taxol Pathway 10-O-Acetyltransferase Shows Regioselective Promiscuity with the Oxetane Hydroxyl of 4-Deacetyltaxanes. J Am Chem Soc 130(50):17187-17194.
9. Chau M, Walker K, Long R, Croteau R (2004) Regioselectivity of taxoid-O-acetyltransferases: heterologous expression and characterization of a new taxadien-5α-ol-O-acetyltransferase. Archives of Biochemistry and Biophysics 430(2):237-246.
10. Cui G, et al. (2015) Functional divergence of diterpene syntheses in the medicinal plant Salvia miltiorrhiza Bunge. Plant Physiol 169(3):1607-1618.
11. Gao W, et al. (2009) A Functional Genomics Approach to Tanshinone Biosynthesis Provides Stereochemical Insights. Org Lett 11(22):5170-5173.
12. Guo J, et al. (2013) CYP76AH1 catalyzes turnover of miltiradiene in tanshinones biosynthesis and enables heterologous production of ferruginol in yeasts. PNAS 110(29):12108-12113.
13. Heskes A M, et al. (2018) Biosynthesis of bioactive diterpenoids in the medicinal plant Vitex agnus-castus. Plant J 93(5):943-958.
14. Zerbe P, et al. (2014) Diterpene synthases of the biosynthetic system of medicinally active diterpenoids in Marrubium vulgare. Plant J 79(6):914-927.
15. Chen X, Berim A, Dayan F E, Gang D R (2017) A (−)-kolavenyl diphosphate synthase catalyzes the first step of salvinorin A biosynthesis in Salvia divinorum. J Exp Bot 68(5):1109-1122.
16. Pelot K A, et al. (2017) Biosynthesis of the psychotropic plant diterpene salvinorin A: Discovery and characterization of the Salvia divinorum clerodienyl diphosphate synthase. Plant J 89(5):885-897.
17. Caniard A, et al. (2012) Discovery and functional characterization of two diterpene synthases for sclareol biosynthesis in Salvia sclarea (L.) and their relevance for perfume manufacture. BMC Plant Biology 12:119.
18. Günnewich N, et al. (2013) A diterpene synthase from the clary sage Salvia sclarea catalyzes the cyclization of geranylgeranyl diphosphate to (8R)-hydroxy-copalyl diphosphate. Phytochemistry 91:93-99.
19. Boachon B, et al. (2018) Phylogenomic Mining of the Mints Reveals Multiple Mechanisms Contributing to the Evolution of Chemical Diversity in Lamiaceae. Molecular Plant. doi:10.1016/j.molp.2018.06.002.
20. Coll J, Tandrón Y A (2008) neo-Clerodane diterpenoids from Ajuga: structural elucidation and biological activity. Phytochem Rev 7(1):25.
21. Klein Gebbinck E A, Jansen B J M, de Groot A (2002) Insect antifeedant activity of clerodane diterpenes and related model compounds. Phytochemistry 61(7):737-770.
22. Li R, Morris-Natschke S L, Lee K-H (2016) Clerodane diterpenes: sources, structures, and biological activities. Nat Prod Rep 33(10):1166-1226.
23. Vestri Alvarenga S A, Pierre Gastmans J, do Vale Rodrigues G, Roberto H. Moreno P, de Paulo Emerenciano V (2001) A computer-assisted approach for chemotaxonomic studies—diterpenes in Lamiaceae. Phytochemistry 56(6):583-595.
24. Loub W D, Farnsworth N R, Soejarto D D, Quinn M L (1985) NAPRALERT: computer handling of natural product research data. J Chem Inf Comput Sci 25(2):99-103.
25. Federhen S (2012) The NCBI Taxonomy database. Nucleic Acids Res 40(D1):D136-D143.
26. Li B. et al. (2016) A large-scale chloroplast phylogeny of the Lamiaceae sheds new light on its subfamilial classification. Scientific Reports 6:34343.
27. Camacho C, et al. (2009) BLAST+: architecture and applications. BMC Bioinformatics 10:421.
28. Pateraki I, et al. (2014) Manoyl Oxide (13R), the Biosynthetic Precursor of Forskolin, Is Synthesized in Specialized Root Cork Cells in Coleus forskohlii. Plant Physiol 164(3):1222-1236.
29. Jia M, Potter K C, Peters R J (2016) Extreme promiscuity of a bacterial and a plant diterpene synthase enables combinatorial biosynthesis. Metabolic Engineering 37:24-34.
30. Zerbe P, et al. (2015) Exploring diterpene metabolism in non-model species: transcriptome-enabled discovery and functional characterization of labda-7,13 E-dienyl diphosphate synthase from Grindelia robusta. The Plant Journal 83(5):783-793.
31. Urones J G, et al. (1994) Compounds with the labdane skeleton from Halimium viscosum. Phytochemistry 35(3):713-719.
32. Suzuki H, Noma M, Kawashima N (1983) Two labdane diterpenoids from Nicotiana setchellii. Phytochemistry 22(5):1294-1295.
33. Roengsumran S, Petsom A, Sommit D, Vilaivan T (1999) Labdane diterpenoids from Croton oblongifolius. Phytochemistry 50(3):449-453.
34. Yamada Y, Komatsu M, Ikeda H (2016) Chemical diversity of labdane-type bicyclic diterpene biosynthesis in Actinomycetales microorganisms. The Journal of Antibiotics 69(7):515-523.
35. Xiang W, Li R-T, Song Q-S, Na Z, Sun H-D ent-Clerodanoids from Isodon scoparius. Helvetica Chimica Acta 87(11):2860-2865.
36. Rudi A, Kashman Y (1992) Chelodane, Barekoxide, and Zaatirin-Three New Diterpenoids from the Marine Sponge Chelonaplysilla erecta. J Nat Prod 55(10):1408-1414.
37. Ohsaki A, et al. (1994) The isolation and in vivo Potent Antitumor activity of clerodane diterpenoid from the oleoresin of the brazilian medicinal plant, copaifera langsdorfi desfon. Bioorganic & Medicinal Chemistry Letters 4(24):2889-2892.
38. Monaco P, Previtera L, Mangoni L (1982) Terpenes from the bled resin of Araucaria hunsteinii. Rendiconto della Academia delle scienze fisiche e matematiche 48:465-470.
39. Barton D H R, Cheung H T, Cross A D, Jackman L M, Martin-Smith M (1961) 1003. Diterpenoid bitter principles. Part III. The constitution of clerodin. J Chem Soc. 5061-5073.
40. Arima Y, Kinoshita M, Akita H (2007) Natural product synthesis from (8aR)- and (8aS)-bicyclofarnesols: synthesis of (+)-wiedendiol A, (+)-norsesterterpene diene ester and (−)-subersic acid. Tetrahedron: Asymmetry 18(14):1701-1711.
41. Wu C-L, Hsiang-Ru Lin (1997) Labdanoids and bis(bibenzyls) from Jungermannia species. Phytochemistry 44(1):101-105.
42. Boalino D M, McLean S, Reynolds W F, Tinto W F (2004) Labdane Diterpenes of Leonurus sibiricus. J Nat Prod 67(4):714-717.
43. Gray C A, Rivett D E A, Davies-Coleman M T (2003) The absolute stereochemistry of a diterpene from Ballota aucheri. Phytochemistry 63(4):409-413.
44. Harris L J, et al. (2005) The Maize An2 Gene is Induced by Fusarium Attack and Encodes an ent-Copalyl Diphosphate Synthase. Plant Mol Biol 59(6):881-894.
45. Zhan X, Bach S S, Hansen N L, Lunde C, Simonsen H T (2015) Additional diterpenes from Physcomitrella patens synthesized by copalyl diphosphate/kaurene synthase (PpCPS/KS). Plant Physiology and Biochemistry 96:110-114.
46. Andersen-Ranberg J, et al. (2016) Expanding the Landscape of Diterpene Structural Diversity through Stereochemically Controlled Combinatorial Biosynthesis. Angew Chem Int Ed 55(6):2142-2146.
47. Vogel B S, Wildung M R, Vogel G, Croteau R (1996) Abietadiene synthase from grand fir (Abies grandis) cDNA isolation, characterization, and bacterial expression of a bifunctional diterpene cyclase involved in resin acid biosynthesis. J Biol Chem 271(38):23262-23268.
48. Bohlmann F, Czerson H (1979) Neue labdan-und pimaren-derivate aus Palafoxia rosea. Phytochemistry 18(1):115-118.
49. Li J-L, et al. (2012) IeCPS2 is potentially involved in the biosynthesis of pharmacologically active Isodon diterpenoids rather than gibberellin. Phytochemistry 76:32-39.
50. Jin B, et al. (2017) Functional diversification of kaurene synthase-like genes. Plant Physiol 174:973-955.
51. Hillwig M L, et al. (2011) Domain loss has independently occurred multiple times in plant terpene synthase evolution. The Plant Journal 68(6):1051-1060.
52. Pelot K A, Hagelthorn D M, Addison J B, Zerbe P (2017) Biosynthesis of the oxygenated diterpene nezukol in the medicinal plant Isodon rubescens is catalyzed by a pair of diterpene synthases. PLOS ONE 12(4):e0176507.
53. Helliwell C A, Chandler P M, Poole A, Dennis E S, Peacock W J (2001) The CYP88A cytochrome P450, ent-kaurenoic acid oxidase, catalyzes three steps of the gibberellin biosynthesis pathway. PNAS 98(4):2065-2070.
54. Han Q-B, et al. (2006) Maoecrystal Z, a Cytotoxic Diterpene from Isodon eriocalyx with a Unique Skeleton. Org Lett 8(21):4727-4730.
55. Li X-N, et al. (2010) Structure and Cytotoxicity of Diterpenoids from Isodon eriocalyx. J Nat Prod 73(11):1803-1809.
56. González A G, Andrés L S, Luis J G, Brito I, Rodríguez M L (1991) Diterpenes from Salvia mellifera. Phytochemistry 30(12):4067-4070.
57. Chen Y-L, et al. (2008) Bioactive Cembrane Diterpenoids of Anisomeles indica. J Nat Prod 71(7):1207-1212.
58. Li L-M, et al. (2009) ent-Kaurane and Cembrane Diterpenoids from Isodon sculponeatus and Their Cytotoxicity. J Nat Prod 72(10):1851-1856.
59. Kirby J, et al. (2010) Cloning of casbene and neocembrene synthases from Euphorbiaceae plants and expression in Saccharomyces cerevisiae. Phytochemistry 71(13):1466-1473.
60. Ennajdaoui H, et al. (2010) Trichome specific expression of the tobacco (Nicotiana sylvestris) cembratrien-ol synthase genes is controlled by both activating and repressing cis-regions. Plant Mol Bio 73(6):673-685.
61. Hamano Y, et al. (2002) Functional Analysis of Eubacterial Diterpene Cyclases Responsible for Biosynthesis of a Diterpene Antibiotic, Terpentecin. J Biol Chem 277(40):37098-37104.
62. Dairi T, et al. (2001) Eubacterial Diterpene Cyclase Genes Essential for Production of the Isoprenoid Antibiotic Terpentecin. J Bacteriol 183(20):6085-6094.
63. Schalk M, et al. (2012) Toward a Biosynthetic Route to Sclareol and Amber Odorants. J Am Chem Soc 134(46):18900-18903.
64. Ikeda H, Shin-ya K, Nagamitsu T, Tomoda H (2016) Biosynthesis of mercapturic acid derivative of the labdane-type diterpene, cyslabdan that potentiates imipenem activity against methicillin-resistant Staphylococcus aureus: cyslabdan is generated by mycothiol-mediated xenobiotic detoxification. J Ind Microbiol Biotechnol 43(2-3):325-342.
65. Keeling C I, Madilao L L, Zerbe P, Dullat H K, Bohlmann J (2011) The Primary Diterpene Synthase Products of Picea abies Levopimaradiene/Abietadiene Synthase (PaLAS) Are Epimers of a Thermally Unstable Diterpenol. J Biol Chem 286(24):21145-21153.
66. Geuskens R B M, Luteijn J M, Schoonhoven L M (1983) Antifeedant activity of some ajugarin derivatives in three lepidopterous species. Experientia 39(4):403-404.
67. Belles X, Camps F, Coll J, Piulachs M D (1985) Insect antifeedant activity of clerodane diterpenoids against larvae of Spodoptera Littoralis (Boisd.) (Lepidoptera). J Chem Ecol 11(10):1439-1445.
68. Challis G L (2008) Genome Mining for Novel Natural Product Discovery. J Med Chem 51(9):2618-2628.
69. Xu H, et al. (2016) Analysis of the Genome Sequence of the Medicinal Plant Salvia miltiorrhiza. Molecular Plant 9(6):949-952.
70. King A J, Brown G D, Gilday A D, Larson T R, Graham I A (2014) Production of Bioactive Diterpenoids in the Euphorbiaceae Depends on Evolutionarily Conserved Gene Clusters. The Plant Cell Online 26(8):3286-3298.
71. Huang A C, et al. (2017) Unearthing a sesterterpene biosynthetic repertoire in the Brassicaceae through genome mining reveals convergent evolution. PNAS 114(29):E6005-E6014.
72. Busta L, Jetter R (2017) Moving beyond the ubiquitous: the diversity and biosynthesis of specialty compounds in plant cuticular waxes. Phytochem Rev: 1-30.
73. Kodama Y, Shumway M, Leinonen R (2012) The sequence read archive: explosive growth of sequencing data. Nucleic Acids Res 40(D1):D54-D56.
74. Benson D A, et al. (2013) GenBank. Nucleic Acids Res 41(D1):D36-D42.
75. Kuhn S, Schlörer N E, Kolshorn H, Stoll R (2012) From chemical shift data through prediction to assignment and NMR LIMS—multiple functionalities of nmrshiftdb2. Journal of Cheminformatics 4(Suppl 1):P52.
76. Fischedick J T, Johnson S R, Ketchum R E B, Croteau R B, Lange B M (2015) NMR spectroscopic search module for Spektraris, an online resource for plant natural product identification—Taxane diterpenoids from Taxus x media cell suspension cultures as a case study. Phytochemistry 113:87-95.
77. Scotti M T, et al. (2018) SistematX, an Online Web-Based Cheminformatics Tool for Data Management of Secondary Metabolites. Molecules 23(1):103.
78. Heller S R, McNaught A, Pletnev I, Stein S, Tchekhovskoi D (2015) InChI, the IUPAC International Chemical Identifier. J Cheminform 7. doi:10.1186/s13321-015-0068-4.
79. Sievers F, et al. (2011) Fast, scalable generation of high-quality protein multiple sequence alignments using Clustal Omega. Molecular Systems Biology 7:539.
80. Stamatakis A (2014) RAxML version 8: a tool for phylogenetic analysis and post-analysis of large phylogenies. Bioinformatics 30(9):1312-1313.
81. Huerta-Cepas J, Serra F, Bork P (2016) ETE 3: Reconstruction, Analysis, and Visualization of Phylogenomic Data. Mol Biol Evol 33(6):1635-1638.
82. Lopez-Perez J L, Theron R, del Olmo E, Diaz D (2007) NAPROC-13: a database for the dereplication of natural product mixtures in bioassay-guided protocols. Bioinformatics 23(23):3256-3257.

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The following statements are intended to describe and summarize various features of the invention according to the foregoing description provided in the specification and figures.

### Statements:

1. An expression system comprising at least one expression cassette having a heterologous promoter operably linked to a nucleic acid segment encoding an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176
2. The expression system of statement 1, wherein at least one expression cassette is within at least one expression vector.
3. The expression system of statement 1 or 2, wherein the expression system comprises two, or three, or four, or five expression cassettes or expression vectors, each expression cassette encoding a separate enzyme.
4. The expression system of statement 1, 2 or 3, wherein the expression system further comprises one or more expression cassettes having a promoter operably linked to a nucleic acid segment encoding an enzyme that can synthesize isopentenyl diphosphate (IPP), dimethylallyl diphosphate (DMAPP), or geranylgeranyl diphosphate (GGPP).
5. The expression system of statement 1-3 or 4, wherein the expression system has at least one expression cassette having a constitutive promoter.
6. The expression system of statement 1-3 or 4, wherein the expression system has at least one expression cassette having an inducible promoter.
7. The expression system of statement 1-5 or 6, wherein the expression system has at least one expression cassette having a CaMV 35S promoter, CaMV 19S promoter, nos promoter, Adh1 promoter, sucrose synthase promoter, α-tubulin promoter, ubiquitin promoter, actin promoter, cab promoter, PEPCase promoter, R gene complex promoter, CYP71D16 trichome-specific promoter, CBTS (cembratrienol synthase) promotor, Z10 promoter from a 10 kD zein protein gene, Z27 promoter from a 27 kD zein protein gene, plastid rRNA-operon (rrn) promoter, light inducible pea rbcS gene, RUBISCO-SSU light-inducible promoter (SSU) from tobacco, or rice actin promoter.
8. A host cell comprising the expression system of statement 1-6 or 7, which is heterologous to the host cell.
9. The host cell of statement 8, which is a plant cell, an algae cell, a fungal cell, a bacterial cell, or an insect cell.
10. The host cell of statement 8 or 9, which is a Nicotiana benthamiana, Nicotiana tabacum, Nicotiana rustica, Nicotiana excelsior, Nicotiana excelsiana, Escherichia coli. Clostridium Ijungdahlii, Clostridium autoethanogenum, Clostridium kluyveri, Corynebacterium glutamicum, Cupriavidus necator, Cupriavidus metallidurans; Pseudomonas fluorescens. Pseudomonas putida, Pseudomonas oleovorans; Delftia acidovorans, Bacillus subtilis, Lactobacillus delbrueckii, Lactococcus lactis, Aspergillus niger, Saccharomyces cerevisae, Candida tropicalis, Candida albicans, Candida cloacae, Candida guilliermondii, Candida intermedia, Candida maltosa, Candida parapsilosis, Candida zeylenoides, Pichia pastoris, Yarrowia lipolytica, Issatchenkia orientalis, Debaryomyces hansenii, Arxula adeninivorans, Kluyveromyces lactis, or Exophiala, Mucor, Trichoderma, Cladosporium, Phanerochaete, Cladophialophora, Paecilomyces. Scedosporium, or Ophiostoma cell.
11. The host cell of statement 8, 9 or 10, which is a Nicotiana benthamiana.
12. A method of synthesizing a terpene comprising incubating a host cell that has the expression system of any of statements 1-7.
13. A method for synthesizing a terpene comprising incubating a host cell comprising a heterologous expression system that includes at least one expression cassette having a heterologous promoter operably linked to a nucleic acid segment encoding an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176.
14. A method for synthesizing a terpene comprising incubating a terpene precursor with an enzyme with at least 90% sequence identity to SEQ ID NO:1, 3, 5, 7, 9, 11, 13, 15, 17, 19, 21, 23, 25, 27, 29, 31, 33, 35, 37, 39, 41, 43, 45, 47, 49, 51, 53, 57, 59, or 176.
15. The method of statement 12, 13 or 14, wherein the terpene is a compound of formula I, II, or III:

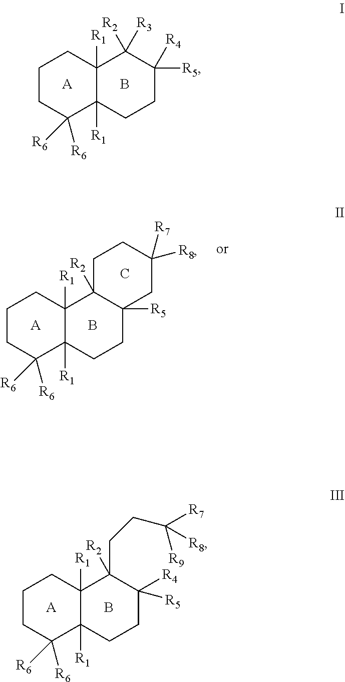

wherein
   each R₁ can separately be hydrogen or lower alkyl;
   R₂ can be hydrogen, lower alkyl, hydroxy, a bond to an adjacent ring carbon, or form a C₄-C₆ cycloheteroalkyl with R₃;
   R₃ can be a branched C₅-C₆ alkyl with 0-2 double bonds, can form a C₄-C₆ cycloheteroalkyl with R₂; can form a cycloalkyl with R₄, or can form a cycloheteroalkyl ring with R₄, wherein the C₅-C₆ alkyl can optionally have one hydroxy, phosphate or diphosphate substituent, and wherein each cycloalkyl or cycloheteroalkyl ring can have 0-2 double bonds, and each cycloalkyl or cycloheteroalkyl ring can have 0-2 alkyl or 0-2 alkene substituents;
   R₄ can be hydrogen, lower alkyl, lower alkene, hydroxy, a carbon bonded to R₉, an oxygen bonded to R₉, form a cycloalkyl ring with R₃, or form a cycloheteroalkyl ring with R₃, wherein each cycloalkyl ring or cycloheteroalkyl ring can have 0-2 double bonds, and each cycloalkyl ring or cycloheteroalkyl ring can have 0-2 alkyl or 0-2 alkene substituents;
   R₅ can be hydrogen, hydroxy, lower alkyl, a lower alkene, a bond with an adjacent carbon, form a cycloalkyl ring with a ring atom of a ring formed by R₃ and R₄, wherein the cycloalkyl ring can have 0-2 double bonds, and the cycloalkyl ring can have 0-2 alkyl or 0-2 alkene substituents;
   each R₆ can separately be hydrogen, lower alkyl, lower alkene, or form a bond with an adjacent carbon;
   R₇ can be lower alkyl, lower alkene, or form a cycloalkyl ring with a R₅,
   R₈ can be lower alkyl, hydroxy, phosphate, diphosphate, or form a bond with an adjacent carbon; and
   R₉ can be hydrogen, lower alkyl, lower alkene, ═CH₂, hydroxy, phosphate, diphosphate, form a bond with an adjacent carbon, form a cycloalkyl ring with R₄, or form a cycloheteroalkyl ring with R₄, wherein each cycloalkyl ring or cycloheteroalkyl ring can have 0-2 double bonds, and each cycloalkyl ring or cycloheteroalkyl ring can have 0-2 alkyl or 0-2 alkene substituents.
16. The method of statement 12-14 or 15 wherein the terpene is a compound with a skeleton selected from Sk1-Sk14:

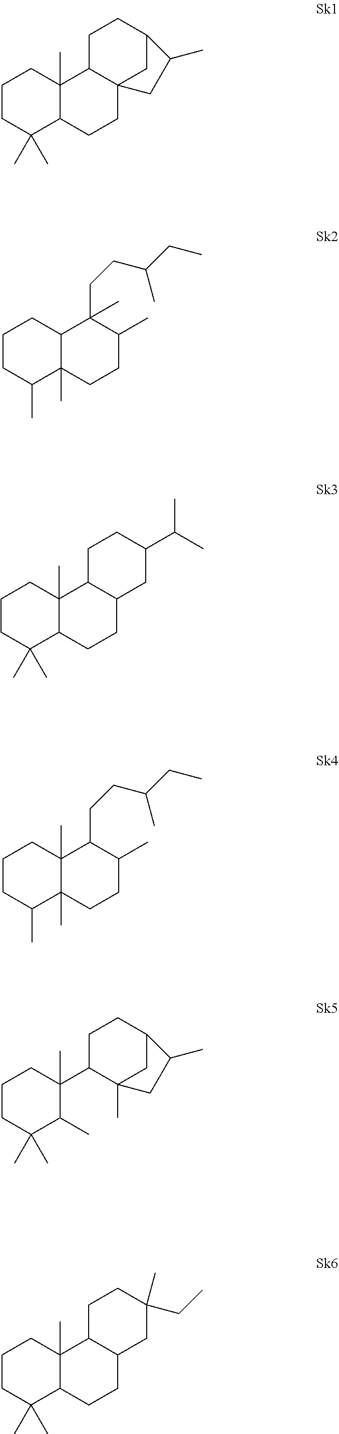

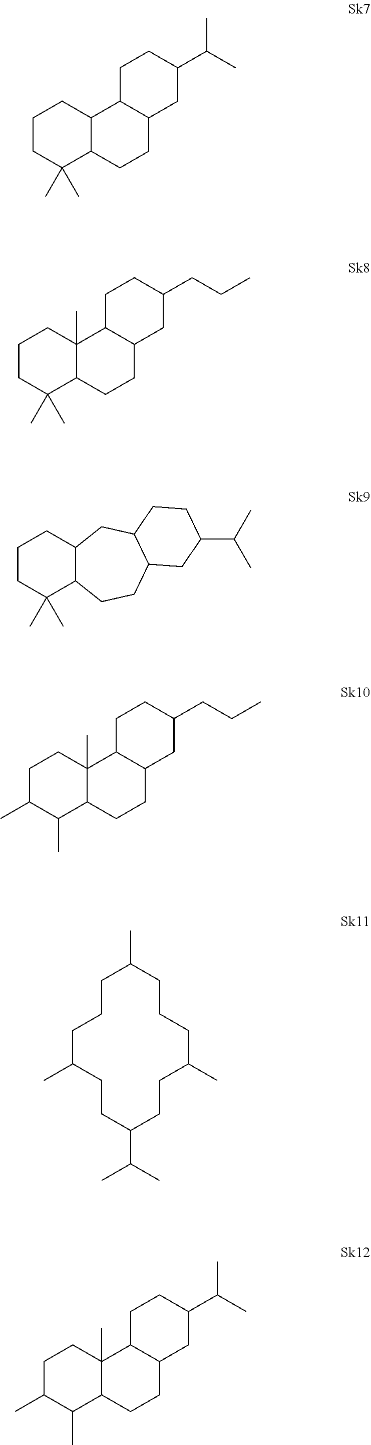

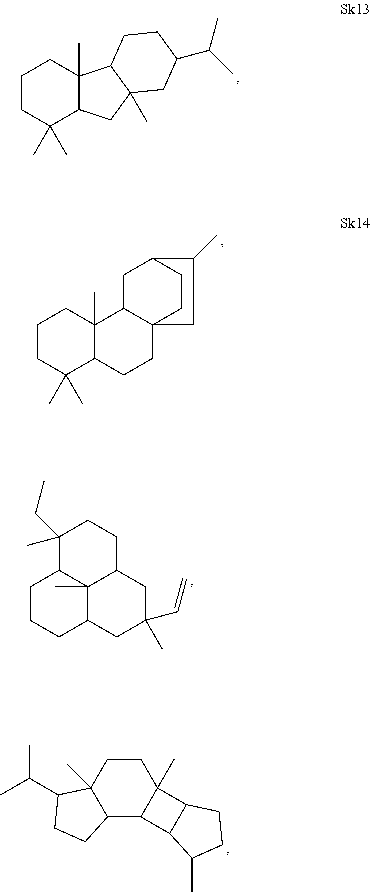

or a combination thereof.
17. The method of statement 12-15 or 16, wherein the terpene is any of the following compounds:

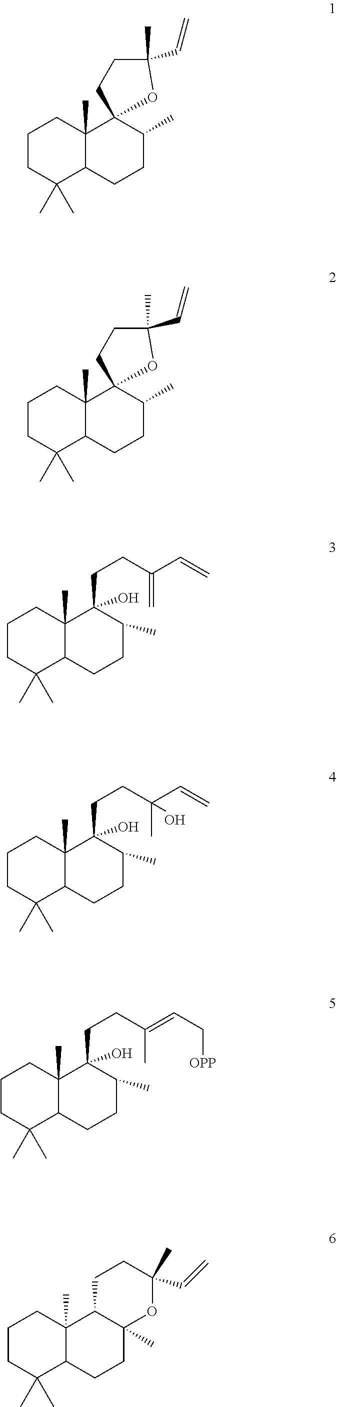

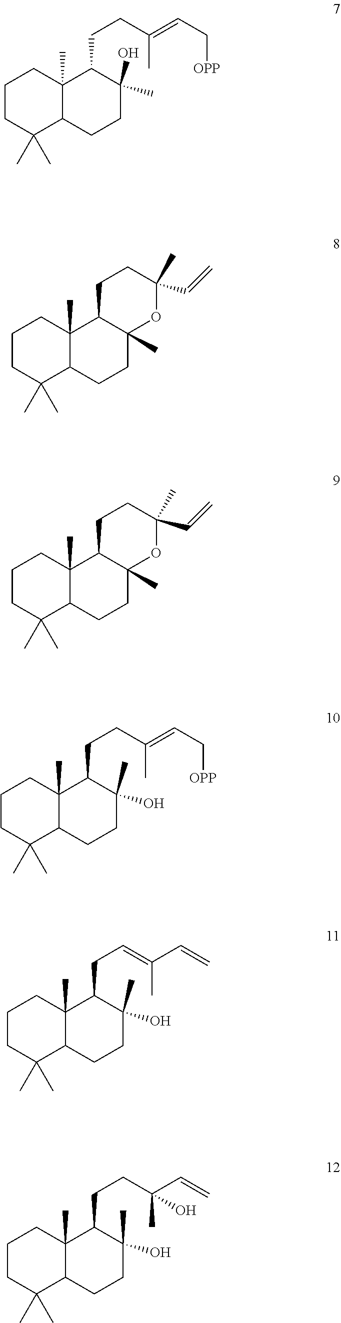

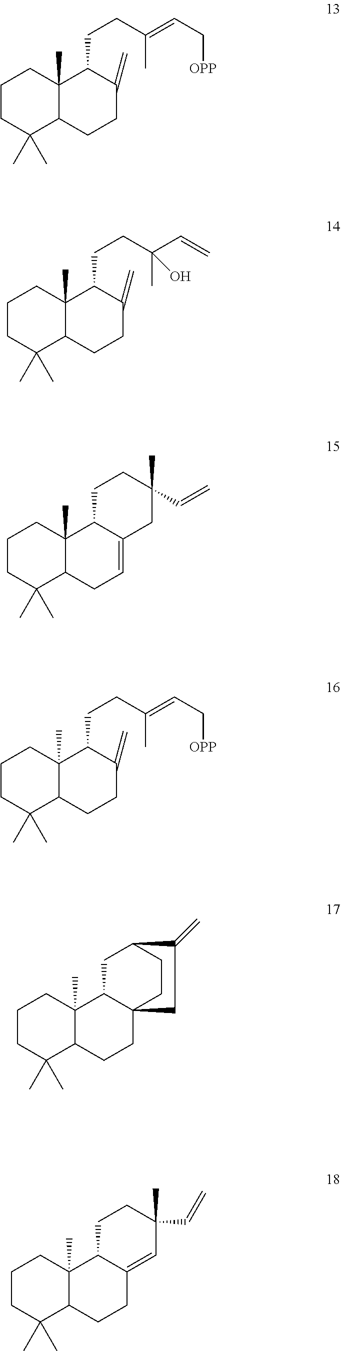

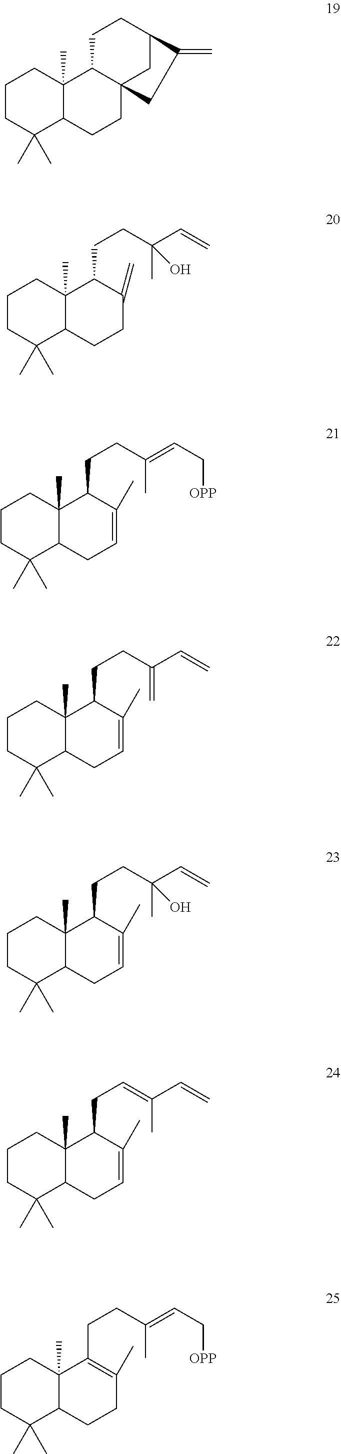

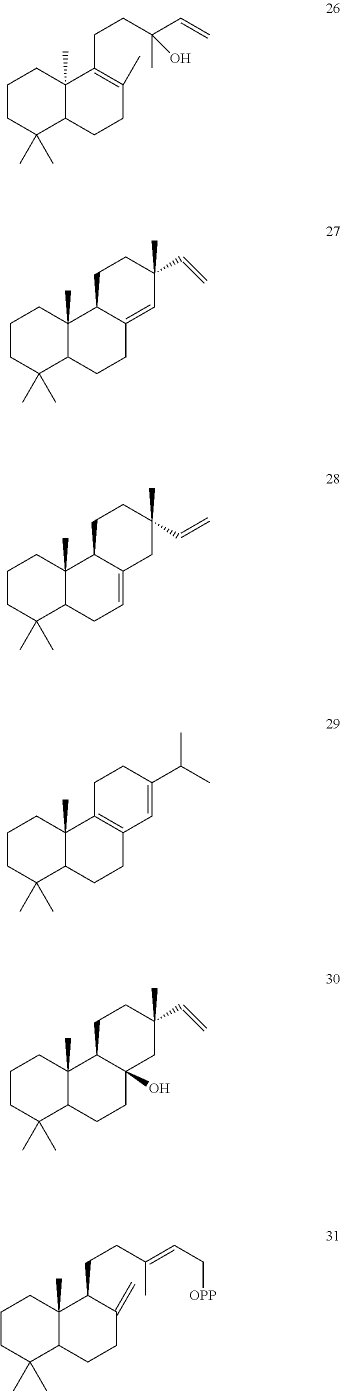

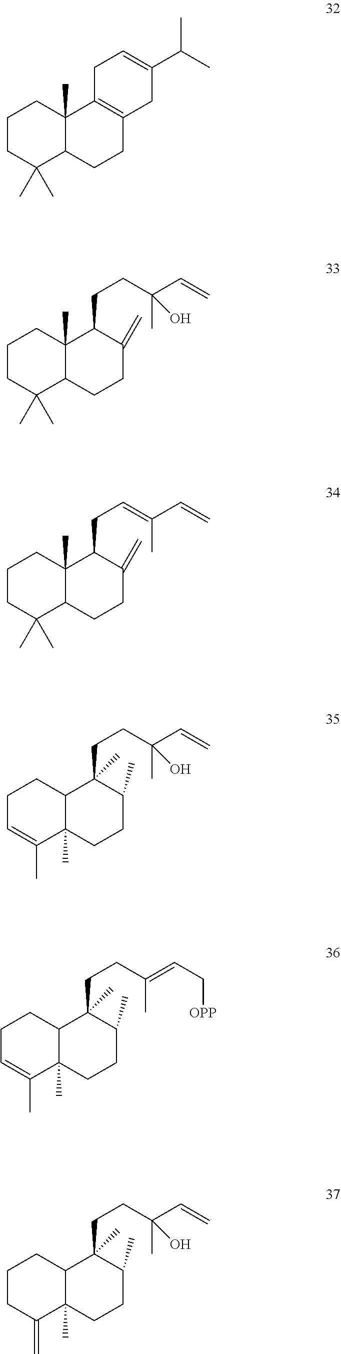

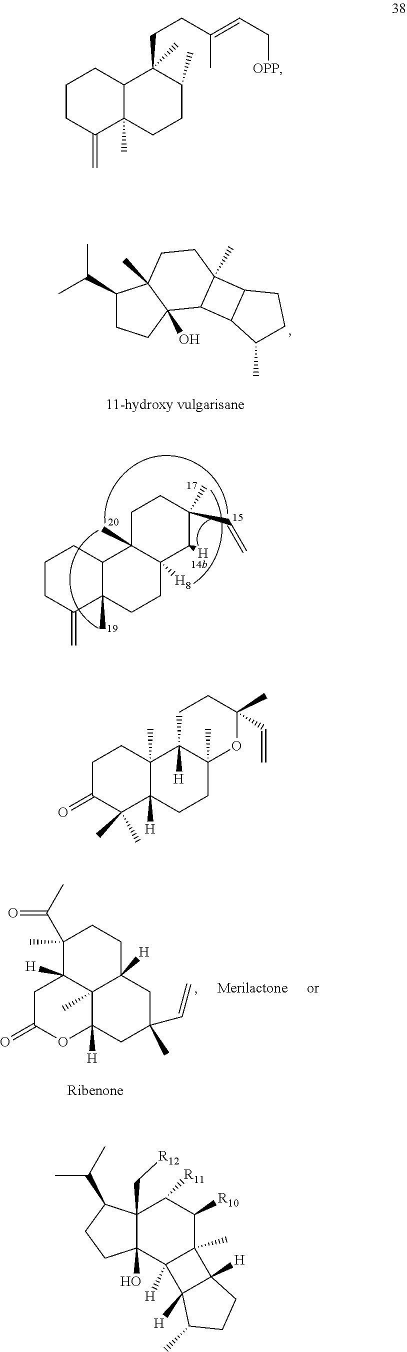

wherein:

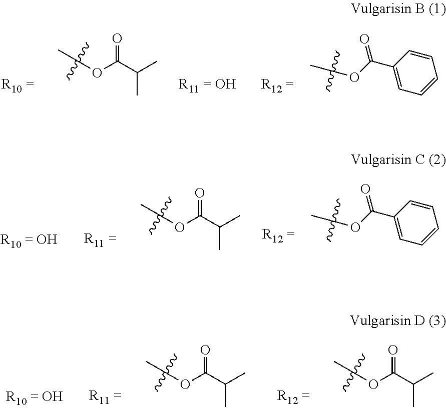

18. The method of statement 12-16 or 17, wherein the terpene is at least one of the following compounds:

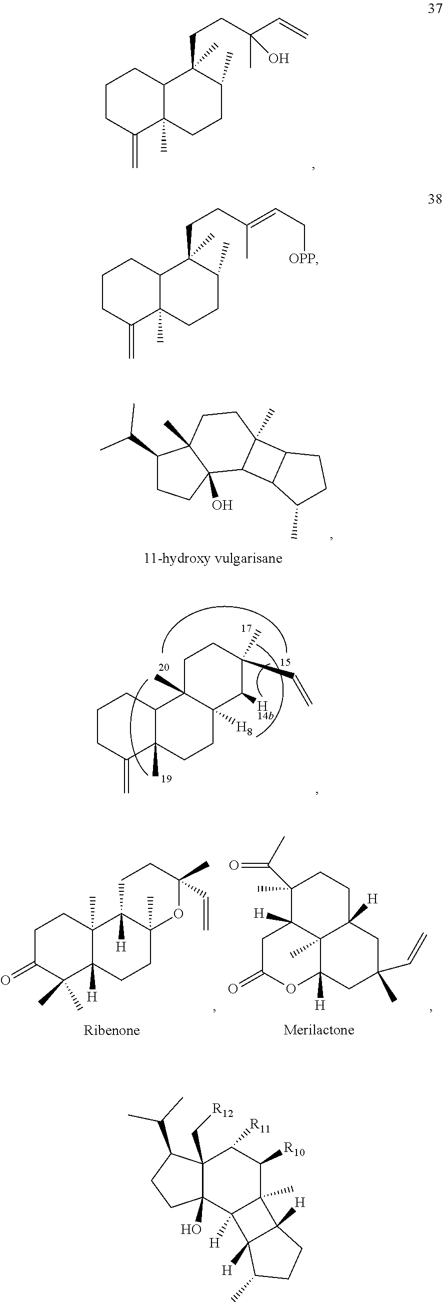

or
   wherein:

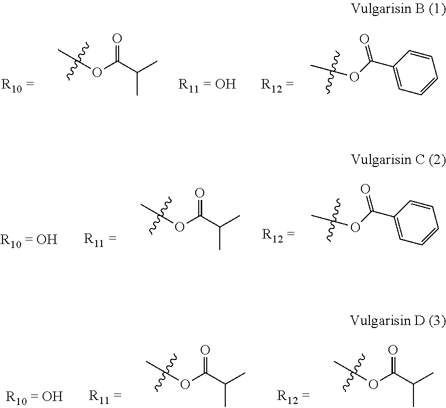

19. The method of statement 12-17 or 18 wherein the terpene precursor is geranylgeranyl diphosphate (GGPP).
20. A compound selected from:

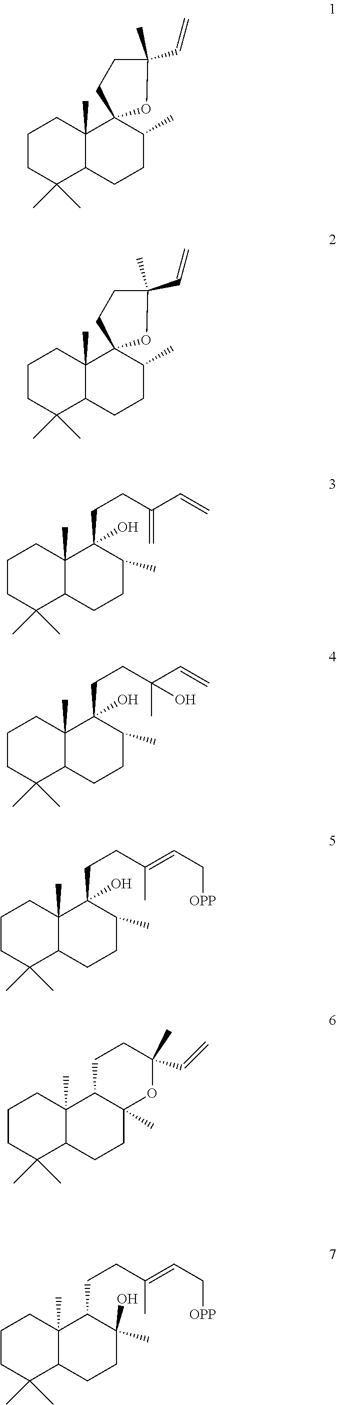

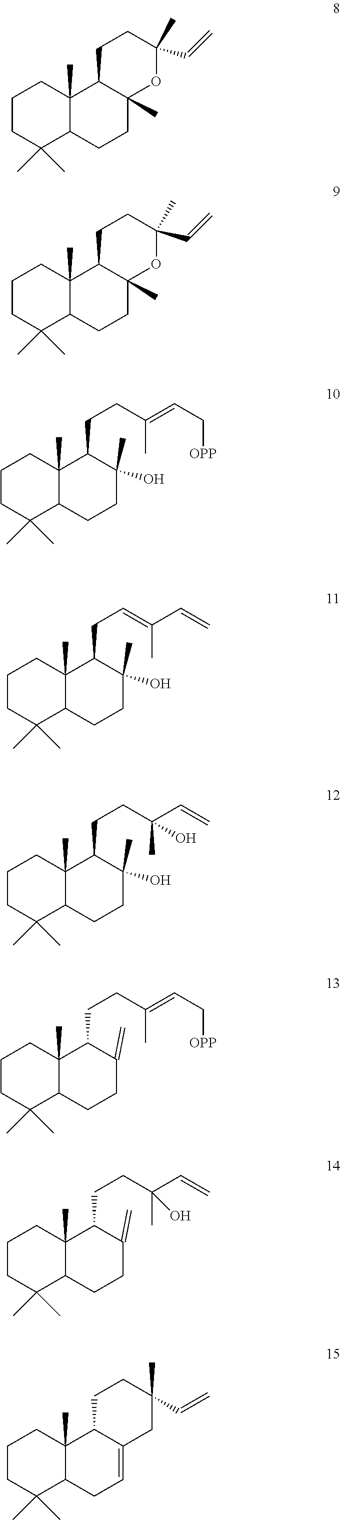

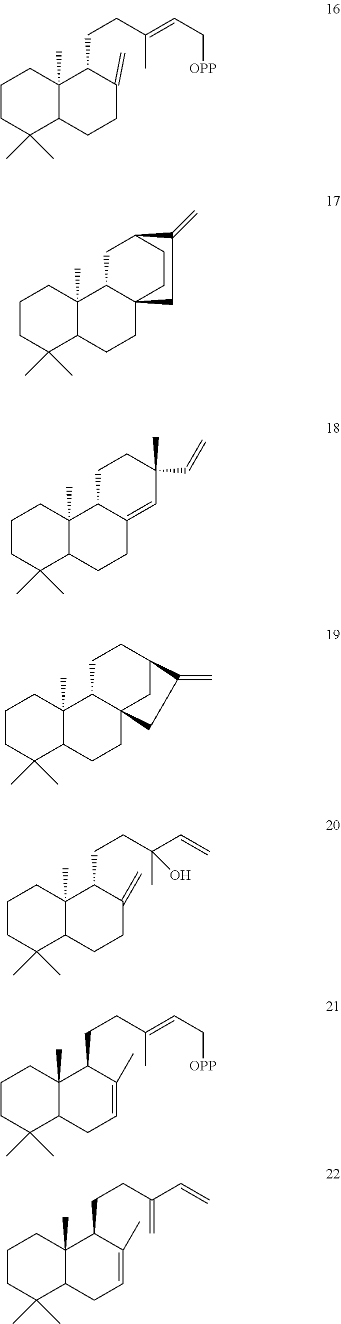

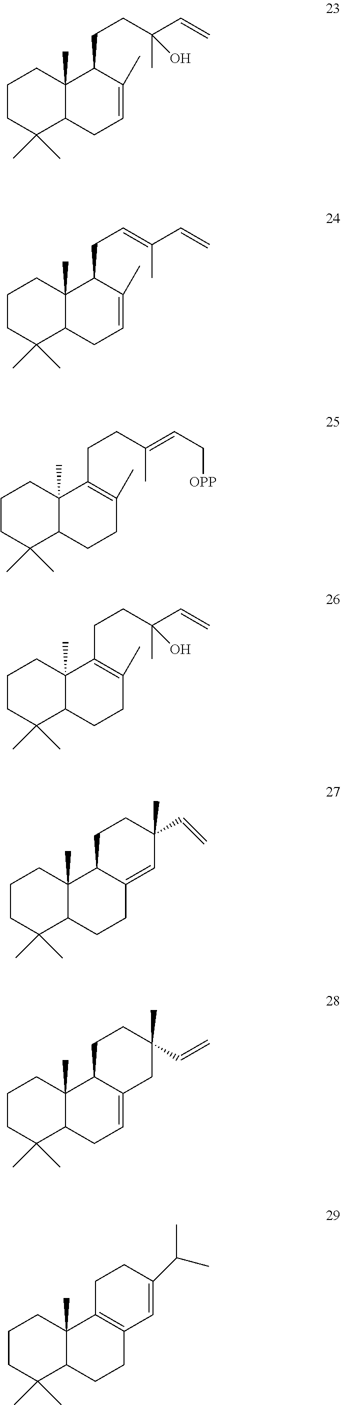

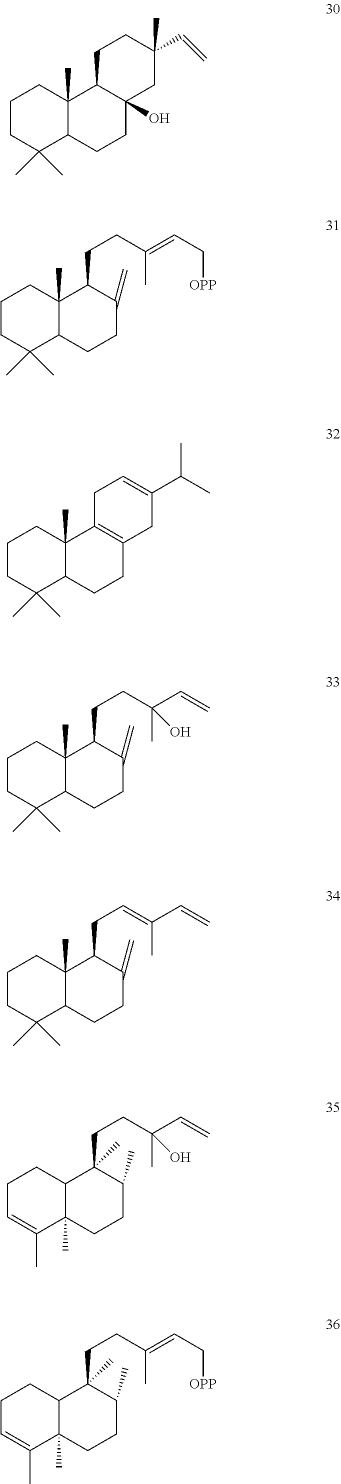

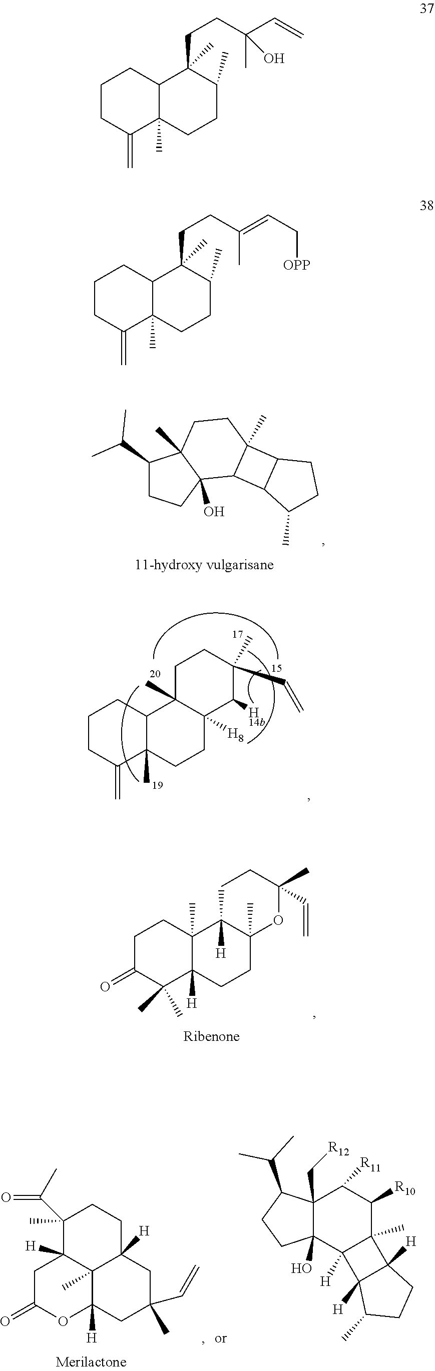

wherein:

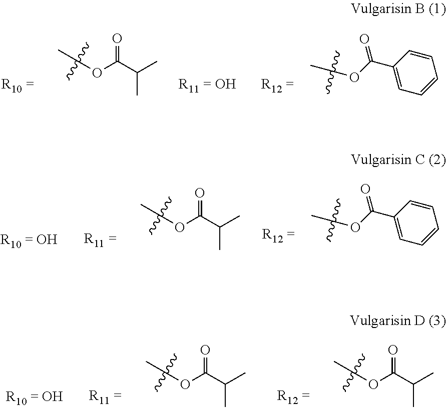

21. A reaction mixture comprising one or more of the following:

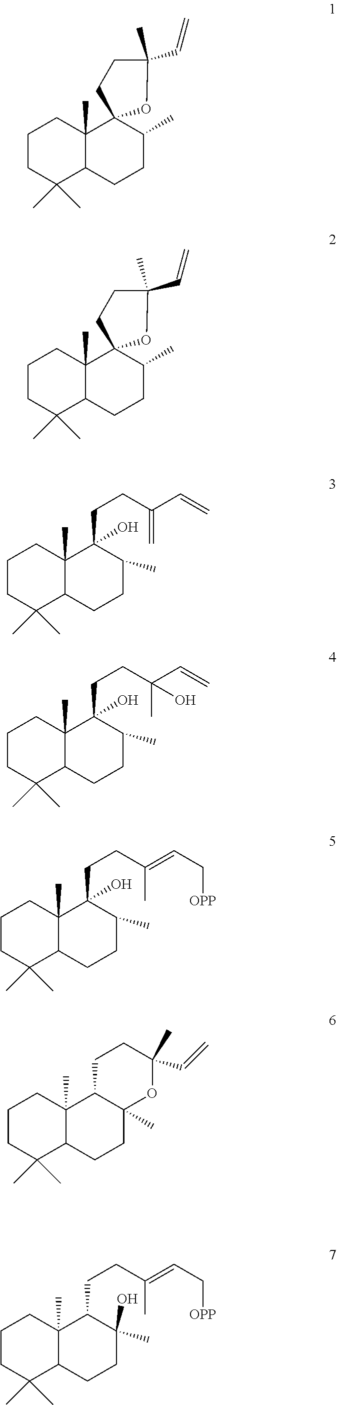

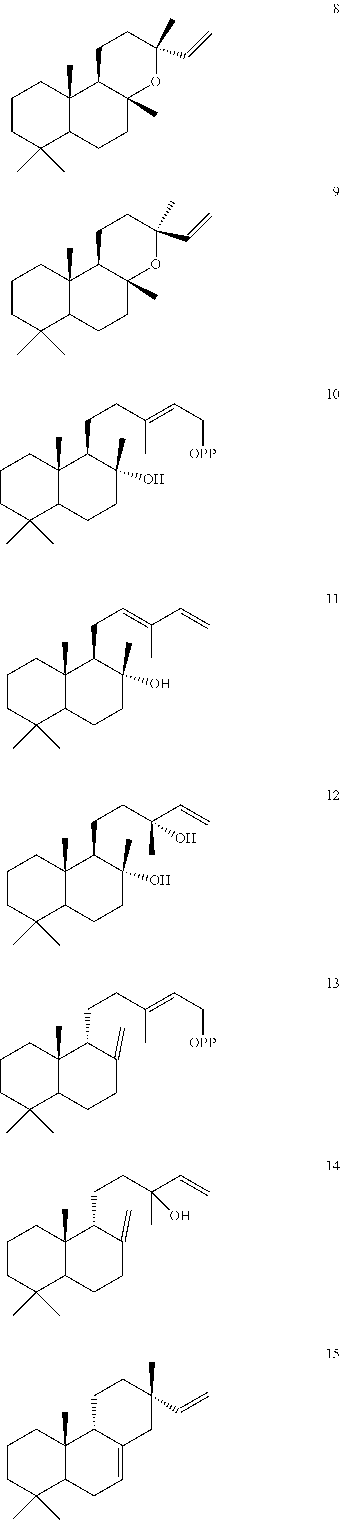

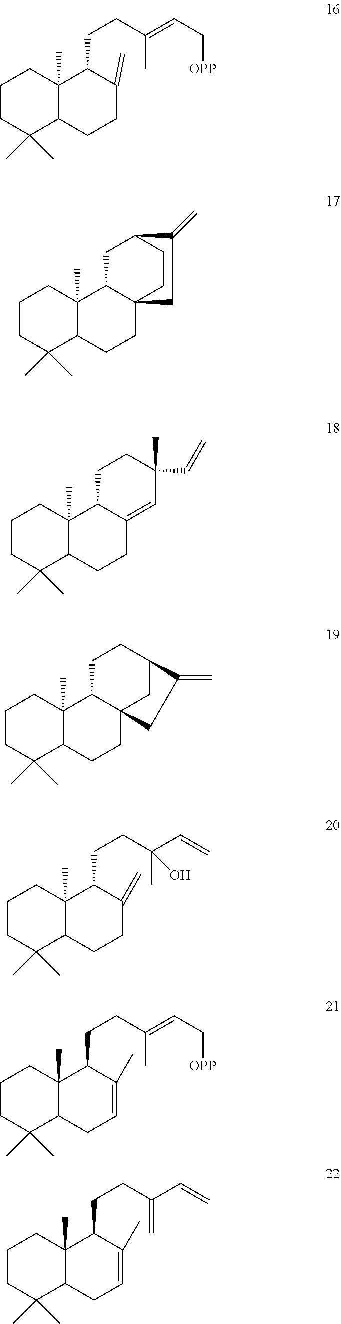

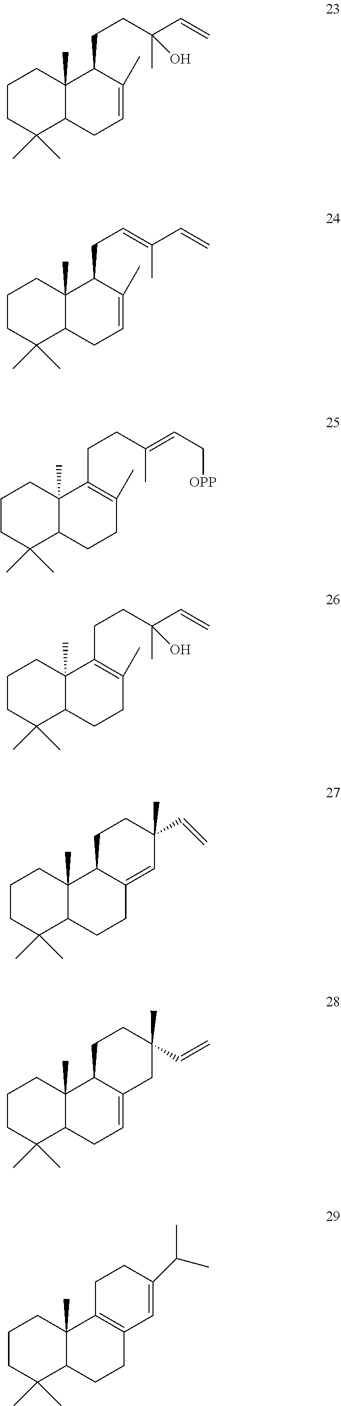

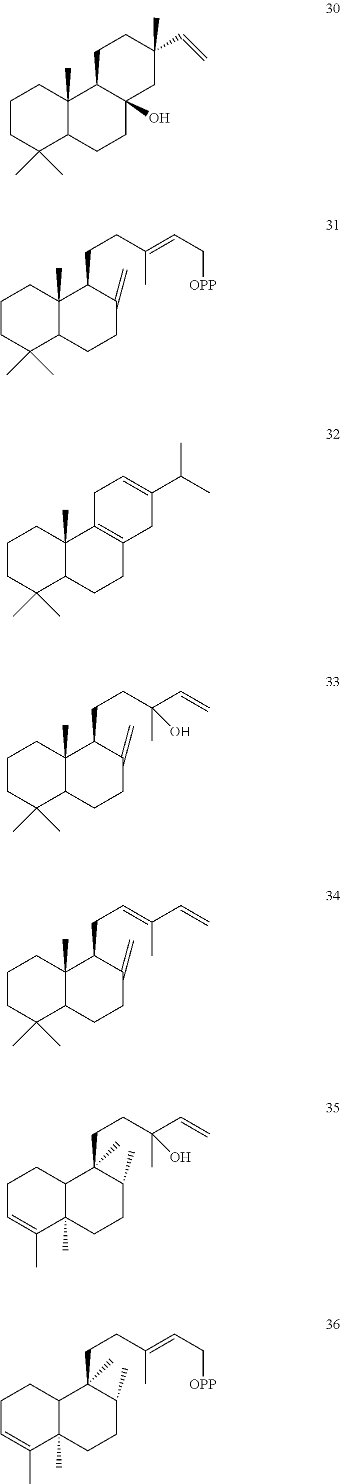

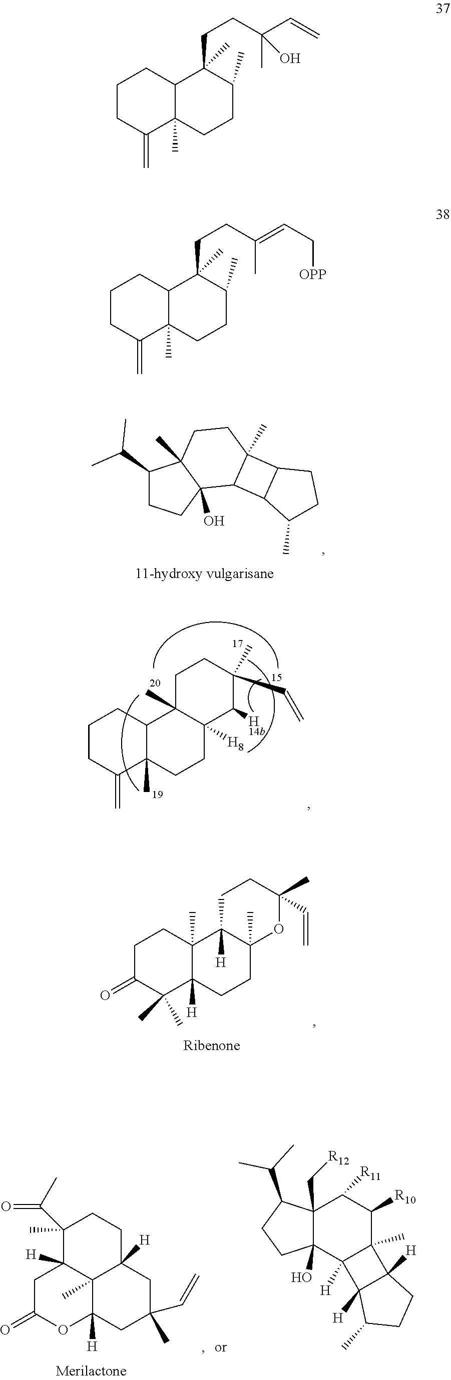

wherein:

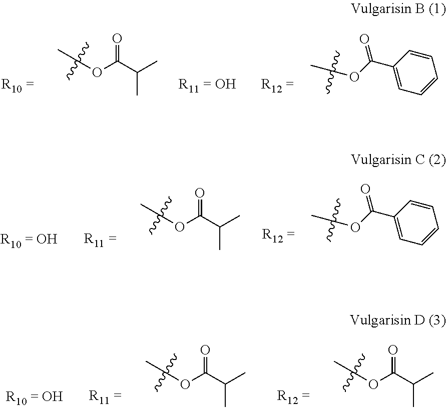

The specific methods, devices and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A method for synthesizing a terpene comprising incubating a terpene precursor with an enzyme with at least 95% sequence identity to the amino acid sequence of SEQ ID NO:1, 3, 5, 7, 9, 11, 13, 15, 17, 19, 21, 25, 27, 29, 31, 33, 35, 37, 39, 41, 43, 45, 47, 49, 51, 53, 57, 59, or 176.

2. The method of claim 1, wherein the precursor is isopentenyl diphosphate (IPP), dimethylallyl diphosphate (DMAPP), geranylgeranyl diphosphate (GGPP), or a combination thereof.

3. The method of claim 1, which comprises incubating a host cell that expresses a heterologous expression system comprising at least one expression cassette having a heterologous promoter operably linked to a nucleic acid segment encoding an enzyme with at least 95% sequence identity to an amino acid sequence of SEQ ID NO: 1, 3, 5, 7, 9, 11, 13, 15, 17, 19, 21, 25, 27, 29, 31, 33, 35, 37, 39, 41, 43, 45, 47, 49, 51, 53, 57, 59, or 176.

4. The method of claim 1, wherein the terpene is a compound of formula I, II, or III: 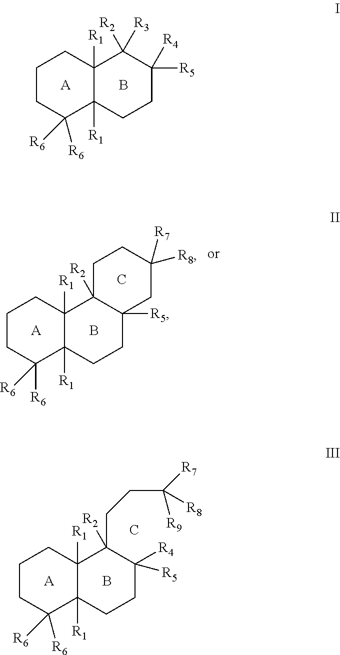
wherein
each R₁ can separately be hydrogen or lower alkyl;
R₂ can be hydrogen, lower alkyl, hydroxy, a bond to an adjacent ring carbon, or form a C₄-C₆ cycloheteroalkyl with R₃;
R₃ can be a branched C₅-C₆ alkyl with 0-2 double bonds, can form a C₄-C₆ cycloheteroalkyl with R₂; can form a cycloalkyl with R₄, or can form a cycloheteroalkyl ring with R₄, wherein the C₅-C₆ alkyl can optionally have one hydroxy, phosphate or diphosphate substituent, and wherein each cycloalkyl or cycloheteroalkyl ring can have 0-2 double bonds, and each cycloalkyl or cycloheteroalkyl ring can have 0-2 alkyl or 0-2 alkene substituents;
R₄ can be hydrogen, lower alkyl, lower alkene, hydroxy, a carbon bonded to R₉, an oxygen bonded to R₉, form a cycloalkyl ring with R₃, or form a cycloheteroalkyl ring with R₃, wherein each cycloalkyl ring or cycloheteroalkyl is ring can have 0-2 double bonds, and each cycloalkyl ring or cycloheteroalkyl ring can have 0-2 alkyl or 0-2 alkene substituents;
R₅ can be hydrogen, hydroxy, lower alkyl, a lower alkene, a bond with an adjacent carbon, form a cycloalkyl ring with a ring atom of a ring formed by R₃ and R₄, wherein the cycloalkyl ring can have 0-2 double bonds, and the cycloalkyl ring can have 0-2 alkyl or 0-2 alkene substituents;
each R₆ can separately be hydrogen, lower alkyl, lower alkene, or form a bond with an adjacent carbon;
R₇ can be lower alkyl, lower alkene, or form a cycloalkyl ring with a R₅,
R₈ can be lower alkyl, hydroxy, phosphate, diphosphate, or form a bond with an adjacent carbon; and
R₉ can be hydrogen, lower alkyl, lower alkene, ═CH₂, hydroxy, phosphate, diphosphate, form a bond with an adjacent carbon, form a cycloalkyl ring with R₄, or form a cycloheteroalkyl ring with R₄, wherein each cycloalkyl ring or cycloheteroalkyl ring can have 0-2 double bonds, and each cycloalkyl ring or cycloheteroalkyl ring can have 0-2 alkyl or 0-2 alkene substituents.

5. The method of claim 1, wherein the terpene is a compound with a skeleton selected from: 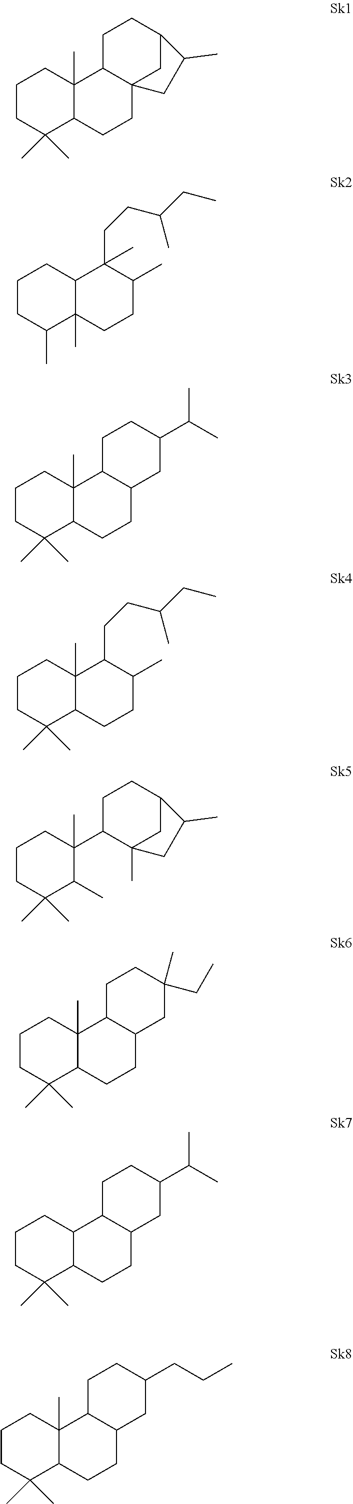 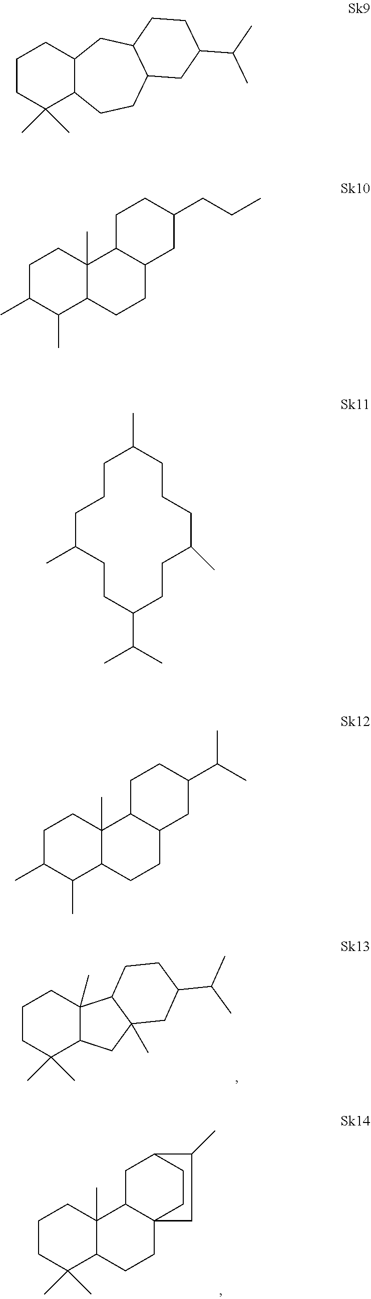 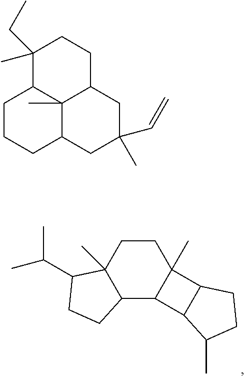
or combination thereof.

6. The method of claim 1, wherein the terpene is one or more of the following compounds: 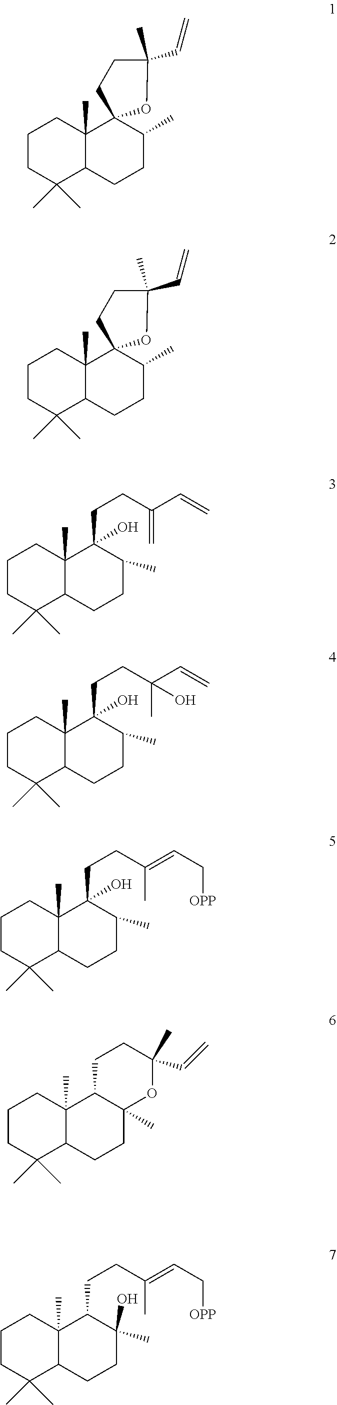 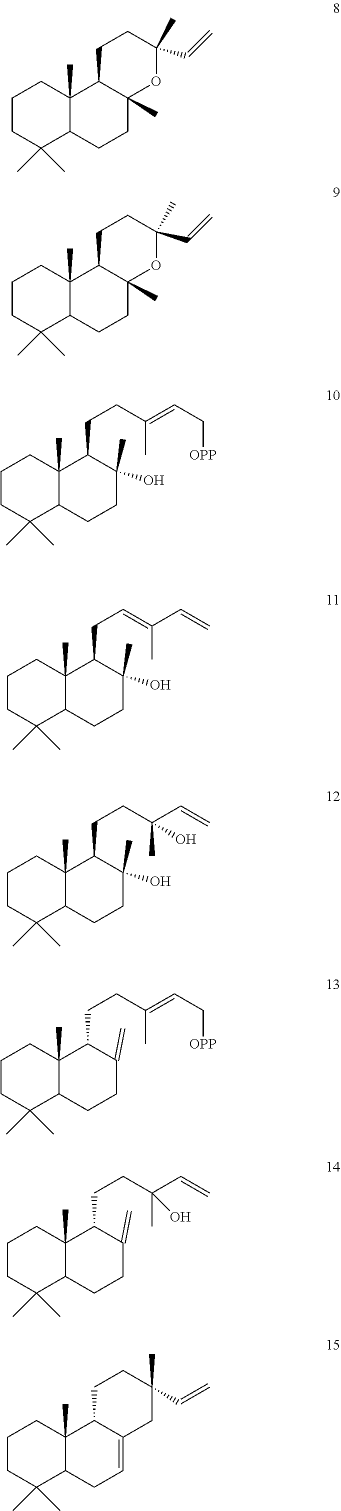 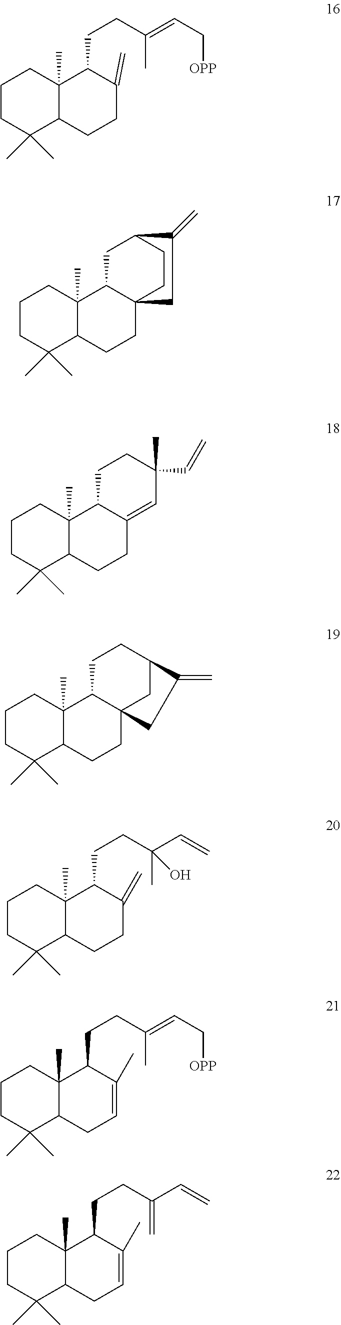 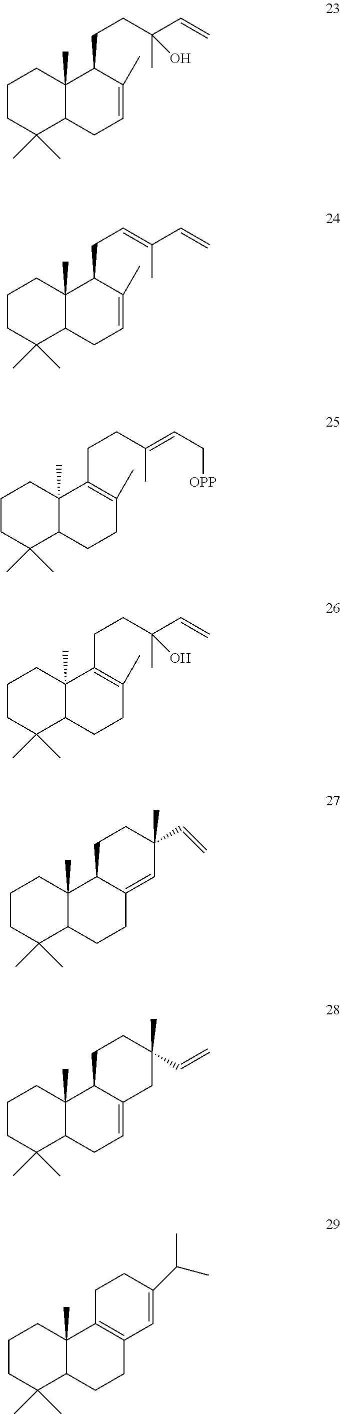 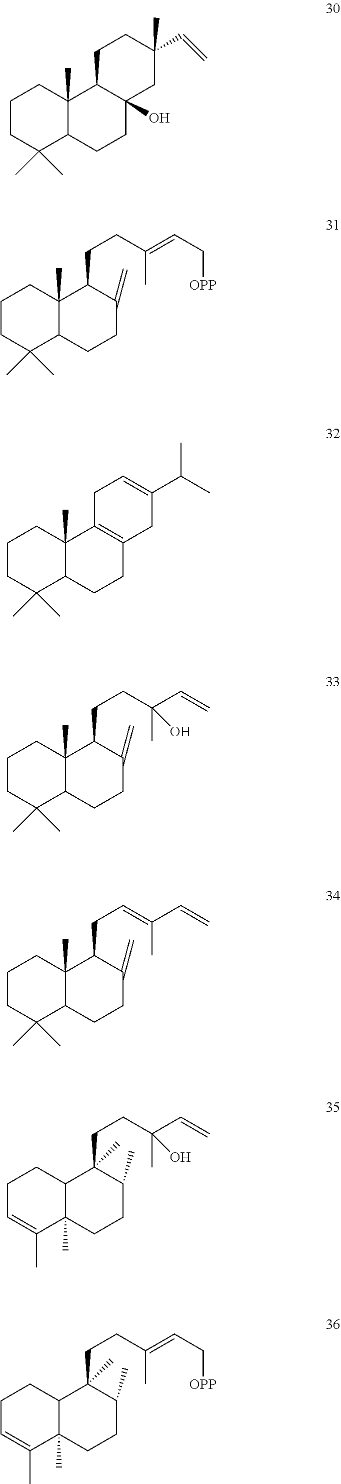 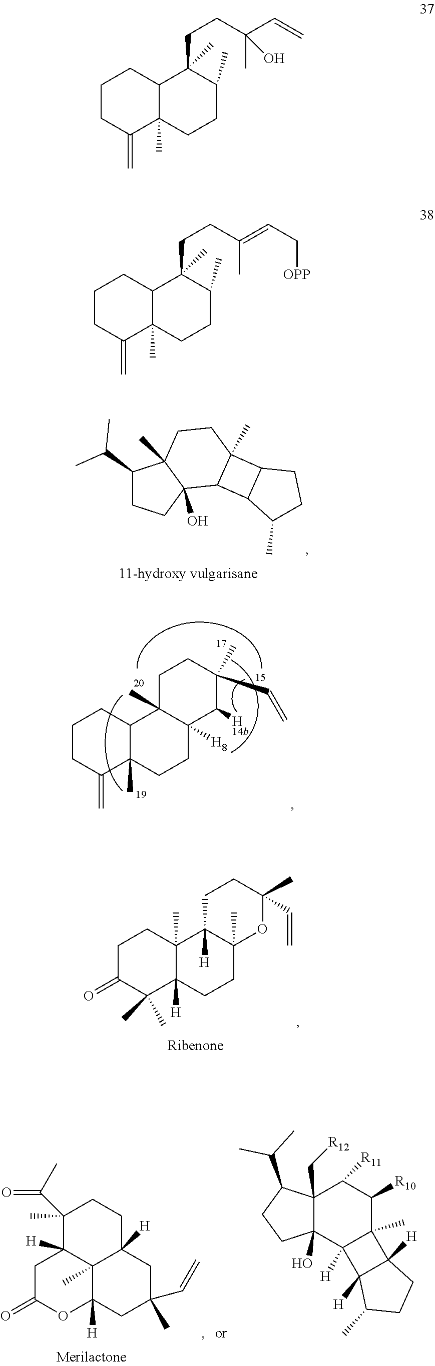
wherein:

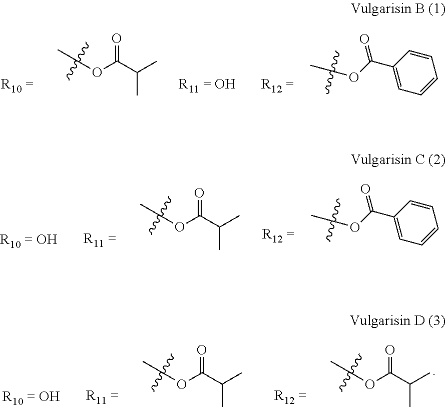

7. A method for synthesizing a terpene comprising incubating a terpene precursor of a terpene of formula I, II, or III, with an enzyme with at least 95% sequence identity to the amino acid sequence of SEQ ID NO:1, 3, 5, 7, 9, 11, 13, 15, 17, 19, 21, 25, 27, 29, 31, 33, 35, 37, 39, 41, 43, 45, 47, 49, 51, 53, 57, 59, or 176, wherein the terpene of formula I, II, or III is: 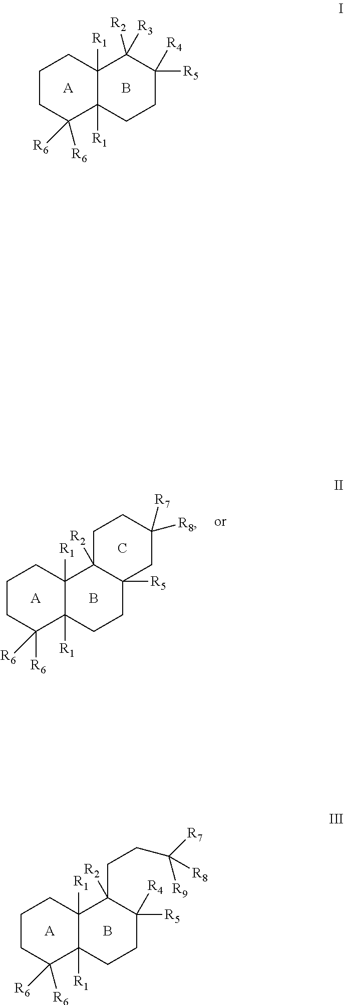
wherein
each R₁ can separately be hydrogen or lower alkyl;
R₂ can be hydrogen, lower alkyl, hydroxy, a bond to an adjacent ring carbon, or form a C₄-C₆ cycloheteroalkyl with R₃;
R₃ can be a branched C₅-C₆ alkyl with 0-2 double bonds, can form a C₄-C₆ cycloheteroalkyl with R₂; can form a cycloalkyl with R₄, or can form a cycloheteroalkyl ring with R₄, wherein the C₅-C₆ alkyl can optionally have one hydroxy, phosphate or diphosphate substituent, and wherein each cycloalkyl or cycloheteroalkyl ring can have 0-2 double bonds, and each cycloalkyl or cycloheteroalkyl ring can have 0-2 alkyl or 0-2 alkene substituents;
R₄ can be hydrogen, lower alkyl, lower alkene, hydroxy, a carbon bonded to R₉, an oxygen bonded to R₉, form a cycloalkyl ring with R₃, or form a cycloheteroalkyl ring with R₃, wherein each cycloalkyl ring or cycloheteroalkyl ring can have 0-2 double bonds, and each cycloalkyl ring or cycloheteroalkyl ring can have 0-2 alkyl or 0-2 alkene substituents;
R₅ can be hydrogen, hydroxy, lower alkyl, a lower alkene, a bond with an adjacent carbon, form a cycloalkyl ring with a ring atom of a ring formed by R₃ and R₄, wherein the cycloalkyl ring can have 0-2 double bonds, and the cycloalkyl ring can have 0-2 alkyl or 0-2 alkene substituents;
each R₆ can separately be hydrogen, lower alkyl, lower alkene, or form a bond with an adjacent carbon;
R₇ can be lower alkyl, lower alkene, or form a cycloalkyl ring with a R₅,
R₈ can be lower alkyl, hydroxy, phosphate, diphosphate, or form a bond with an adjacent carbon; and
R₉ can be hydrogen, lower alkyl, lower alkene, ═CH₂, hydroxy, phosphate, diphosphate, form a bond with an adjacent carbon, form a cycloalkyl ring with R₄, or form a cycloheteroalkyl ring with R₄, wherein each cycloalkyl ring or cycloheteroalkyl ring can have 0-2 double bonds, and each cycloalkyl ring or cycloheteroalkyl ring can have 0-2 alkyl or 0-2 alkene substituents.

8. A method for synthesizing a terpene comprising incubating a terpene precursor with an enzyme with at least 95% sequence identity to the amino acid sequence of SEQ ID NO:1, 3, 5, 7, 9, 11, 13, 15, 17, 19, 21, 25, 27, 29, 31, 33, 35, 37, 39, 41, 43, 45, 47, 49, 51, 53, 57, 59, or 176, wherein the terpene precursor comprises a diphosphate.
